(19) United States
(12) Patent Application Publication
(10) Pub. No.: US 2025/0101025 A1
(43) Pub. Date: Mar. 27, 2025

(54) NOVEL PLK1 DEGRADATION INDUCING COMPOUND

(71) Applicant: UPPTHERA, INC., Incheon (KR)

(72) Inventors: Soo Hee Ryu, Incheon (KR); Im Suk Min, Gyeonggi-do (KR); Han Kyu Lee, Gyeonggi-do (KR); Seong Hoon Kim, Incheon (KR); Hye Guk Ryu, Incheon (KR); Keum Young Kang, Incheon (KR); Sang Youn Kim, Incheon (KR); So Hyun Chung, Incheon (KR); Jun Kyu Lee, Gyeonggi-do (KR); Gibbeum Lee, Gyeonggi-do (KR)

(73) Assignee: UPPTHERA, INC., Incheon (KR)

(21) Appl. No.: 18/580,506

(22) PCT Filed: Aug. 10, 2022
(86) PCT No.: PCT/KR2022/011962

§ 371 Date: Jan. 18, 2024

(30) Foreign Application Priority Data
Aug. 10, 2021 (KR) 10-2021-0105358
Aug. 12, 2021 (KR) 10-2021-0106488
Sep. 3, 2021 (KR) 10-2021-0117389
Sep. 24, 2021 (KR) 10-2021-0126757
Jan. 20, 2022 (KR) 10-2022-0008456
Feb. 17, 2022 (KR) 10-2022-0020996
May 3, 2022 (KR) 10-2022-0054880
Jun. 21, 2022 (KR) 10-2022-0075838

(51) Int. Cl. C07D 487/04; A61K 31/551; C07D 519/00

(52) U.S. Cl. CPC C07D 487/04; A61K 31/551; C07D 519/00

(57) ABSTRACT

The present disclosure relates to a novel PLK1 degradation inducing compound, a method for preparing the same, and the use thereof. The compounds of the present disclosure exhibit an effect of inducing PLK1 degradation. Therefore, the compounds of the present disclosure may be effectively utilized for preventing or treating PLK1-related diseases.

### TECHNICAL FIELD

The present disclosure relates to a novel PLK1 degradation inducing compound, a method for preparing the same, and the use thereof. It can specifically act on abnormal cells, etc. and can be usefully used in the treatment of various diseases through efficient degradation of PLK1.

### BACKGROUND ART

Polo-like kinase 1 (PLK1) is a serine/threonine kinase involved in the conversion of G2/M phase during cell growth and division. PLK1 is expressed and activated in a pulse form from the S phase to the G2/M phase, and rapidly degrades as mitosis ends.

PLK1 is overexpressed in various carcinomas such as colon cancer, lung cancer, bladder cancer, and melanoma, etc., and cancer cells overexpressing PLK1 tend to show resistance to various types of anticancer drugs. As the PLK1 dependence in various carcinomas was revealed as described above, there have been attempts to develop PLK1 inhibitor compounds such as volasertib (also known as BI6727), etc.

However, the conventional PLK1 inhibitors do not sufficiently inhibit PLK1 activity at concentrations that are clinically safe. Thus, there is a problem that even if the cell cycle of cancer cells is temporarily delayed, some cancer cells eventually restart the cell cycle, which may not obtain sufficient clinical effects (see Gheghiani et al., Cell Reports, 2017, etc.). In fact, many pharmaceutical companies such as Boehringer Ingelheim, GlaxoSmithKline, etc., have attempted to develop small-molecular compound-based PLK1 inhibitors, but most of them have failed or stopped in the clinical trial stage, and thus there are no commercially available PLK1 inhibitors to date. It shows that pharmacological mechanism that follows the method of inhibiting enzyme activity by binding to the active site of PLK1 like the small molecule compound inhibitors is not sufficiently effective in the development of new drugs intended to derive anticancer effects by inhibiting PLK1 activity of cancer cells.

Recently, a proteolysis targeting chimera (PROTAC) has been proposed as a small molecule-based platform technology capable of inducing proteolysis of a target protein in the body. The PROTAC is a bifunctional compound in which a ligand molecule that binds to disease-related target protein and an E3 ubiquitin ligase binding moiety are linked by a chemical linker. Theoretically, the PROTAC compound is capable of inducing degradation of the target protein by placing the disease-related target protein near the E3 ubiquitin ligase. Based on this new mechanism different from the existing inhibitors, a lot of PROTAC compounds have been developed as therapeutic agents for cancer and inflammatory diseases, etc., and being studied with various extensibility (e.g. as payloads of ADC (Antibody-Drug Conjugates)). However, it does not show activity in all ranges of binding moieties or linkers, and in order for PROTAC to exhibit the desired level of efficacy, it is known through several studies that each binding moiety and linker must have an appropriately linked structure (see US2020-0325130A). In particular, in the case of the CRBN (Cereblon) E3 ligase targeting moiety, depending on the type of the binding moiety or the structure of the compound linked thereto, there is a risk of degrading CRBN neo-substrate (GSPT1, IKZF1/3, etc.) or showing off-target toxicity accordingly. Therefore, it is important to select appropriate binding moieties and optimize the structure of the entire compound so as not to exhibit unexpected toxicity during PROTAC drug development.

In the case of the PROTAC compound using PLK1 as a target protein, Chinese Patent Laid-Open No. 106543185 A discloses some bifunctional compounds in which a volasertib derivative compound and a binding moiety for the E3 ubiquitin ligase CRBN are linked by a chemical linker. However, the related art document merely describes some limited forms of synthesis examples of PROTAC compounds, wherein in general, the target degradation activity and selectivity of PROTAC may vary significantly depending on selection of the target protein moiety, the E3 ubiquitin ligase binding moiety, and the like (see Burslem and Crews, 2017, etc.).

Further, the PROTAC compound described in the above-described document is characterized by a compound that simultaneously degrades PLK1 and BRD4, and degrade various proteins such as other PLK family proteins and BRD4, etc.), which may cause side effects due to off-target toxicities at the time of drug development. In particular, it is known that strong inhibition of BRD4 activity inevitably accompanies on-target toxicity such as blood toxicity and gastrointestinal toxicity along with pharmacological effects. Therefore, the PROTAC compound described in the above document would expect to face greater clinical side effects as more BRD4 protein gets degraded (see Bolden et al. Cell Reports, 2014).

Moreover, according to the document published by the inventors of the above document (see Mu et al. BBRC, 2019), it can be confirmed that the PROTAC compound, which simultaneously degrades PLK1 and BRD4, has much stronger BRD4 degradation ability than PLK1 degradation ability at the cellular level, and the cell cycle thereof almost stops in the G1 phase, etc., that is, the PROTAC compound actually acts only as a BRD4 inhibitor regardless of the way that the conventional PLK1 inhibitors exert pharmacological effects.

Therefore, there is an unsatisfied demand for effective PLK1 degradation inducing compound with no or minimal side effects. (e.g. off-target toxicity)

### DISCLOSURE OF INVENTION

### Technical Problem

An object of the present disclosure is to provide novel PLK1 degradation inducing compounds.

Another object of the present disclosure is to provide a method for preparing the compounds.

Still another object of the present disclosure is to provide a use of the compounds.

### Solution to Problem

In order to achieve the above-described objects, the present inventors made efforts to study, and as a result, found that novel PROTAC compounds of the present invention specifically act on abnormal cells overexpressing PLK1 through appropriate structural combination and optimization of E3 Ligase binder, Target binding moiety, and Linker to induce effective PLK1 degradation and minimize side effects, and completed the present invention.

### Selective PLK1 Degradation Inducing Compounds

The present disclosure provides novel compounds that induce effective polo-like kinase 1 (PLK1) degradation. Specifically, the present disclosure provides a bifunctional compound in which a PLK1 binding moiety and an E3 ubiquitin ligase-binding moiety are linked by a chemical linker.

In one general aspect, there is provided a compound represented by the following Formula I, a steroisomer thereof or a pharmaceutically acceptable salt thereof:

ULM-Linker-PTM [Formula I]
in the Formula I above,
ULM is a moiety represented by the following Formula 1;

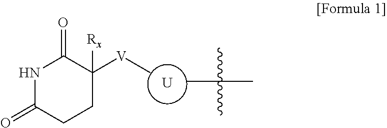

PTM is a moiety represented by the following Formula 2;

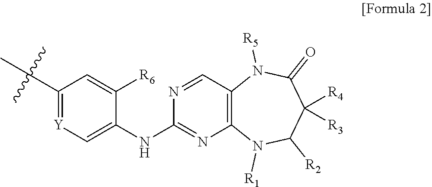

Linker is a group that chemically links ULM and PTM;
Rx is —H or —C₁₋₄alkyl;
V is —NH—C(═O)—, —(CH₂)v-NH—, —(CH₂)v-N—C₁₋₄alkyl-, —O—, —C(═O)— or —C(═NH)— {wherein the N atom of —(CH₂)v-NH— in the V may be linked with the Rx to form a 5- to 6-membered ring, and the v is 0, 1, 2, 3 or 4};
ring U is phenyl, pyridinyl or pyrimidinyl {wherein at least one H of the phenyl, pyridinyl or pyrimidinyl ring may be substituted with R_{U}};
R_{U} is —C₁₋₄alkyl, —C₁₋₄hydroxyalkyl, —C₁₋₄aminoalkyl, —C₁₋₄haloalkyl, —C₁₋₄alkoxy, —NH₂, —OH or -halo {wherein the R_{U} may be linked with the N atom of —(CH₂)v-NH— in the V to form 5- to 6-membered ring [wherein at least one H of the 5- to 6-membered ring may be substituted with —C₁₋₄alkyl or ═O], and the R_{U} may be linked with the N atom of —C(═NH)— in the V to form 5- to 6-membered ring [wherein at least one H of the 5- to 6-membered ring may be substituted with —C₁₋₄alkyl]};
Y is CR₇;
R₁ is —C₁₋₄alkyl or 3- to 7-membered cycloalkyl;
R₂ is —H;
R₃ and R₄ are each independently —H, —C₁₋₄alkyl or -halo;
R₅ is —C₁₋₄alkyl;
R₆ is —C₁₋₄alkyl or —C₁₋₄alkoxy; and
R₇ is —H or -halo.

In one embodiment of the present disclosure,
ULM is a moiety represented by following Formula 1-1 or Formula 1-2;

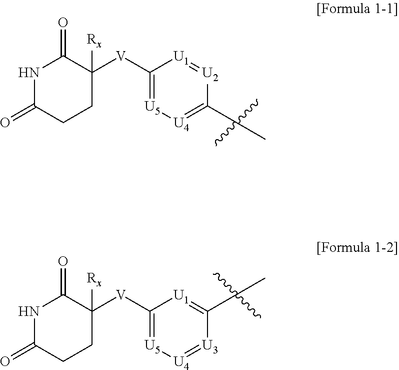

Rx is —H or —C₁₋₄alkyl;
V is —NH—C(═O)—, —(CH₂)v-NH—, —(CH₂)v-N—C₁₋₄alkyl-, —O— or —C(═NH)— {wherein the N atom of —(CH₂)v-NH— in the V may be linked with the Rx to form a 5- to 6-membered ring, and the v is 0, 1 or 2};
U₁ to U₅ are each independently CR_{U} or N {wherein R_{U} of the U₁ may be linked with the N atom of —(CH₂)v-NH— in the V to form a 5- to 6-membered ring [wherein at least one H of the 5- to 6-membered ring may be substituted with —C₁₋₄alkyl or ═O], and R_{U} of the U₁ may be linked with the N atom of —C(═NH)— in the V to form a 5- to 6-membered ring [wherein at least one H of the 5- to 6-membered ring may be substituted with —C₁₋₄alkyl]}; and
R_{U} is —C₁₋₄alkyl, —C₁₋₄haloalkyl, —NH₂ or -halo.

In one embodiment of the present disclosure,
ULM is

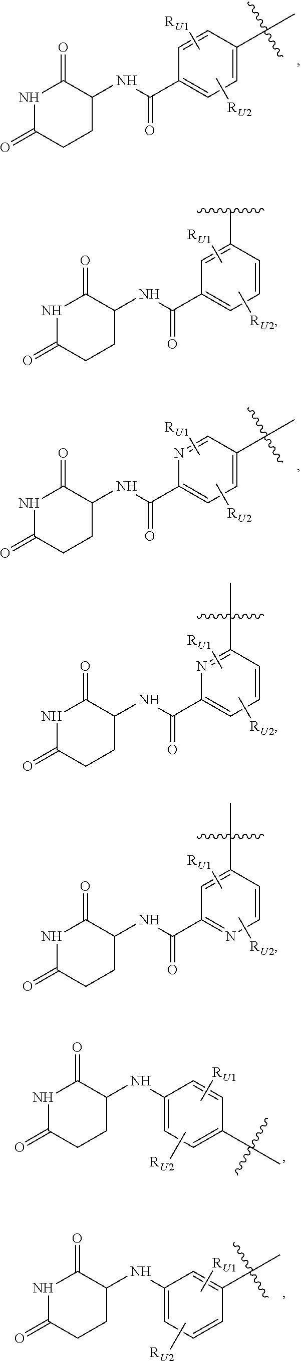

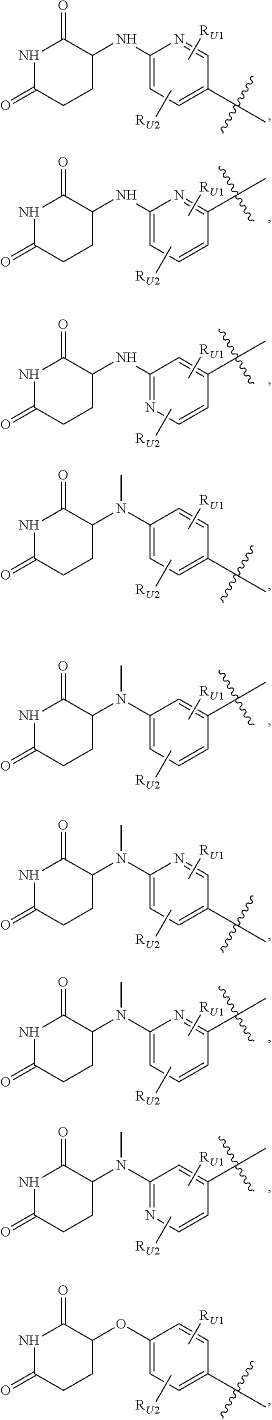

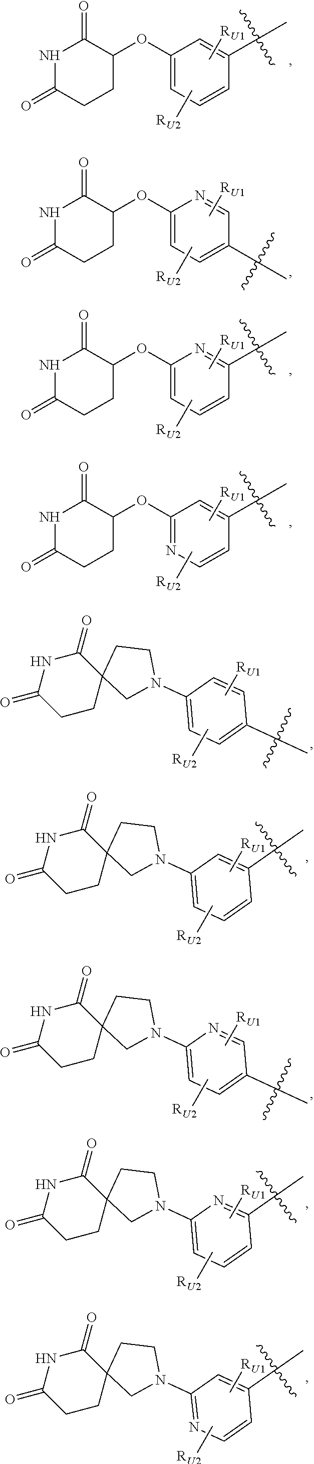

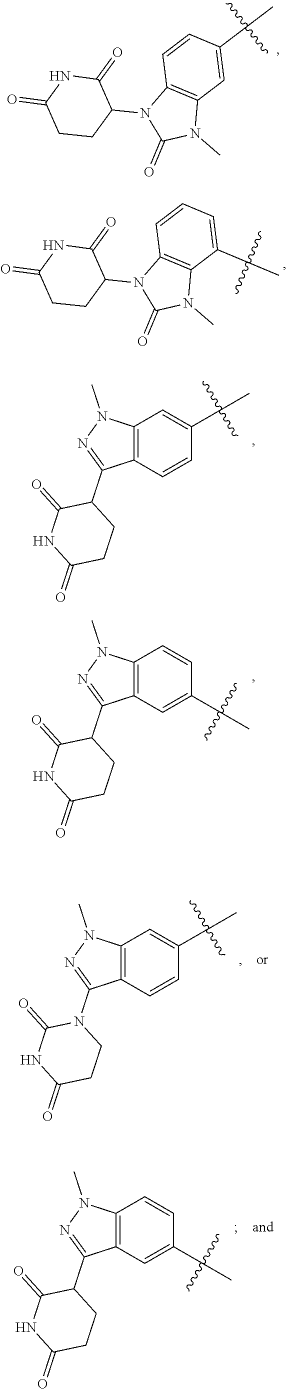

R_{U1} and R_{U2} are each independently —C₁₋₄alkyl, —C₁₋₄haloalkyl or -halo.

In one embodiment of the present disclosure,
ULM is

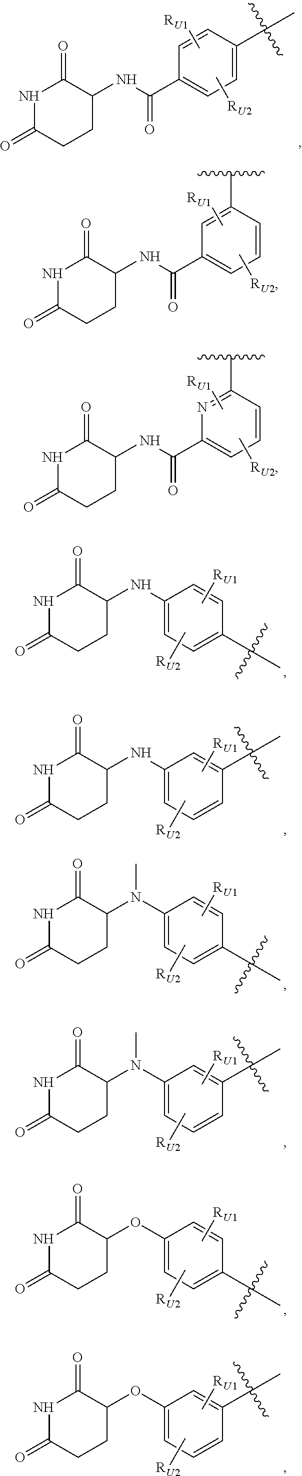

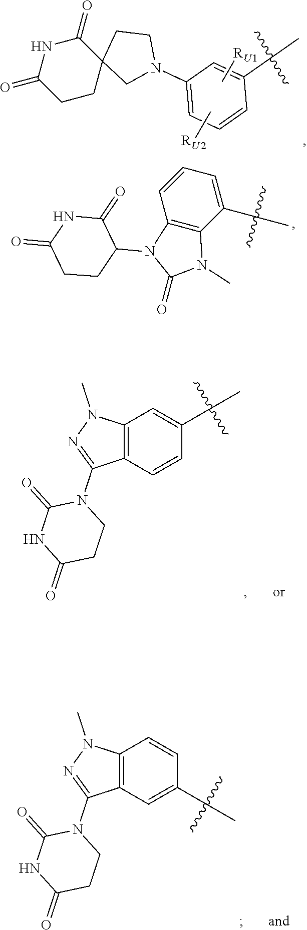

R_{U1} and R_{U2} are each independently —C₁₋₄haloalkyl or -halo.

In one embodiment,
PTM is

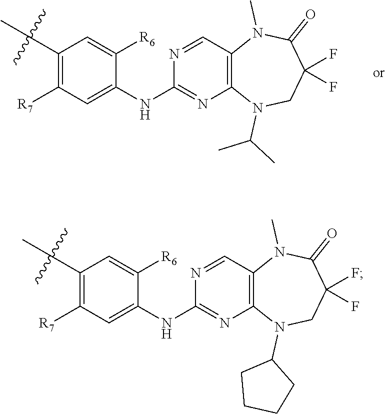

R₆ is —C₁₋₄alkoxy; and
R₇ is —H or -halo.

In one embodiment of the present disclosure,
Linker is -L_{U}-L₁-L₂-L₃-L_{P}-;
L_{U} is —(CH₂)x-, —(CH₂)x-NH—, —(CH₂)x-O—, —C(═O)—, phenyl or nothing (null) {wherein L_{U} is linked with ULM [wherein, when the L_{U} is nothing (null), L₁ is directly linked with ULM], and the x is 0, 1, 2, 3 or 4};
L₁ is heterocycloalkyl or nothing (null) {wherein, when the L₁ is nothing (null), L_{U} and L₂ are directly linked, the heterocycloalkyl contains at least one N atom in the ring, and at least one H of the heterocycloalkyl ring may be substituted with —C₁₋₄alkyl, —C₁₋₄haloalkyl, —C₁₋₄alkoxy, —OH, -halo or ═O};
L₂ is —(CH₂)y₁-, -(CD₂)y₁-, —(CH₂)y₂-C(═O)—(CH₂)y₃-, —(CH₂)y₂-NH—(CH₂)y₃- or —(H)y₂-N(C₁₋₄alkyl)-(CH₂)y₃-{wherein the y₁ to y₃ are each independently 0, 1, 2, 3, 4, 5 or 6};
L₃ is cycloalkyl, heterocycloalkyl or nothing (null) {wherein, when the L₃ is nothing (null), L₂ and Lₚ are directly linked, the heterocycloalkyl contains at least one N atom in the ring, and at least one H of the cycloalkyl or heterocycloalkyl ring may be substituted with —C₁₋₄alkyl, —C₁₋₄haloalkyl or -halo}; and

L_{P} is —(CH₂)p-NH—C(═O)— or —(CH₂)p-O— {wherein —(C═O)— or —O— of the L_{P} is linked with PTM, and p is 0, 1 or 2}.

In one embodiment of the present disclosure,
L_{U} is —(CH₂)x- or —(CH₂)x-NH— {wherein L_{U} is linked with ULM, and the x is 0 or 1};
L₁ is 4- to 12-membered heterocycloalkyl or nothing (null) {wherein, when the L₁ is nothing (null), L_{U} and L₂ are directly linked, the 4- to 12-membered heterocycloalkyl is single ring, bridged bicyclic ring or spiro ring, the 4- to 12-membered heterocycloalkyl contains at least one N atom in the ring, the N atom is directly linked with L_{U} or ULM, and at least one H of the 4- to 12-membered heterocycloalkyl ring may be substituted with —C₁₋₄alkyl, —OH or -halo};
L₂ is —(CH₂)y₁-, —(CH₂)y₂-C(═O)—(CH₂)y₃-, —(CH₂)y₂-NH—(CH₂)y₃- or —(CH₂)y₂-N(C₁ 4 alkyl)-(CH₂)y₃-{wherein the y₁ to y₃ are each independently 0, 1, 2 or 3};
L₃ is 4- to 6-membered cycloalkyl or 4- to 12-membered heterocycloalkyl {wherein the 4- to 12-membered heterocycloalkyl is single ring, bridged bicyclic ring or spiro ring, the 4- to 12-membered heterocycloalkyl contains at least one N atom in the ring, and at least one H of the 4- to 6-membered cycloalkyl or 4- to 12-membered heterocycloalkyl ring may be substituted with —C₁₋₄alkyl, —C₁₋₄haloalkyl or -halo}; and
L_{P} is —(CH₂)p-NH—C(═O)— {wherein —(C═O)— of the L_{P} is linked with PTM, and p is 0 or 1}.

In a certain embodiment of the present disclosure, the compound represented by Formula I is a compound that is selected from the group consisting of Compound 1 to 44.

In the present disclosure, a pharmaceutically acceptable salt refers to any organic or inorganic acid addition salt with a concentration that is relatively non-toxic, is harmless, and has effective action to patients, wherein side effects caused by this salt does not deteriorate beneficial efficacy of the compound represented by Formula I. For example, the pharmaceutically acceptable salt may be an inorganic acid such as hydrochloric acid, phosphoric acid, sulfuric acid, nitric acid, or the like, or an organic acid such as methanesulfonic acid, p-toluenesulfonic acid, acetic acid, trifluoroacetic acid, maleic acid, succinic acid, oxalic acid, benzoic acid, tartaric acid, fumaric acid, manderic acid, propionic acid, citric acid, lactic acid, glycolic acid, gluconic acid, galacturonic acid, glutamic acid, glutaric acid, glucuronic acid, aspartic acid, ascorbic acid, carbonic acid, vanillic acid or hydroiodic acid, but is not limited thereto.

### Use of the Selective PLK1 Degradation Inducing Compounds

An embodiment of the present disclosure is a composition for inducing PLK1 degradation including a compound represented by Formula I or a pharmaceutically acceptable salt thereof. The Formula I is the same as defined above.

In the experimental examples of the present disclosure, it was confirmed that the compounds of the present disclosure effectively induce the protein degradation of PLK1.

The PLK1 degradation-inducing PROTAC compound of the present disclosure is capable of fundamentally degrading the target protein, PLK1 in view of the mechanism of action, thereby achieving an excellent PLK1 inhibitory effect as compared to the conventional PLK1 small molecule inhibitor that inhibits the simple activity of PLK1.

Accordingly, the composition including the compound represented by Formula I of the present disclosure or a pharmaceutically acceptable salt thereof may be effectively employed for selective degradation of PLK1.

An embodiment of the present disclosure is a composition for preventing or treating PLK1-related diseases including the compound represented by Formula I or the pharmaceutically acceptable salt thereof. An another embodiment of the present disclosure is a method for the prevention or treatment of PLK-related diseases comprising administering the composition to a subject in need thereof. The Formula I is the same as defined above.

In the present disclosure, the PLK1-related disease refers to any disease or condition capable of being treated, alleviated, delayed, inhibited or prevented from induction of degradation or inhibition of activity of PLK1. In an embodiment, the PLK1-related disease may be a cancer (malignant tumor), a benign tumor, a neurological disease, or other genetic or non-genetic diseases caused by excessive cell division.

The cancer includes all cancers capable of exhibiting prophylactic or therapeutic efficacy due to inhibition of PLK1 activity, and may be solid cancer or blood cancer. For example, the cancer may be one or more selected from the group consisting of squamous cell carcinoma, small cell lung cancer, non-small cell lung cancer, lung adenocarcinoma, lung squamous cell carcinoma, peritoneal cancer, skin cancer, skin or intraocular melanoma, rectal cancer, anal muscle cancer, esophageal cancer, small intestine cancer, endocrine cancer, parathyroid cancer, adrenal cancer, soft tissue sarcoma, urethral cancer, chronic or acute leukemia, lymphocytic lymphoma, hepatocellular carcinoma, gastrointestinal cancer, gastric cancer, pancreatic cancer, glioblastoma, cervical cancer, ovarian cancer, liver cancer, bladder cancer, liver tumor, breast cancer, colon cancer, colorectal cancer, endometrial or uterine cancer, salivary gland cancer, kidney cancer, prostate cancer, vulvar cancer, thyroid cancer, head and neck cancer, brain cancer, osteosarcoma, solid tumor, blood cancer, bone cancer, large cell lymphoma, adrenocorticoid tumor, t cell lymphoma/leukemia, neuroendocrine cancer, neuroendocrine tumor, cholangiocarcinoma, neuroblastoma, glioblastoma, glioma, and the like, but is not limited thereto. The cancer includes not only primary cancer but also metastatic cancer.

The benign tumors include all benign tumors capable of exhibiting prophylactic or therapeutic efficacy due to the inhibition of PLK1 activity, such as benign tumors in pre-cancer stages, and may be solid tumors or blood tumors. For example, the tumor may be one or more selected from the group consisting of Barrett's esophagus, colon adenoma and polyp, breast fibroadenoma and cyst, monoclonal gammopathy of undetermined significance (MGUS), monoclonal lymphocytosis, and the like, but is not limited thereto.

The neurological diseases include all neurological diseases capable of exhibiting prophylactic or therapeutic efficacy due to the inhibition of PLK1 activity, and specifically, may be one or more selected from the group consisting of central nervous system disease, neurodegenerative disease, Alzheimer's disease, Parkinson's disease, multiple sclerosis, Huntington's disease, senile dementia, epilepsy, Lou Gehrig, stroke, and nerve damage and axonal degeneration-related disorders following brain or spinal cord injury, but is not limited thereto.

The pharmaceutical composition of the present disclosure may further include one or more active ingredients exhibiting the same or similar medicinal effects in addition to the compound represented by Formula I above, or the pharmaceutically acceptable salt thereof.

An embodiment of the present disclosure is a method of degrading PLK1 by administering a compound represented by Formula I or a pharmaceutically acceptable salt thereof to mammals including humans.

Another embodiment of the present disclosure is a method of degrading PLK1 by administering the compound represented by Formula I or the pharmaceutically acceptable salt thereof to a sample in vitro. The sample may be a cell, a cell culture, a body fluid or tissue of a mammal including a human, but is not limited thereto.

### Advantageous Effects of Invention

The compounds of the present disclosure exhibit an effect of inducing PLK1 degradation. Therefore, the compounds of the present disclosure may be effectively utilized for preventing or treating PLK1-related diseases.

### BRIEF DESCRIPTION OF DRAWINGS

FIG. 1 shows the lucifease assay results by treating Compound 1 to Compound 41 of the present disclosure.

### BEST MODE FOR CARRYING OUT THE INVENTION

Unless otherwise defined, all technical and scientific terms used herein have the same meaning as commonly understood by one of ordinary skill in the art to which this disclosure belongs. The terminology used in the description is for describing particular embodiments only and is not intended to be limiting of the disclosure.

The present disclosure provides synthetic methods for Compound 1 to 44 shown in the table below.

TABLE 1

| Com- | |
|---|---|
| pound | Structure |
| 1 | 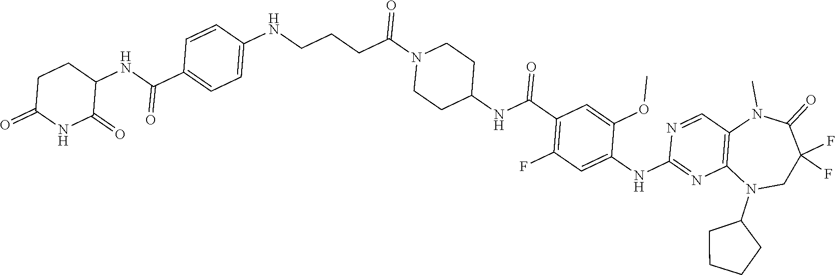 [SMILES: COc1cc(C(=O)NC2CCN(C(=O)CCCNc3ccc(C(=O)NC4CCC(=O)NC4=O)cc3)CC2)c(F)cc1Nc1ncc2c(n1)N(C1CCCC1)CC(F)(F)C(=O)N2C] |
| 2 | 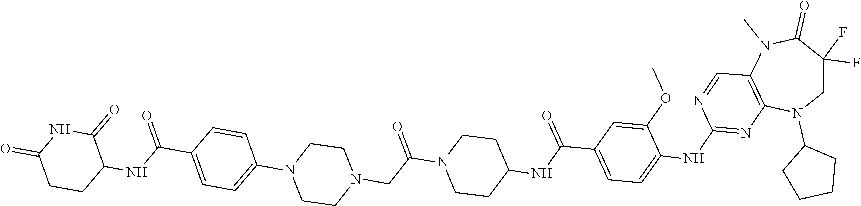 [SMILES: COc1cc(C(=O)NC2CCN(C(=O)CN3CCN(c4ccc(C(=O)NC5CCC(=O)NC5=O)cc4)CC3)CC2)ccc1Nc1ncc2c(n1)N(C1CCCC1)CC(F)(F)C(=O)N2C] |
| 3 | 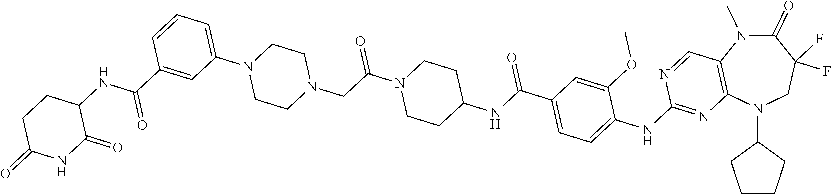 [SMILES: COc1cc(C(=O)NC2CCN(C(=O)CN3CCN(c4cccc(C(=O)NC5CCC(=O)NC5=O)c4)CC3)CC2)ccc1Nc1ncc2c(n1)N(C1CCCC1)CC(F)(F)C(=O)N2C] |
| 4 | 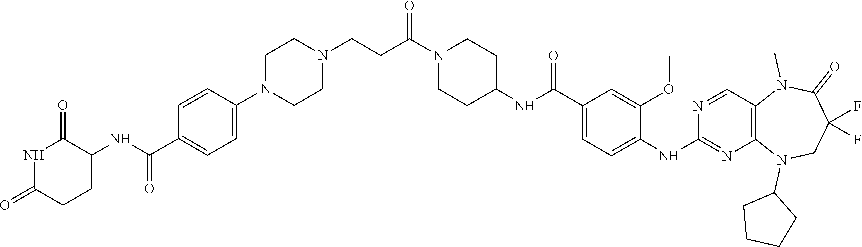 [SMILES: COc1cc(C(=O)NC2CCN(C(=O)CCN3CCN(c4ccc(C(=O)NC5CCC(=O)NC5=O)cc4)CC3)CC2)ccc1Nc1ncc2c(n1)N(C1CCCC1)CC(F)(F)C(=O)N2C] |
| 5 | 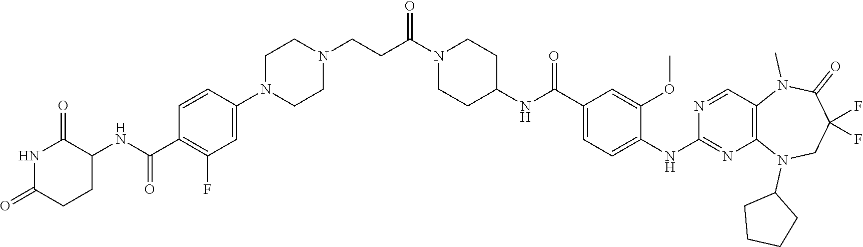 [SMILES: COc1cc(C(=O)NC2CCN(C(=O)CCN3CCN(c4ccc(C(=O)NC5CCC(=O)NC5=O)c(F)c4)CC3)CC2)ccc1Nc1ncc2c(n1)N(C1CCCC1)CC(F)(F)C(=O)N2C] |
| 6 | 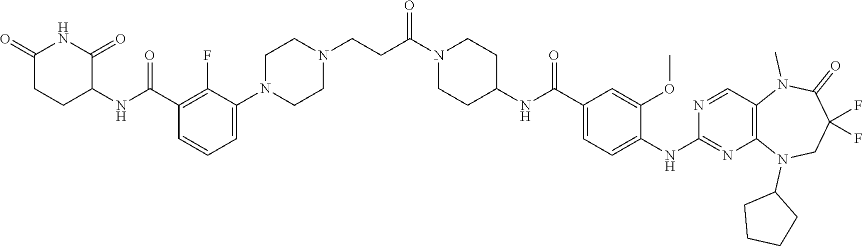 [SMILES: COc1cc(C(=O)NC2CCN(C(=O)CCN3CCN(c4cccc(C(=O)NC5CCC(=O)NC5=O)c4F)CC3)CC2)ccc1Nc1ncc2c(n1)N(C1CCCC1)CC(F)(F)C(=O)N2C] |
| 7 | 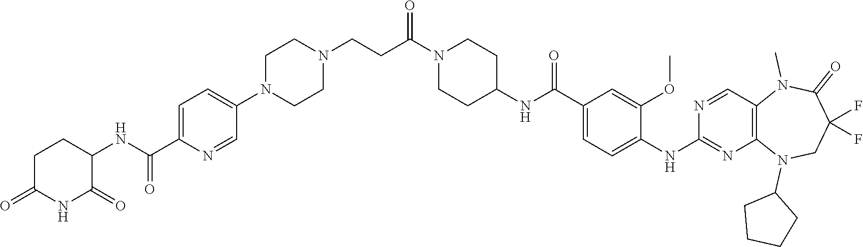 [SMILES: COc1cc(C(=O)NC2CCN(C(=O)CCN3CCN(c4ccc(C(=O)NC5CCC(=O)NC5=O)nc4)CC3)CC2)ccc1Nc1ncc2c(n1)N(C1CCCC1)CC(F)(F)C(=O)N2C] |
| 8 | 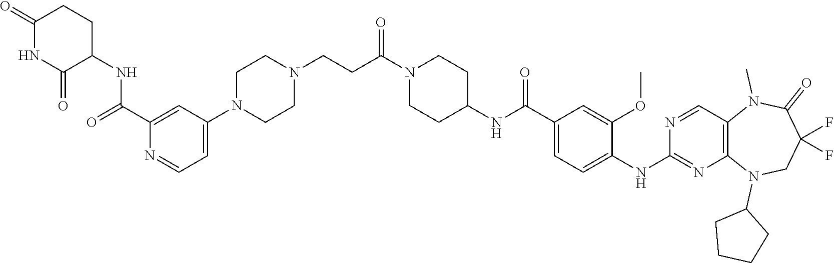 [SMILES: COc1cc(C(=O)NC2CCN(C(=O)CCN3CCN(c4ccnc(C(=O)NC5CCC(=O)NC5=O)c4)CC3)CC2)ccc1Nc1ncc2c(n1)N(C1CCCC1)CC(F)(F)C(=O)N2C] |
| 9 | 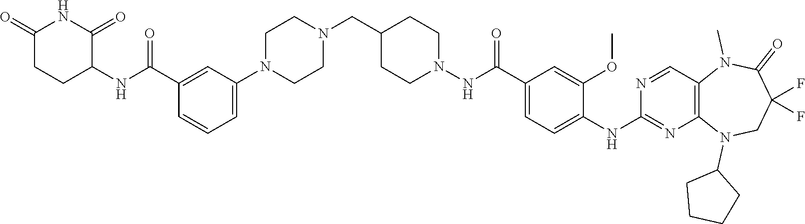 [SMILES: COc1cc(C(=O)NN2CCC(CN3CCN(c4cccc(C(=O)NC5CCC(=O)NC5=O)c4)CC3)CC2)ccc1Nc1ncc2c(n1)N(C1CCCC1)CC(F)(F)C(=O)N2C] |
| 10 | 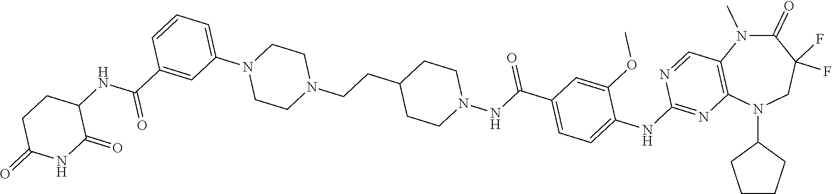 [SMILES: COc1cc(C(=O)NN2CCC(CCN3CCN(c4cccc(C(=O)NC5CCC(=O)NC5=O)c4)CC3)CC2)ccc1Nc1ncc2c(n1)N(C1CCCC1)CC(F)(F)C(=O)N2C] |
| 11 | 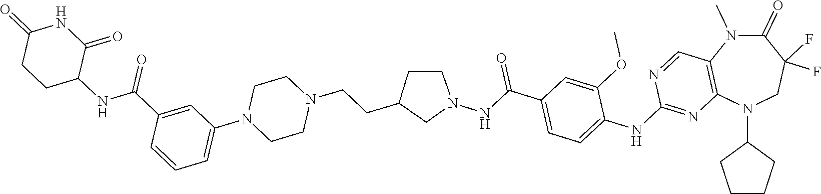 [SMILES: COc1cc(C(=O)NN2CCC(CCN3CCN(c4cccc(C(=O)NC5CCC(=O)NC5=O)c4)CC3)C2)ccc1Nc1ncc2c(n1)N(C1CCCC1)CC(F)(F)C(=O)N2C] |
| 12 | 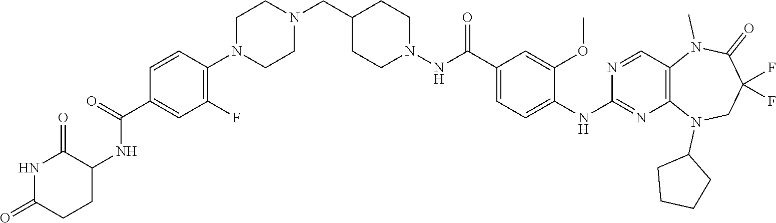 [SMILES: COc1cc(C(=O)NN2CCC(CN3CCN(c4ccc(C(=O)NC5CCC(=O)NC5=O)cc4F)CC3)CC2)ccc1Nc1ncc2c(n1)N(C1CCCC1)CC(F)(F)C(=O)N2C] |
| 13 | 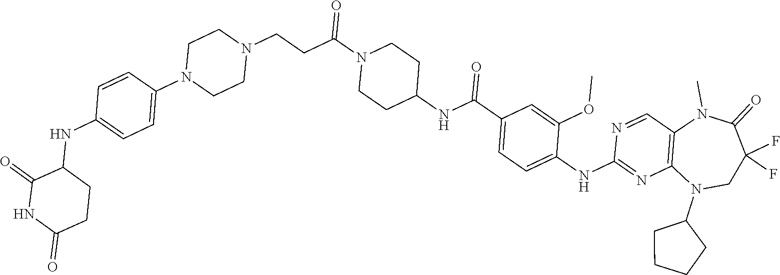 [SMILES: COc1cc(C(=O)NC2CCN(C(=O)CCN3CCN(c4ccc(NC5CCC(=O)NC5=O)cc4)CC3)CC2)ccc1Nc1ncc2c(n1)N(C1CCCC1)CC(F)(F)C(=O)N2C] |
| 14 | 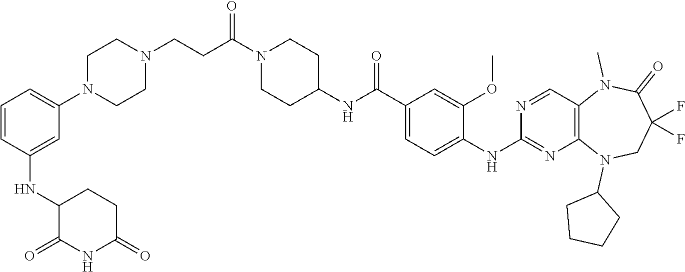 [SMILES: COc1cc(C(=O)NC2CCN(C(=O)CCN3CCN(c4cccc(NC5CCC(=O)NC5=O)c4)CC3)CC2)ccc1Nc1ncc2c(n1)N(C1CCCC1)CC(F)(F)C(=O)N2C] |
| 15 | 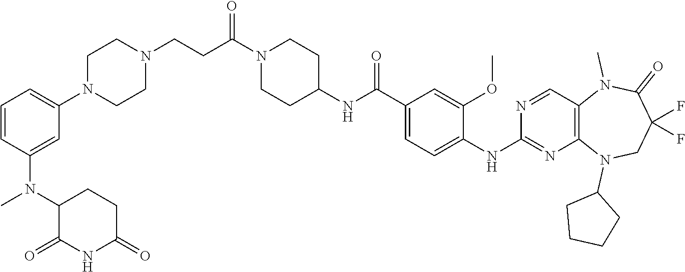 [SMILES: COc1cc(C(=O)NC2CCN(C(=O)CCN3CCN(c4cccc(N(C)C5CCC(=O)NC5=O)c4)CC3)CC2)ccc1Nc1ncc2c(n1)N(C1CCCC1)CC(F)(F)C(=O)N2C] |
| 16 | 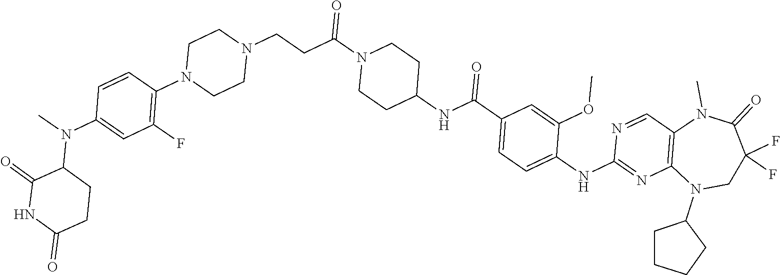 [SMILES: COc1cc(C(=O)NC2CCN(C(=O)CCN3CCN(c4ccc(N(C)C5CCC(=O)NC5=O)cc4F)CC3)CC2)ccc1Nc1ncc2c(n1)N(C1CCCC1)CC(F)(F)C(=O)N2C] |
| 17 | 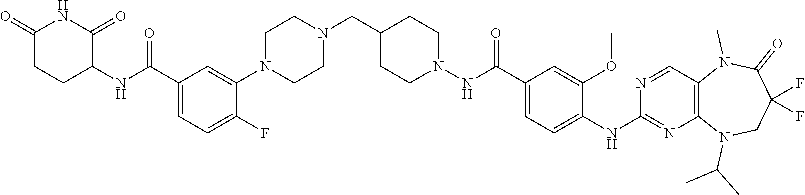 [SMILES: COc1cc(C(=O)NN2CCC(CN3CCN(c4cc(C(=O)NC5CCC(=O)NC5=O)ccc4F)CC3)CC2)ccc1Nc1ncc2c(n1)N(C(C)C)CC(F)(F)C(=O)N2C] |
| 18 | 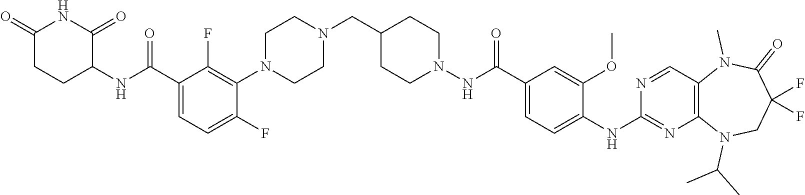 [SMILES: COc1cc(C(=O)NN2CCC(CN3CCN(c4c(F)ccc(C(=O)NC5CCC(=O)NC5=O)c4F)CC3)CC2)ccc1Nc1ncc2c(n1)N(C(C)C)CC(F)(F)C(=O)N2C] |
| 19 | 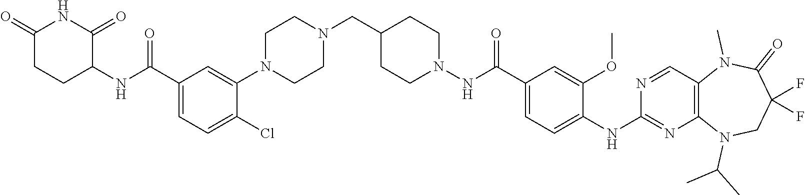 [SMILES: COc1cc(C(=O)NN2CCC(CN3CCN(c4cc(C(=O)NC5CCC(=O)NC5=O)ccc4Cl)CC3)CC2)ccc1Nc1ncc2c(n1)N(C(C)C)CC(F)(F)C(=O)N2C] |
| 20 | 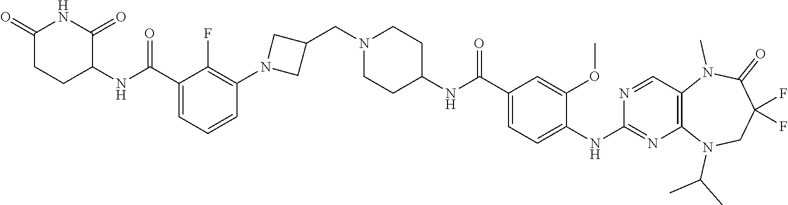 [SMILES: COc1cc(C(=O)NC2CCN(CC3CN(c4cccc(C(=O)NC5CCC(=O)NC5=O)c4F)C3)CC2)ccc1Nc1ncc2c(n1)N(C(C)C)CC(F)(F)C(=O)N2C] |
| 21 | 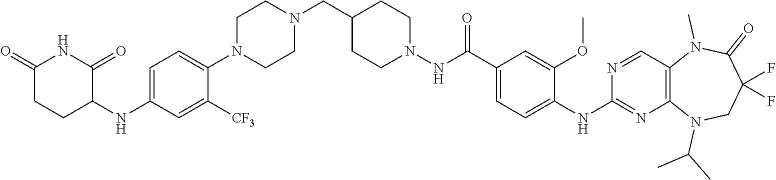 [SMILES: COc1cc(C(=O)NN2CCC(CN3CCN(c4ccc(NC5CCC(=O)NC5=O)cc4C(F)(F)F)CC3)CC2)ccc1Nc1ncc2c(n1)N(C(C)C)CC(F)(F)C(=O)N2C] |
| 22 | 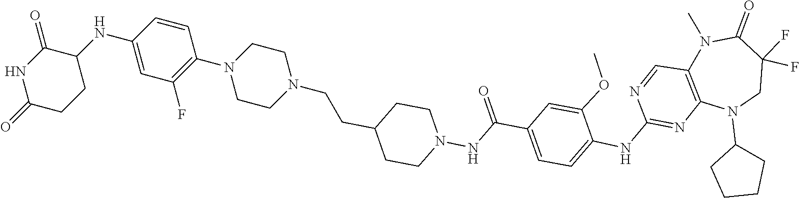 [SMILES: COc1cc(C(=O)NN2CCC(CCN3CCN(c4ccc(NC5CCC(=O)NC5=O)cc4F)CC3)CC2)ccc1Nc1ncc2c(n1)N(C1CCCC1)CC(F)(F)C(=O)N2C] |
| 23 | 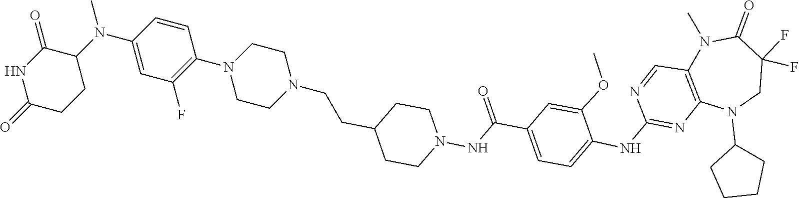 [SMILES: COc1cc(C(=O)NN2CCC(CCN3CCN(c4ccc(N(C)C5CCC(=O)NC5=O)cc4F)CC3)CC2)ccc1Nc1ncc2c(n1)N(C1CCCC1)CC(F)(F)C(=O)N2C] |
| 24 | 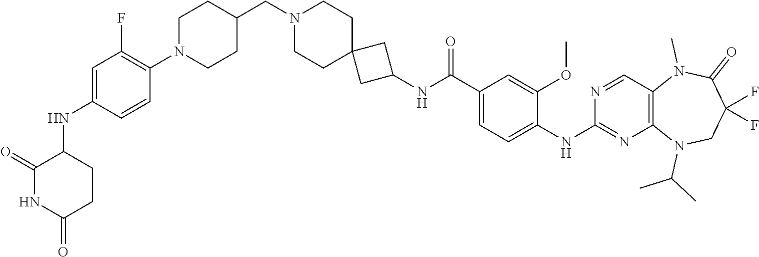 [SMILES: COc1cc(C(=O)NC2CC3(CCN(CC4CCN(c5ccc(NC6CCC(=O)NC6=O)cc5F)CC4)CC3)C2)ccc1Nc1ncc2c(n1)N(C(C)C)CC(F)(F)C(=O)N2C] |
| 25 | 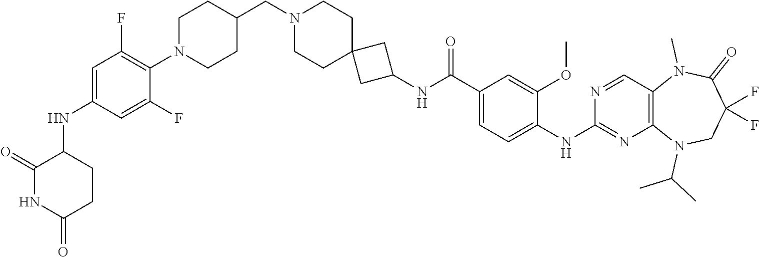 [SMILES: COc1cc(C(=O)NC2CC3(CCN(CC4CCN(c5c(F)cc(NC6CCC(=O)NC6=O)cc5F)CC4)CC3)C2)ccc1Nc1ncc2c(n1)N(C(C)C)CC(F)(F)C(=O)N2C] |
| 26 | 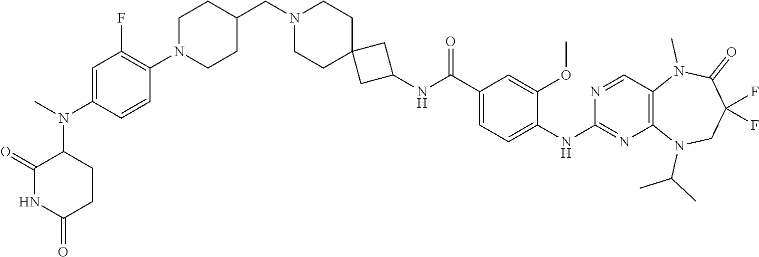 [SMILES: COc1cc(C(=O)NC2CC3(CCN(CC4CCN(c5ccc(N(C)C6CCC(=O)NC6=O)cc5F)CC4)CC3)C2)ccc1Nc1ncc2c(n1)N(C(C)C)CC(F)(F)C(=O)N2C] |
| 27 | 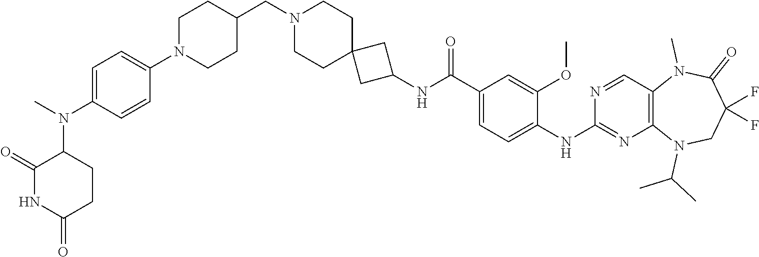 [SMILES: COc1cc(C(=O)NC2CC3(CCN(CC4CCN(c5ccc(N(C)C6CCC(=O)NC6=O)cc5)CC4)CC3)C2)ccc1Nc1ncc2c(n1)N(C(C)C)CC(F)(F)C(=O)N2C] |
| 28 | 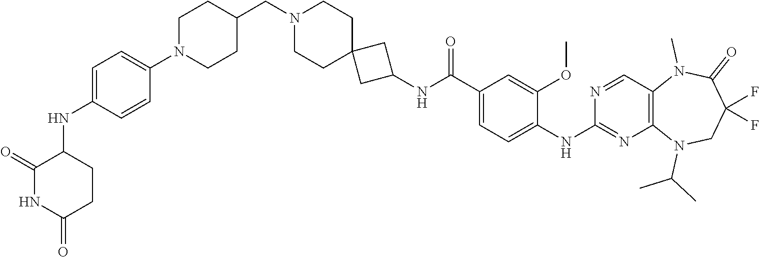 [SMILES: COc1cc(C(=O)NC2CC3(CCN(CC4CCN(c5ccc(NC6CCC(=O)NC6=O)cc5)CC4)CC3)C2)ccc1Nc1ncc2c(n1)N(C(C)C)CC(F)(F)C(=O)N2C] |
| 29 | 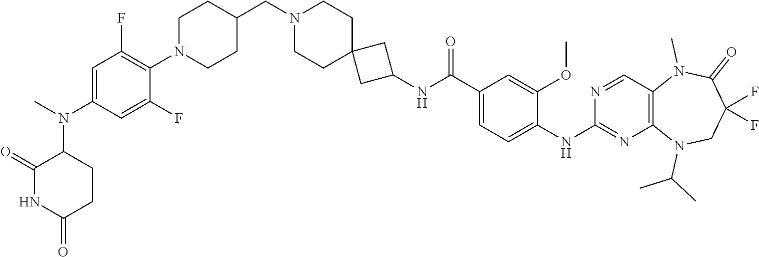 [SMILES: COc1cc(C(=O)NC2CC3(CCN(CC4CCN(c5c(F)cc(N(C)C6CCC(=O)NC6=O)cc5F)CC4)CC3)C2)ccc1Nc1ncc2c(n1)N(C(C)C)CC(F)(F)C(=O)N2C] |
| 30 | 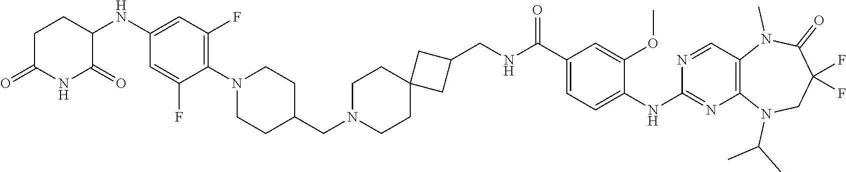 [SMILES: COc1cc(C(=O)NCC2CC3(CCN(CC4CCN(c5c(F)cc(NC6CCC(=O)NC6=O)cc5F)CC4)CC3)C2)ccc1Nc1ncc2c(n1)N(C(C)C)CC(F)(F)C(=O)N2C] |
| 31 | 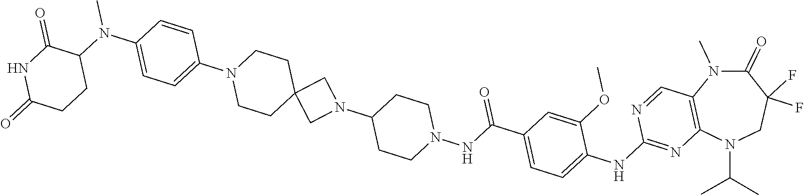 [SMILES: COc1cc(C(=O)NN2CCC(N3CC4(CCN(c5ccc(N(C)C6CCC(=O)NC6=O)cc5)CC4)C3)CC2)ccc1Nc1ncc2c(n1)N(C(C)C)CC(F)(F)C(=O)N2C] |
| 32 | 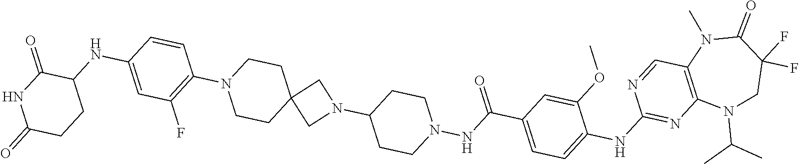 [SMILES: COc1cc(C(=O)NN2CCC(N3CC4(CCN(c5ccc(NC6CCC(=O)NC6=O)cc5F)CC4)C3)CC2)ccc1Nc1ncc2c(n1)N(C(C)C)CC(F)(F)C(=O)N2C] |
| 33 | 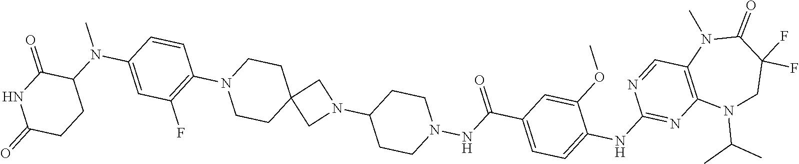 [SMILES: COc1cc(C(=O)NN2CCC(N3CC4(CCN(c5ccc(N(C)C6CCC(=O)NC6=O)cc5F)CC4)C3)CC2)ccc1Nc1ncc2c(n1)N(C(C)C)CC(F)(F)C(=O)N2C] |
| 34 | 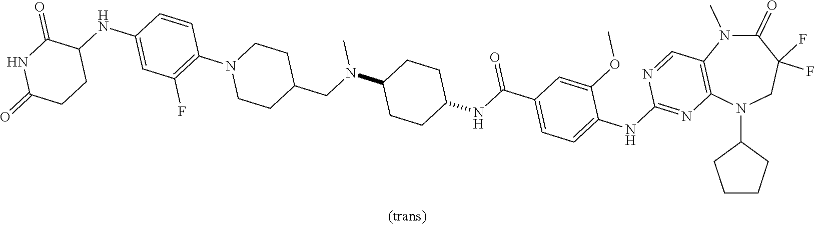 |
| 35 | 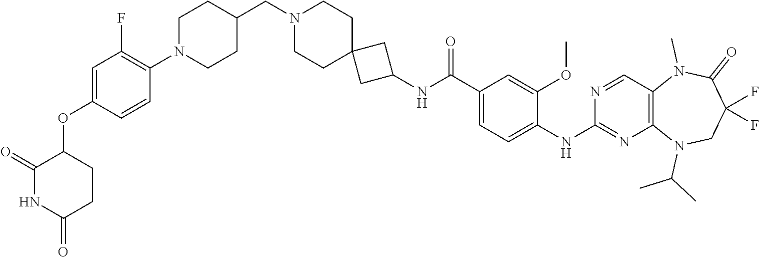 [SMILES: COc1cc(C(=O)NC2CC3(CCN(CC4CCN(c5ccc(OC6CCC(=O)NC6=O)cc5F)CC4)CC3)C2)ccc1Nc1ncc2c(n1)N(C(C)C)CC(F)(F)C(=O)N2C] |
| 36 | 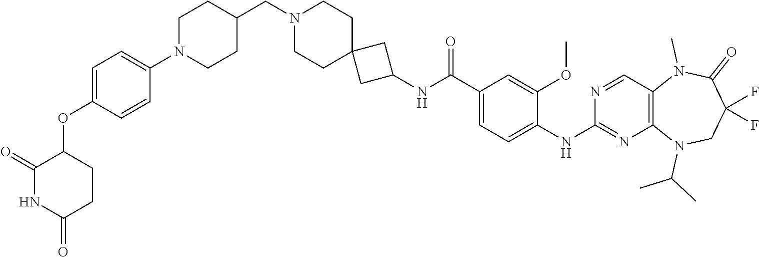 [SMILES: COc1cc(C(=O)NC2CC3(CCN(CC4CCN(c5ccc(OC6CCC(=O)NC6=O)cc5)CC4)CC3)C2)ccc1Nc1ncc2c(n1)N(C(C)C)CC(F)(F)C(=O)N2C] |
| 37 | 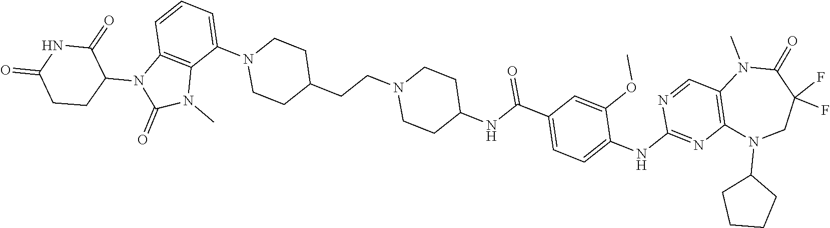 [SMILES: COc1cc(C(=O)NC2CCN(CCC3CCN(c4cccc5c4n(C)c(=O)n5C4CCC(=O)NC4=O)CC3)CC2)ccc1Nc1ncc2c(n1)N(C1CCCC1)CC(F)(F)C(=O)N2C] |
| 38 | 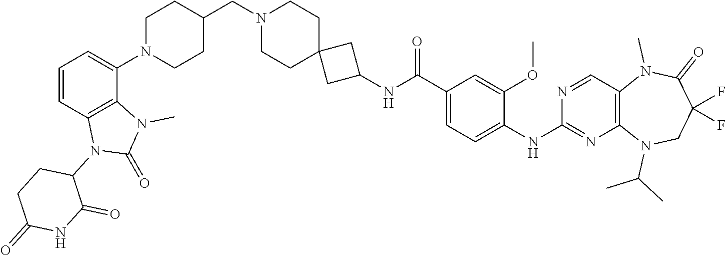 [SMILES: COc1cc(C(=O)NC2CC3(CCN(CC4CCN(c5cccc6c5n(C)c(=O)n6C5CCC(=O)NC5=O)CC4)CC3)C2)ccc1Nc1ncc2c(n1)N(C(C)C)CC(F)(F)C(=O)N2C] |
| 39 | 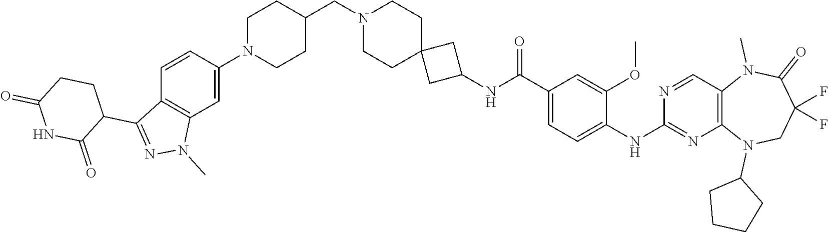 [SMILES: COc1cc(C(=O)NC2CC3(CCN(CC4CCN(c5ccc6c(C7CCC(=O)NC7=O)nn(C)c6c5)CC4)CC3)C2)ccc1Nc1ncc2c(n1)N(C1CCCC1)CC(F)(F)C(=O)N2C] |
| 40 | 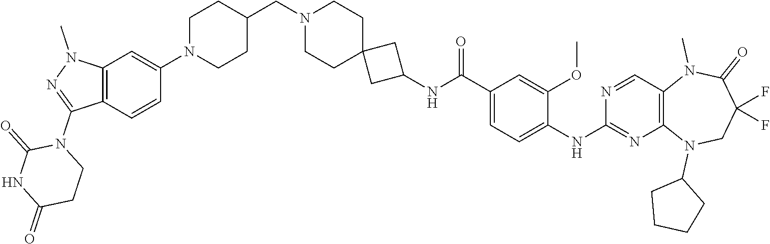 [SMILES: COc1cc(C(=O)NC2CC3(CCN(CC4CCN(c5ccc6c(N7CCC(=O)NC7=O)nn(C)c6c5)CC4)CC3)C2)ccc1Nc1ncc2c(n1)N(C1CCCC1)CC(F)(F)C(=O)N2C] |
| 41 | 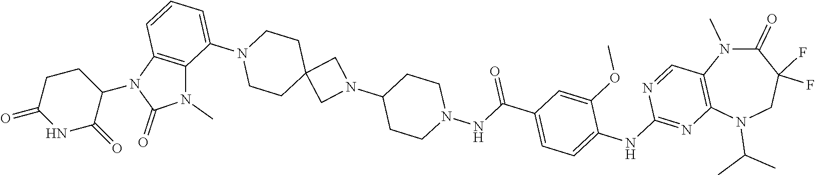 [SMILES: COc1cc(C(=O)NN2CCC(N3CC4(CCN(c5cccc6c5n(C)c(=O)n6C5CCC(=O)NC5=O)CC4)C3)CC2)ccc1Nc1ncc2c(n1)N(C(C)C)CC(F)(F)C(=O)N2C] |
| 42 | 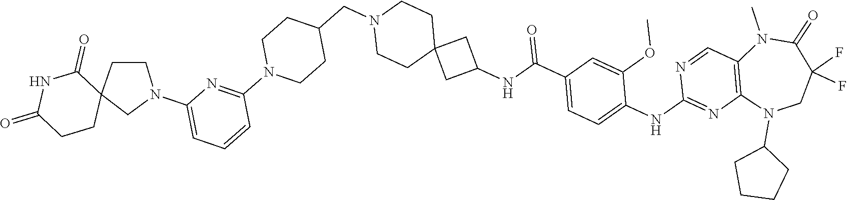 [SMILES: COc1cc(C(=O)NC2CC3(CCN(CC4CCN(c5cccc(N6CCC7(CCC(=O)NC7=O)C6)n5)CC4)CC3)C2)ccc1Nc1ncc2c(n1)N(C1CCCC1)CC(F)(F)C(=O)N2C] |
| 43 | 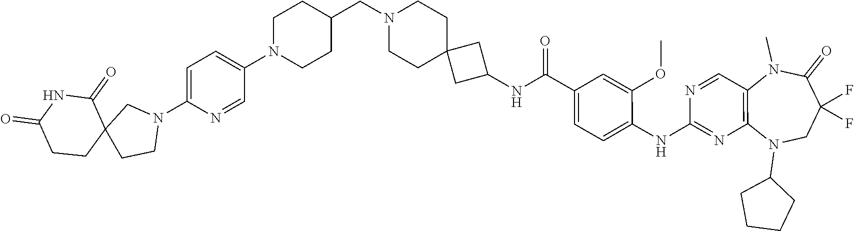 [SMILES: COc1cc(C(=O)NC2CC3(CCN(CC4CCN(c5ccc(N6CCC7(CCC(=O)NC7=O)C6)nc5)CC4)CC3)C2)ccc1Nc1ncc2c(n1)N(C1CCCC1)CC(F)(F)C(=O)N2C] |
| 44 | 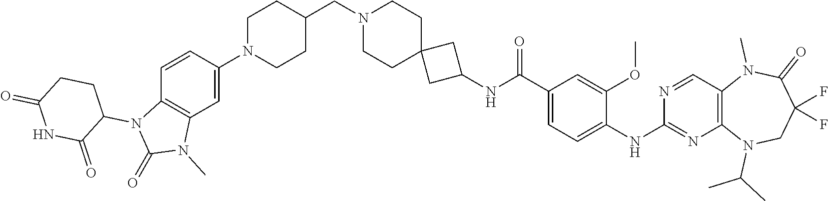 [SMILES: COc1cc(C(=O)NC2CC3(CCN(CC4CCN(c5ccc6c(c5)n(C)c(=O)n6C5CCC(=O)NC5=O)CC4)CC3)C2)ccc1Nc1ncc2c(n1)N(C(C)C)CC(F)(F)C(=O)N2C] |

### Example 1. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(4-((4-((2,6-dioxopiperidin-3-yl)carbamoyl)phenyl)amino)butanoyl)piperidin-4-yl)-2-fluoro-5-methoxybenzamide (Compound 1)

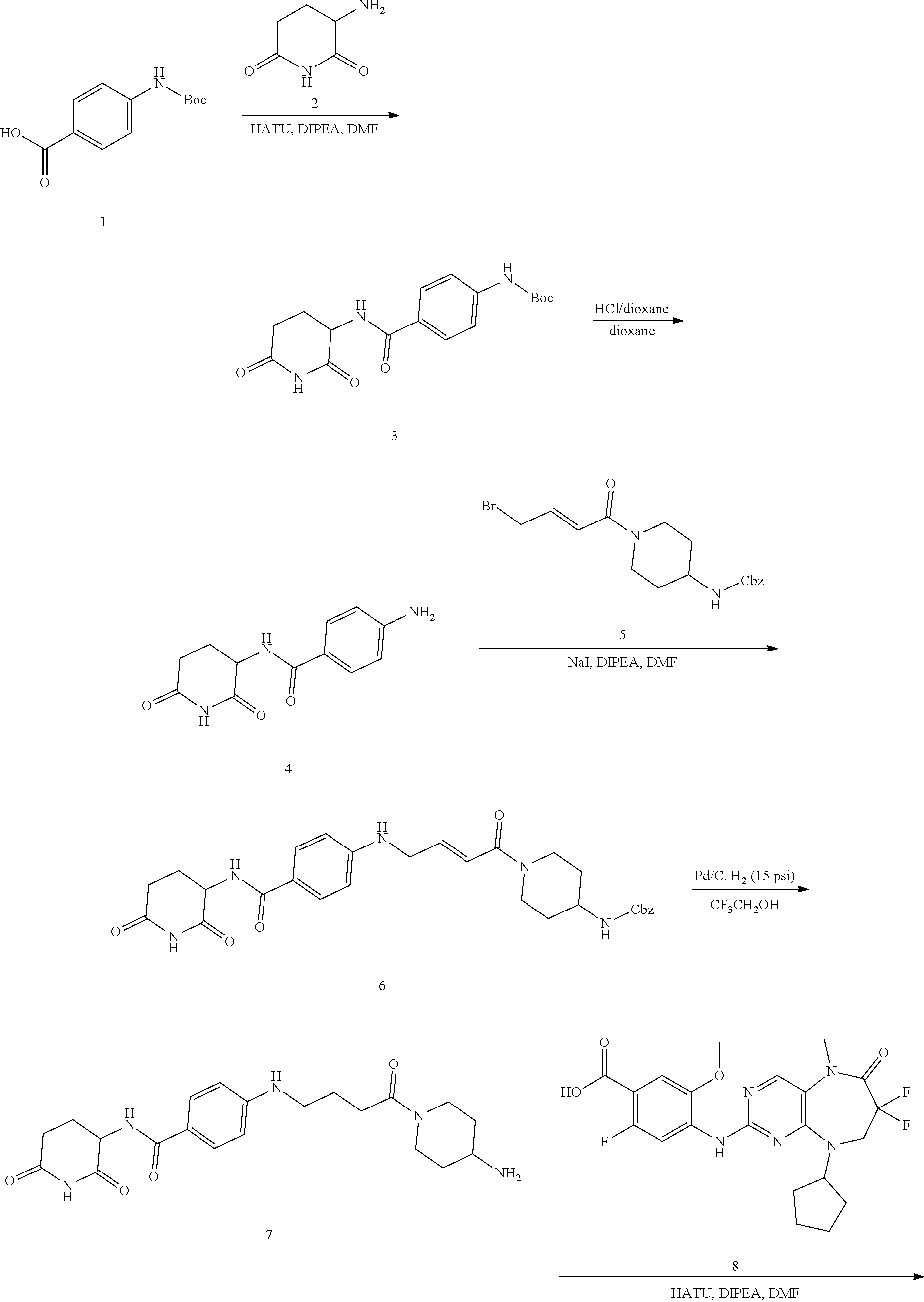

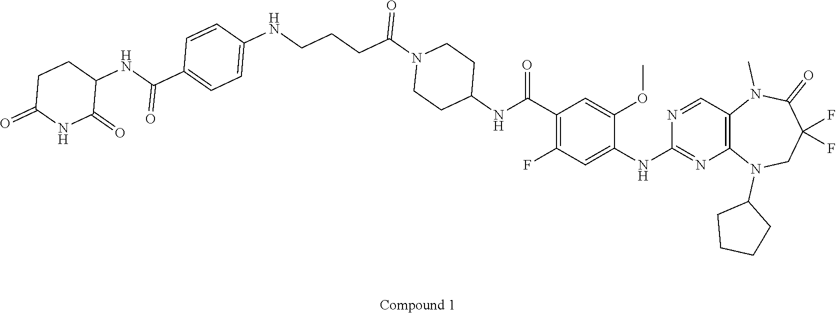 [SMILES: COc1cc(C(=O)NC2CCN(C(=O)CCCNc3ccc(C(=O)NC4CCC(=O)NC4=O)cc3)CC2)c(F)cc1Nc1ncc2c(n1)N(C1CCCC1)CC(F)(F)C(=O)N2C]

### Step 1. Synthesis of tert-butyl (4-((2,6-dioxopiperidin-3-yl)carbamoyl)phenyl)carbamate (3)

To a solution of 4-((tert-butoxycarbonyl)amino)benzoic acid (1 g, 4.21 mmol) in DMF (10 mL) were added DIPEA (1.63 g, 12.64 mmol, 2.20 mL) and HATU (3.21 g, 8.43 mmol). Then 3-aminopiperidine-2,6-dione (693.74 mg, 4.21 mmol, HCl salt) was added to the mixture after 0.5 h at 25° C. The mixture was stirred at 25° C. for 2 h. LCMS showed ˜70% of desired mass was detected. The reaction mixture was diluted with water (50 mL) and extracted with EtOAc (20 mL×3). White solid was precipitated from the combined organic layers. The mixture was filtered. The filter cake was dried under reduced pressure to afford tert-butyl (4-((2,6-dioxopiperidin-3-yl)carbamoyl)phenyl)carbamate (0.8 g, 2.14 mmol, 50.87% yield, 93.1% purity) as a white solid. MS (M+H)⁺=348.2

¹H NMR (400 MHz, DMSO-d₆) δ=10.84 (s, 1H), 9.64 (s, 1H), 8.59 (d, J=8.0 Hz, 1H), 7.79 (d, J=8.8 Hz, 2H), 7.54 (d, J=8.8 Hz, 2H), 4.79-4.72 (m, 1H), 2.84-2.75 (m, 1H), 2.56-2.52 (m, 1H), 2.14-2.09 (m, 1H), 1.98-1.94 (m, 1H), 1.49 (s, 9H).

### Step 2. Synthesis of 4-amino-N-(2,6-dioxopiperidin-3-yl)benzamide (4)

To a solution of tert-butyl (4-((2,6-dioxopiperidin-3-yl)carbamoyl)phenyl)carbamate (0.8 g, 2.30 mmol) in dioxane (2 mL) was added HCl/dioxane (4 M, 5 mL). The mixture was stirred at 25° C. for 1 h. LCMS showed the starting material was consumed completely. The mixture was concentrated under reduced pressure to afford 4-amino-N-(2,6-dioxopiperidin-3-yl)benzamide (660 mg, crude, HCl salt) as a white solid, which was used for the next step directly. MS (M+H)⁺=248.3

### Step 3. Synthesis of benzyl (E)-(1-(4-((4-((2,6-dioxopiperidin-3-yl)carbamoyl)phenyl) amino)but-2-enoyl)piperidin-4-yl)carbamate (6)

To a solution of 4-amino-N-(2,6-dioxopiperidin-3-yl)benzamide (200 mg, 704.94 μmol, HCl) in DMF (3 mL) were added NaI (31.70 mg, 211.48 μmol), benzyl (E)-(1-(4-bromobut-2-enoyl)piperidin-4-yl)carbamate (322.52 mg, 845.93 μmol) and DIPEA (273.33 mg, 2.11 mmol, 368.37 L). The mixture was stirred at 25° C. for 14 h. TLC (EtOAc:methanol=10:1) indicated several new spots formed. The reaction mixture was diluted with water (30 mL) and extracted with EtOAc (10 mL×3). The combined organic layers were washed with saturated brine (10 mL), dried over anhydrous sodium sulfate, filtered and concentrated under reduced pressure to give a residue. The residue was purified by flash silica gel chromatography (ISCO®; 20 g SepaFlash® Silica Flash Column, eluent of 0˜40% petroleum ether:EtOAc/methanol (v/v=1/1) gradient@80 mL/min) to afford benzyl (E)-(1-(4-((4-((2,6-dioxopiperidin-3-yl)carbamoyl)phenyl)amino)but-2-enoyl)piperidin-4-yl)carbamate (0.2 g, 365.23 μmol, 51.81% yield) as yellow oil, which was used for the next step directly. MS (M+H)⁺=548.3

### Step 4. Synthesis of 4-((4-(4-aminopiperidin-1-yl)-4-oxobutyl)amino)-N-(2,6-dioxopiperidin-3-yl)benzamide (7)

To a mixture of Pd/C (20 mg, 18.26 μmol, 10% purity) in CF₃CH₂OH (5 mL) was added benzyl (E)-(1-(4-((4-((2,6-dioxopiperidin-3-yl)carbamoyl)phenyl)amino)but-2-enoyl)piperidin-4-yl)carbamate (0.1 g, 182.61 μmol) at 25° C. The mixture was stirred at 25° C. for 12 h under H₂ (15 Psi). LCMS showed the reaction was completed. The mixture was filtered to remove the catalyst. The filtrate was concentrated under reduced pressure to afford 4-((4-(4-aminopiperidin-1-yl)-4-oxobutyl)amino)-N-(2,6-dioxopiperidin-3-yl)benzamide (80 mg, 92.23 μmol, 50.51% yield, 47.9% purity) as yellow oil, which was used for the next step directly. MS (M+H)⁺=416.2

### Step 5. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(4-((4-((2,6-dioxopiperidin-3-yl)carbamoyl)phenyl)amino)butanoyl)piperidin-4-yl)-2-fluoro-5-methoxybenzamide (Compound 1)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxybenzoic acid (80 mg, 171.89 μmol) in DMF (2 mL) were added HATU (98.03 mg, 257.83 μmol) and DIPEA (66.65 mg, 515.66 μmol, 89.82 L). Then 4-[[4-(4-amino-1-piperidyl)-4-oxo-butyl]amino]-N-(2,6-dioxo-3-piperidyl)benzamide (78.56 mg, 189.07 μmol) was added to the mixture after 0.5 h. The mixture was stirred at 25° C. for 12 h. LCMS showed a 60% peak with desired mass. The reaction mixture was diluted with water (20 mL) and extracted with EtOAc (20 mL×3). The combined organic layers were washed with saturated brine (10 mL), dried over anhydrous sodium sulfate, filtered and concentrated under reduced pressure to give a residue. The residue was purified by prep-HPLC (column: Phenomenex Synergi C18 150×25 mm×10 μm; mobile phase: [water(FA)-ACN]; B %: 39%-69%, 10 min) and then prep-HPLC (column: Waters Xbridge 150×25 mm×5 μm; mobile phase: [water(NH₄HCO₃)-ACN]; B %: 32%-62%, 8 min) followed by lyophilization to afford 4-((9-cyclopentyl-7,7-difluoro-5-methyl-6-oxo-6,7,8,9-tetrahydro-5H-pyrimido[4,5-b][1,4]diazepin-2-yl)amino)-N-(1-(4-((4-((2,6-dioxopiperidin-3-yl)carbamoyl)phenyl)amino)butanoyl)piperidin-4-yl)-2-fluoro-5-methoxybenzamide (25.2 mg, 26.90 μmol, 15.65% yield, 92.1% purity) as a white solid. MS (M+H)⁺=863.3

¹H NMR (400 MHz, DMSO-d₆) δ=10.79 (s, 1H), 8.29-8.23 (m, 3H), 8.03 (s, 1H), 7.97-7.95 (m, 1H), 7.64 (d, J=8.8 Hz, 2H), 7.19 (d, J=6.8 Hz, 1H), 6.57 (d, J=8.8 Hz, 2H), 6.25 (t, J=5.6 Hz, 1H), 4.85-4.79 (m, 1H), 4.74-4.67 (m, 1H), 4.34-4.29 (m, 1H), 4.10-3.98 (m, 3H), 3.91-3.84 (m, 4H), 3.27 (s, 3H), 3.13-3.07 (m, 3H), 2.76-2.71 (m, 1H), 2.52 (s, 3H), 2.44-2.40 (m, 2H), 2.13-2.03 (m, 1H), 1.98-1.91 (m, 3H), 1.83-1.71 (m, 5H), 1.66-1.57 (m, 4H), 1.50-1.37 (m, 2H).

### Example 2. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-(4-(4-((2,6-dioxopiperidin-3-yl)carbamoyl)phenyl)piperazin-1-yl)acetyl)piperidin-4-yl)-3-methoxybenzamide (Compound 2)

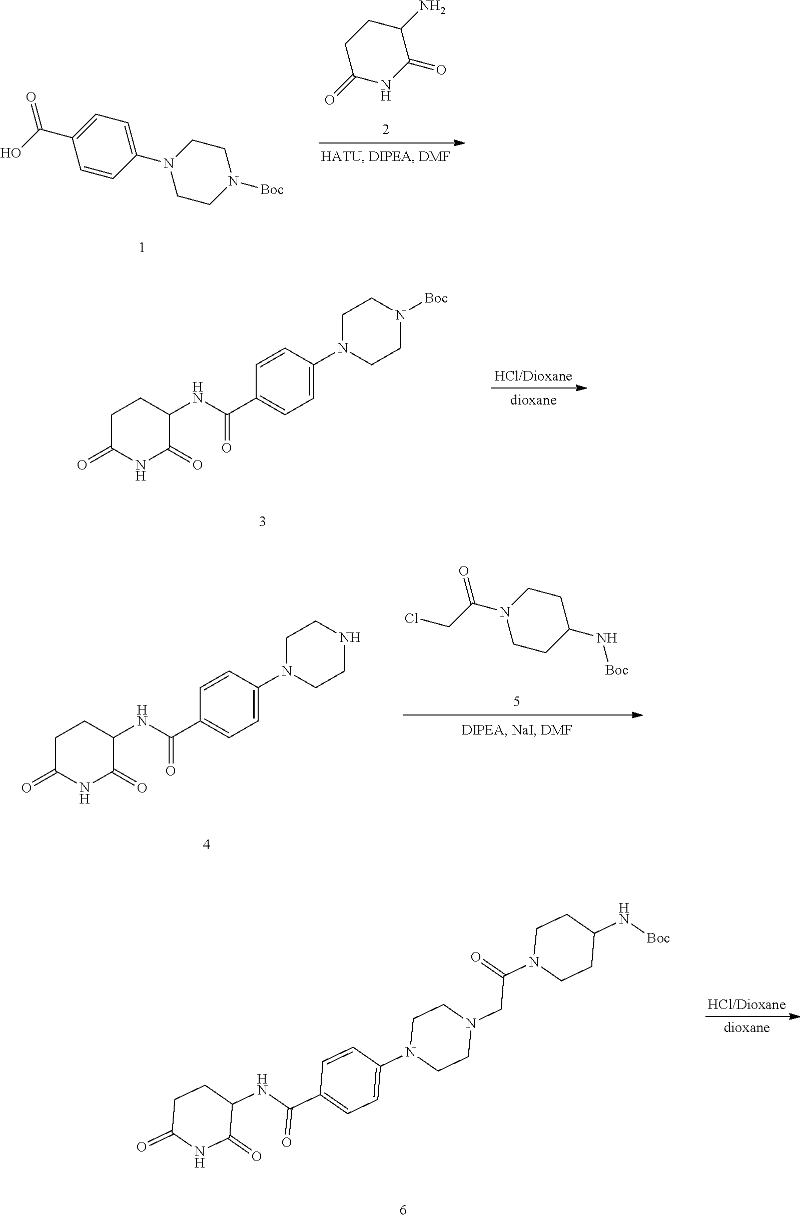 [SMILES: CC(C)(C)OC(=O)N1CCN(c2ccc(C(=O)O)cc2)CC1 ; NC1CCC(=O)NC1=O ; CC(C)(C)OC(=O)N1CCN(c2ccc(C(=O)NC3CCC(=O)NC3=O)cc2)CC1 ; O=C1CCC(NC(=O)c2ccc(N3CCNCC3)cc2)C(=O)N1 ; CC(C)(C)OC(=O)NC1CCN(C(=O)CCl)CC1 ; CC(C)(C)OC(=O)NC1CCN(C(=O)CN2CCN(c3ccc(C(=O)NC4CCC(=O)NC4=O)cc3)CC2)CC1]

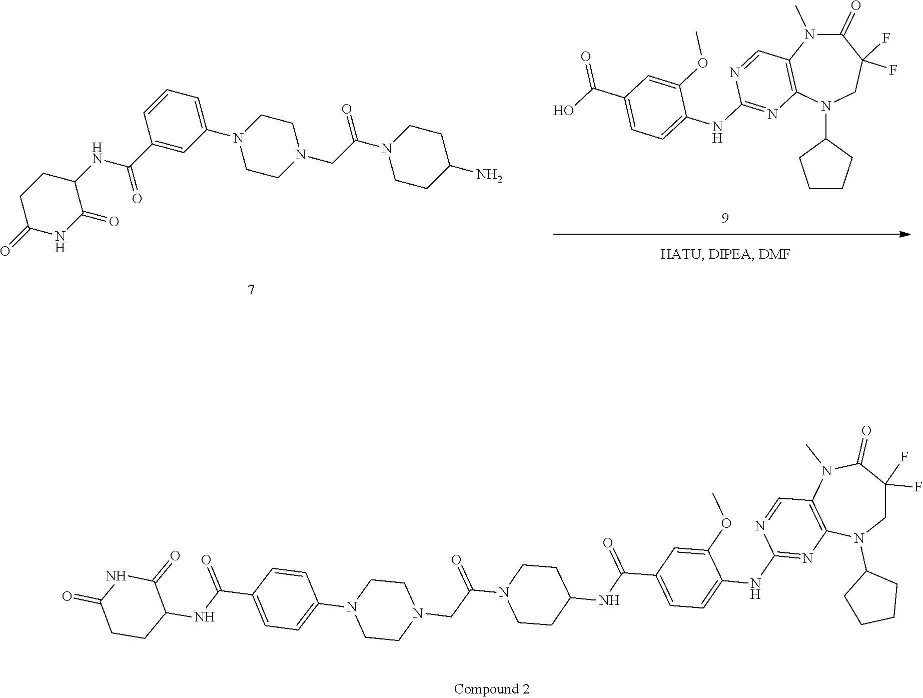 [SMILES: NC1CCN(C(=O)CN2CCN(c3cccc(C(=O)NC4CCC(=O)NC4=O)c3)CC2)CC1 ; COc1cc(C(=O)O)ccc1Nc1ncc2c(n1)N(C1CCCC1)CC(F)(F)C(=O)N2C ; COc1cc(C(=O)NC2CCN(C(=O)CN3CCN(c4ccc(C(=O)NC5CCC(=O)NC5=O)cc4)CC3)CC2)ccc1Nc1ncc2c(n1)N(C1CCCC1)CC(F)(F)C(=O)N2C]

### Step 1. Synthesis of tert-butyl 4-(4-((2,6-dioxopiperidin-3-yl)carbamoyl)phenyl)piperazine-1-carboxylate (3)

To a solution of 4-(4-tert-butoxycarbonylpiperazin-1-yl)benzoic acid (0.8 g, 2.61 mmol) in DMF (5 mL) were added HATU (1.09 g, 2.87 mmol) and DIPEA (675.00 mg, 5.22 mmol, 909.70 L). the mixture was stirred at 20° C. for 10 min and a solution of 3-aminopiperidine-2,6-dione (515.76 mg, 3.13 mmol, HCl salt) in DMF (5 mL) with DIPEA (675.00 mg, 5.22 mmol, 909.70 μL) was added and the resulting mixture was stirred at 20° C. for 1 h. LCMS showed starting material was consumed completely and a main peak (96%) with desired mass. The reaction mixture was diluted with H₂O (15 mL) and a lot of white solid was precipitated, the white solid was collected by filtration and dried in vacuum to afford tert-butyl 4-(4-((2,6-dioxopiperidin-3-yl)carbamoyl)phenyl)piperazine-1-carboxylate (470 mg, 1.09 mmol, 41.92% yield, 97% purity) as a white solid. MS (M+H)⁺=417.3

### Step 2. Synthesis of N-(2,6-dioxopiperidin-3-yl)-4-(piperazin-1-yl)benzamide (4)

To a solution of tert-butyl 4-(4-((2,6-dioxopiperidin-3-yl)carbamoyl)phenyl)piperazine-1-carboxylate (470 mg, 1.13 mmol) in dioxane (4 mL) was added HCl/dioxane (4 M, 12 mL) at 20° C. and the resulting mixture was stirred at 20° C. for 0.5 h. LCMS showed starting material was consumed completely and a main peak (94%) with desired mass. The reaction mixture was concentrated in vacuum to afford N-(2,6-dioxopiperidin-3-yl)-4-(piperazin-1-yl)benzamide (400 mg, crude, HCl salt) as a white solid. MS (M+H)⁺=317.4

### Step 3. Synthesis of tert-butyl (1-(2-(4-(4-((2,6-dioxopiperidin-3-yl)carbamoyl)phenyl)piperazin-1-yl)acetyl)piperidin-4-yl)carbamate (6)

To a solution of N-(2,6-dioxopiperidin-3-yl)-4-(piperazin-1-yl)benzamide (200 mg, 566.87 μmol, HCl salt) and tert-butyl N-[1-(2-chloroacetyl)-4-piperidyl]carbamate (172.57 mg, 623.56 μmol) in DMF (4 mL) were added DIPEA (219.79 mg, 1.70 mmol, 296.22 μL) and NaI (16.99 mg, 113.37 μmol) at 20° C. and the resulting mixture was stirred at 80° C. for 16 h. LCMS showed starting material was consumed completely and a peak (74%) with desired mass. The reaction mixture was diluted with H₂O (12 mL) and extracted with EtOAc (12 mL×3). The organic layer was washed with brine (12 mL×3), dried over Na₂SO₄, filtered and concentrated. The residue was purified by flash silica gel chromatography (12 g SepaFlash® Silica Flash Column, Eluent of 0˜100% EtOAc/Petroleum ether gradient@100 mL/min) to afford tert-butyl (1-(2-(4-(4-((2,6-dioxopiperidin-3-yl)carbamoyl)phenyl)piperazin-1-yl)acetyl)piperidin-4-yl)carbamate (180 mg, 320.13 μmol, 56.47% yield, 99% purity) as an off-white solid. MS (M+H)⁺=557.3

### Step 4. Synthesis of 3-(4-(2-(4-aminopiperidin-1-yl)-2-oxoethyl)piperazin-1-yl)-N-(2,6-dioxopiperidin-3-yl)benzamide (7)

To a solution of tert-butyl (1-(2-(4-(4-((2,6-dioxopiperidin-3-yl)carbamoyl)phenyl)piperazin-1-yl)acetyl)piperidin-4-yl)carbamate (180 mg, 323.36 μmol) in dioxane (4 mL) was added HCl/dioxane (4 M, 8 mL) at 20° C. and the resulting mixture was stirred at 20° C. for 1 h. LCMS showed starting material was consumed completely and a main peak (95%) with desired mass. The reaction mixture was concentrated in vacuum to afford 3-(4-(2-(4-aminopiperidin-1-yl)-2-oxoethyl)piperazin-1-yl)-N-(2,6-dioxopiperidin-3-yl)benzamide (160 mg, crude, HCl salt) as an off-white solid. MS (M+H)⁺=457.4

### Step 5. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-(4-(4-((2,6-dioxopiperidin-3-yl)carbamoyl)phenyl)piperazin-1-yl)acetyl)piperidin-4-yl)-3-methoxybenzamide (Compound 2)

To a solution of 4-[(9-cyclopentyl-7,7-difluoro-5-methyl-6-oxo-8H-pyrimido[4,5-b][1,4]diazepin-2-yl) amino]-3-methoxy-benzoic acid (80 mg, 178.80 μmol) in DMF (2 mL) were added HATU (74.78 mg, 196.68 μmol) and DIPEA (46.22 mg, 357.59 μmol, 62.29 μL). The mixture was stirred at 20° C. for 10 min, then a solution of 3-(4-(2-(4-aminopiperidin-1-yl)-2-oxoethyl)piperazin-1-yl)-N-(2,6-dioxopiperidin-3-yl)benzamide (96.96 mg, 196.68 μmol, HCl salt) with DIPEA (46.22 mg, 357.59 μmol, 62.29 μL) in DMF (2 mL) was added and the resulting mixture was stirred at 20° C. for another 1 h. LCMS showed starting material was consumed completely and a peak (64%) with desired mass. The reaction mixture was diluted with H₂O (10 mL) and extracted with EtOAc (10 mL×6). The organic layer was dried over Na₂SO₄, filtered and concentrated. The residue was purified by prep-HPLC (column: Waters Xbridge 150×25 mm×5 μm; mobile phase: [water (NH₄HCO₃)-ACN]; B %: 29%-59%, 10 min) and the eluent was lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-(4-(4-((2,6-dioxopiperidin-3-yl)carbamoyl)phenyl)piperazin-1-yl)acetyl)piperidin-4-yl)-3-methoxybenzamide (24.3 mg, 27.15 μmol, 15.19% yield, 99% purity) as a white solid. MS (M+H)⁺=886.3

¹H NMR (400 MHz, DMSO-d₆) δ=10.82 (s, 1H), 8.47 (d, J=8.3 Hz, 1H), 8.23 (s, 1H), 8.14 (d, J=8.2 Hz, 1H), 7.99 (s, 1H), 7.81-7.68 (m, 3H), 7.06-6.92 (m, 4H), 4.80-4.64 (m, 2H), 4.47-4.18 (m, 1H), 4.13-3.80 (m, 7H), 3.32 (br s, 7H), 3.19-2.91 (m, 4H), 2.84-2.73 (m, 1H), 2.57 (br d, J=4.3 Hz, 5H), 2.11 (dq, J=4.2, 12.7 Hz, 1H), 1.99-1.85 (m, 3H), 1.82-1.70 (m, 2H), 1.68 (br s, 2H), 1.62-1.41 (m, 6H).

### Example 3. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-(4-(3-((2,6-dioxopiperidin-3-yl)carbamoyl)phenyl)piperazin-1-yl)acetyl)piperidin-4-yl)-3-methoxybenzamide (Compound 3)

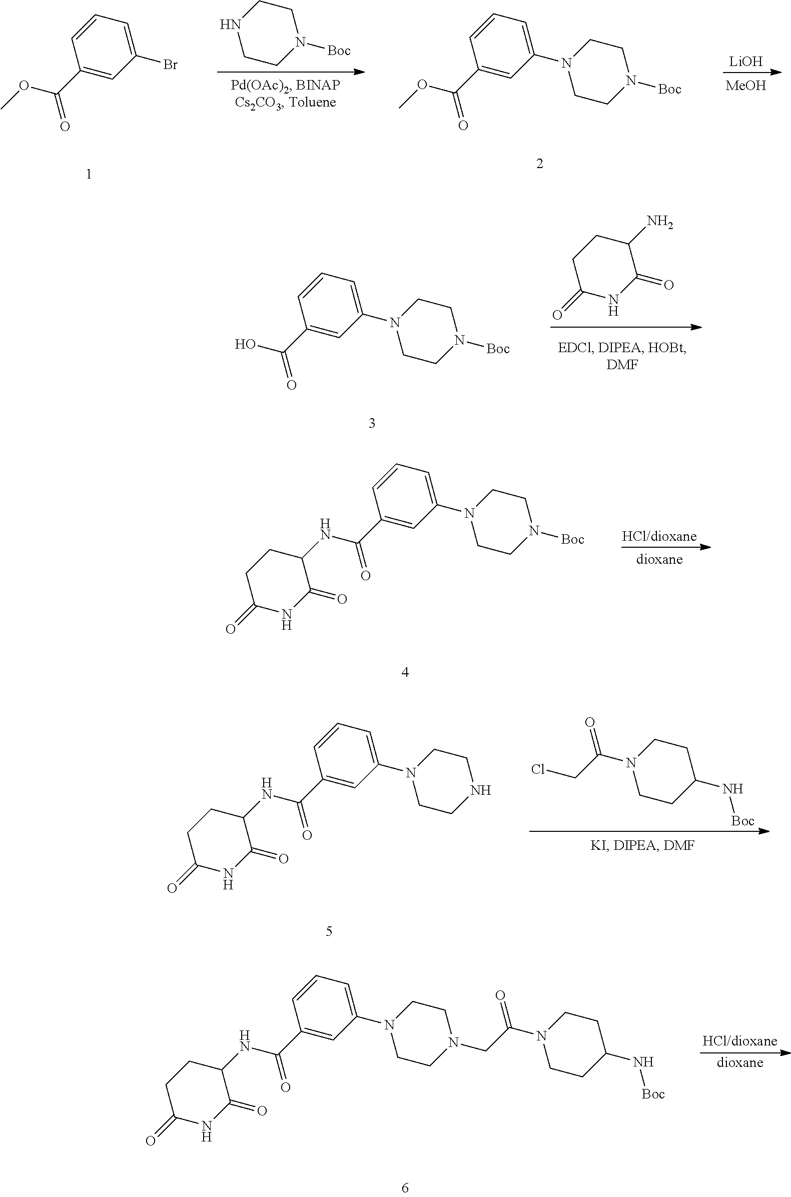

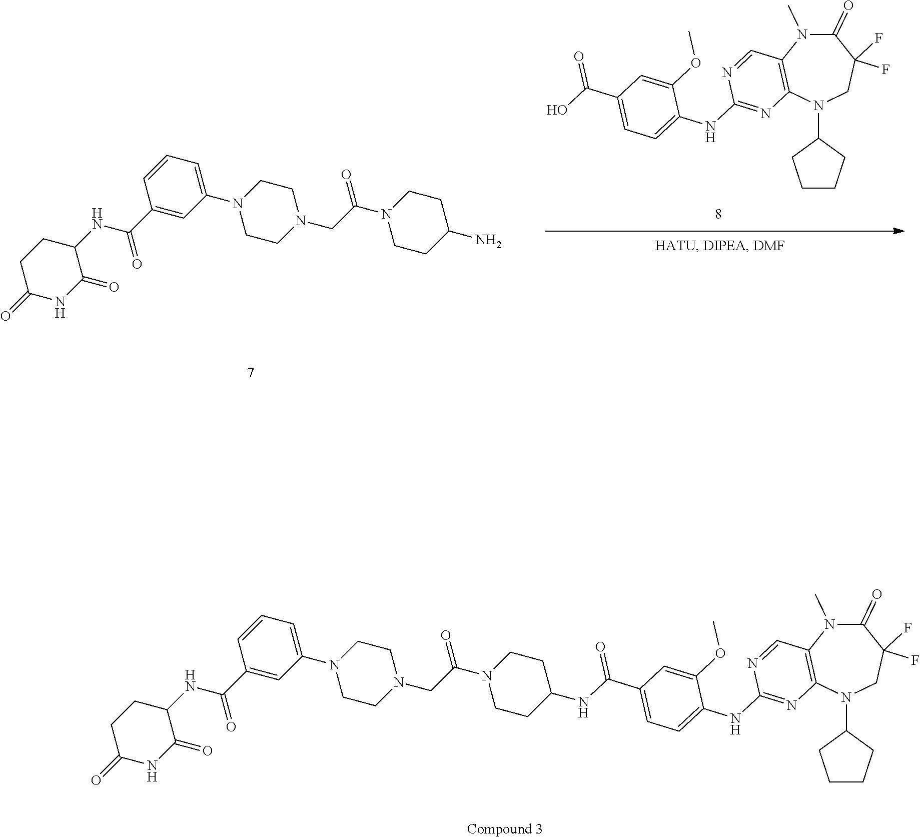 [SMILES: NC1CCN(C(=O)CN2CCN(c3cccc(C(=O)NC4CCC(=O)NC4=O)c3)CC2)CC1 ; COc1cc(C(=O)O)ccc1Nc1ncc2c(n1)N(C1CCCC1)CC(F)(F)C(=O)N2C ; COc1cc(C(=O)NC2CCN(C(=O)CN3CCN(c4cccc(C(=O)NC5CCC(=O)NC5=O)c4)CC3)CC2)ccc1Nc1ncc2c(n1)N(C1CCCC1)CC(F)(F)C(=O)N2C]

### Step 1. Synthesis of tert-butyl 4-(3-(methoxycarbonyl)phenyl)piperazine-1-carboxylate (2)

A mixture of methyl 3-bromobenzoate (2 g, 9.30 mmol), tert-butyl piperazine-1-carboxylate (2.00 g, 10.74 mmol), Pd(OAc)₂ (250.00 mg, 1.11 mmol), BINAP (600.00 mg, 963.59 μmol) and Cs₂CO₃ (6.50 g, 19.95 mmol) in toluene (50 mL) was degassed and purged with N₂ for 3 times, then the mixture was stirred at 100° C. for 16 hr under N₂ atmosphere. LCMS showed a main peak with desired mass. The reaction mixture was filtered and the filtrate was concentrated under reduced pressure. The residue was purified by flash silica gel chromatography (Biotage; 40 g SepaFlash® Silica Flash Column, Eluent of 0˜20% EtOAc:Petroleum ether gradient, 60 mL/min) to afford tert-butyl 4-(3-(methoxycarbonyl)phenyl)piperazine-1-carboxylate (2.4 g, 7.49 mmol, 80.55% yield) as a light yellow oil. MS (M+H)⁺=321.3

### Step 2. Synthesis of 3-(4-(tert-butoxycarbonyl)piperazin-1-yl)benzoic acid (3)

To a solution of tert-butyl 4-(3-(methoxycarbonyl)phenyl)piperazine-1-carboxylate (2.4 g, 7.49 mmol) in MeOH (30 mL) was added a solution of LiOH (500 mg, 20.88 mmol) in H₂O (30 mL) at 20° C. The mixture was stirred at 20° C. for 12 hr. LCMS showed a main peak with desired mass. The reaction mixture was concentrated under reduced pressure. The reaction mixture was diluted with H₂O (30 mL), and adjust pH to 7 with HCl (1M) slowly at 0° C., then the resulting mixture was extracted with EtOAc (60 mL×3), dried over Na₂SO₄, filtered and the filtrate was concentrated under reduced pressure to afford 3-(4-(tert-butoxycarbonyl)piperazin-1-yl)benzoic acid (2.2 g, crude) as a light yellow solid. MS (M−56+H)⁺=251.2

### Step 3. Synthesis of tert-butyl 4-(3-((2,6-dioxopiperidin-3-yl)carbamoyl)phenyl)piperazine-1-carboxylate (4)

To a solution of 3-(4-(tert-butoxycarbonyl)piperazin-1-yl)benzoic acid (2.1 g, 6.85 mmol) in DMF (20 mL) was added EDCI (2.10 g, 10.95 mmol), DIPEA (4.96 g, 38.36 mmol, 6.68 mL), HOBt (1.15 g, 8.48 mmol) and 3-aminopiperidine-2,6-dione (1.40 g, 8.51 mmol, HCl salt) at 25° C. The mixture was stirred at 25° C. for 16 hr. LCMS showed a main peak with the desired mass. The reaction mixture was diluted with H₂O (30 mL), and extracted with EtOAc (80 mL×2). The combined organic layers were washed with brine (60 mL×3), dried over Na₂SO₄, filtered and the filtrate was concentrated under reduced pressure. The crude product was triturated with MTBE (30 mL) at 25° C. for 0.5 hr, filtrated. The filter cake was washed with MTBE (10 mL×2), then dried under reduced pressure to afford tert-butyl 4-(3-((2,6-dioxopiperidin-3-yl)carbamoyl)phenyl)piperazine-1-carboxylate (2.8 g, 6.72 mmol, 98.08% yield) as a white solid. MS (M+H)⁺=417.4

¹H NMR (400 MHz, DMSO-d₆) δ=10.87 (s, 1H), 8.70 (d, J=8.4 Hz, 1H), 7.43 (d, J=1.1 Hz, 1H), 7.38-7.28 (m, 2H), 7.17-7.11 (m, 1H), 4.92-4.65 (m, 1H), 3.54-3.41 (m, 4H), 3.22-3.09 (m, 4H), 2.86-2.76 (m, 1H), 2.59-2.53 (m, 1H), 2.19-2.07 (m, 1H), 2.02-1.94 (m, 1H), 1.43 (s, 9H)

### Step 4. Synthesis of N-(2,6-dioxopiperidin-3-yl)-3-(piperazin-1-yl)benzamide (5)

To a solution of tert-butyl 4-(3-((2,6-dioxopiperidin-3-yl)carbamoyl)phenyl)piperazine-1-carboxylate (2.8 g, 6.72 mmol) in dioxane (30 mL) were added HCl/dioxane (4 M, 30 mL). The mixture was stirred at 25° C. for 48 hr. LCMS showed a main peak (95%) with desired mass. The reaction mixture was concentrated under reduced pressure to afford N-(2,6-dioxopiperidin-3-yl)-3-(piperazin-1-yl)benzamide (2.30 g, crude, HCl salt) as a white solid. MS (M+H)⁺=317.3

¹H NMR (400 MHz, DMSO-d₆) δ=10.86 (s, 1H), 9.48 (br s, 2H), 8.83 (d, J=8.3 Hz, 1H), 7.47 (s, 1H), 7.42-7.33 (m, 2H), 7.21-7.15 (m 1H), 4.80-4.73 (m, 1H), 3.47-3.38 (m, 4H), 3.27-3.15 (m, 4H), 2.86-2.75 (m, 1H), 2.59-2.52 (m, 1H), 2.21-2.08 (m, 1H), 2.01-1.93 (m, 1H).

### Step 5. Synthesis of tert-butyl (1-(2-(4-(3-((2,6-dioxopiperidin-3-yl)carbamoyl)phenyl)piperazin-1-yl)acetyl)piperidin-4-yl)carbamate (6)

To a solution of N-(2,6-dioxopiperidin-3-yl)-3-(piperazin-1-yl)benzamide (450 mg, 1.28 mmol, HCl salt) in DMF (8 mL) were added KI (45.00 mg, 271.08 μmol), DIPEA (890.40 mg, 6.89 mmol, 1.2 mL) and tert-butyl (1-(2-chloroacetyl)piperidin-4-yl)carbamate (560 mg, 2.02 mmol) at 25° C. The mixture was stirred at 60° C. for 16 hr. LCMS showed a main peak with desired mass. The reaction mixture was diluted with H₂O (10 mL), and extracted with EtOAc (40 mL×2). The combined organic layers were washed with brine (30 mL×3), dried over Na₂ SO₄, filtered and the filtrate was concentrated under reduced pressure. The residue was purified by flash silica gel chromatography (Biotage; 10 g SepaFlash® Silica Flash Column, Eluent of 0˜20% Methanol:EtOAc gradient, 60 mL/min) to afford tert-butyl (1-(2-(4-(3-((2,6-dioxopiperidin-3-yl)carbamoyl)phenyl)piperazin-1-yl)acetyl)piperidin-4-yl)carbamate (500 mg, 898.23 μmol, 70.42% yield) as a light yellow solid. MS (M+H)⁺=557.5

¹H NMR (400 MHz, DMSO-d₆) δ=10.87 (s, 1H), 8.69 (d, J=8.3 Hz, 1H), 7.70 (d, J=8.2 Hz, 1H), 7.40 (s, 1H), 7.34-7.25 (m, 2H), 7.15-7.09 (m, 1H), 4.89-4.65 (m, 1H), 3.91-3.74 (m, 3H), 3.23 (br s, 4H), 2.97 (s, 2H), 2.86-2.77 (m, 2H), 2.64-2.51 (m, 6H), 2.21-2.05 (m, 1H), 2.00-1.94 (m, 1H), 1.71-1.63 (m, 2H), 1.39 (s, 9H), 1.37-1.28 (m, 2H).

### Step 6. Synthesis of 3-(4-(2-(4-aminopiperidin-1-yl)-2-oxoethyl)piperazin-1-yl)-N-(2,6-dioxopiperidin-3-yl)benzamide (7)

To a solution of tert-butyl (1-(2-(4-(3-((2,6-dioxopiperidin-3-yl)carbamoyl)phenyl)piperazin-1-yl)acetyl)piperidin-4-yl)carbamate (250 mg, 449.11 μmol) in dioxane (10 mL) were added HCl/dioxane (4 M, 10 mL). The mixture was stirred at 25° C. for 3 hr. LCMS showed a main peak with the desired mass. The reaction mixture was concentrated under reduced pressure to afford 3-(4-(2-(4-aminopiperidin-1-yl)-2-oxoethyl)piperazin-1-yl)-N-(2,6-dioxopiperidin-3-yl)benzamide (200 mg, crude, 2HCl salt) as a light yellow solid. MS (M+H)⁺=457.5

### Step 7. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-(4-(3-((2,6-dioxopiperidin-3-yl)carbamoyl)phenyl)piperazin-1-yl)acetyl)piperidin-4-yl)-3-methoxybenzamide (Compound 3)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (60 mg, 134.10 μmol) in DMF (2 mL) were added HATU (76 mg, 199.88 μmol), DIPEA (89.04 mg, 688.93 μmol, 120 μL) and 3-(4-(2-(4-aminopiperidin-1-yl)-2-oxoethyl)piperazin-1-yl)-N-(2,6-dioxopiperidin-3-yl)benzamide (80 mg, 151.10 μmol, 2HCl salt) at 25° C. The mixture was stirred at 25° C. for 16 hr under N₂ atmosphere. LCMS showed a peak (78%) with desired mass. The mixture was filtered and the filtrate was purified by prep-HPLC (column: Unisil 3-100 C18 Ultra 150×50 mm×3 μm; mobile phase: [water (FA)-ACN]; B %: 11%-41%, 7 min; Column Temp: 30° C.) and re-purified by prep-HPLC (column: Waters Xbridge 150×25 mm×5 μm; mobile phase: [water (NH₄HCO₃)-ACN]; B %: 20%-60%, 9 min; Column Temp: 30° C.), the eluent was lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-(4-(3-((2,6-dioxopiperidin-3-yl)carbamoyl)phenyl)piperazin-1-yl)acetyl)piperidin-4-yl)-3-methoxybenzamide (73.4 mg, 80.36 μmol, 59.93% yield, 97% purity) as a white solid. MS (M+H)⁺=886.7

¹H NMR (400 MHz, DMSO-d₆) δ=10.87 (s, 1H), 8.69 (d, J=8.3 Hz, 1H), 8.24 (s, 1H), 8.15 (d, J=8.1 Hz, 1H), 8.00 (s, 1H), 7.72 (d, J=8.1 Hz, 1H), 7.41 (s, 1H), 7.36-7.27 (m, 2H), 7.16-7.09 (m, 1H), 7.03 (s, 1H), 6.96 (d, J=8.2 Hz, 1H), 4.82-4.67 (m, 2H), 4.40-4.16 (m, 1H), 4.16-4.00 (m, 2H), 4.00-3.90 (m, 2H), 3.89 (s, 3H), 3.33-3.20 (m, 7H), 3.13-2.88 (m, 4H), 2.85-2.76 (m, 1H), 2.66-2.59 (m, 4H), 2.58-2.55 (m, 1H), 2.19-2.06 (m, 1H), 2.05-1.88 (m, 3H), 1.87-1.66 (m, 4H), 1.61-1.44 (m, 6H).

### Example 4. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(4-(4-((2,6-dioxopiperidin-3-yl)carbamoyl)phenyl)piperazin-1-yl)propanoyl)piperidin-4-yl)-3-methoxybenzamide (Compound 4)

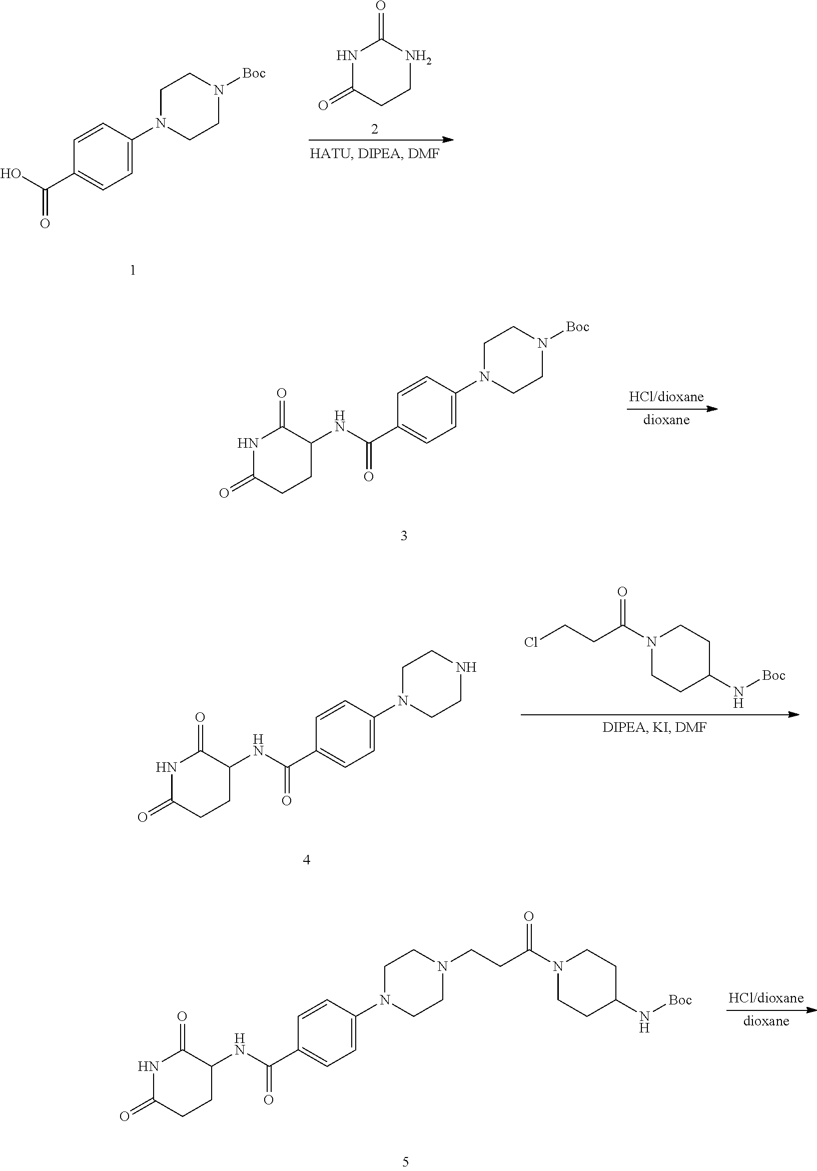 [SMILES: CC(C)(C)OC(=O)N1CCN(c2ccc(C(=O)O)cc2)CC1 ; O=C1CCNC(=O)N1 ; CC(C)(C)OC(=O)N1CCN(c2ccc(C(=O)NC3CCC(=O)NC3=O)cc2)CC1 ; O=C1CCC(NC(=O)c2ccc(N3CCNCC3)cc2)C(=O)N1 ; CC(C)(C)OC(=O)NC1CCN(C(=O)CCCl)CC1 ; CC(C)(C)OC(=O)NC1CCN(C(=O)CCN2CCN(c3ccc(C(=O)NC4CCC(=O)NC4=O)cc3)CC2)CC1]

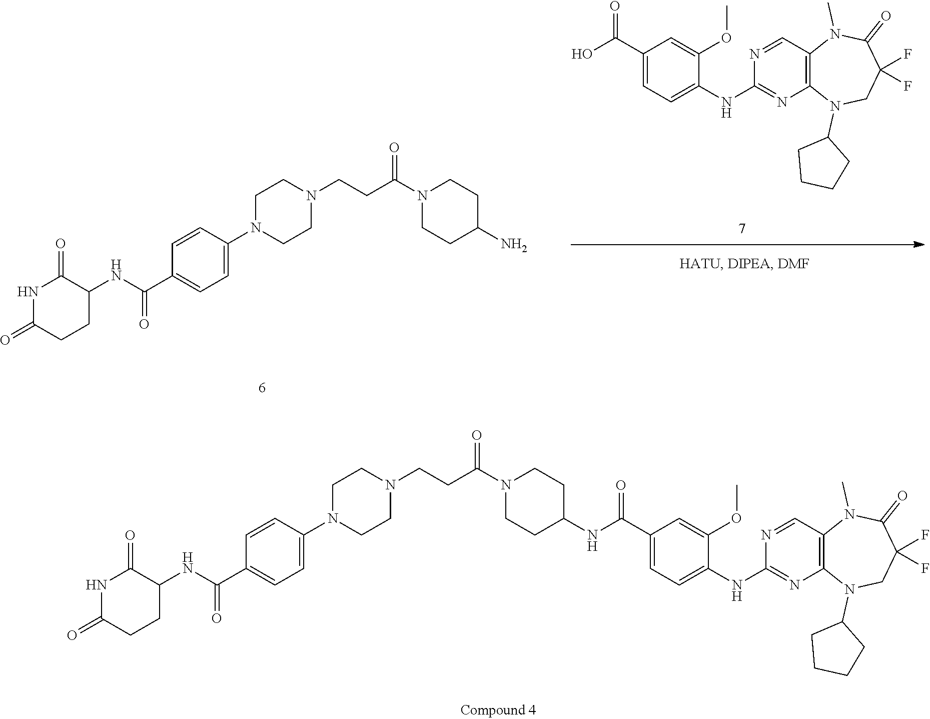 [SMILES: NC1CCN(C(=O)CCN2CCN(c3ccc(C(=O)NC4CCC(=O)NC4=O)cc3)CC2)CC1 ; COc1cc(C(=O)O)ccc1Nc1ncc2c(n1)N(C1CCCC1)CC(F)(F)C(=O)N2C ; COc1cc(C(=O)NC2CCN(C(=O)CCN3CCN(c4ccc(C(=O)NC5CCC(=O)NC5=O)cc4)CC3)CC2)ccc1Nc1ncc2c(n1)N(C1CCCC1)CC(F)(F)C(=O)N2C]

### Step 1. Synthesis of tert-butyl 4-(4-((2,6-dioxopiperidin-3-yl)carbamoyl)phenyl)piperazine-1-carboxylate (3)

To a solution of 4-(4-tert-butoxycarbonylpiperazin-1-yl) benzoic acid (2 g, 6.53 mmol) in DMF (20 mL) were added HATU (3.72 g, 9.79 mmol,) and DIPEA (1.69 g, 13.06 mmol, 2.27 mL) at 25° C. The mixture was stirred at 25° C. for 10 min and a solution of 3-aminopiperidine-2, 6-dione (1.40 g, 8.49 mmol, HCl) in DMF (15 mL) with DIPEA (1.69 g, 13.06 mmol, 2.27 mL) was added. The mixture was stirred at 25° C. for 14 h. TLC (Petroleum ether:EtOAc=3:1) showed the starting material was consumed and new spots were formed. The mixture was diluted with H₂O (30 mL) and EtOAc (10 mL) and extracted with EtOAc (10 mL×3). The combined organic layers were washed with H₂O (20 mL) and concentrated under reduced pressure. The crude was diluted with MTBE (50 mL) and the mixture was stirred at 25° C. for 1 h. The mixture was filtered and the filter cake was washed with MTBE (10 mL). The filter cake was collected and dried under reduced pressure to afford tert-butyl 4-(4-((2,6-dioxopiperidin-3-yl)carbamoyl)phenyl)piperazine-1-carboxylate (2.48 g, 5.84 mmol, 89.39% yield, 98% purity) as a white solid. MS (M+H)⁺=417.3

### Step 2. Synthesis of N-(2,6-dioxopiperidin-3-yl)-4-(piperazin-1-yl)benzamide (4)

To a solution of tert-butyl 4-(4-((2,6-dioxopiperidin-3-yl)carbamoyl)phenyl)piperazine-1-carboxylate (1 g, 2.40 mmol) in dioxane (10 mL) was added HCl/dioxane (4 M, 20 mL) at 25° C. The mixture was stirred at 25° C. for 1 h. LCMS showed the desired mass. The reaction mixture was concentrated under reduced pressure to afford N-(2,6-dioxopiperidin-3-yl)-4-(piperazin-1-yl)benzamide (850 mg, crude, HCl) as a white solid. MS (M+H)⁺=317.1

### Step 3. Synthesis of tert-butyl (1-(3-(4-(4-((2,6-dioxopiperidin-3-yl)carbamoyl)phenyl)piperazin-1-yl)propanoyl) piperidin-4-yl)carbamate (5)

To a solution of N-(2,6-dioxopiperidin-3-yl)-4-(piperazin-1-yl)benzamide (600 mg, 1.70 mmol, HCl) and tert-butyl N-[1-(3-chloropropanoyl)-4-piperidyl]carbamate (716.96 mg, 2.47 mmol) in DMF (8 mL) were added DIPEA (659.37 mg, 5.10 mmol, 888.65 μL) and KI (62.97 mg, 379.33 μmol). The mixture was stirred at 80° C. for 12 h. LCMS showed 26% of N-(2,6-dioxopiperidin-3-yl)-4-(piperazin-1-yl)benzamide remained and 46% peak with desired mass. The mixture was diluted with H₂O (30 mL) and extracted with EtOAc (30 mL×3). The combined organic layers were washed with brine (20 mL×2). The combined organic layers was concentrated under reduced pressure. The residue was purified by flash silica gel chromatography (10 g SepaFlash® Silica Flash Column, Eluent of 50-100% EtOAc/Petroleum ether to 0-10% Methanol/EtOAc gradient@100 mL/min) to afford tert-butyl (1-(3-(4-(4-((2,6-dioxopiperidin-3-yl)carbamoyl)phenyl)piperazin-1-yl)propanoyl)piperidin-4-yl)carbamate (320 mg, 560.74 μmol, 32.97% yield) as a yellow solid. MS (M+H)⁺=571.4

### Step 4. Synthesis of 4-(4-(3-(4-aminopiperidin-1-yl)-3-oxopropyl)piperazin-1-yl)-N-(2,6-dioxopiperidin-3-yl)benzamide (6)

To a solution of tert-butyl (1-(3-(4-(4-((2,6-dioxopiperidin-3-yl)carbamoyl)phenyl)piperazin-1-yl)propanoyl)piperidin-4-yl)carbamate (320 mg, 560.74 μmol) in dioxane (3 mL) was added HCl/dioxane (4 M, 9 mL) at 25° C. The reaction mixture was stirred at 25° C. for 1 h. LCMS showed the starting material was consumed and 91% peak with desired mass. The mixture was concentrated under reduced pressure to afford 4-(4-(3-(4-aminopiperidin-1-yl)-3-oxopropyl)piperazin-1-yl)-N-(2,6-dioxopiperidin-3-yl)benzamide (280 mg, crude, HCl salt) as a yellow solid. MS (M+H)⁺=471.2

### Step 5. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(4-(4-((2,6-dioxopiperidin-3-yl)carbamoyl)phenyl)piperazin-1-yl)propanoyl)piperidin-4-yl)-3-methoxybenzamide (Compound 4)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (70 mg, 156.45 μmol) in DMF (1 mL) were added HATU (89.23 mg, 234.67 μmol) and DIPEA (44.52 mg, 344.47 μmol, 60 μL) and the mixture was stirred at 25° C. for 15 min. Then a solution of 4-(4-(3-(4-aminopiperidin-1-yl)-3-oxopropyl)piperazin-1-yl)-N-(2,6-dioxopiperidin-3-yl)benzamide (100 mg, 197.23 μmol, HCl salt) and DIPEA (44.52 mg, 344.47 μmol, 60 μL) in DMF (1 mL) was added and the mixture was stirred at 25° C. for 1 h. LCMS showed the desired mass was detected. The mixture was diluted with H₂O (10 mL) and extracted with EtOAc (10 mL×3). The combined organic layer was washed with brine (10 mL×3), dried over Na₂SO₄ and filtered. The filtrate was concentrated under reduced pressure. The crude was purified by prep-HPLC (column: Waters Xbridge 150×25 mm×5 μm; mobile phase: [water (NH₄HCO₃)-ACN]; B %: 33%-63%, 9 min) and the eluent was lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(4-(4-((2,6-dioxopiperidin-3-yl)carbamoyl)phenyl)piperazin-1-yl)propanoyl)piperidin-4-yl)-3-methoxybenzamide (22 mg, 22.98 μmol, 14.69% yield, 94% purity) as a white solid. MS (M+H)⁺=900.1

¹H NMR (400 MHz, DMSO-d₆) δ=10.82 (s, 1H), 8.46 (d, J=8.3 Hz, 1H), 8.30-8.25 (m, 2H), 8.15 (br d, J=7.8 Hz, 1H), 7.97 (s, 1H), 7.75 (d, J=8.9 Hz, 2H), 7.51-7.46 (m, 2H), 6.98 (d, J=8.9 Hz, 2H), 4.80-4.70 (m, 2H), 4.43-4.34 (m, 1H), 4.10-3.90 (m, 7H), 3.30 (s, 3H), 3.28-3.24 (m, 4H), 3.18-3.09 (m, 1H), 2.84-2.73 (m, 1H), 2.62-2.53 (m, 11H), 2.17-2.04 (m, 1H), 1.99-1.78 (m, 5H), 1.73-1.66 (m, 1H), 1.65-1.34 (m, 6H).

### Example 5. Synthesis of 4-(4-(3-(4-(4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzamido)piperidin-1-yl)-3-oxopropyl)piperazin-1-yl)-N-(2,6-dioxopiperidin-3-yl)-2-fluorobenzamide (Compound 5)

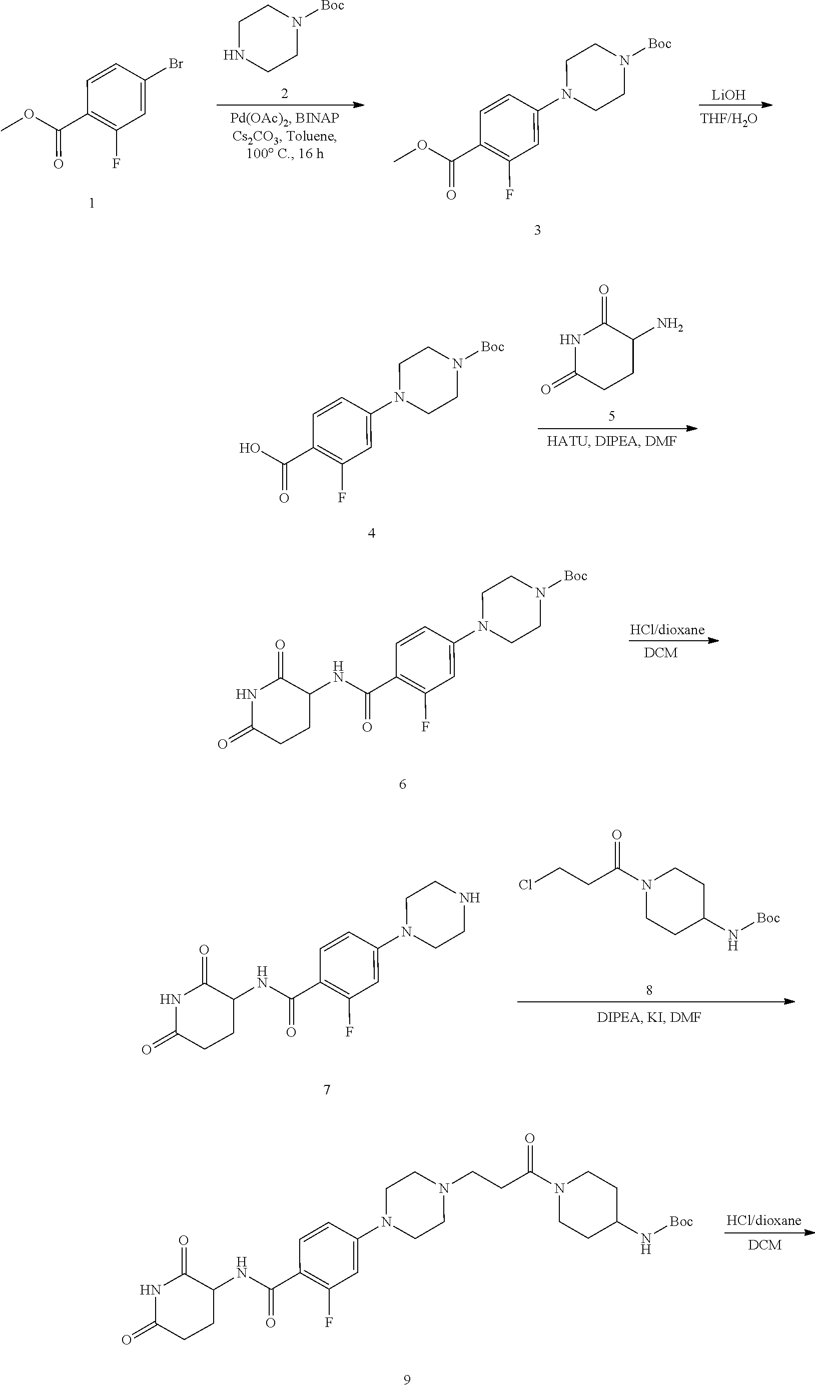

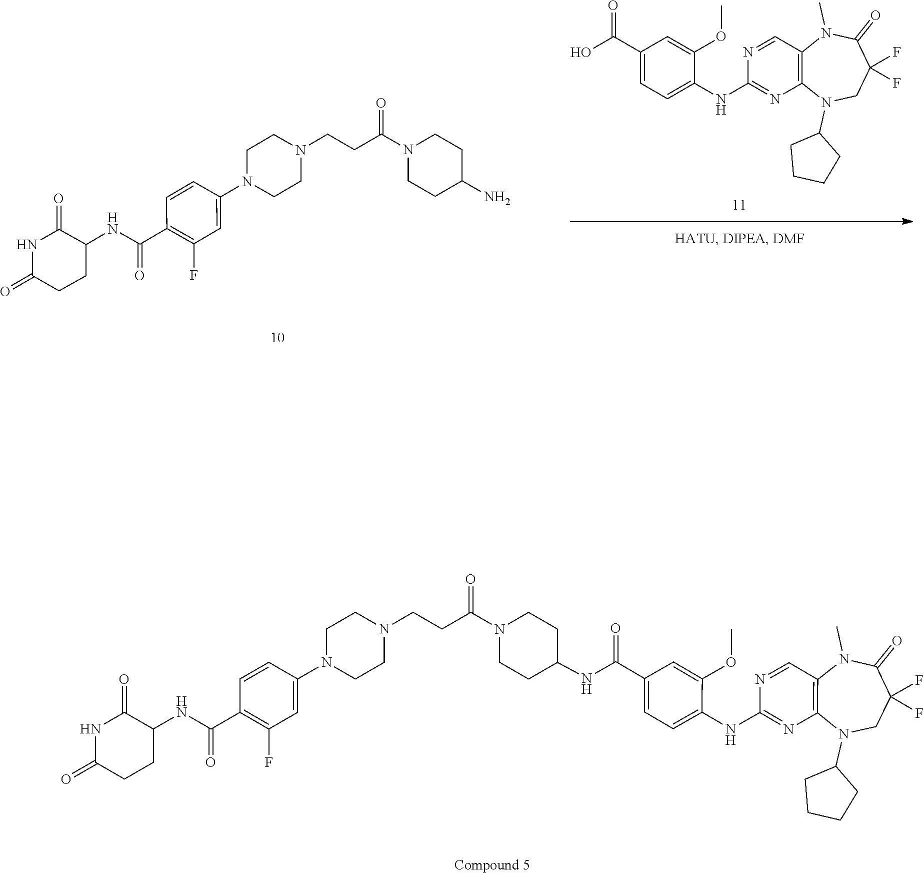 [SMILES: NC1CCN(C(=O)CCN2CCN(c3ccc(C(=O)NC4CCC(=O)NC4=O)c(F)c3)CC2)CC1 ; COc1cc(C(=O)O)ccc1Nc1ncc2c(n1)N(C1CCCC1)CC(F)(F)C(=O)N2C ; COc1cc(C(=O)NC2CCN(C(=O)CCN3CCN(c4ccc(C(=O)NC5CCC(=O)NC5=O)c(F)c4)CC3)CC2)ccc1Nc1ncc2c(n1)N(C1CCCC1)CC(F)(F)C(=O)N2C]

### Step 1. Synthesis of tert-butyl 4-(3-fluoro-4-(methoxycarbonyl)phenyl)piperazine-1-carboxylate (3)

To a solution of methyl 4-bromo-2-fluorobenzoate (1.5 g, 6.44 mmol) and tert-butyl piperazine-1-carboxylate (1.20 g, 6.44 mmol) in toluene (20 mL) were added Pd(OAc)₂ (144.51 mg, 643.68 μmol), BINAP (801.61 mg, 1.29 mmol) and Cs₂CO₃ (6.29 g, 19.31 mmol). The mixture was stirred at 100° C. under N₂ condition for 16 h. LC-MS showed methyl 4-bromo-2-fluorobenzoate was consumed completely and the desired mass. The reaction mixture was concentrated under reduced pressure to remove solvent. The residue was purified by column chromatography (SiO₂, Petroleum ether/EtOAc=20-50%) to afford tert-butyl 4-(3-fluoro-4-(methoxycarbonyl)phenyl)piperazine-1-carboxylate (1.8 g, 3.40 mmol, 52.89% yield, 64% purity) as a white solid. MS (M+H−56)⁺=283.0

### Step 2. Synthesis of 4-(4-(tert-butoxycarbonyl)piperazin-1-yl)-2-fluorobenzoic acid (4)

To a solution of tert-butyl 4-(3-fluoro-4-(methoxycarbonyl)phenyl)piperazine-1-carboxylate (1 g, 2.96 mmol) in THF (8 mL) and H₂O (4 mL) was added LiOH (353.87 mg, 14.78 mmol). The mixture was stirred at 25° C. for 1 h. LC-MS showed one main peak with desired mass. The reaction was adjusted to pH=5 and extracted with EtOAc (20 mL×3). The combined organic layers were washed with brine 20 mL, dried over anhydrous sodium sulfate, filtered and concentrated under reduced pressure to afford 4-(4-(tert-butoxycarbonyl)piperazin-1-yl)-2-fluorobenzoic acid (850 mg, 2.62 mmol, 88.68% yield) as a white solid, which was used directly for next step. MS (M+H−56)+=269.1

### Step 3. Synthesis of tert-butyl 4-(4-((2,6-dioxopiperidin-3-yl)carbamoyl)-3-fluorophenyl) piperazine-1-carboxylate (6)

To a solution of 4-(4-(tert-butoxycarbonyl)piperazin-1-yl)-2-fluorobenzoic acid (400 mg, 1.23 mmol) and 3-aminopiperidine-2,6-dione (158.01 mg, 1.23 mmol) in DMF (7 mL) were added HATU (703.38 mg, 1.85 mmol) and DIPEA (478.17 mg, 3.70 mmol, 644.43 μL). The mixture was stirred at 25° C. for 2 h. LC-MS showed main peak with desired mass. The reaction mixture was diluted with water 20 mL and extracted with EtOAc (10 mL×3). The combined organic layers were washed with brine 10 mL, dried over anhydrous sodium sulfate, filtered and concentrated under reduced pressure to give a residue. The residue was purified by column chromatography (SiO₂, MeOH/EtOAc=0-10%) to afford tert-butyl 4-(4-((2,6-dioxopiperidin-3-yl)carbamoyl)-3-fluorophenyl)piperazine-1-carboxylate (450 mg, 1.02 mmol, 82.31% yield, 98% purity) as a light yellow solid. MS (M+H)⁺=435.3

### Step 4. Synthesis of N-(2,6-dioxopiperidin-3-yl)-2-fluoro-4-(piperazin-1-yl)benzamide (7)

A mixture of tert-butyl 4-(4-((2,6-dioxopiperidin-3-yl)carbamoyl)-3-fluorophenyl)piperazine-1-carboxylate (450 mg, 1.04 mmol) and HCl/dioxane (4 M, 3 mL) in DCM (3 mL) was stirred at 25° C. for 1 h. LC-MS showed the reaction was completed. The reaction mixture was concentrated under reduced pressure to afford N-(2,6-dioxopiperidin-3-yl)-2-fluoro-4-(piperazin-1-yl)benzamide (450 mg, crude, HCl salt) as a light yellow solid, which was used directly for next step. MS (M+H)⁺=335.2

### Step 5. Synthesis of tert-butyl (1-(3-(4-(4-((2,6-dioxopiperidin-3-yl)carbamoyl)-3-fluorophenyl) piperazin-1-yl)propanoyl)piperidin-4-yl)carbamate (9)

To a solution of N-(2,6-dioxopiperidin-3-yl)-2-fluoro-4-(piperazin-1-yl)benzamide (200 mg, 611.16 μmol, HCl salt) and tert-butyl (1-(3-chloropropanoyl)piperidin-4-yl)carbamate (226.62 mg, 677.81 μmol) in DMF (8 mL) was added DIPEA (236.96 mg, 1.83 mmol, 319.36 μL) and KI (101.45 mg, 611.16 μmol). The mixture was stirred at 80° C. for 3 h. LC-MS showed N-(2,6-dioxopiperidin-3-yl)-2-fluoro-4-(piperazin-1-yl)benzamide was consumed completely and the desired mass. The reaction mixture was diluted with water 20 mL and extracted with EtOAc (10 mL×3). The combined organic layers were washed with brine 10 mL, dried over anhydrous sodium sulfate, filtered and concentrated under reduced pressure to give a residue. The residue was purified by column chromatography (SiO₂, MeOH/EtOAc=0-10%) to afford tert-butyl (1-(3-(4-(4-((2,6-dioxopiperidin-3-yl)carbamoyl)-3-fluorophenyl)piperazin-1-yl)propanoyl)piperidin-4-yl)carbamate (130 mg, 172.25 μmol, 28.18% yield, 78% purity) as a light yellow solid. MS (M+H)⁺=589.2

### Step 6. Synthesis of 4-(4-(3-(4-aminopiperidin-1-yl)-3-oxopropyl)piperazin-1-yl)-N-(2,6-dioxopiperidin-3-yl)-2-fluorobenzamide (10)

A mixture of tert-butyl (1-(3-(4-(4-((2,6-dioxopiperidin-3-yl)carbamoyl)-3-fluorophenyl)piperazin-1-yl)propanoyl)piperidin-4-yl)carbamate (130 mg, 220.84 μmol) and HCl/dioxane (4 M, 2 mL) in DCM (2 mL) was stirred at 25° C. for 0.5 h. LC-MS showed the reaction was completed. The reaction mixture was concentrated under reduced pressure to afford 4-(4-(3-(4-aminopiperidin-1-yl)-3-oxopropyl)piperazin-1-yl)-N-(2,6-dioxopiperidin-3-yl)-2-fluorobenzamide (130 mg, crude, HCl salt) as a light yellow solid, which was used directly for next step. MS (M+H)⁺=489.3

### Step 7. Synthesis of 4-(4-(3-(4-(4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzamido)piperidin-1-yl)-3-oxopropyl)piperazin-1-yl)-N-(2,6-dioxopiperidin-3-yl)-2-fluorobenzamide (Compound 5)

To a solution of 4-(4-(3-(4-aminopiperidin-1-yl)-3-oxopropyl)piperazin-1-yl)-N-(2,6-dioxopiperidin-3-yl)-2-fluorobenzamide (130 mg, 247.61 μmol, HCl salt) and 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (110.79 mg, 247.61 μmol) in DMF (4 mL) were added HATU (141.22 mg, 371.42 μmol) and DIPEA (96.01 mg, 742.84 mol, 129.39 L). The mixture was stirred at 25° C. for 2 h. LC-MS showed 4-(4-(3-(4-aminopiperidin-1-yl)-3-oxopropyl)piperazin-1-yl)-N-(2,6-dioxopiperidin-3-yl)-2-fluorobenzamide was consumed completely and one main peak with desired mass. The reaction mixture was diluted with water 20 mL and extracted with EtOAc (10 mL×3). The combined organic layers were washed with brine 10 mL, dried over anhydrous sodium sulfate, filtered and concentrated under reduced pressure to give a residue. The residue was purified by column chromatography (SiO₂, MeOH/EtOAc=0-10%). Then the residue was purified by prep-HPLC (neutral condition: column: Waters Xbridge 150×25 mm×5 um; mobile phase: [water (NH₄HCO₃)-ACN]; B %: 36%-66%, 10 min) and the eluent was lyophilized to afford 4-(4-(3-(4-(4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzamido)piperidin-1-yl)-3-oxopropyl)piperazin-1-yl)-N-(2,6-dioxopiperidin-3-yl)-2-fluorobenzamide (53.3 mg, 56.90 mol, 22.98% yield, 98% purity) as a white solid. MS (M+H)⁺=918.4

¹H NMR (400 MHz, DMSO-d₆) δ=10.85 (s, 1H), 8.33-8.25 (m, 2H), 8.16 (d, J=7.8 Hz, 1H), 8.08-8.01 (m, 1H), 7.98 (s, 1H), 7.64 (t, J=9.0 Hz, 1H), 7.53-7.46 (m, 2H), 6.87-6.74 (m, 2H), 4.81-4.69 (m, 2H), 4.41-4.32 (m, 1H), 4.07-4.03 (m, 3H), 4.02-3.96 (m, 1H), 3.94 (s, 3H), 3.34 (s, 3H), 3.32-3.28 (m, 4H), 3.15-3.06 (m, 1H), 2.83-2.73 (m, 1H), 2.64-2.53 (m, 11H), 2.13-2.06 (m, 1H), 2.05-1.80 (m, 5H), 1.77-1.66 (m, 1H), 1.65-1.56 (m, 4H), 1.53-1.36 (m, 2H).

### Example 6. Synthesis of 3-(4-(3-(4-(4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzamido)piperidin-1-yl)-3-oxopropyl)piperazin-1-yl)-N-(2,6-dioxopiperidin-3-yl)-2-fluorobenzamide (Compound 6)

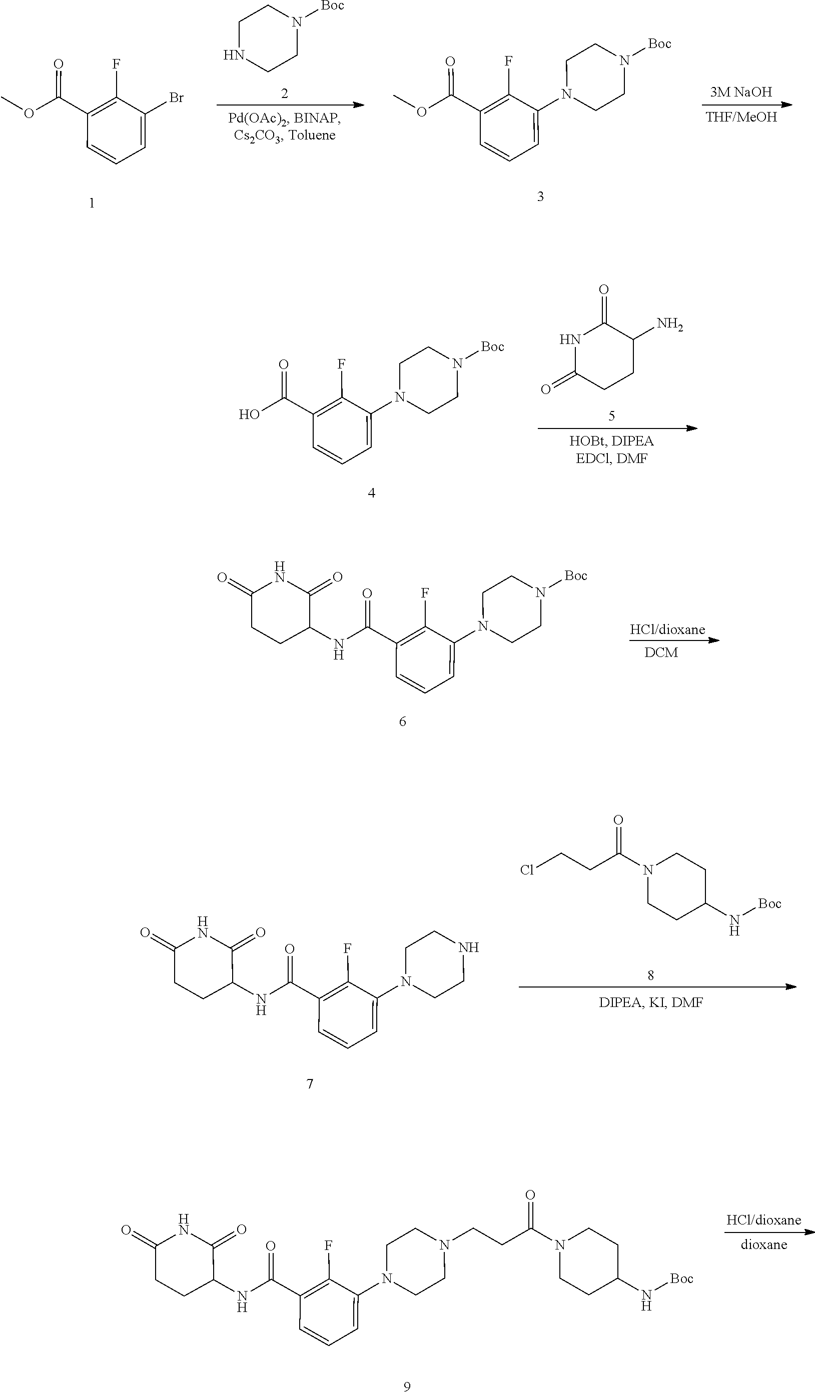

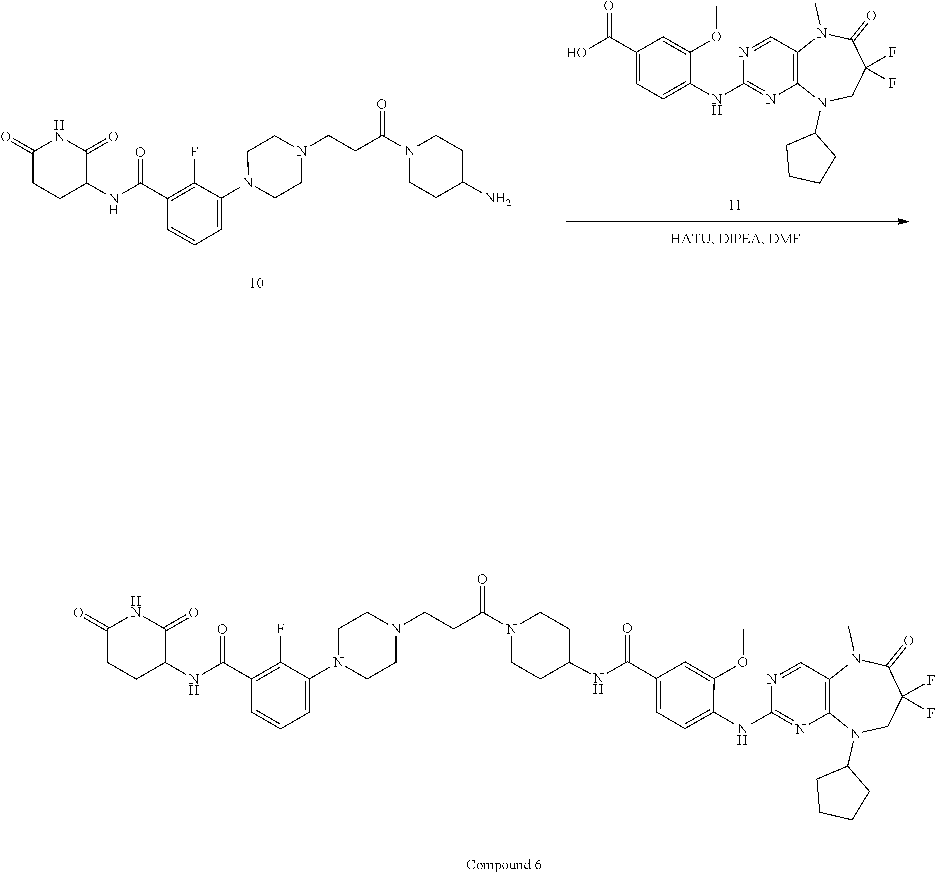 [SMILES: NC1CCN(C(=O)CCN2CCN(c3cccc(C(=O)NC4CCC(=O)NC4=O)c3F)CC2)CC1 ; COc1cc(C(=O)O)ccc1Nc1ncc2c(n1)N(C1CCCC1)CC(F)(F)C(=O)N2C ; COc1cc(C(=O)NC2CCN(C(=O)CCN3CCN(c4cccc(C(=O)NC5CCC(=O)NC5=O)c4F)CC3)CC2)ccc1Nc1ncc2c(n1)N(C1CCCC1)CC(F)(F)C(=O)N2C]

### Step 1. Synthesis of tert-butyl 4-(2-fluoro-3-(methoxycarbonyl)phenyl)piperazine-1-carboxylate (3)

To a solution of methyl 3-bromo-2-fluorobenzoate (1 g, 4.29 mmol) and tert-butyl piperazine-1-carboxylate (879 mg, 4.72 mmol) in toluene (15 mL) were added Cs₂CO₃ (4.19 g, 12.87 mmol), BINAP (267.20 mg, 429.12 μmol) and Pd(OAc)₂ (48.17 mg, 214.56 μmol) and the mixture was stirred at 100° C. for 14 h. LCMS showed the starting material was consumed and 76% peak with desired mass was detected after work up. The mixture was concentrated under reduced pressure to give a residue. The residue was purified by flash silica gel chromatography (10 g SepaFlash® Silica Flash Column, Eluent of 0˜20% EtOAc/Petroleum ether @50 mL/min) to afford tert-butyl 4-(2-fluoro-3-(methoxycarbonyl)phenyl)piperazine-1-carboxylate (0.97 g, 2.18 mmol, 50.77% yield, 76% purity) as a white solid. MS (M+H)⁺=338.4

### Step 2. Synthesis of 3-(4-(tert-butoxycarbonyl)piperazin-1-yl)-2-fluorobenzoic acid (4)

To a solution of tert-butyl 4-(2-fluoro-3-(methoxycarbonyl)phenyl)piperazine-1-carboxylate (0.97 g, 2.87 mmol) in MeOH (10 mL) and THF (10 mL) was added NaOH (3 M, 1.43 mL). The mixture was stirred at 25° C. for 14 h. LCMS showed the starting material was consumed and 85% peak with desired mass was detected. The mixture was concentrated under reduced pressure to afford 3-(4-(tert-butoxycarbonyl)piperazin-1-yl)-2-fluorobenzoic acid (1 g, crude) as a white solid. MS (M+H)⁺=347.1

### Step 3. Synthesis of tert-butyl 4-(3-((2,6-dioxopiperidin-3-yl)carbamoyl)-2-fluorophenyl)piperazine-1-carboxylate (6)

To a solution of 3-(4-(tert-butoxycarbonyl)piperazin-1-yl)-2-fluorobenzoic acid (1 g, 2.89 mmol) and 3-aminopiperidine-2,6-dione (570.29 mg, 3.46 mmol, HCl) and EDCI (830.29 mg, 4.33 mmol) and HOBt (780.32 mg, 5.77 mmol) in DMF (10 mL) was added DIPEA (1.12 g, 8.66 mmol, 1.51 mL) and the mixture was stirred at 25° C. for 14 h. LCMS showed the starting material was consumed and 88% peak with desired mass was detected. The mixture was diluted with H₂O (20 mL) and extracted with EtOAc (30 mL×3), the combined organic layers were washed with brine (20 mL), dried over Na₂SO₄ and filtered. The filtrate was concentrated under reduced pressure. The residue was purified by flash silica gel chromatography (10 g SepaFlash® Silica Flash Column, Eluent of 0˜50% EtOAc/Petroleum ether @50 mL/min) to afford tert-butyl 4-(3-((2,6-dioxopiperidin-3-yl)carbamoyl)-2-fluorophenyl)piperazine-1-carboxylate (1.06 g, 2.44 mmol, 84.50% yield, 100% purity) as a white solid. MS (M+H)⁺=434.8

### Step 4. Synthesis of N-(2,6-dioxopiperidin-3-yl)-2-fluoro-3-(piperazin-1-yl)benzamide (7)

To a solution of tert-butyl 4-(3-((2,6-dioxopiperidin-3-yl)carbamoyl)-2-fluorophenyl)piperazine-1-carboxylate (1 g, 2.30 mmol) in dioxane (10 mL) was added HCl/dioxane (4 M, 9 mL) and the mixture was stirred at 25° C. for 1 h. LCMS showed the starting material was consumed and 96% peak with desired mass. The mixture was concentrated under reduced pressure to afford N-(2,6-dioxopiperidin-3-yl)-2-fluoro-3-(piperazin-1-yl)benzamide (910 mg, crude, HCl) as a white solid. MS (M+H)⁺=335.0

### Step 5. Synthesis of tert-butyl (1-(3-(4-(3-((2,6-dioxopiperidin-3-yl)carbamoyl)-2-fluorophenyl)piperazin-1-yl)propanoyl)piperidin-4-yl)carbamate (9)

To a solution of N-(2,6-dioxopiperidin-3-yl)-2-fluoro-3-(piperazin-1-yl)benzamide (910 mg, crude, HCl) and tert-butyl (1-(3-chloropropanoyl)piperidin-4-yl)carbamate (927.71 mg, 3.19 mmol) in DMF (10 mL) were added DIPEA (964.60 mg, 7.46 mmol, 1.30 mL) and KI (20.40 mg, 122.89 μmol). The mixture was stirred at 80° C. for 14 h. LCMS showed the starting material was consumed. The mixture was diluted with H₂O (15 mL) and extracted with EtOAc (25 mL×3), the combined organic layers were washed with brine (30 mL), dried over Na₂SO₄ and filtered. The filtrate was concentrated under reduced pressure. The residue was purified by flash silica gel chromatography (10 g SepaFlash® Silica Flash Column, Eluent of 0˜100% EtOAc/Petroleum ether to 0-30% Methanol/EtOAc gradient@100 mL/min) to afford tert-butyl (1-(3-(4-(3-((2,6-dioxopiperidin-3-yl)carbamoyl)-2-fluorophenyl)piperazin-1-yl)propanoyl)piperidin-4-yl)carbamate (0.53 g, 900.34 μmol, 36.69% yield) as a brown solid. MS (M+H)⁺=589.3

### Step 6. Synthesis of 3-(4-(3-(4-aminopiperidin-1-yl)-3-oxopropyl)piperazin-1-yl)-N-(2,6-dioxopiperidin-3-yl)-2-fluorobenzamide (10)

To a solution of tert-butyl (1-(3-(4-(3-((2,6-dioxopiperidin-3-yl)carbamoyl)-2-fluorophenyl)piperazin-1-yl)propanoyl)piperidin-4-yl)carbamate (0.53 g, 900.34 μmol) in dioxane (5 mL) was added HCl/dioxane (4 M, 8 mL). The mixture was stirred at 25° C. for 1 h. LCMS showed the starting material was consumed and 96% peak with desired mass was detected. The mixture was concentrated under reduced pressure to afford 3-(4-(3-(4-aminopiperidin-1-yl)-3-oxopropyl)piperazin-1-yl)-N-(2,6-dioxopiperidin-3-yl)-2-fluorobenzamide (470 mg, crude, HCl) as a brown solid. MS (M+H)⁺=489.2

### Step 7. Synthesis of 3-(4-(3-(4-(4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzamido)piperidin-1-yl)-3-oxopropyl)piperazin-1-yl)-N-(2,6-dioxopiperidin-3-yl)-2-fluorobenzamide (Compound 6)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (60 mg, 134.10 μmol) in DMF (0.8 mL) were added HATU (76 mg, 199.88 μmol) and DIPEA (14.84 mg, 114.82 μmol, 20 μL) and the mixture was stirred at 25° C. for 15 min. Then a solution of 3-(4-(3-(4-aminopiperidin-1-yl)-3-oxopropyl)piperazin-1-yl)-N-(2,6-dioxopiperidin-3-yl)-2-fluorobenzamide (120 mg, crude, HCl) and DIPEA (14.84 mg, 114.82 μmol, 20 μL) in DMF (0.7 mL) was added and the mixture was stirred at 25° C. for 1 h. LCMS showed 94% of the desired mass was detected after work up. The mixture was diluted with H₂O (10 mL) and extracted with EtOAc (10 mL×3), the combined organic layer was washed with brine (10 mL×3), dried over Na₂SO₄ and filtered. The filtrate was concentrated under reduced pressure. The residue was purified by prep-HPLC (column: Waters Xbridge 150*25 mm*5 m; mobile phase: [water (NH₄HCO₃)-ACN]; B %: 34%-64%, 10 min) and the eluent was lyophilized to afford 3-(4-(3-(4-(4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzamido)piperidin-1-yl)-3-oxopropyl)piperazin-1-yl)-N-(2,6-dioxopiperidin-3-yl)-2-fluorobenzamide (35.2 mg, 35.28 mol, 26.31% yield, 92% purity) as a white solid. MS (M+H)⁺=918.2

¹H NMR (400 MHz, DMSO-d₆) δ=10.86 (s, 1H), 8.62-8.56 (m, 1H), 8.31-8.24 (m, 2H), 8.15 (d, J=7.6 Hz, 1H), 7.97 (s, 1H), 7.51-7.46 (m, 2H), 7.20-7.11 (m, 3H), 4.82-4.71 (m, 2H), 4.44-4.35 (m, 1H), 4.10-4.00 (m, 3H), 3.98-3.91 (m, 4H), 3.31-3.28 (s, 3H), 3.17-3.09 (m, 1H), 3.07-2.97 (m, 4H), 2.84-2.74 (m, 1H), 2.71-2.53 (m, 11H), 2.12-2.02 (m, 1H), 2.01-1.80 (m, 4H), 1.75-1.66 (m, 2H), 1.65-1.53 (m, 4H), 1.52-1.33 (m, 2H).

### Example 7. Synthesis of 5-(4-(3-(4-(4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzamido)piperidin-1-yl)-3-oxopropyl)piperazin-1-yl)-N-(2,6-dioxopiperidin-3-yl)picolinamide (Compound 7)

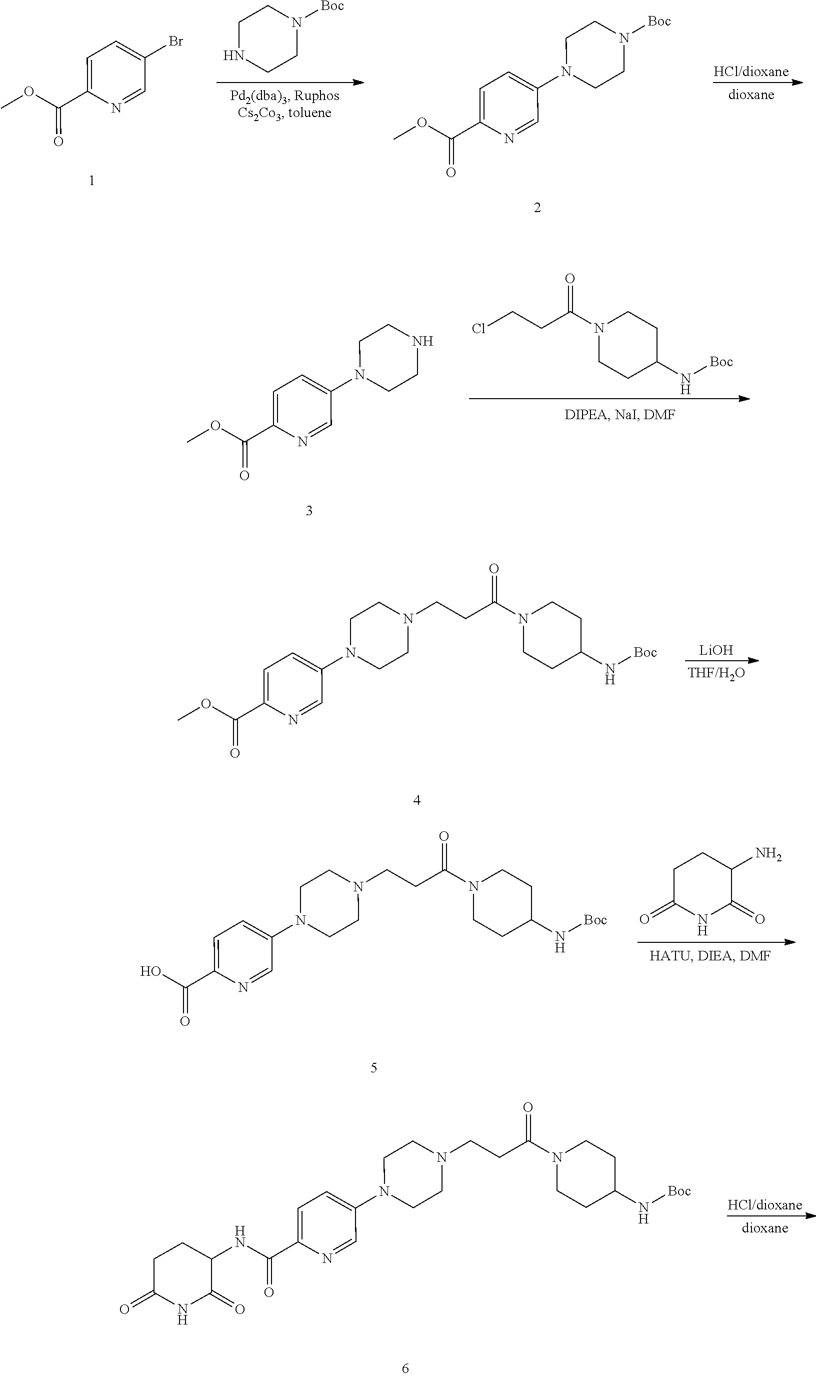

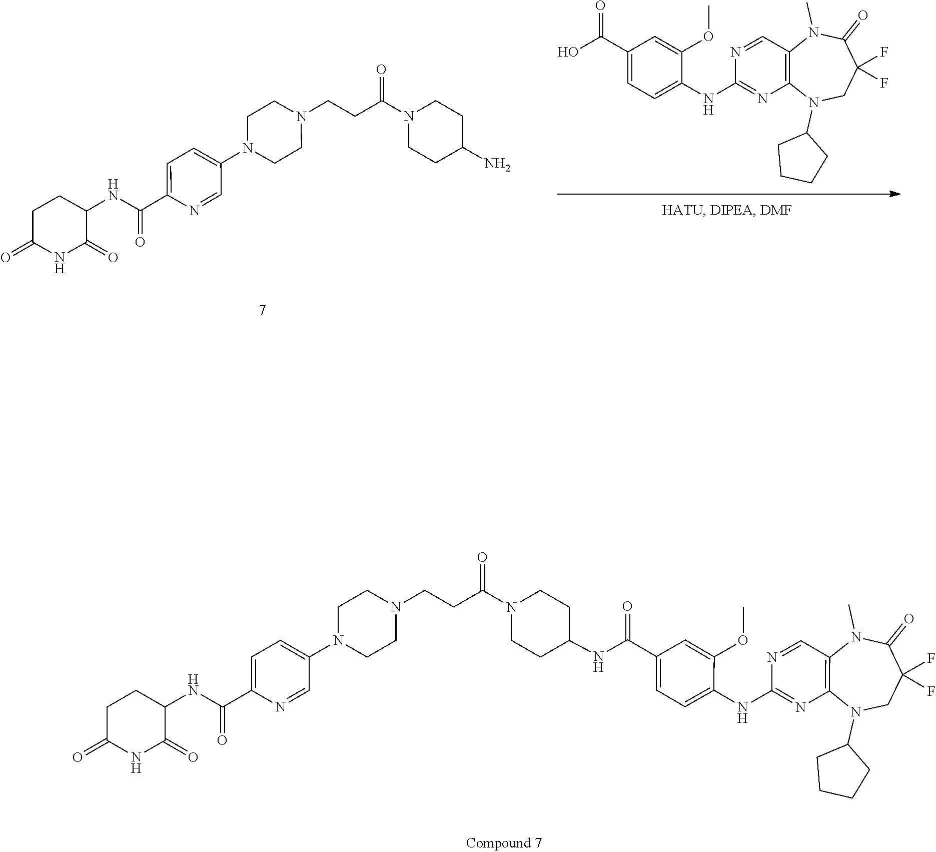 [SMILES: NC1CCN(C(=O)CCN2CCN(c3ccc(C(=O)NC4CCC(=O)NC4=O)nc3)CC2)CC1 ; COc1cc(C(=O)O)ccc1Nc1ncc2c(n1)N(C1CCCC1)CC(F)(F)C(=O)N2C ; COc1cc(C(=O)NC2CCN(C(=O)CCN3CCN(c4ccc(C(=O)NC5CCC(=O)NC5=O)nc4)CC3)CC2)ccc1Nc1ncc2c(n1)N(C1CCCC1)CC(F)(F)C(=O)N2C]

### Step 1. Synthesis of tert-butyl 4-(6-(methoxycarbonyl)pyridin-3-yl)piperazine-1-carboxylate (2)

To a solution of methyl 5-bromopicolinate (1 g, 4.63 mmol) and tert-butyl piperazine-1-carboxylate (862.14 mg, 4.63 mmol) in toluene (20 mL) were added Pd₂ (dba)₃ (105.97 mg, 115.72 μmol), RuPhos (216.00 mg, 462.89 μmol) and Cs₂CO₃ (4.52 g, 13.89 mmol) under N₂, the mixture was stirred at 100° C. for 16 hrs. LCMS showed a major peak with desired mass. The mixture was filtered and the filtrate was concentrated under vacuum to give tert-butyl 4-(6-(methoxycarbonyl)pyridin-3-yl)piperazine-1-carboxylate (1.5 g, crude) as a yellow solid, which was used for the next step directly. MS (M+H)⁺=322.0

### Step 2. Synthesis of methyl 5-(piperazin-1-yl)picolinate (3)

To a solution of tert-butyl 4-(6-(methoxycarbonyl)pyridin-3-yl)piperazine-1-carboxylate (1 g, 3.11 mmol) in dioxane (6 mL) was added HCl/dioxane (4 M, 6 mL), the mixture was stirred at 25° C. for 1 hr. LCMS showed the starting material was consumed completely and the desired mass. The mixture was filtered, the filter cake was diluted with MeOH (10 mL) and concentrated to afford methyl 5-(piperazin-1-yl)picolinate (1 g, crude, HCl) as a yellow solid. MS (M+H)⁺=221.9

### Step 3. Synthesis of methyl 5-(4-(3-(4-((tert-butoxycarbonyl)amino)piperidin-1-yl)-3-oxopropyl) piperazin-1-yl)picolinate (4)

To a solution of methyl 5-(piperazin-1-yl)picolinate (1 g, 3.88 mmol, HCl) and tert-butyl (1-(3-chloropropanoyl)piperidin-4-yl)carbamate (3.38 g, 11.64 mmol) in DMF (20 mL) were added DIPEA (1.50 g, 11.64 mmol, 2.03 mL) and NaI (58.16 mg, 388.02 μmol), the mixture was stirred at 80° C. for 16 hr. LCMS showed desired mass. The mixture was diluted with water (20 mL) and extracted with EtOAc (20 mL×3). The combined organic layer was dried over Na₂SO₄, filtered and concentrated under vacuum. The crude product was purified by flash silica gel chromatography (Biotage, 4 g SepaFlash® Silica Flash Column, Eluent of 4-100% EtOAc/Petroleum ether gradient@45 mL/min) to afford methyl 5-(4-(3-(4-((tert-butoxycarbonyl)amino)piperidin-1-yl)-3-oxopropyl)piperazin-1-yl)picolinate (580 mg, 1.21 mmol, 31.12% yield, 99% purity) as a yellow powder. MS (M+H)⁺=476.4

### Step 4. Synthesis of 5-(4-(3-(4-((tert-butoxycarbonyl)amino)piperidin-1-yl)-3-oxopropyl) piperazin-1-yl)picolinic acid (5)

To a solution of methyl 5-(4-(3-(4-((tert-butoxycarbonyl)amino)piperidin-1-yl)-3-oxopropyl)piperazin-1-yl)picolinate (300 mg, 630.81 μmol) in THF (10 mL) and H₂O (5 mL) was added LiOH (30.21 mg, 1.26 mmol) at 0° C., the mixture was stirred at 25° C. for 4 h. LCMS showed a main peak with desired mass. The mixture was concentrated, followed by lyophilization to afford 5-(4-(3-(4-((tert-butoxycarbonyl)amino)piperidin-1-yl)-3-oxopropyl)piperazin-1-yl)pic olinic acid (280 mg, crude) as yellow powder, used directly. MS (M+H)⁺=462.0

### Step 5. Synthesis of tert-butyl (1-(3-(4-(6-((2,6-dioxopiperidin-3-yl)carbamoyl)pyridin-3-yl) piperazin-1-yl)propanoyl)piperidin-4-yl)carbamate (6)

To a solution of 5-(4-(3-(4-((tert-butoxycarbonyl)amino)piperidin-1-yl)-3-oxopropyl)piperazin-1-yl)pic olinic acid (100 mg, 213.45 μmol Li) in DMF (2 mL) were added HATU (121.74 mg, 320.17 μmol) and DIPEA (82.76 mg, 640.35 μmol, 111.54 L), stirred for 1 min, then 3-aminopiperidine-2,6-dione (42.16 mg, 256.14 μmol, HCl) was added. The mixture was stirred at 25° C. for 2 h. LCMS showed a main peak with desired mass. The mixture was diluted with water (3 mL) and extracted with EtOAc (5 mL×3). The organic layer was dried over Na₂SO₄, filtered and concentrated under vacuum. The crude product was purified by flash silica gel chromatography (Biotage, 4 g SepaFlash® Silica Flash Column, Eluent of 4-50% MeOH/EtOAc gradient@25 mL/min) to afford tert-butyl (1-(3-(4-(6-((2,6-dioxopiperidin-3-yl)carbamoyl)pyridin-3-yl)piperazin-1-yl)propanoyl)piperidin-4-yl)carbamate (110 mg, 153.94 μmol, 72.12% yield, 80% purity) as a yellow powder. MS (M+H)⁺=572.1

### Step 6. Synthesis of 5-(4-(3-(4-aminopiperidin-1-yl)-3-oxopropyl)piperazin-1-yl)-N-(2,6-dioxopiperidin-3-yl)picolinamide (7)

To a solution of tert-butyl (1-(3-(4-(6-((2,6-dioxopiperidin-3-yl)carbamoyl)pyridin-3-yl)piperazin-1-yl)propanoyl)piperidin-4-yl)carbamate (110 mg, 192.42 μmol) in dioxane (4 mL) was added HCl/dioxane (4 M, 4 mL, 83.15), the reaction mixture was stirred at 25° C. for 1 hr. LCMS showed the starting material was consumed completely and the desired mass. The mixture was concentrated under vacuum to afford 5-(4-(3-(4-aminopiperidin-1-yl)-3-oxopropyl)piperazin-1-yl)-N-(2,6-dioxopiperidin-3-yl)picolinamide (110 mg, crude, HCl) as white powder. MS (M+H)⁺=472.0

### Step 7. Synthesis of 5-(4-(3-(4-(4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzamido)piperidin-1-yl)-3-oxopropyl)piperazin-1-yl)-N-(2,6-dioxopiperidin-3-yl)picolinamide (Compound 7)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (60 mg, 134.10 μmol) in DMF (2 mL) were added HATU (76.48 mg, 201.15 μmol) and DIPEA (51.99 mg, 402.29 μmol, 70.07 μL). The mixture was stirred at 25° C. for 1 min, then 5-(4-(3-(4-aminopiperidin-1-yl)-3-oxopropyl)piperazin-1-yl)-N-(2,6-dioxopiperidin-3-yl)picolinamide (68.12 mg, 134.10 μmol, HCl) was added. The reaction mixture was stirred at 25° C. for 1 hr. LCMS showed desired mass. The mixture was diluted with water (3 mL) and extracted with EtOAc (5 mL×3). The organic layer was dried over Na₂SO₄, filtered and concentrated under vacuum. The crude product was purified by Prep-HPLC (column: Phenomenex Synergi Polar-RP 100×25 mm×4 μm; mobile phase: [water(TFA)-ACN]; B %: 28%-48%, 7 min) and Prep-HPLC (column: Waters Xbridge 150×25 mm×5 m; mobile phase: [water(NH₄HCO₃)-ACN]; B %: 25%-58%, 8 min), and then lyophilized to afford 5-(4-(3-(4-(4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzamido)piperidin-1-yl)-3-oxopropyl)piperazin-1-yl)-N-(2,6-dioxopiperidin-3-yl)picolinamide (47.2 mg, 51.55 μmol, 38.44% yield, 98.4% purity) as a white powder. MS (M+H)⁺=901.3

¹H NMR (400 MHz, DMSO-d₆) δ=10.85 (s, 1H), 8.73 (d, J=8.31 Hz, 1H), 8.33 (d, J=2.69 Hz, 1H), 8.26-8.30 (m, 2H), 8.16 (d, J=7.70 Hz, 1H), 7.97 (s, 1H), 7.87 (d, J=8.80 Hz, 1H), 7.47-7.51 (m, 2H), 7.43 (dd, J=8.93, 2.81 Hz, 1H), 4.70-4.82 (m, 2H), 4.40 (d, J=12.23 Hz, 1H), 3.95-4.12 (m, 4H), 3.94 (s, 3H), 3.34-3.37 (m, 4H), 3.31 (s, 3H), 3.14 (t, J=12.29 Hz, 1H), 2.74-2.84 (m, 1H), 2.66-2.74 (m, 1H), 2.53-2.66 (m, 10H), 2.12-2.19 (m, 1H), 1.87-2.06 (m, 4H), 1.72-1.85 (m, 2H), 1.61-1.70 (m, 2H), 1.49-1.58 (m, 2H), 1.36-1.53 (m, 2H).

### Example 8. Synthesis of 4-(4-(3-(4-(4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzamido)piperidin-1-yl)-3-oxopropyl)piperazin-1-yl)-N-(2,6-dioxopiperidin-3-yl)picolinamide (Compound 8)

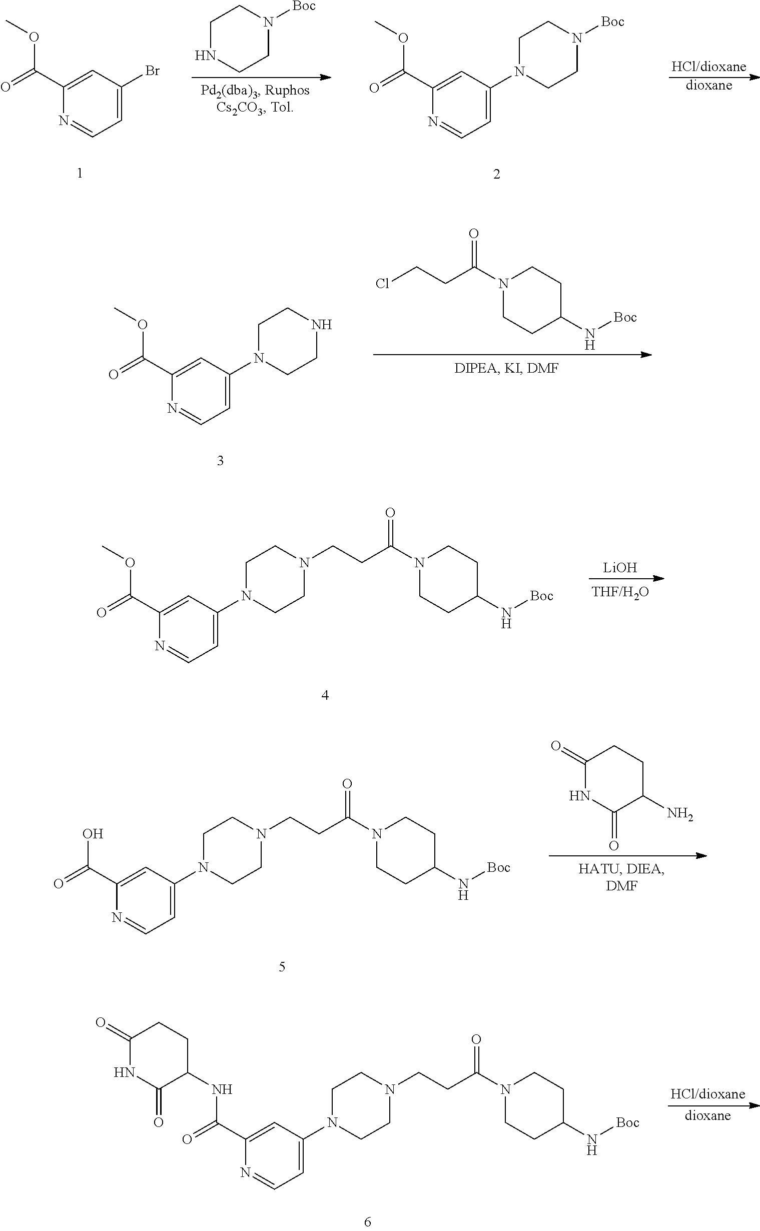

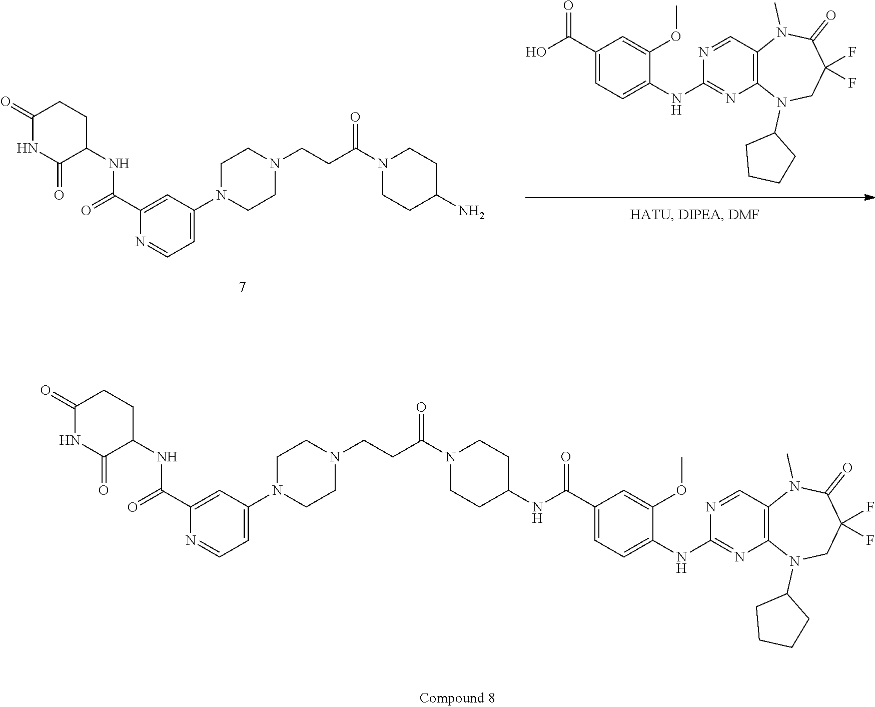 [SMILES: NC1CCN(C(=O)CCN2CCN(c3ccnc(C(=O)NC4CCC(=O)NC4=O)c3)CC2)CC1 ; COc1cc(C(=O)O)ccc1Nc1ncc2c(n1)N(C1CCCC1)CC(F)(F)C(=O)N2C ; COc1cc(C(=O)NC2CCN(C(=O)CCN3CCN(c4ccnc(C(=O)NC5CCC(=O)NC5=O)c4)CC3)CC2)ccc1Nc1ncc2c(n1)N(C1CCCC1)CC(F)(F)C(=O)N2C]

### Step 1. Synthesis of tert-butyl 4-(2-(methoxycarbonyl)pyridin-4-yl)piperazine-1-carboxylate (2)

To a solution of methyl 4-bromopicolinate (1 g, 4.63 mmol) and tert-butyl piperazine-1-carboxylate (862.14 mg, 4.63 mmol) in toluene (20 mL), were added Pd₂ (dba)₃ (105.97 mg, 115.72 μmol), RuPhos (216.00 mg, 462.89 μmol) and Cs₂CO₃ (4.52 g, 13.89 mmol) under N₂, the mixture was stirred at 100° C. for 16 hr. LCMS showed a main peak with desired mass. The mixture was filtered and the filtrate was concentrated under vacuum to afford the crude product. The crude product was purified by flash silica gel chromatography (Biotage, 4 g SepaFlash® Silica Flash Column, Eluent of 4-100% EtOAc/Petroleum ether gradient@40 mL/min) to afford tert-butyl 4-(2-(methoxycarbonyl)pyridin-4-yl)piperazine-1-carboxylate (780 mg, 1.53 mmol, 33.03% yield, 63% purity) as yellow oil. MS (M+H)⁺=322.0

### Step 2. Synthesis of methyl 4-(piperazin-1-yl)picolinate (3)

To a solution of tert-butyl 4-(2-(methoxycarbonyl)pyridin-4-yl)piperazine-1-carboxylate (780 mg, 2.43 mmol) in dioxane (4 mL) was added HCl/dioxane (4 M, 4 mL). The mixture was stirred at 25° C. for 2 hr. LCMS showed desired mass and the starting material consumed completely. The mixture was filtered. The filter cake was concentrated to afford methyl 4-(piperazin-1-yl)picolinate (540 mg, crude, HCl) as a yellow solid. MS (M+H)⁺=221.9

### Step 3. Synthesis of methyl 4-(4-(3-(4-((tert-butoxycarbonyl)amino)piperidin-1-yl)-3-oxopropyl)piperazin-1-yl)picolinate (4)

To a solution of methyl 4-(piperazin-1-yl)picolinate (540 mg, 2.10 mmol, HCl) and tert-butyl (1-(3-chloropropanoyl)piperidin-4-yl)carbamate (1.83 g, 6.29 mmol) in DMF (15 mL) were added DIPEA (812.42 mg, 6.29 mmol, 1.09 mL) and KI (69.57 mg, 419.07 μmol), the mixture was stirred at 80° C. for 16 hr. LCMS showed desired mass. The mixture was diluted with water (3 mL) and extracted with EtOAc (5 mL×3). The organic layer was dried over Na₂SO₄, filtered and concentrated under vacuum to afford the crude product. The crude product was purified by flash silica gel chromatography (Biotage, 4 g SepaFlash® Silica Flash Column, Eluent of 4-90% EtOAc/Petroleum ether gradient@20 mL/min) to afford methyl 4-(4-(3-(4-((tert-butoxycarbonyl)amino)piperidin-1-yl)-3-oxopropyl)piperazin-1-yl)picolinate (150 mg, 315.40 μmol, 15.05% yield) as yellow oil. MS (M+H)⁺=476.1

### Step 4. Synthesis of 4-(4-(3-(4-((tert-butoxycarbonyl)amino)piperidin-1-yl)-3-oxopropyl) piperazin-1-yl)picolinic acid (5)

To a solution of methyl 4-(4-(3-(4-((tert-butoxycarbonyl)amino)piperidin-1-yl)-3-oxopropyl)piperazin-1-yl)picolinate (150 mg, 315.40 μmol) in H₂O (3 mL) and THF (6 mL) was added LiOH (26.47 mg, 630.81 μmol). The mixture was stirred at 25° C. for 4 hr. LCMS showed a main peak with desired mass. The mixture was concentrated and then lyophilized to afford 4-(4-(3-(4-((tert-butoxycarbonyl)amino)piperidin-1-yl)-3-oxopropyl)piperazin-1-yl)pic olinic acid (130 mg, crude) as yellow powder, used directly. MS (M+H)⁺=462.1

### Step 5. Synthesis of tert-butyl (1-(3-(4-(2-((2,6-dioxopiperidin-3-yl)carbamoyl)pyridin-4-yl)piperazin-1-yl)propanoyl)piperidin-4-yl)carbamate (6)

To a solution of 4-(4-(3-(4-((tert-butoxycarbonyl)amino)piperidin-1-yl)-3-oxopropyl)piperazin-1-yl)pic olinic acid (130 mg, 277.48 μmol) in DMF (3 mL) were added HATU (158.26 mg, 416.23 μmol) and DIPEA (107.59 mg, 832.45 μmol, 145.00 L), then 3-aminopiperidine-2,6-dione (54.81 mg, 332.98 μmol, HCl) was added. The mixture was stirred at 25° C. for 16 hr. LCMS showed desired mass. The mixture was diluted with water (3 mL) and extracted with EtOAc (5 mL×3). The organic layer was dried over Na₂SO₄, filtered and concentrated under vacuum to afford tert-butyl (1-(3-(4-(2-((2,6-dioxopiperidin-3-yl)carbamoyl)pyridin-4-yl)piperazin-1-yl)propanoyl)piperidin-4-yl)carbamate (130 mg, crude) as yellow oil. MS (M+H)⁺=572.1

### Step 6. Synthesis of 4-(4-(3-(4-aminopiperidin-1-yl)-3-oxopropyl)piperazin-1-yl)-N-(2,6-dioxopiperidin-3-yl)picolinamide (7)

To a solution of tert-butyl (1-(3-(4-(2-((2,6-dioxopiperidin-3-yl)carbamoyl)pyridin-4-yl)piperazin-1-yl)propanoyl)piperidin-4-yl)carbamate (130 mg, 227.41 μmol) in dioxane (2 mL) was added HCl/dioxane (4 M, 56.85 L), the mixture was stirred at 25° C. for 1 hr. LCMS showed the desired mass. The mixture was concentrated under vacuum to afford 4-(4-(3-(4-aminopiperidin-1-yl)-3-oxopropyl)piperazin-1-yl)-N-(2,6-dioxopiperidin-3-yl)picolinamide (100 mg, crude, HCl) as a yellow powder. MS (M+H)⁺=472.0

### Step 7. Synthesis of 4-(4-(3-(4-(4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzamido)piperidin-1-yl)-3-oxopropyl)piperazin-1-yl)-N-(2,6-dioxopiperidin-3-yl)picolinamide (Compound 8)

To a solution of 4-[(9-cyclopentyl-7,7-difluoro-5-methyl-6-oxo-8H-pyrimido[4,5-b][1,4]diazepin-2-yl) amino]-3-methoxy-benzoic acid (85 mg, 189.97 μmol) in DMF (2 mL) were added HATU (108.35 mg, 284.96 μmol) and DIPEA (73.66 mg, 569.92 μmol, 99.27 μL), then 4-(4-(3-(4-aminopiperidin-1-yl)-3-oxopropyl)piperazin-1-yl)-N-(2,6-dioxopiperidin-3-yl)picolinamide (96.51 mg, 189.97 μmol, HCl) was added. The mixture was stirred at 25° C. for 16 hr. LCMS showed a main peak with desired mass. The mixture was diluted with water (3 mL) and extracted with EtOAc (5 mL×3). The organic layer was dried over Na₂SO₄, filtered and concentrated under vacuum. The crude product was purified by Prep-HPLC (column: Phenomenex Synergi Polar-RP 100×25 mm×4 m; mobile phase: [water(TFA)-ACN]; B %: 23%-43%, 7 min) and Prep-HPLC (column: Waters Xbridge 150×25 mm×5 m; mobile phase: [water(NH₄HCO₃)-ACN]; B %: 30%-60%, 8 min) and then lyophilized to afford 4-(4-(3-(4-(4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzamido)piperidin-1-yl)-3-oxopropyl)piperazin-1-yl)-N-(2,6-dioxopiperidin-3-yl)picolinamide (32.9 mg, 36.33 μmol, 19.13% yield, 99.5% purity) as a white powder. MS (M+H)⁺=901.0

¹H NMR (400 MHz, DMSO-d₆) δ=10.86 (s, 1H), 8.96 (d, J=8.31 Hz, 1H), 8.27-8.32 (m, 2H), 8.24 (d, J=5.99 Hz, 1H), 8.16 (d, J=7.46 Hz, 1H), 7.98 (s, 1H), 7.46-7.52 (m, 3H), 7.04 (dd, J=5.99, 2.57 Hz, 1H), 4.71-4.82 (m, 2H), 4.41 (d, J=13.20 Hz, 1H), 3.95-4.13 (m, 4H), 3.95 (s, 3H), 3.40 (d, J=4.65 Hz, 4H), 3.34 (s, 3H), 3.06-3.20 (m, 1H), 2.75-2.86 (m, 1H), 2.69 (dd, J=3.61, 1.65 Hz, 1H), 2.53-2.67 (m, 10H), 2.14-2.26 (m, 1H), 1.87-2.06 (m, 4H), 1.82-1.72 (m, 2H), 1.62-1.69 (m, 2H), 1.60-1.68 (m, 2H), 1.34-1.53 (m, 2H).

### Example 9. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(4-((4-(3-((2,6-dioxopiperidin-3-yl)carbamoyl)phenyl)piperazin-1-yl)methyl)piperidin-1-yl)-3-methoxybenzamide (Compound 9)

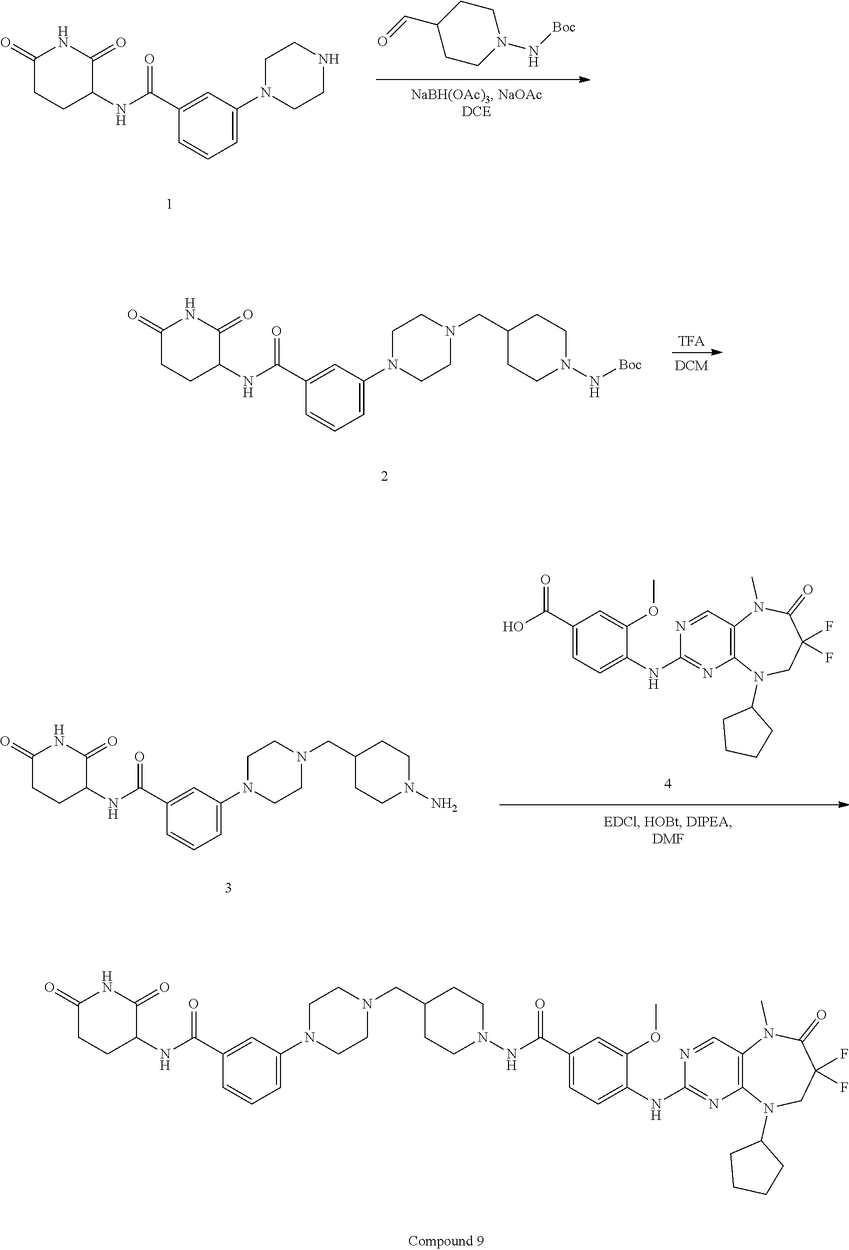 [SMILES: O=C1CCC(NC(=O)c2cccc(N3CCNCC3)c2)C(=O)N1 ; CC(C)(C)OC(=O)NN1CCC(C=O)CC1 ; CC(C)(C)OC(=O)NN1CCC(CN2CCN(c3cccc(C(=O)NC4CCC(=O)NC4=O)c3)CC2)CC1 ; NN1CCC(CN2CCN(c3cccc(C(=O)NC4CCC(=O)NC4=O)c3)CC2)CC1 ; COc1cc(C(=O)O)ccc1Nc1ncc2c(n1)N(C1CCCC1)CC(F)(F)C(=O)N2C ; COc1cc(C(=O)NN2CCC(CN3CCN(c4cccc(C(=O)NC5CCC(=O)NC5=O)c4)CC3)CC2)ccc1Nc1ncc2c(n1)N(C1CCCC1)CC(F)(F)C(=O)N2C]

### Step 1. Synthesis of tert-butyl (4-((4-(3-((2,6-dioxopiperidin-3-yl)carbamoyl)phenyl)piperazin-1-yl)methyl)piperidin-1-yl)carbamate (2)

To a solution of N-(2,6-dioxopiperidin-3-yl)-3-(piperazin-1-yl)benzamide (300 mg, 850.30 μmol, HCl) in DCE (10 mL) was added tert-butyl (4-formylpiperidin-1-yl)carbamate (240 mg, 1.05 mmol) and NaOAc (100 mg, 1.22 mmol), the mixture was stirred at 25° C. for 1 hr, then NaBH(OAc)₃ (700 mg, 3.30 mmol) was added at 25° C. and the resulting mixture was stirred at 25° C. for 15 hr. LCMS showed a peak (78%) with the desired mass. The reaction mixture was diluted with H₂O (10 mL) at 0° C., and adjusted the pH around 9 with saturated NaHCO₃ at 0° C., then extracted with EtOAc 60 mL (20 mL×3). The combined organic layers were washed with brine (30 mL×3), dried over anhydrous Na₂SO₄, filtered. The filtrate was concentrated under reduced pressure. The residue was purified by flash silica gel chromatography (Biotage; 10 g SepaFlash® Silica Flash Column, Eluent of 0˜25% Methanol:EtOAc gradient, 60 mL/min) to afford tert-butyl (4-((4-(3-((2,6-dioxopiperidin-3-yl)carbamoyl)phenyl)piperazin-1-yl)methyl)piperidin-1-yl)carbamate (160 mg, 302.66 μmol, 35.59% yield) as a light yellow solid. MS (M+H)⁺=529.3

¹H NMR (400 MHz, DMSO-d₆) δ=10.89 (s, 1H), 8.70 (d, J=8.4 Hz, 1H), 7.98-7.74 (m, 1H), 7.43-7.34 (m, 1H), 7.32-7.22 (m, 2H), 7.10 (d, J=7.3 Hz, 1H), 4.82-4.73 (m, 1H), 3.56-3.33 (m, 4H), 3.23-3.17 (m, 4H), 2.90-2.83 (m, 2H), 2.82-2.74 (m, 1H), 2.59-2.53 (m, 1H), 2.46-2.41 (m, 2H), 2.18-2.13 (m, 2H), 2.03-1.96 (m, 1H), 1.86-1.85 (m, 1H), 1.73-1.65 (m, 2H), 1.42 (s, 1H), 1.38 (s, 9H), 1.21-1.11 (m, 2H).

### Step 2. Synthesis of 3-(4-((1-aminopiperidin-4-yl)methyl)piperazin-1-yl)-N-(2,6-dioxopiperidin-3-yl)benzamide (3)

To a solution of tert-butyl (4-((4-(3-((2,6-dioxopiperidin-3-yl)carbamoyl)phenyl)piperazin-1-yl)methyl)piperidin-1-yl)carbamate (150 mg, 283.75 μmol) in DCM (6 mL) were added TFA (8 mL). The mixture was stirred at 25° C. for 2 hr. LCMS showed a main peak with the desired mass. The reaction mixture was concentrated under reduced pressure to afford 3-(4-((1-aminopiperidin-4-yl)methyl)piperazin-1-yl)-N-(2,6-dioxopiperidin-3-yl)benzamide (150 mg, crude, 2TFA) as a yellow oil. MS (M+H)⁺=429.2

### Step 3. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(4-((4-(3-((2,6-dioxopiperidin-3-yl)carbamoyl)phenyl)piperazin-1-yl)methyl)piperidin-1-yl)-3-methoxybenzamide (Compound 9)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (60 mg, 134.10 μmol) in DMF (2 mL) were added EDCI (50 mg, 260.82 μmol), HOBt (30 mg, 222.02 μmol), DIPEA (148.40 mg, 1.15 mmol, 200 μL) and 3-(4-((1-aminopiperidin-4-yl)methyl)piperazin-1-yl)-N-(2,6-dioxopiperidin-3-yl)benzamide (100 mg, 152.31 μmol, 2TFA salt) at 25° C. The mixture was stirred at 25° C. for 16 h. LCMS showed a peak (58%) with the desired mass. The mixture was filtered and the filtrate was purified by prep-HPLC (column: Phenomenex Synergi Polar-RP 100×25 mm×4 μm; mobile phase: [water (TFA)-ACN]; B %: 27%-47%, 7 min; Column Temp: 30° C.) and the eluent was lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-N-(4-((4-(3-((2,6-dioxopiperidin-3-yl)carbamoyl)phenyl)piperazin-1-yl)methyl)piperidin-1-yl)-3-methoxybenzamide (45.7 mg, 45.61 μmol, 34.01% yield, 97% purity, TFA salt) as a white solid. MS (M+H)⁺=858.4

¹H NMR (400 MHz, CD₃OD) δ=8.16 (s, 1H), 8.13 (d, J=8.4 Hz, 1H), 7.59 (d, J=1.8 Hz, 1H), 7.55-7.51 (m, 2H), 7.47-7.41 (m, 2H), 7.29-7.23 (m, 1H), 5.11-5.03 (m, 1H), 4.92-4.90 (m, 1H), 4.18 (t, J=12.2 Hz, 2H), 4.03 (s, 3H), 4.00-3.84 (m, 2H), 3.83-3.66 (m, 2H), 3.42 (s, 3H), 3.28-3.17 (m, 6H), 2.87-2.70 (m, 4H), 2.27-2.20 (m, 2H), 2.14-2.01 (m, 3H), 2.01-1.81 (m, 5H), 1.81-1.55 (m, 7H).

### Example 10. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(4-(2-(4-(3-((2,6-dioxopiperidin-3-yl)carbamoyl)phenyl)piperazin-1-yl)ethyl)piperidin-1-yl)-3-methoxybenzamide (Compound 10)

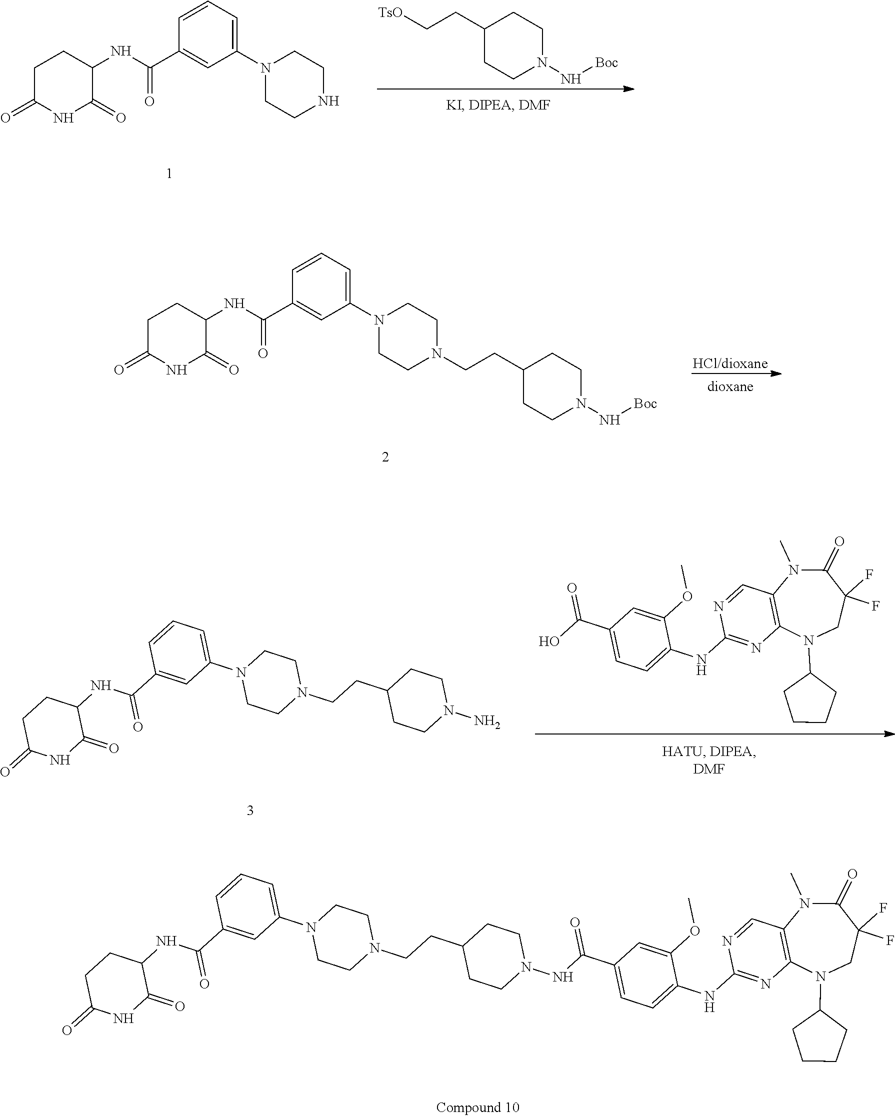 [SMILES: O=C1CCC(NC(=O)c2cccc(N3CCNCC3)c2)C(=O)N1 ; Cc1ccc(S(=O)(=O)OCCC2CCN(NC(=O)OC(C)(C)C)CC2)cc1 ; CC(C)(C)OC(=O)NN1CCC(CCN2CCN(c3cccc(C(=O)NC4CCC(=O)NC4=O)c3)CC2)CC1 ; NN1CCC(CCN2CCN(c3cccc(C(=O)NC4CCC(=O)NC4=O)c3)CC2)CC1 ; COc1cc(C(=O)O)ccc1Nc1ncc2c(n1)N(C1CCCC1)CC(F)(F)C(=O)N2C ; COc1cc(C(=O)NN2CCC(CCN3CCN(c4cccc(C(=O)NC5CCC(=O)NC5=O)c4)CC3)CC2)ccc1Nc1ncc2c(n1)N(C1CCCC1)CC(F)(F)C(=O)N2C]

### Step 1. Synthesis of tert-butyl (4-(2-(4-(3-((2,6-dioxopiperidin-3-yl)carbamoyl)phenyl)piperazin-1-yl)ethyl)piperidin-1-yl)carbamate (2)

To a solution of N-(2,6-dioxopiperidin-3-yl)-3-(piperazin-1-yl)benzamide (400 mg, 1.13 mmol, HCl) in DMF (8 mL) were added DIPEA (890.43 mg, 6.89 mmol, 1.20 mL), KI (100 mg, 602.40 μmol) and 2-(1-((tert-butoxycarbonyl)amino)piperidin-4-yl)ethyl 4-methylbenzenesulfonate (600.02 mg, 1.51 mmol) at 25° C. The mixture was stirred at 60° C. for 16 hr. LCMS showed a peak (30%) with desired mass. The reaction mixture was diluted with H₂O (10 mL), and extracted with EtOAc (30 mL×2). The combined organic layers were washed with brine (30 mL×3), dried over anhydrous Na₂SO₄, filtered and the filtrate was concentrated under reduced pressure. The residue was purified by flash silica gel chromatography (Biotage; 10 g SepaFlash® Silica Flash Column, Eluent of 0˜25% Methanol:EtOAc gradient, 60 mL/min) to afford tert-butyl (4-(2-(4-(3-((2,6-dioxopiperidin-3-yl)carbamoyl)phenyl)piperazin-1-yl)ethyl)piperidin-1-yl)carbamate (200 mg, 368.55 μmol, 32.51% yield) as a light yellow solid. MS (M+H)⁺=543.5

¹H NMR (400 MHz, DMSO-d₆) δ=10.89 (s, 1H), 8.70 (d, J=8.3 Hz, 1H), 7.99-7.76 (m, 1H), 7.39 (s, 1H), 7.34-7.25 (m, 2H), 7.17-7.05 (m, 1H), 4.85-4.69 (m, 1H), 3.36-3.35 (m, 4H), 3.19-3.16 (m, 4H), 2.89-2.75 (m, 3H), 2.59-2.55 (m, 1H), 2.45-2.38 (m, 2H), 2.36-2.30 (m, 2H), 2.17-2.06 (m, 1H), 2.01-1.94 (m, 1H), 1.68-1.60 (m, 2H), 1.44-1.39 (m, 2H), 1.37 (s, 9H), 1.24-1.15 (m, 3H).

### Step 2. Synthesis of 3-(4-(2-(1-aminopiperidin-4-yl)ethyl)piperazin-1-yl)-N-(2,6-dioxopiperidin-3-yl)benzamide (3)

To a solution of tert-butyl (4-(2-(4-(3-((2,6-dioxopiperidin-3-yl)carbamoyl)phenyl)piperazin-1-yl)ethyl)piperidin-1-yl)carbamate (180 mg, 331.69 μmol) in dioxane (5 mL) was added HCl/dioxane (4 M, 10 mL). The mixture was stirred at 25° C. for 16 hr. LCMS showed a peak (84%) with the desired mass. The reaction mixture was concentrated under reduced pressure to afford 3-(4-(2-(1-aminopiperidin-4-yl)ethyl)piperazin-1-yl)-N-(2,6-dioxopiperidin-3-yl)benzamide (160 mg, crude, 2HCl salt) as a light yellow solid. MS (M+H)⁺=443.4

¹H NMR (400 MHz, DMSO-d₆) δ=10.86 (s, 1H), 10.84-10.70 (m, 1H), 8.76 (d, J=8.4 Hz, 1H), 7.46 (s, 1H), 7.38 (d, J=4.9 Hz, 2H), 7.23-7.17 (m, 1H), 4.80-4.72 (m, 1H), 3.97-3.79 (m, 3H), 3.24-2.99 (m, 8H), 2.91-2.73 (m, 2H), 2.59-2.56 (m, 1H), 2.19-2.11 (m, 1H), 2.05-1.93 (m, 2H), 1.87-1.66 (m, 4H), 1.50-1.22 (m, 3H).

### Step 3. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(4-(2-(4-(3-((2,6-dioxopiperidin-3-yl)carbamoyl)phenyl)piperazin-1-yl)ethyl)piperidin-1-yl)-3-methoxybenzamide (Compound 10)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (60 mg, 134.10 μmol) in DMF (2 mL) were added HATU (70 mg, 184.10 μmol), DIPEA (111.30 mg, 861.17 μmol, 150 μL) and 3-(4-(2-(1-aminopiperidin-4-yl)ethyl)piperazin-1-yl)-N-(2,6-dioxopiperidin-3-yl)benzamide (80 mg, 155.20 μmol, 2HCl salt) at 25° C. The mixture was stirred at 25° C. for 16 hr. LCMS showed a peak (40%) with desired mass. The mixture was filtered and the filtrate was purified by prep-HPLC (column: Phenomenex Synergi Polar-RP 100×25 mm×4 μm; mobile phase: [water (TFA)-ACN]; B %: 28%-48%, 7 min; Column Temp: 30° C.) and re-purified by prep-HPLC (column: Waters Xbridge 150×25 mm×5 μm; mobile phase: [water (NH₄HCO₃)-ACN]; B %: 28%-66%, 9 min; Column Temp: 30° C.), the eluent was lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-N-(4-(2-(4-(3-((2,6-dioxopiperidin-3-yl)carbamoyl)phenyl)piperazin-1-yl)ethyl)piperidin-1-yl)-3-methoxybenzamide (30.7 mg, 33.80 μmol, 25.20% yield, 96% purity) as a white solid. MS (M+H)⁺=872.7

¹H NMR (400 MHz, DMSO-d₆) δ=10.87 (s, 1H), 9.39-9.19 (m, 1H), 8.77-8.60 (m, 1H), 8.36-8.16 (m, 2H), 7.97 (s, 1H), 7.49-7.36 (m, 3H), 7.34-7.27 (m, 2H), 7.15-7.09 (m, 1H), 4.83-4.72 (m, 2H), 4.12-4.00 (m, 2H), 3.93 (s, 3H), 3.33-3.32 (m, 3H), 3.24-3.17 (m 4H), 3.06-2.98 (m, 2H), 2.84-2.73 (m, 3H), 2.58-2.53 (m, 5H), 2.42-2.35 (m, 2H), 2.18-2.09 (m, 1H), 2.01-1.90 (m, 3H), 1.76-1.57 (m, 8H), 1.48-1.26 (m, 5H).

### Example 11. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(3-(2-(4-(3-((2,6-dioxopiperidin-3-yl)carbamoyl)phenyl)piperazin-1-yl)ethyl)pyrrolidin-1-yl)-3-methoxybenzamide (Compound 11)

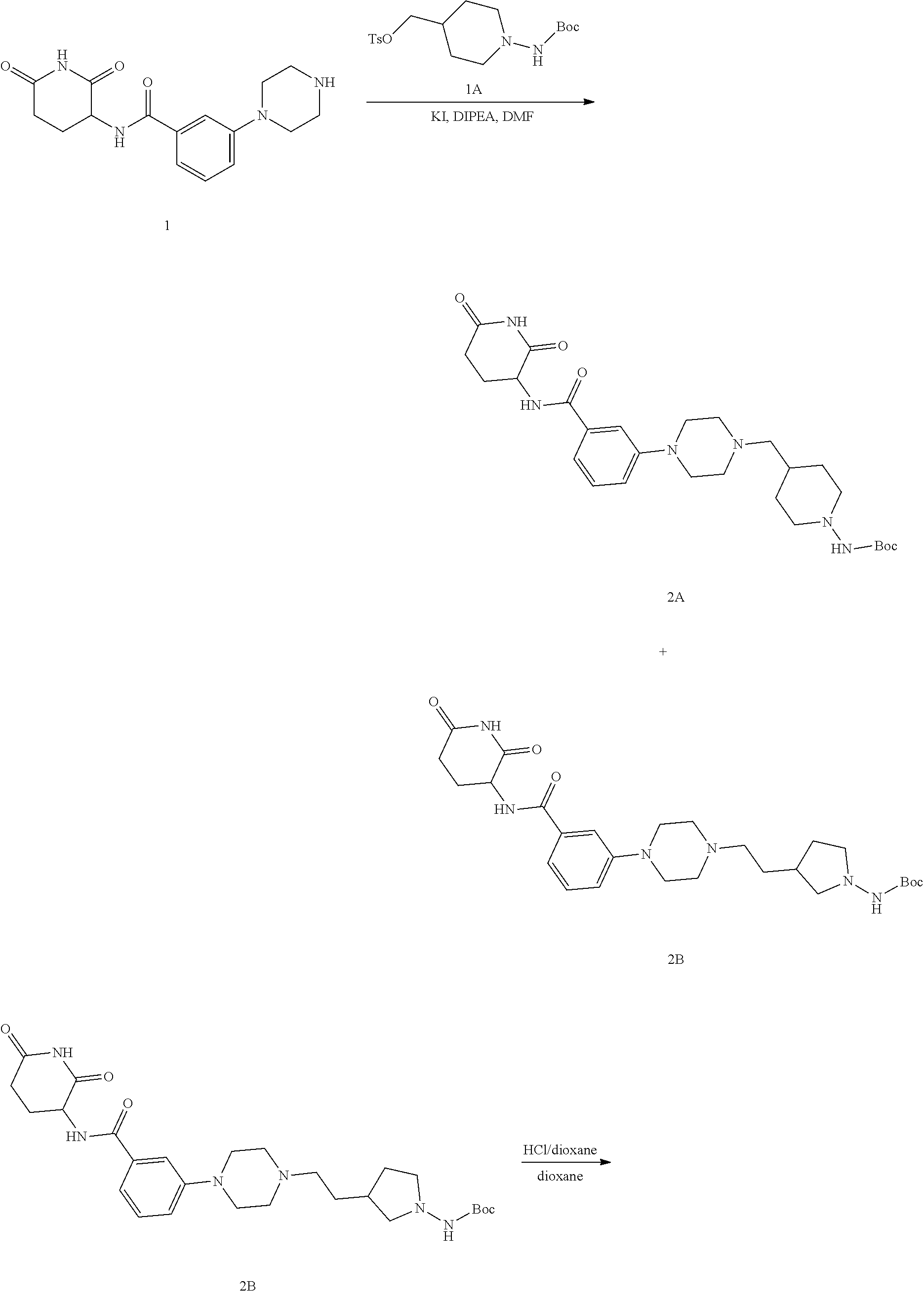 [SMILES: Cc1ccc(S(=O)(=O)OCC2CCN(NC(=O)OC(C)(C)C)CC2)cc1 ; O=C1CCC(NC(=O)c2cccc(N3CCNCC3)c2)C(=O)N1 ; CC(C)(C)OC(=O)NN1CCC(CN2CCN(c3cccc(C(=O)NC4CCC(=O)NC4=O)c3)CC2)CC1 ; CC(C)(C)OC(=O)NN1CCC(CCN2CCN(c3cccc(C(=O)NC4CCC(=O)NC4=O)c3)CC2)C1 ; CC(C)(C)OC(=O)NN1CCC(CCN2CCN(c3cccc(C(=O)NC4CCC(=O)NC4=O)c3)CC2)C1]

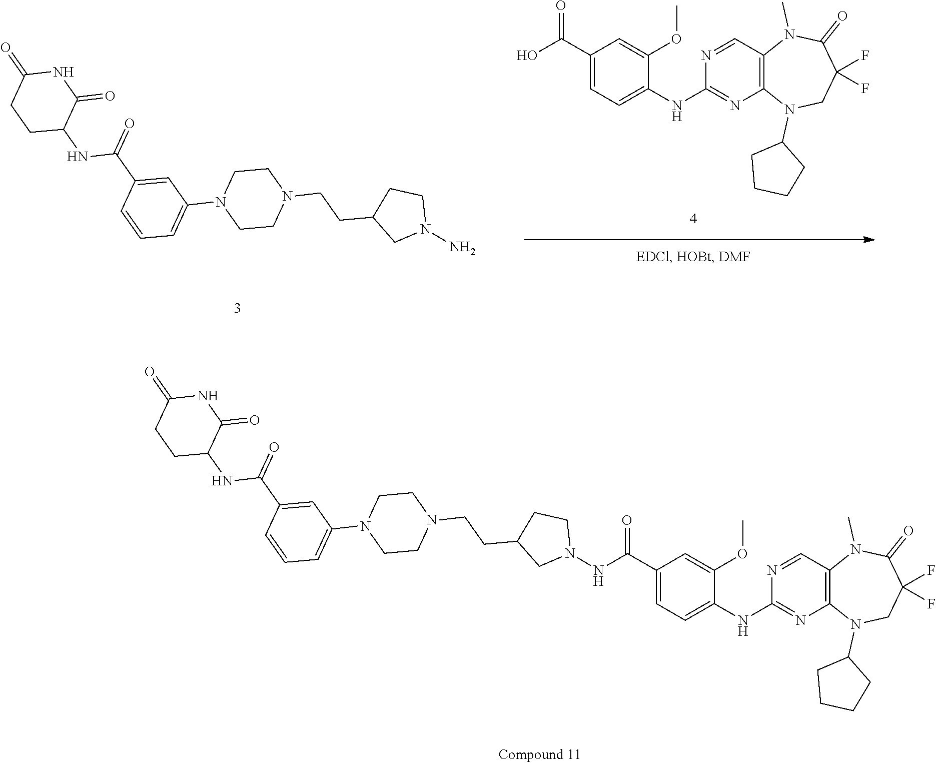 [SMILES: NN1CCC(CCN2CCN(c3cccc(C(=O)NC4CCC(=O)NC4=O)c3)CC2)C1 ; COc1cc(C(=O)O)ccc1Nc1ncc2c(n1)N(C1CCCC1)CC(F)(F)C(=O)N2C ; COc1cc(C(=O)NN2CCC(CCN3CCN(c4cccc(C(=O)NC5CCC(=O)NC5=O)c4)CC3)C2)ccc1Nc1ncc2c(n1)N(C1CCCC1)CC(F)(F)C(=O)N2C]

### Step 1. Synthesis of tert-butyl (4-((4-(3-((2,6-dioxopiperidin-3-yl)carbamoyl)phenyl)piperazin-1-yl)methyl)piperidin-1-yl)carbamate (2A) and tert-butyl (3-(2-(4-(3-((2,6-dioxopiperidin-3-yl)carbamoyl)phenyl)piperazin-1-yl)ethyl)pyrrolidin-1-yl)carbamate (2B)

To a solution of N-(2,6-dioxopiperidin-3-yl)-3-(piperazin-1-yl)benzamide (500 mg, 1.42 mmol, HCl) in DMF (8 mL) were added KI (60 mg, 361.44 μmol), DIPEA (1.04 g, 8.04 mmol, 1.4 mL) and (1-((tert-butoxycarbonyl)amino)piperidin-4-yl)methyl 4-methylbenzenesulfonate (500.00 mg, 1.30 mmol) at 25° C. The mixture was stirred at 60° C. for 16 hr under N₂ atmosphere. LCMS showed main peak with the desired mass. The reaction mixture was diluted with H₂O (10 mL), and extracted with EtOAc (30 mL×2). The combined organic layers were washed with brine (30 mL×3), dried over Na₂SO₄, filtered and the filtrate was concentrated under reduced pressure. The residue was purified by flash silica gel chromatography (Biotage; 10 g SepaFlash® Silica Flash Column, Eluent of 0˜25% Methanol:EtOAc gradient, 60 mL/min) to afford Compound tert-butyl (4-((4-(3-((2,6-dioxopiperidin-3-yl)carbamoyl)phenyl)piperazin-1-yl)methyl)piperidin-1-yl)carbamate (130 mg, 245.91 μmol, 17.35% yield) as a light yellow solid and compound tert-butyl (3-(2-(4-(3-((2,6-dioxopiperidin-3-yl)carbamoyl)phenyl)piperazin-1-yl)ethyl)pyrrolidin-1-yl)carbamate (260 mg, 491.83 μmol, 34.70% yield) as a light yellow solid, which was confirmed by 2D NMR. MS (M+H)⁺=529.5

¹H NMR (400 MHz, DMSO-d₆) δ=10.87 (s, 1H), 8.69 (d, J=8.3 Hz, 1H), 7.88 (s, 1H), 7.39 (s, 1H), 7.33-7.22 (m, 2H), 7.13-7.07 (m, 1H), 4.82-4.70 (m, 1H), 3.40-3.35 (m, 4H), 3.19-3.16 (m, 4H), 2.93 (t, J=8.1 Hz, 1H), 2.87-2.77 (m, 2H), 2.77-2.72 (m, 1H), 2.58-2.53 (m, 1H), 2.49-2.44 (m, 2H), 2.35-2.24 (m, 2H), 2.16-2.01 (m, 2H), 1.96-1.90 (m, 1H), 1.54-1.46 (m, 2H), 1.37 (s, 9H), 1.34-1.24 (m, 1H).

### Step 2. Synthesis of 3-(4-(2-(1-aminopyrrolidin-3-yl)ethyl)piperazin-1-yl)-N-(2,6-dioxopiperidin-3-yl)benzamide (3)

To a solution of tert-butyl (3-(2-(4-(3-((2,6-dioxopiperidin-3-yl)carbamoyl)phenyl)piperazin-1-yl)ethyl)pyrrolidin-1-yl)carbamate (150 mg, 283.75 μmol) in dioxane (10 mL) were added HCl/dioxane (4 M, 10 mL). The mixture was stirred at 25° C. for 3 hr. LCMS showed a main peak with the desired mass. The reaction mixture was concentrated under reduced pressure to afford 3-(4-(2-(1-aminopyrrolidin-3-yl)ethyl)piperazin-1-yl)-N-(2,6-dioxopiperidin-3-yl)benz amide (130 mg, crude, 2HCl salt) as a light yellow solid. MS (M+H)⁺=429.2

### Step 3. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(3-(2-(4-(3-((2,6-dioxopiperidin-3-yl)carbamoyl)phenyl)piperazin-1-yl)ethyl)pyrrolidin-1-yl)-3-methoxybenzamide (Compound 11)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (80 mg, 178.80 μmol) in DMF (2 mL) were added EDCI (60 mg, 312.99 μmol), HOBt (30 mg, 222.02 μmol) and 3-(4-(2-(1-aminopyrrolidin-3-yl)ethyl)piperazin-1-yl)-N-(2,6-dioxopiperidin-3-yl)benz amide (100.00 mg, 199.43 μmol, 2HCl salt) at 25° C. The mixture was stirred at 25° C. for 16 hr. LCMS showed a peak (57%) with desired mass. The mixture was filtered and the filtrate was purified by prep-HPLC (column: Phenomenex Synergi Polar-RP 100×25 mm×4 μm; mobile phase: [water (TFA)-ACN]; B %: 28%-48%, 7 min; Column Temp: 30° C.) to afford 4-((9-cyclopentyl-7,7-difluoro-5-methyl-6-oxo-6,7,8,9-tetrahydro-5H-pyrimido[4,5-b][1,4]diazepin-2-yl)amino)-N-(3-(2-(4-(3-((2,6-dioxopiperidin-3-yl)carbamoyl)phenyl)piperazin-1-yl)ethyl)pyrrolidin-1-yl)-3-methoxybenzamide (78.5 mg, 77.53 μmol, 43.36% yield, 96% purity, TFA salt) as a white solid. MS (M+H)⁺=858.4

¹H NMR (400 MHz, MeOD) δ=8.24-8.11 (m, 2H), 7.61-7.50 (m, 3H), 7.49-7.39 (m, 2H), 7.31-7.23 (m, 1H), 5.10-5.01 (m, 1H), 4.98-4.91 (m, 1H), 4.23-4.12 (m 2H), 4.02 (s, 3H), 3.99-3.61 (m, 4H), 3.42 (s, 3H), 3.38-3.33 (m, 4H), 3.32-3.26 (m, 4H), 3.25-3.15 (m, 2H), 3.09-3.00 (m, 1H), 2.92-2.81 (m, 1H), 2.78-2.70 (m, 1H), 2.53-2.41 (m, 1H), 2.31-2.18 (m, 3H), 2.09-1.97 (m, 4H), 1.89-1.79 (m, 2H), 1.77-1.68 (m, 4H).

### Example 12. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(4-((4-(4-((2,6-dioxopiperidin-3-yl)carbamoyl)-2-fluorophenyl)piperazin-1-yl)methyl)piperidin-1-yl)-3-methoxybenzamide (Compound 12)

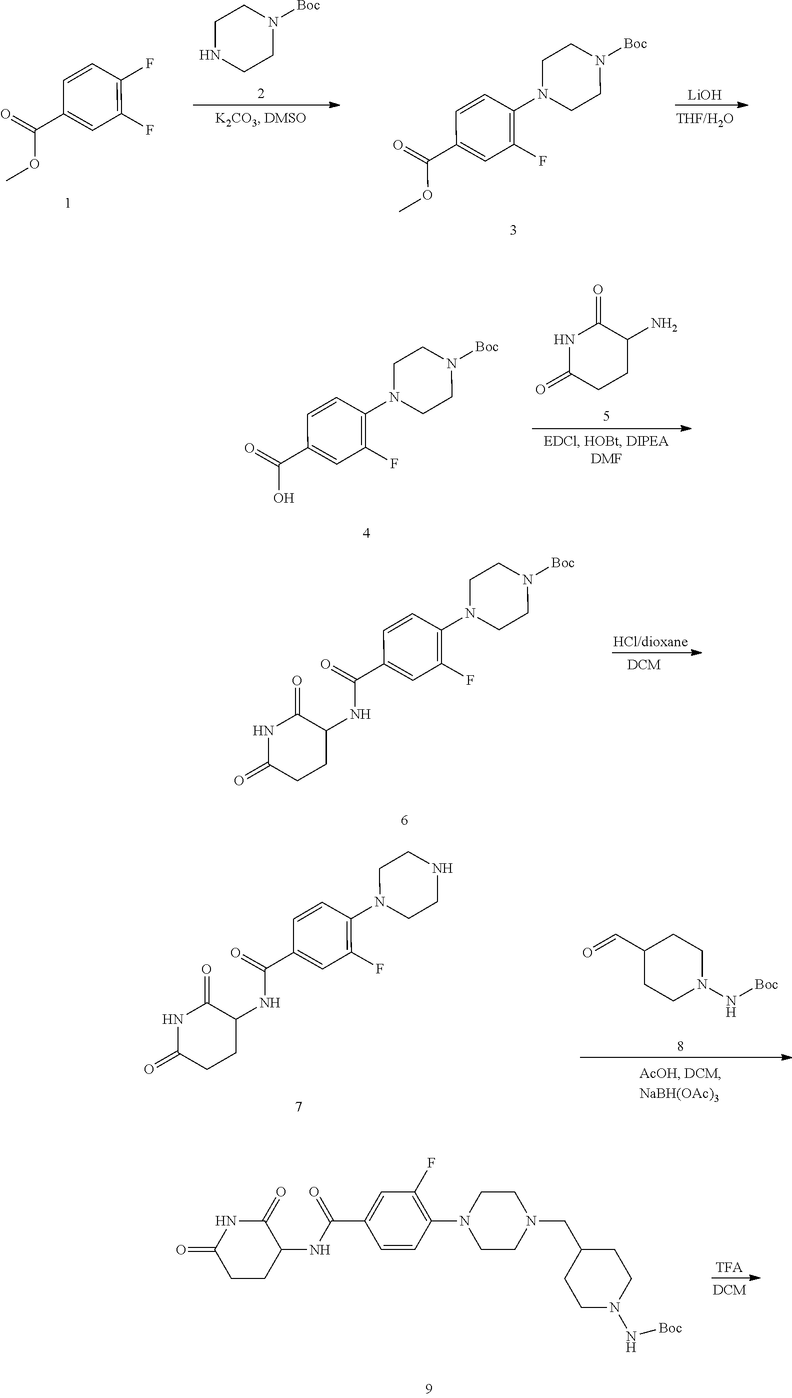

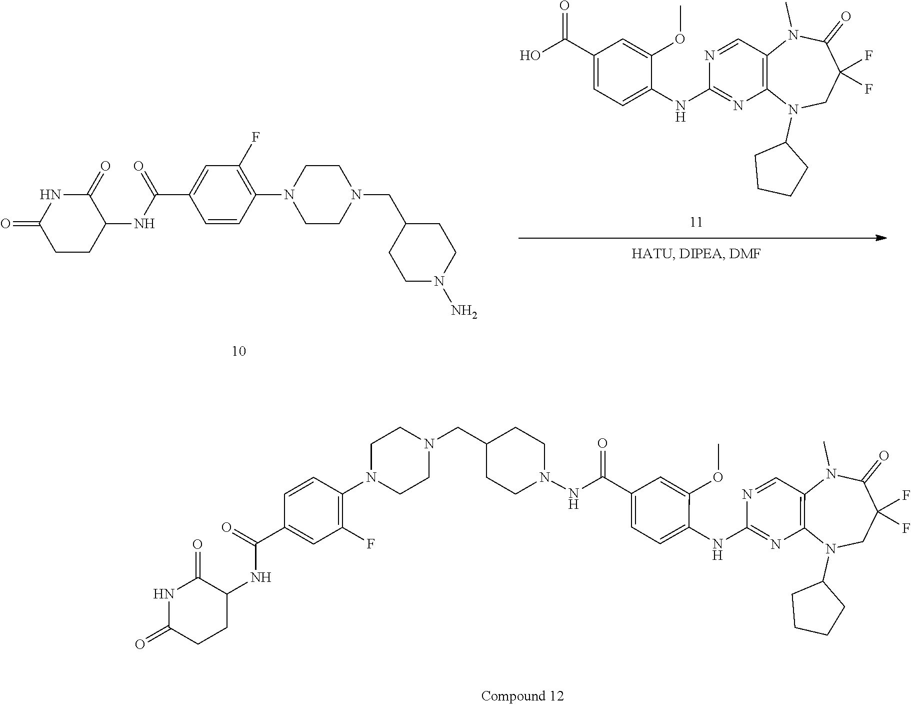 [SMILES: NN1CCC(CN2CCN(c3ccc(C(=O)NC4CCC(=O)NC4=O)cc3F)CC2)CC1 ; COc1cc(C(=O)O)ccc1Nc1ncc2c(n1)N(C1CCCC1)CC(F)(F)C(=O)N2C ; COc1cc(C(=O)NN2CCC(CN3CCN(c4ccc(C(=O)NC5CCC(=O)NC5=O)cc4F)CC3)CC2)ccc1Nc1ncc2c(n1)N(C1CCCC1)CC(F)(F)C(=O)N2C]

### Step 1. Synthesis of tert-butyl 4-(2-fluoro-4-(methoxycarbonyl)phenyl)piperazine-1-carboxylate (3)

To a solution of methyl 3,4-difluorobenzoate (2 g, 11.62 mmol) and tert-butyl piperazine-1-carboxylate (2.60 g, 13.94 mmol) in DMSO (20 mL) was added K₂CO₃ (3.21 g, 23.24 mmol), the mixture was stirred at 100° C. for 16 h. LCMS showed a main peak with desired mass. The reaction mixture was diluted with brine (50 mL) and extracted with EtOAc (50 mL×3). The combined organic layers were washed with brine (30 mL×2), dried over Na₂SO₄, filtered and concentrated under reduced pressure. The residue was purified by flash silica gel chromatography (Biotage; 10 g SepaFlash® Silica Flash Column, Eluent of 10-65% EtOAc/Petroleum ether @40 mL/min) to afford tert-butyl 4-(2-fluoro-4-(methoxycarbonyl)phenyl)piperazine-1-carboxylate (3 g, 8.69 mmol, 74.78% yield, 98% purity) as a white solid. MS (M+H)⁺=338.8

### Step 2. Synthesis of 4-(4-(tert-butoxycarbonyl)piperazin-1-yl)-3-fluorobenzoic acid (4)

To a solution of tert-butyl 4-(2-fluoro-4-(methoxycarbonyl)phenyl)piperazine-1-carboxylate (3 g, 8.87 mmol) in THF (30 mL) and H₂O (10 mL) was added LiOH·H₂O (744.09 mg, 17.73 mmol), the mixture was stirred at 20° C. for 16 h. LCMS showed a main peak (97%) with desired mass. The mixture was diluted with water (30 mL). The aqueous layer was adjusted to pH=6 using HCl (1 N), extracted with EtOAc (40 mL×3). The combined organic layers was dried over Na₂SO₄, filtered and concentrated under reduced pressure to afford 4-(4-(tert-butoxycarbonyl)piperazin-1-yl)-3-fluorobenzoic acid (2.8 g, crude) as a white solid. MS (M−100+H)⁺=225.1

### Step 3. Synthesis of tert-butyl 4-(4-((2,6-dioxopiperidin-3-yl)carbamoyl)-2-fluorophenyl)piperazine-1-carboxylate (6)

To a solution of 4-(4-(tert-butoxycarbonyl)piperazin-1-yl)-3-fluorobenzoic acid (2.6 g, 8.02 mmol), 3-aminopiperidine-2,6-dione (1.45 g, 8.82 mmol, HCl salt) in DMF (40 mL) were added EDCI (2.31 g, 12.02 mmol), HOBt (1.62 g, 12.02 mmol), DIPEA (3.11 g, 24.05 mmol, 4.19 mL), the mixture was stirred at 20° C. for 16 h. LCMS showed a main peak with desired mass. The reaction mixture was diluted with brine (100 mL) and extracted with EtOAc (100 mL×3). The combined organic layers were washed with brine (100 mL×2), dried over Na₂SO₄, filtered and concentrated under reduced pressure. The residue was triturated with MTBE (20 mL) at 20° C. for 10 min to afford tert-butyl 4-(4-((2,6-dioxopiperidin-3-yl)carbamoyl)-2-fluorophenyl)piperazine-1-carboxylate (3 g, 6.70 mmol, 83.56% yield, 97% purity) as a white solid. MS (M+H)⁺=435.2

### Step 4. Synthesis of N-(2,6-dioxopiperidin-3-yl)-3-fluoro-4-(piperazin-1-yl)benzamide (7)

To a solution of tert-butyl 4-[4-[(2,6-dioxo-3-piperidyl)carbamoyl]-2-fluoro-phenyl]piperazine-1-carboxylate (200 mg, 460.34 μmol) in dioxane (2 mL) was added HCl/dioxane (4 M, 2 mL), the mixture was stirred at 20° C. for 1 h. LCMS showed a main peak with desired mass. The mixture was concentrated under reduced pressure to afford N-(2,6-dioxopiperidin-3-yl)-3-fluoro-4-(piperazin-1-yl)benzamide (150 mg, crude) as a white solid, which was used into the next step directly. MS (M+H)⁺=335.1

### Step 5. Synthesis of tert-butyl (4-((4-(4-((2,6-dioxopiperidin-3-yl)carbamoyl)-2-fluorophenyl) piperazin-1-yl)methyl)piperidin-1-yl)carbamate (9)

A mixture of N-(2,6-dioxopiperidin-3-yl)-3-fluoro-4-(piperazin-1-yl)benzamide (150 mg, 404.52 μmol, HCl salt) and tert-butyl (4-formylpiperidin-1-yl)carbamate (92.35 mg, 404.52 μmol), AcOH (12.15 mg, 202.26 μmol, 11.57 L) in DCM (4 mL) was stirred at 20° C. for 1 h, NaBH(OAc)₃ (257.21 mg, 1.21 mmol) was added, the mixture was stirred at 20° C. for 16 h. LCMS showed a peak (27%) with desired mass. The reaction mixture was diluted with H₂O (10 mL). The organic phase was separated, the aqueous phase was extracted with EtOAc (10 mL×3). The combined organic layers were washed with brine (20 mL×2), dried over Na₂SO₄, filtered. The filtrate was concentrated under reduced pressure. The residue was purified by flash silica gel chromatography (ISCO®; 40 g SepaFlash® Silica Flash Column, Eluent of 5-20% EtOAc/MeOH @100 mL/min) to afford tert-butyl (4-((4-(4-((2,6-dioxopiperidin-3-yl)carbamoyl)-2-fluorophenyl)piperazin-1-yl)methyl) piperidin-1-yl)carbamate (130 mg, 195.01 μmol, 48.21% yield, 82% purity) as a yellow solid. MS (M+H)⁺=547.4

### Step 6. Synthesis of 4-(4-((1-aminopiperidin-4-yl)methyl)piperazin-1-yl)-N-(2,6-dioxopiperidin-3-yl)-3-fluorobenzamide (10)

To a solution of tert-butyl (4-((4-(4-((2,6-dioxopiperidin-3-yl)carbamoyl)-2-fluorophenyl)piperazin-1-yl)methyl) piperidin-1-yl)carbamate (130 mg, 237.82 μmol) in DCM (5 mL) was added TFA (1.54 g, 13.51 mmol, 1 mL), the mixture was stirred at 20° C. for 0.5 h. LCMS showed a main peak with desired mass. The reaction mixture was concentrated in vacuo to afford 4-(4-((1-aminopiperidin-4-yl)methyl)piperazin-1-yl)-N-(2,6-dioxopiperidin-3-yl)-3-fluorobenzamide (100 mg, crude, TFA salt) as a brown solid. MS (M+H)⁺=447.3

### Step 7. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(4-((4-(4-((2,6-dioxopiperidin-3-yl)carbamoyl)-2-fluorophenyl)piperazin-1-yl)methyl)piperidin-1-yl)-3-methoxybenzamide (Compound 12)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (79.82 mg, 178.40 μmol) in DMF (2 mL) were added HATU (101.75 mg, 267.60 μmol) and DIPEA (69.17 mg, 535.20 mol, 93.22 L), the mixture was stirred at 20° C. for 1 h. Then 4-(4-((1-aminopiperidin-4-yl)methyl)piperazin-1-yl)-N-(2,6-dioxopiperidin-3-yl)-3-fluorobenzamide (100 mg, 178.40 μmol, TFA salt) was added and the resulting mixture was stirred at 20° C. for 2 h. LCMS showed a main peak with desired mass. The reaction mixture was diluted with brine (10 mL), extracted with EtOAc (10 mL×3). The combined organic layers were washed with brine (20 mL×2), dried over Na₂SO₄, filtered. The filtrate was concentrated under reduced pressure. The residue was purified by prep-TLC (SiO₂, DCM:MeOH=10:1) and re-purified by reversed-phase HPLC (column: Waters Xbridge 150×25 mm×5 μm; mobile phase: [water (NH₄HCO₃)-ACN]; B %: 37%-67%, 9 min). The eluent was lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-N-(4-((4-(4-((2,6-dioxopiperidin-3-yl)carbamoyl)-2-fluorophenyl)piperazin-1-yl)methyl)piperidin-1-yl)-3-methoxybenzamide (41 mg, 42.13 μmol, 23.61% yield, 90% purity) as a white solid. MS (M+H)⁺=876.5

¹H NMR (400 MHz, DMSO-d₆) δ=10.85 (s, 1H), 9.31 (s, 1H), 8.66 (d, J=8.4 Hz, 1H), 8.31-8.23 (m, 2H), 7.96 (s, 1H), 7.69-7.59 (m, 2H), 7.46-7.38 (m, 2H), 7.09 (t, J=8.7 Hz, 1H), 4.82-4.70 (m, 2H), 4.04 (t, J=14.1 Hz, 2H), 3.93 (s, 3H), 3.33 (s, 3H), 3.18-3.09 (m, 4H), 3.06-2.98 (m, 2H), 2.88-2.72 (m, 3H), 2.58-2.52 (m, 5H), 2.27-2.18 (m, 2H), 2.16-2.04 (m, 1H), 2.01-1.88 (m, 3H), 1.82-1.66 (m, 4H), 1.65-1.51 (m, 5H), 1.34-1.16 (m, 2H).

### Example 13. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(4-(4-((2,6-dioxopiperidin-3-yl)amino)phenyl)piperazin-1-yl)propanoyl)piperidin-4-yl)-3-methoxybenzamide (Compound 13)

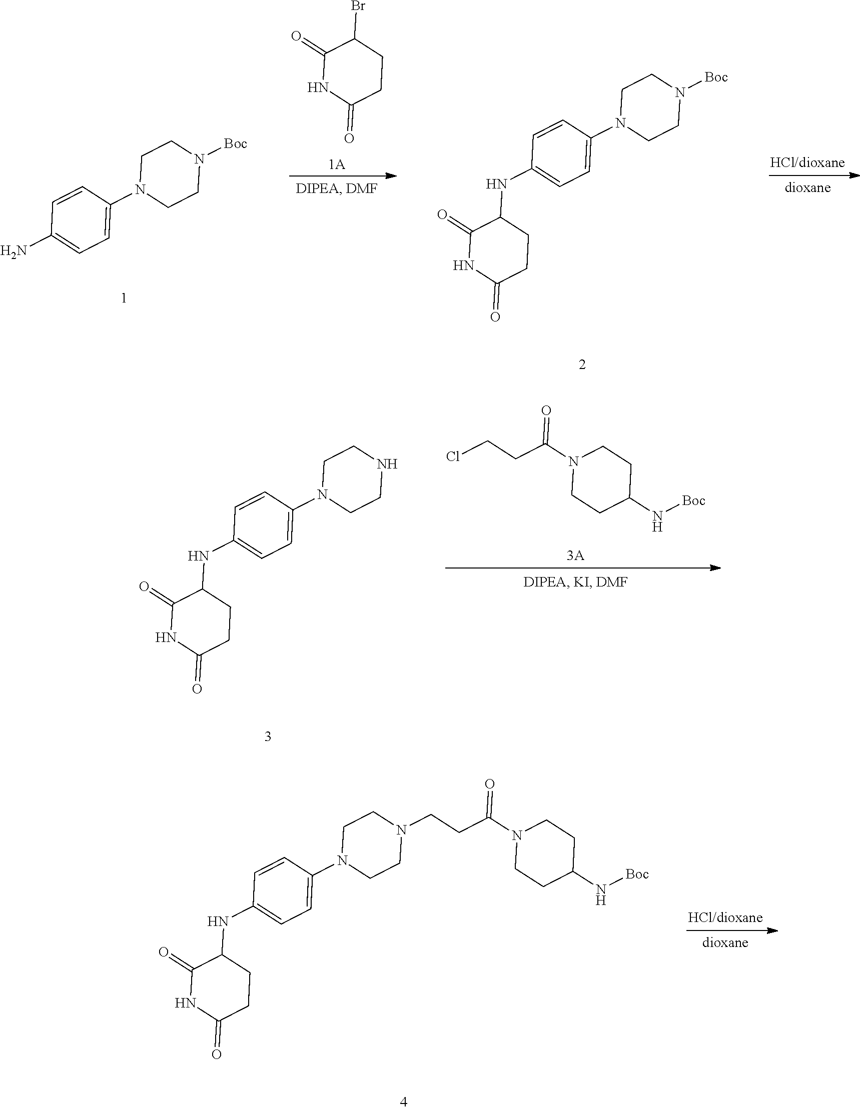 [SMILES: CC(C)(C)OC(=O)N1CCN(c2ccc(N)cc2)CC1 ; O=C1CCC(Br)C(=O)N1 ; CC(C)(C)OC(=O)N1CCN(c2ccc(NC3CCC(=O)NC3=O)cc2)CC1 ; O=C1CCC(Nc2ccc(N3CCNCC3)cc2)C(=O)N1 ; CC(C)(C)OC(=O)NC1CCN(C(=O)CCCl)CC1 ; CC(C)(C)OC(=O)NC1CCN(C(=O)CCN2CCN(c3ccc(NC4CCC(=O)NC4=O)cc3)CC2)CC1]

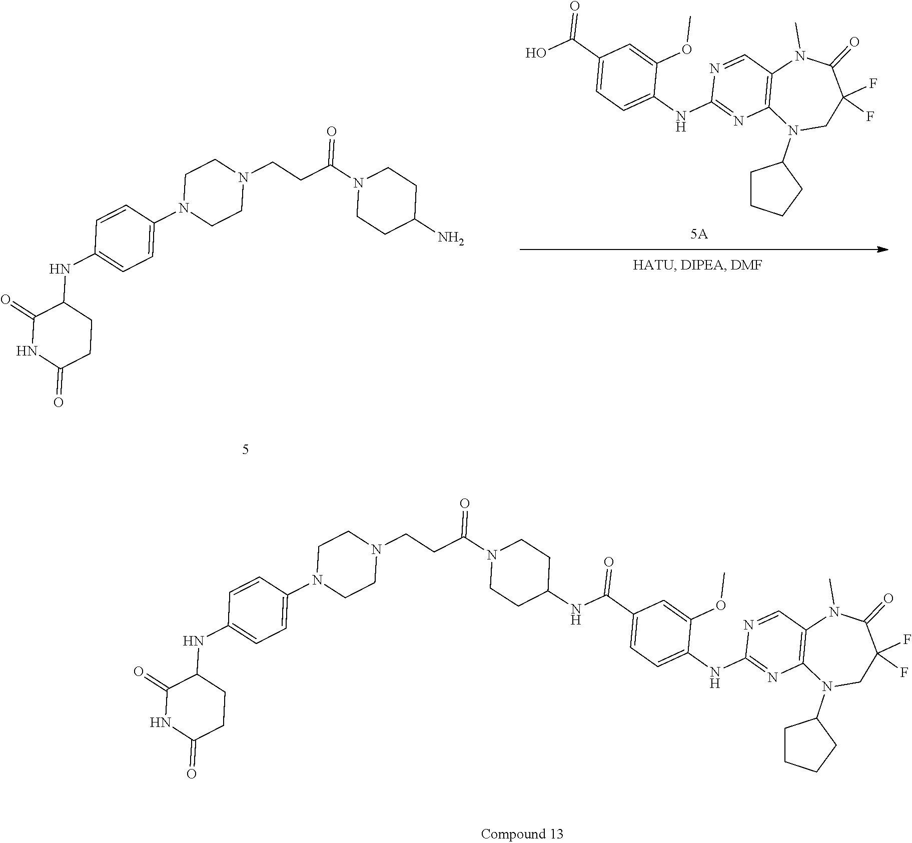 [SMILES: NC1CCN(C(=O)CCN2CCN(c3ccc(NC4CCC(=O)NC4=O)cc3)CC2)CC1 ; COc1cc(C(=O)O)ccc1Nc1ncc2c(n1)N(C1CCCC1)CC(F)(F)C(=O)N2C ; COc1cc(C(=O)NC2CCN(C(=O)CCN3CCN(c4ccc(NC5CCC(=O)NC5=O)cc4)CC3)CC2)ccc1Nc1ncc2c(n1)N(C1CCCC1)CC(F)(F)C(=O)N2C]

### Step 1. Synthesis of tert-butyl 4-(4-((2,6-dioxopiperidin-3-yl)amino)phenyl)piperazine-1-carboxylate (2)

To a solution of tert-butyl 4-(4-aminophenyl)piperazine-1-carboxylate (1 g, 3.61 mmol) in DMF (10 mL) were added 3-bromopiperidine-2,6-dione (761.50 mg, 3.97 mmol) and DIPEA (1.40 g, 10.82 mmol, 1.88 mL), the mixture was stirred at 80° C. for 16 hr. LCMS showed a main peak with desired mass. The mixture was diluted with water (3 mL), extracted with EtOAc (5 mL×3). The organic layer was dried over Na₂ SO₄, filtered and concentrated under vacuum. The crude product was purified by flash silica gel chromatography (Biotage, 4 g SepaFlash® Silica Flash Column, Eluent of 4-80% EtOAc/Petroleum ether gradient@20 mL/min) to afford tert-butyl 4-(4-((2,6-dioxopiperidin-3-yl)amino)phenyl)piperazine-1-carboxylate (0.96 g, 2.08 mmol, 57.58% yield, 84% purity) as a brown powder. MS (M+H)⁺=388.9

### Step 2. Synthesis of 3-((4-(piperazin-1-yl)phenyl)amino)piperidine-2,6-dione (3)

To a solution of tert-butyl 4-(4-((2,6-dioxopiperidin-3-yl)amino)phenyl)piperazine-1-carboxylate (500 mg, 1.29 mmol) in dioxane (8 mL) was added HCl/dioxane (4 M, 321.78 L), the mixture was stirred at 25° C. for 2 hr. LCMS showed the starting material was consumed completely and a main peak with desired mass. The mixture was concentrated under vacuum to afford 3-((4-(piperazin-1-yl)phenyl)amino)piperidine-2,6-dione (550 mg, crude) as a brown powder. MS (M+H)⁺=288.9

### Step 3. Synthesis of tert-butyl (1-(3-(4-(4-((2,6-dioxopiperidin-3-yl)amino)phenyl)piperazin-1-yl)propanoyl)piperidin-4-yl)carbamate (4)

To a solution of 3-((4-(piperazin-1-yl)phenyl)amino)piperidine-2,6-dione (550 mg, 1.69 mmol, HCl) in DMF (15 mL) were added tert-butyl (1-(3-chloropropanoyl)piperidin-4-yl)carbamate (541.63 mg, 1.86 mmol), DIPEA (656.55 mg, 5.08 mmol, 884.84 μL) and KI (56.22 mg, 338.66 μmol), the mixture was stirred at 80° C. for 16 hr. LCMS showed a peak (40%) with desired mass. The mixture was diluted with water (3 mL), extracted with EtOAc (5 mL×3). The organic layer was dried over Na₂SO₄, filtered and concentrated under vacuum. The crude product was purified by flash silica gel chromatography (Biotage, 4 g SepaFlash® Silica Flash Column, Eluent of 4-80% EtOAc/Petroleum ether gradient@20 mL/min) to afford tert-butyl (1-(3-(4-(4-((2,6-dioxopiperidin-3-yl)amino)phenyl)piperazin-1-yl)propanoyl)piperidin-4-yl)carbamate (260 mg, 354.54 μmol, 20.94% yield, 74% purity) as a brown powder. MS (M+H)⁺=543.1

### Step 4. Synthesis of 3-((4-(4-(3-(4-aminopiperidin-1-yl)-3-oxopropyl)piperazin-1-yl)phenyl)amino)piperidine-2,6-dione (5)

To a solution of tert-butyl (1-(3-(4-(4-((2,6-dioxopiperidin-3-yl)amino)phenyl)piperazin-1-yl)propanoyl)piperidin-4-yl)carbamate (260 mg, 479.11 μmol) in dioxane (6 mL) was added HCl/dioxane (4 M, 6 mL), the mixture was stirred at 25° C. for 1 hr. LCMS showed a major peak with desired mass, the mixture was concentrated under vacuum to afford 3-((4-(4-(3-(4-aminopiperidin-1-yl)-3-oxopropyl)piperazin-1-yl)phenyl)amino)piperidine-2,6-dione (300 mg, crude, HCl) as a green powder. MS (M+H)⁺=443.0

### Step 5. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(4-(4-((2,6-dioxopiperidin-3-yl)amino)phenyl)piperazin-1-yl)propanoyl)piperidin-4-yl)-3-methoxybenzamide (Compound 13)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (100 mg, 223.50 μmol) in DMF (2 mL) were added HATU (127.47 mg, 335.24 μmol) and DIPEA (86.66 mg, 670.49 μmol, 116.79 L), then 3-((4-(4-(3-(4-aminopiperidin-1-yl)-3-oxopropyl)piperazin-1-yl)phenyl)amino)piperidine-2,6-dione (128.47 mg, 268.20 μmol, HCl salt) was added and the resulting mixture was stirred at 25° C. for 16 hr. LCMS showed a main peak with desired mass. The mixture was diluted with water (5 mL), extracted with EtOAc (10 mL×3). The organic layer was dried over Na₂SO₄, filtered and concentrated under vacuum. the crude product was purified by Prep-HPLC (column: Phenomenex Synergi Polar-RP 100*25 mm*4 m; mobile phase: [water (TFA)-ACN]; B %: 26%-46%, 7 min) and Prep-HPLC (column: Waters Xbridge 150*25 mm*5 m; mobile phase: [water(NH₄ HCO₃)-ACN]; B %: 28%-58%, 8 min), the eluent was lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(4-(4-((2,6-dioxopiperidin-3-yl)amino)phenyl)piperazin-1-yl)propanoyl)piperidin-4-yl)-3-methoxybenzamide (40.4 mg, 44.15 μmol, 19.76% yield, 95.3% purity) as a red powder. MS (M+H)⁺=872.1

¹H NMR (400 MHz, DMSO-d₆) δ=10.74 (s, 1H), 8.33-8.20 (m, 2H), 8.15 (d, J=6.75 Hz, 1H), 7.96 (s, 1H), 7.56-7.43 (m, 2H), 6.75 (d, J=7.75 Hz, 2H), 6.60 (d, J=8.13 Hz, 2H), 5.37 (d, J=7.00 Hz, 1H), 4.83-4.70 (m, 1H), 4.39 (d, J=10.01 Hz, 1H), 4.18 (s, 1H), 4.04 (t, J=13.82 Hz, 3H), 3.99-3.93 (m, 4H), 3.31 (s, 3H), 3.22-3.04 (m, 2H), 2.98-2.93 (m, 4H), 2.69 (d, J=12.76 Hz, 3H), 2.63-2.56 (m, 5H), 2.14-2.06 (m, 1H), 2.00-1.77 (m, 6H), 1.76-1.52 (m, 7H) 1.52-1.35 (m, 2H).

### Example 14. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(4-(3-((2,6-dioxopiperidin-3-yl)amino)phenyl)piperazin-1-yl)propanoyl)piperidin-4-yl)-3-methoxybenzamide (Compound 14)

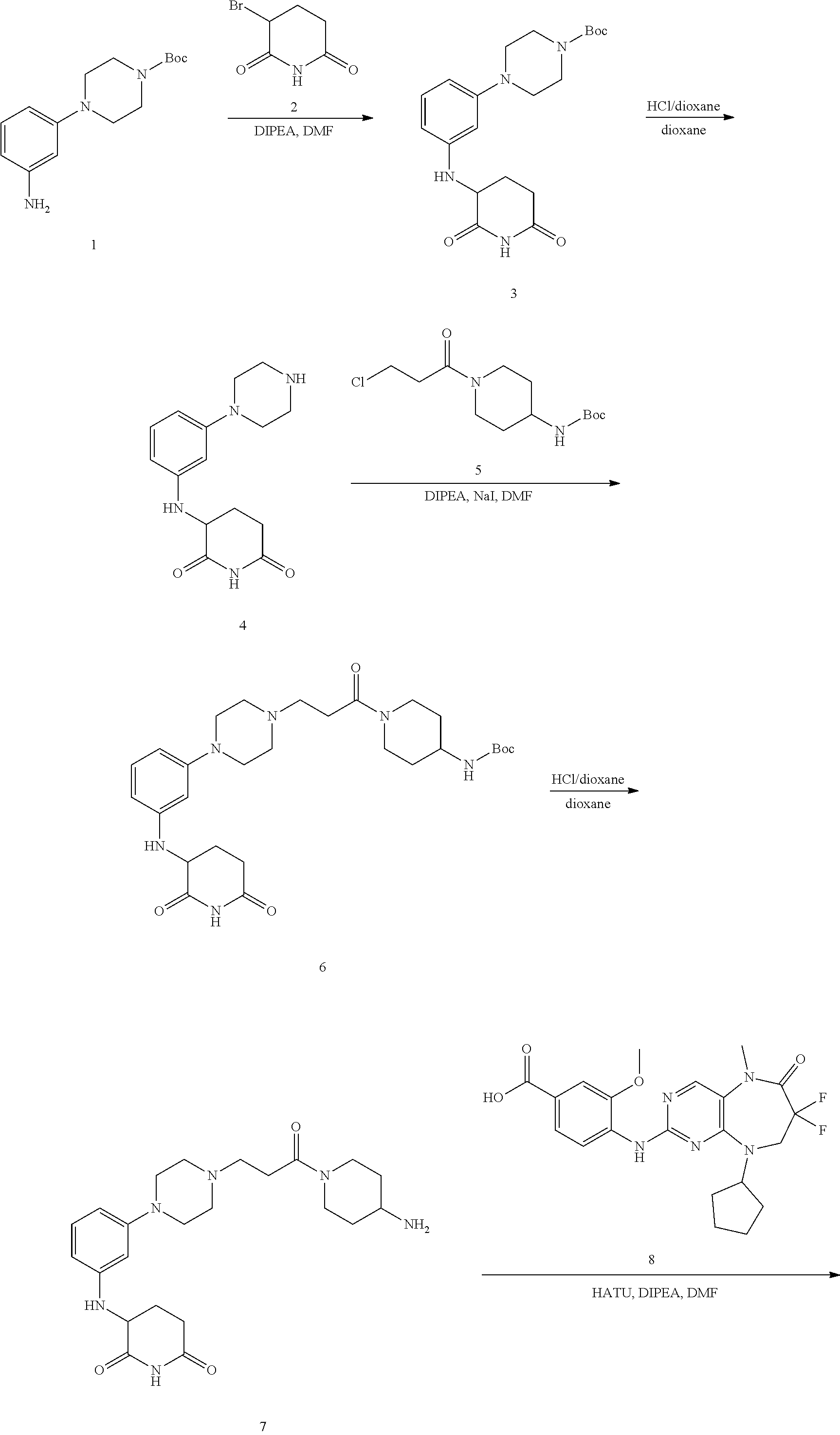

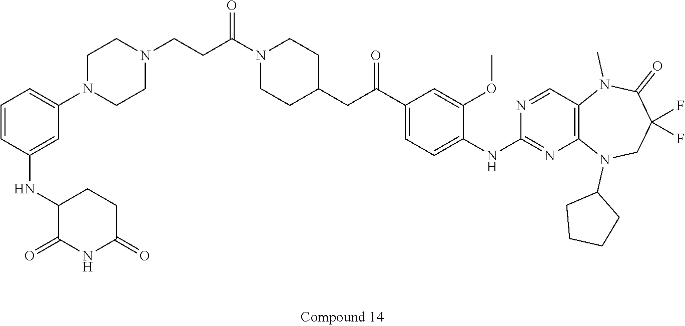 [SMILES: COc1cc(C(=O)CC2CCN(C(=O)CCN3CCN(c4cccc(NC5CCC(=O)NC5=O)c4)CC3)CC2)ccc1Nc1ncc2c(n1)N(C1CCCC1)CC(F)(F)C(=O)N2C]

### Step 1. Synthesis of tert-butyl 4-(3-((2,6-dioxopiperidin-3-yl)amino)phenyl)piperazine-1-carboxylate (3)

To a solution of tert-butyl 4-(3-aminophenyl)piperazine-1-carboxylate (1 g, 3.61 mmol) in DMF (10 mL) were added DIPEA (1.40 g, 10.82 mmol, 1.88 mL) and 3-bromopiperidine-2,6-dione (692.28 mg, 3.61 mmol), the mixture was stirred at 50° C. for 16 h. LCMS showed 42% of the starting material was remained and a peak (48%) with desired mass. The reaction mixture was diluted with H₂O (30 mL), extracted with EtOAc (30 mL×3). The combined organic layers were washed with brine (60 mL×2), dried over Na₂SO₄, filtered. The filtrate was concentrated under reduced pressure. The residue was purified by flash silica gel chromatography (Biotage; 10 g SepaFlash® Silica Flash Column, Eluent of 10-65% EtOAc/Petroleum ether @40 mL/min) to afford tert-butyl 4-(3-((2,6-dioxopiperidin-3-yl)amino)phenyl)piperazine-1-carboxylate (450 mg, 1.11 mmol, 30.84% yield, 96% purity) as a yellow solid. MS (M+H)⁺=389.0

### Step 2. Synthesis of 3-((3-(piperazin-1-yl)phenyl)amino)piperidine-2,6-dione (4)

To a solution of tert-butyl 4-(3-((2,6-dioxopiperidin-3-yl)amino)phenyl)piperazine-1-carboxylate (450 mg, 1.16 mmol) in dioxane (4 mL) was added HCl/dioxane (4 M, 4 mL), the mixture was stirred at 20° C. for 10 min. LCMS showed a main peak with desired mass. The mixture was concentrated under reduced pressure to afford 3-((3-(piperazin-1-yl)phenyl)amino)piperidine-2,6-dione (380 mg, crude, HCl salt) as a white solid, which was used into the next step directly. MS (M+H)⁺=289.0

### Step 3. Synthesis of tert-butyl (1-(3-(4-(3-((2,6-dioxopiperidin-3-yl)amino)phenyl)piperazin-1-yl)propanoyl)piperidin-4-yl)carbamate (6)

To a solution of 3-((3-(piperazin-1-yl)phenyl)amino)piperidine-2,6-dione (200 mg, 615.75 μmol, HCl salt) in DMF (5 mL) were added tert-butyl (1-(3-chloropropanoyl)piperidin-4-yl)carbamate (268.58 mg, 923.63 μmol), DIPEA (238.75 mg, 1.85 mmol, 321.76 μL) and NaI (9.23 mg, 61.58 μmol), the mixture was stirred at 60° C. for 24 h. LCMS showed a main peak with desired mass. The reaction mixture was diluted with H₂O (15 mL), extracted with EtOAc (15 mL×3). The combined organic layers were washed with brine (30 mL×2), dried over Na₂SO₄, filtered, and the filtrate was concentrated under reduced pressure. The residue was purified by flash silica gel chromatography (Biotage; 10 g SepaFlash® Silica Flash Column, Eluent of 50-100% EtOAc/Petroleum ether to 10% Methanol/EtOAc gradient @60 mL/min) to afford tert-butyl (1-(3-(4-(3-((2,6-dioxopiperidin-3-yl)amino)phenyl)piperazin-1-yl)propanoyl)piperidin-4-yl)carbamate (100 mg, 178.75 μmol, 29.03% yield, 97% purity) as a yellow oil. MS (M+H)⁺=543.3

### Step 4. Synthesis of 3-((3-(4-(3-(4-aminopiperidin-1-yl)-3-oxopropyl)piperazin-1-yl)phenyl)amino) piperidine-2,6-dione (7)

To a solution of tert-butyl (1-(3-(4-(3-((2,6-dioxopiperidin-3-yl)amino)phenyl)piperazin-1-yl)propanoyl)piperidin-4-yl)carbamate (100 mg, 184.27 μmol) in dioxane (4 mL) was added HCl/dioxane (4 M, 4 mL), the mixture was stirred at 20° C. for 1 h. LCMS showed a main peak with desired mass. The mixture was concentrated under reduced pressure to afford 3-((3-(4-(3-(4-aminopiperidin-1-yl)-3-oxopropyl)piperazin-1-yl)phenyl)amino)piperidine-2,6-dione (88 mg, crude, HCl salt) as a white solid. MS (M+H)⁺=443.1

### Step 5. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(4-(3-((2,6-dioxopiperidin-3-yl)amino)phenyl)piperazin-1-yl)propanoyl)piperidin-4-yl)-3-methoxybenzamide (Compound 14)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (74.73 mg, 167.01 μmol) in DMF (2 mL) were added HATU (95.25 mg, 250.51 μmol) and DIPEA (64.75 mg, 501.03 μmol, 87.27 μL), the mixture was stirred at 20° C. for 0.5 h. Then 3-((3-(4-(3-(4-aminopiperidin-1-yl)-3-oxopropyl)piperazin-1-yl)phenyl)amino)piperidine-2,6-dione (80 mg, 167.01 μmol, HCl salt) was added and the resulting mixture was stirred at 20° C. for 16 h. LCMS showed a main peak with desired mass. The reaction mixture was diluted with brine (10 mL) and extracted with EtOAc (10 mL×3). The combined organic layers were washed with brine (30 mL×2), dried over Na₂SO₄, filtered and concentrated under reduced pressure. The residue was purified by prep-TLC (SiO₂, DCM:MeOH=10:1) and re-purified by reversed-phase HPLC (column: Waters Xbridge 150×25 mm×5 μm; mobile phase: [water (NH₄HCO₃)-ACN]; B %: 32%-62%, 8 min). The eluent was lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(4-(3-((2,6-dioxopiperidin-3-yl)amino)phenyl)piperazin-1-yl)propanoyl)piperidin-4-yl)-3-methoxybenzamide (50 mg, 55.05 μmol, 32.96% yield, 96% purity) as a white solid. MS (M+H)⁺=872.1

¹H NMR (400 MHz, DMSO-d₆) δ=10.77 (s, 1H), 8.31-8.24 (m, 2H), 8.17 (d, J=7.8 Hz, 1H), 7.98 (s, 1H), 7.53-7.45 (m, 2H), 6.91 (t, J=8.1 Hz, 1H), 6.31-6.10 (m, 3H), 5.63 (d, J=7.6 Hz, 1H), 4.84-4.69 (m, 1H), 4.45-4.27 (m, 2H), 4.13-4.00 (m, 3H), 4.00-3.90 (m, 4H), 3.33 (s, 3H), 3.18-3.02 (m, 5H), 2.81-2.53 (m, 11H), 2.15-2.04 (m, 1H), 2.00-1.79 (m, 5H), 1.76-1.67 (m, 2H), 1.66-1.55 (m, 4H), 1.54-1.34 (m, 2H).

### Example 15. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(4-(3-((2,6-dioxopiperidin-3-yl)(methyl)amino)phenyl)piperazin-1-yl)propanoyl)piperidin-4-yl)-3-methoxybenzamide (Compound 15)

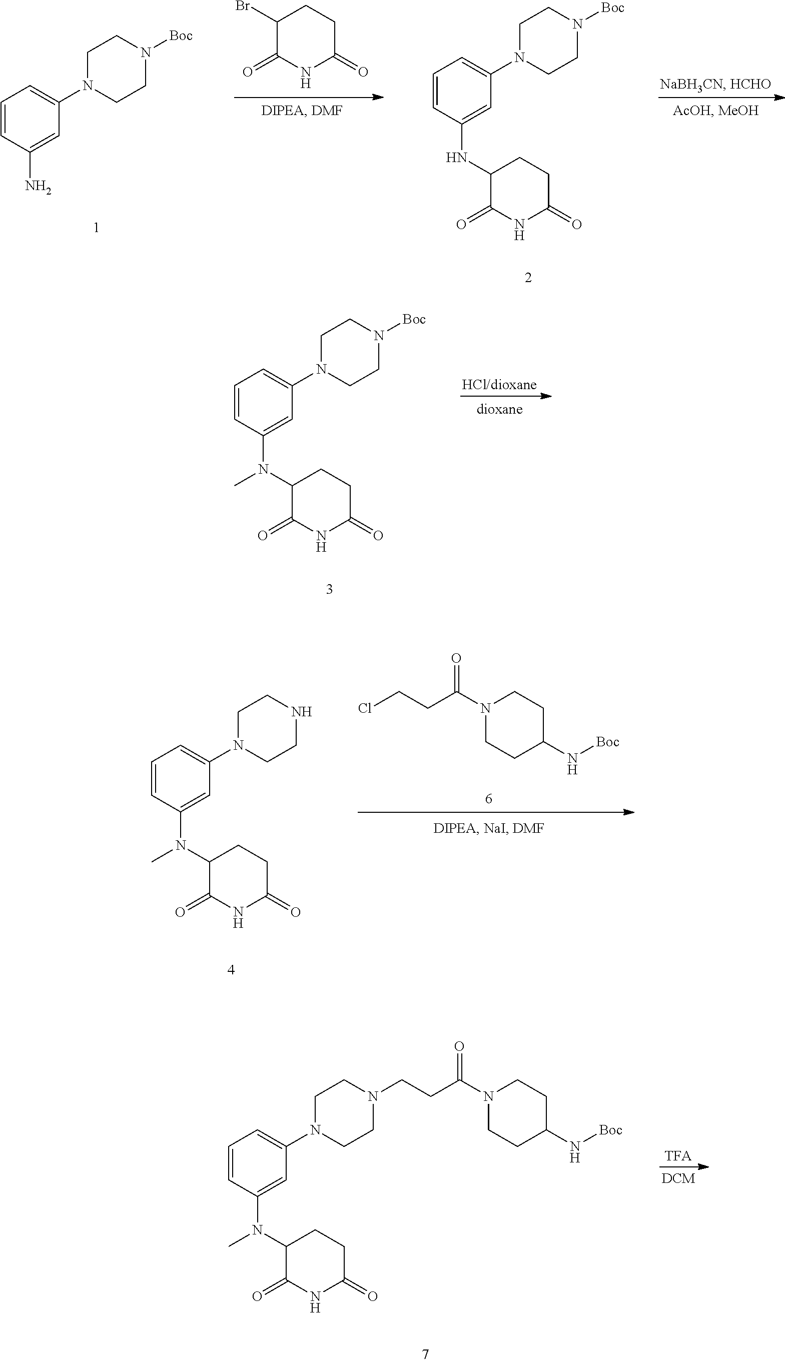

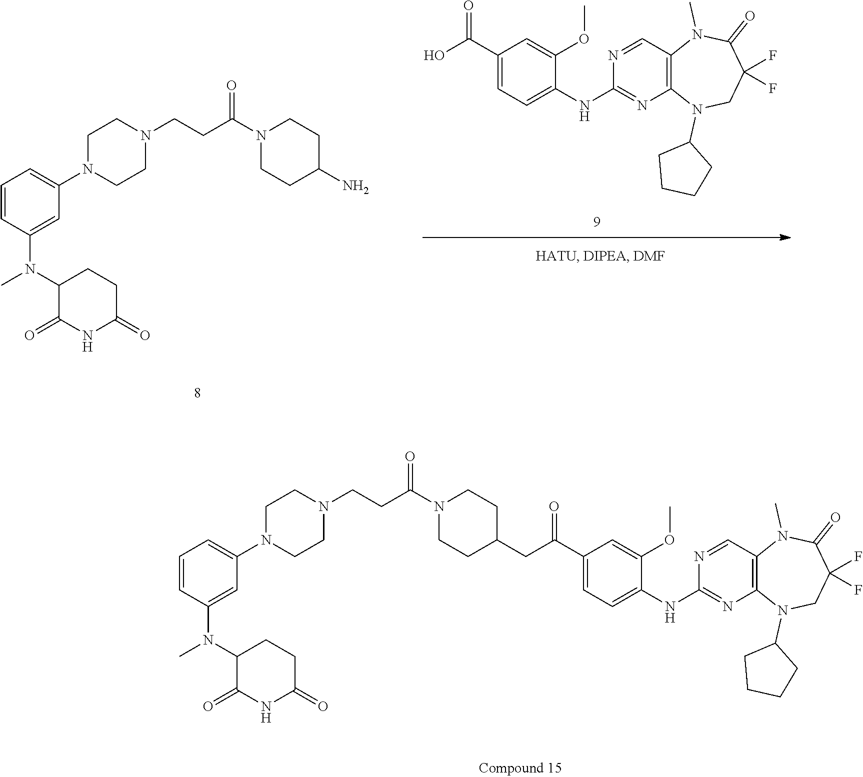 [SMILES: CN(c1cccc(N2CCN(CCC(=O)N3CCC(N)CC3)CC2)c1)C1CCC(=O)NC1=O ; COc1cc(C(=O)O)ccc1Nc1ncc2c(n1)N(C1CCCC1)CC(F)(F)C(=O)N2C ; COc1cc(C(=O)CC2CCN(C(=O)CCN3CCN(c4cccc(N(C)C5CCC(=O)NC5=O)c4)CC3)CC2)ccc1Nc1ncc2c(n1)N(C1CCCC1)CC(F)(F)C(=O)N2C]

### Step 1. Synthesis of tert-butyl 4-[3-[(2,6-dioxo-3-piperidyl) amino]phenyl]piperazine-1-carboxylate (2)

To a solution of tert-butyl 4-(3-aminophenyl)piperazine-1-carboxylate (1 g, 3.61 mmol) in DMF (10 mL) was added DIPEA (1.40 g, 10.82 mmol, 1.88 mL) and tert-butyl 4-(3-aminophenyl)piperazine-1-carboxylate (1.09 g, 5.70 mmol). The mixture was stirred at 50° C. for 16 hrs. LCMS showed a peak with desired mass. The reaction mixture was diluted with water (10 mL) and extracted with EtOAc (30 mL×3). The combined organic layers were washed with brine (20 mL×2), dried over (Na₂SO₄), filtered and concentrated under reduced pressure. The residue was purified by flash silica gel chromatography (20 g SepaFlash® silica Flash Column, Eluent of 20-30% EtOAc/Petroleum ether gradient@60 mL/min) to afford tert-butyl 4-[3-[(2,6-dioxo-3-piperidyl) amino]phenyl]piperazine-1-carboxylate (500 mg, 1.29 mmol, 35.70% yield) as a yellow solid. MS (M+H)⁺=389.3

### Step 2. Synthesis of tert-butyl 4-[3-[(2,6-dioxo-3-piperidyl)-methyl-amino]phenyl]piperazine-1-carboxylate (3)

To a solution of tert-butyl 4-[3-[(2, 6-dioxo-3-piperidyl)amino]phenyl]piperazine-1-carboxylate (500 mg, 1.29 mmol) and HCHO (193.24 mg, 1.93 mmol, 177.28 μL, 37% purity) in MeOH (5 mL) were added HOAc (7.73 mg, 128.71 μmol, 7.36 μL) and sodium cyanoborohydride (121.33 mg, 1.93 mmol). The mixture was stirred at 25° C. for 2 hrs. LCMS showed a main peak with desired mass. The reaction mixture was concentrated under reduced pressure to remove MeOH. The residue was purified by flash silica gel chromatography (20 g SepaFlash® silica Flash Column, Eluent of 40-70% EtOAc/Petroleum ether gradient @60 mL/min) to afford tert-butyl 4-[3-[(2,6-dioxo-3-piperidyl)-methyl-amino]phenyl]piperazine-1-carboxylate (300 mg, 693.19 μmol, 53.86% yield, 93% purity) as a white solid. MS (M+H)⁺=403.3

### Step 3. Synthesis of 3-(methyl(3-(piperazin-1-yl)phenyl)amino)piperidine-2,6-dione (4)

To a solution of tert-butyl 4-[3-[(2,6-dioxo-3-piperidyl)-methyl-amino]phenyl]piperazine-1-carboxylate (250 mg, 621.14 μmol) in dioxane (1 mL) was added HCl/dioxane (4 M, 10.00 mL). The mixture was stirred at 25° C. for 16 hrs. LCMS showed a peak with desired mass. The reaction solution was concentrated to afford 3-(methyl(3-(piperazin-1-yl)phenyl)amino)piperidine-2,6-dione (200 mg, crude, HCl salt) as a white solid. MS (M+H)⁺=303.3

### Step 4. Synthesis of tert-butyl (1-(3-(4-(3-((2,6-dioxopiperidin-3-yl)(methyl)amino)phenyl)piperazin-1-yl)propanoyl)piperidin-4-yl)carbamate (7)

To a solution of 3-(methyl(3-(piperazin-1-yl)phenyl)amino)piperidine-2,6-dione (200 mg, 661.44 μmol, HCl salt) in DMF (2 mL) were added NaI (99.15 mg, 661.44 μmol), DIPEA (427.43 mg, 3.31 mmol, 576.05 μL) and tert-butyl N-[1-(3-chloropropanoyl)-4-piperidyl]carbamate (192.34 mg, 661.44 μmol) and the resulting mixture was stirred at 80° C. for 16 hrs. LCMS showed a peak with desired mass. The reaction mixture was diluted with water (20 mL) and extracted with EtOAc (20 mL×2). The combined organic layers were washed with brine (10 mL×2), dried over Na₂SO₄ filtered and concentrated under reduced pressure. The residue was purified by prep-HPLC (column: Phenomenex luna C18 150×40 mm×15 μm; mobile phase: [water (TFA)-ACN]; B %: 13%-43%, 10 min) and the eluent was lyophilized to afford tert-butyl (1-(3-(4-(3-((2,6-dioxopiperidin-3-yl)(methyl)amino)phenyl)piperazin-1-yl)propanoyl) piperidin-4-yl)carbamate (160 mg, 287.41 μmol, 43.45% yield) as a white solid. MS (M+H)⁺=557.1

### Step 5. Synthesis of 3-((3-(4-(3-(4-aminopiperidin-1-yl)-3-oxopropyl)piperazin-1-yl)phenyl)(methyl)amino)piperidine-2,6-dione (8)

To a solution of tert-butyl (1-(3-(4-(3-((2,6-dioxopiperidin-3-yl)(methyl)amino)phenyl)piperazin-1-yl)propanoyl) piperidin-4-yl)carbamate (80 mg, 143.71 μmol) in DCM (1 mL) was added TFA (262.17 mg, 2.30 mmol, 170.24 μL). The mixture was stirred at 25° C. for 1 hrs. LCMS showed a peak with desired mass. The reaction solution was concentrated to afford 3-((3-(4-(3-(4-aminopiperidin-1-yl)-3-oxopropyl)piperazin-1-yl)phenyl)(methyl)amino)piperidine-2,6-dione (65 mg, crude, TFA salt) as a yellow solid. MS (M+H)⁺=457.1

### Step 6. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(4-(3-((2,6-dioxopiperidin-3-yl)(methyl)amino)phenyl)piperazin-1-yl)propanoyl)piperidin-4-yl)-3-methoxybenzamide (Compound 15)

To a solution of 3-((3-(4-(3-(4-aminopiperidin-1-yl)-3-oxopropyl)piperazin-1-yl)phenyl)(methyl)amino)piperidine-2,6-dione (65 mg, 142.36 μmol, TFA) in DMF (1 mL) were added HATU (64.96 mg, 170.84 μmol), DIPEA (92.00 mg, 711.81 μmol, 123.99 μL) and 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (63.70 mg, 142.36 μmol). The mixture was stirred at 25° C. for 1 hrs. LCMS showed a peak with desired mass. The reaction mixture was purified by prep-HPLC (column: Waters Xbridge 150×25 mm×5 m; mobile phase: [water (NH₄HCO₃)-ACN]; B %: 36%-66%, 8 min) and the eluent was lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(4-(3-((2,6-dioxopiperidin-3-yl)(methyl)amino)phen yl)piperazin-1-yl)propanoyl)piperidin-4-yl)-3-methoxybenzamide (32 mg, 33.95 μmol, 23.85% yield, 94% purity) as a white solid. MS (M+H)⁺=886.6

¹H NMR (400 MHz, DMSO-d₆) δ=ppm 10.76 (s, 1H), 8.32-8.24 (m, 2H), 8.15 (br d, J=7.70 Hz, 1H), 7.96 (s, 1H), 7.53-7.44 (m, 2H), 6.99 (t, J=8.13 Hz, 1H), 6.36-6.22 (m, 3H), 4.99-4.72 (m, 1H), 4.81-4.71 (m, 1H), 4.39 (br d, J=12.23 Hz, 1H), 4.04 (br t, J=14.00 Hz, 3H), 3.98-3.90 (m, 4H), 3.27 (s, 3H), 3.17-3.08 (m, 5H), 2.91-2.77 (m, 1H), 2.77-2.55 (m, 13H), 2.31-2.22 (m, 1H), 2.02-1.81 (m, 5H) 1.77-1.66 (m, 2H), 1.65-1.55 (m, 4H), 1.51-1.33 (m, 2H).

### Example 16. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(4-(4-((2,6-dioxopiperidin-3-yl)(methyl)amino)-2-fluorophenyl)piperazin-1-yl)propanoyl)piperidin-4-yl)-3-methoxybenzamide (Compound 16)

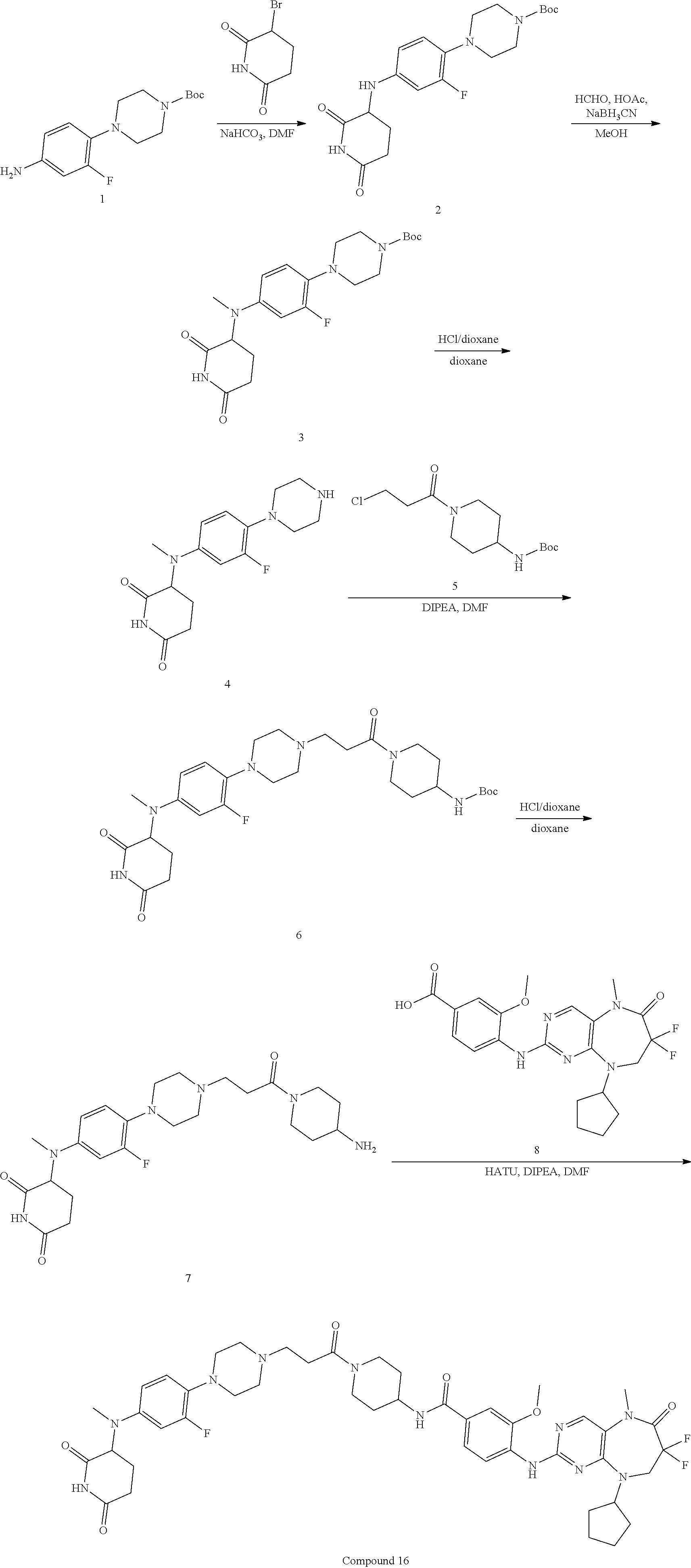

### Step 1. Synthesis of tert-butyl 4-(4-((2,6-dioxopiperidin-3-yl)amino)-2-fluorophenyl)piperazine-1-carboxylate (2)

To a stirred solution of tert-butyl 4-(4-amino-2-fluorophenyl)piperazine-1-carboxylate (1 g, 3.39 mmol) in DMF (20 mL) were added 3-bromopiperidine-2,6-dione (1.95 g, 10.16 mmol) and NaHCO₃ (2.84 g, 33.86 mmol, 1.32 mL), the mixture was stirred at 85° C. for 16 h. LCMS showed a main peak with desired mass. The reaction mixture was diluted with brine (60 mL), and extracted with EtOAc (50 mL×5). The combined organic layers were washed with brine (200 mL×2), dried over Na₂SO₄, filtered. The filtrate was concentrated under reduced pressure. The residue was purified by flash silica gel chromatography (Biotage; 20 g SepaFlash® Silica Flash Column, Eluent of 20˜70% EtOAc/Petroleum ether to 10% Methanol/EtOAc gradient@40 mL/min) to afford tert-butyl 4-(4-((2,6-dioxopiperidin-3-yl)amino)-2-fluorophenyl)piperazine-1-carboxylate (670 mg, 1.57 mmol, 46.25% yield, 95% purity) as a blue solid. MS (M+H)⁺=406.9

### Step 2. Synthesis of tert-butyl 4-(4-((2,6-dioxopiperidin-3-yl)(methyl)amino)-2-fluorophenyl) piperazine-1-carboxylate (3)

To a solution of tert-butyl 4-(4-((2,6-dioxopiperidin-3-yl)amino)-2-fluorophenyl)piperazine-1-carboxylate (620 mg, 1.53 mmol) and formaldehyde (185.68 mg, 2.29 mmol, 170.35 μL, 37% purity) in MeOH (10 mL) was added acetic acid (9.16 mg, 152.54 μmol, 8.72 L), the mixture was stirred at 20° C. for 0.5 h. NaBH₃CN (143.79 mg, 2.29 mmol) was added and the resulting mixture was stirred at 20° C. for 16 hr. LCMS showed a main peak with desired mass. The reaction mixture was diluted with H₂O (20 mL), and extracted with EtOAc (20 mL×3). The combined organic layers were washed with brine (20 mL×2), dried over Na₂SO₄, filtered. The filtrate was concentrated under reduced pressure. The residue was purified by flash silica gel chromatography (Biotage; 20 g SepaFlash® Silica Flash Column, Eluent of 20˜70% EtOAc/Petroleum ether@40 mL/min) to afford tert-butyl 4-(4-((2,6-dioxopiperidin-3-yl)(methyl)amino)-2-fluorophenyl)piperazine-1-carboxylate (200 mg, 437.60 μmol, 28.69% yield, 92% purity) as a blue solid. MS (M+H)⁺=420.8

### Step 3. Synthesis of 3-((3-fluoro-4-(piperazin-1-yl)phenyl)(methyl)amino)piperidine-2,6-dione (4)

To a solution of tert-butyl 4-(4-((2,6-dioxopiperidin-3-yl)(methyl)amino)-2-fluorophenyl)piperazine-1-carboxylate (100 mg, 237.83 μmol) in dioxane (2 mL) was added HCl/dioxane (4 M, 2 mL), the mixture was stirred at 20° C. for 0.5 h. LCMS showed a main peak with desired mass. The mixture was concentrated under reduced pressure to afford 3-((3-fluoro-4-(piperazin-1-yl)phenyl)(methyl)amino)piperidine-2,6-dione (80 mg, crude, HCl salt) as a blue solid. MS (M+H)⁺=321.1

### Step 4. Synthesis of tert-butyl (1-(3-(4-(4-((2,6-dioxopiperidin-3-yl)(methyl)amino)-2-fluorophenyl) piperazin-1-yl)propanoyl)piperidin-4-yl)carbamate (6)

To a solution of 3-((3-fluoro-4-(piperazin-1-yl)phenyl)(methyl)amino)piperidine-2,6-dione (80 mg, 224.20 μmol, HCl salt) in DMF (1 mL) were added tert-butyl (1-(3-chloropropanoyl)piperidin-4-yl)carbamate (97.79 mg, 336.30 μmol) and DIPEA (86.93 mg, 672.60 μmol, 117.16 L), the mixture was stirred at 80° C. for 16 h. LCMS showed a main peak with desired mass. The reaction mixture was diluted with H₂O (3 mL), extracted with EtOAc (3 mL×3), the combined organic layer was washed with brine (10 mL×3), dried over Na₂SO₄, filtered. The filtrate was concentrated in vacuo. The residue was purified by flash silica gel chromatography (Biotage; 10 g SepaFlash® Silica Flash Column, Eluent of 20-100% EtOAc/Petroleum ether to 10% Methanol/EtOAc gradient@40 mL/min) to afford tert-butyl (1-(3-(4-(4-((2,6-dioxopiperidin-3-yl)(methyl)amino)-2-fluorophenyl)piperazin-1-yl)propanoyl)piperidin-4-yl)carbamate (100 mg, 161.83 μmol, 72.18% yield, 93% purity) as a brown solid. MS (M+H)⁺=575.4

### Step 5. Synthesis of 3-((4-(4-(3-(4-aminopiperidin-1-yl)-3-oxopropyl)piperazin-1-yl)-3-fluorophenyl)(methyl)amino)piperidine-2,6-dione (7)

To a solution of tert-butyl (1-(3-(4-(4-((2,6-dioxopiperidin-3-yl)(methyl)amino)-2-fluorophenyl)piperazin-1-yl)propanoyl)piperidin-4-yl)carbamate (100 mg, 174.01 μmol) in dioxane (2 mL) was added HCl/dioxane (4 M, 43.50 L), the mixture was stirred at 20° C. for 1 h. LCMS showed a main peak with desired mass. The mixture was concentrated under reduced pressure to afford 3-((4-(4-(3-(4-aminopiperidin-1-yl)-3-oxopropyl)piperazin-1-yl)-3-fluorophenyl)(methyl)amino)piperidine-2,6-dione (80 mg, 156.55 μmol, 89.96% yield, HCl salt) as a white solid, which was used into the next step directly. MS (M+H)⁺=475.3

### Step 6. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(4-(4-((2,6-dioxopiperidin-3-yl)(methyl)amino)-2-fluorophenyl)piperazin-1-yl)propanoyl)piperidin-4-yl)-3-methoxybenzamide (Compound 16)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (70.04 mg, 156.55 μmol) in DMF (2 mL) were added HATU (89.29 mg, 234.82 μmol) and DIPEA (60.70 mg, 469.64 μmol, 81.80 μL), the mixture was stirred at 20° C. for 0.5 h. 3-((4-(4-(3-(4-aminopiperidin-1-yl)-3-oxopropyl)piperazin-1-yl)-3-fluorophenyl)(methyl)amino)piperidine-2,6-dione (80 mg, 156.55 μmol, HCl salt) was added and the resulting mixture was stirred at 20° C. for 1 h. LCMS showed a main peak with desired mass. The reaction mixture was diluted with brine (5 mL), and extracted with EtOAc (5 mL×3). The combined organic layers were washed with brine (10 mL×2), dried over Na₂SO₄, filtered and concentrated under reduced pressure. The residue was purified by prep-TLC (SiO₂, DCM:MeOH=10:1) and re-purified by reversed-phase HPLC (column: Waters Xbridge 150×25 mm×5 μm; mobile phase: [water (NH₄HCO 3)-ACN]; B %: 30%-63%, 9 min). The eluent was lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(4-(4-((2,6-dioxopiperidin-3-yl)(methyl)amino)-2-fluorophenyl)piperazin-1-yl)propanoyl)piperidin-4-yl)-3-methoxybenzamide (51.2 mg, 54.94 μmol, 35.09% yield, 97% purity) as a white solid. MS (M+H)⁺=904.5

¹H NMR (400 MHz, DMSO-d₆) δ=10.78 (s, 1H), 8.32-8.24 (m, 2H), 8.15 (d, J=7.6 Hz, 1H), 7.97 (s, 1H), 7.53-7.45 (m, 2H), 6.88 (t, J=9.6 Hz, 1H), 6.72-6.63 (m, 1H), 6.57-6.48 (m, 1H), 4.85-4.71 (m, 2H), 4.43-4.35 (m, 1H), 4.04 (t, J=13.9 Hz, 3H), 3.98-3.89 (m, 4H), 3.31 (s, 3H), 3.19-3.08 (m, 1H), 2.93-2.77 (m, 5H), 2.73-2.63 (m, 5H), 2.59-2.54 (m, 7H), 2.31-2.20 (m, 1H), 1.98-1.80 (m, 5H), 1.77-1.52 (m, 7H), 1.51-1.34 (m, 2H).

### Example 17. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(4-((4-(4-((2,6-dioxopiperidin-3-yl)amino)-2-fluorophenyl)piperazin-1-yl)methyl)piperidin-1-yl)-3-methoxybenzamide (Compound 17)

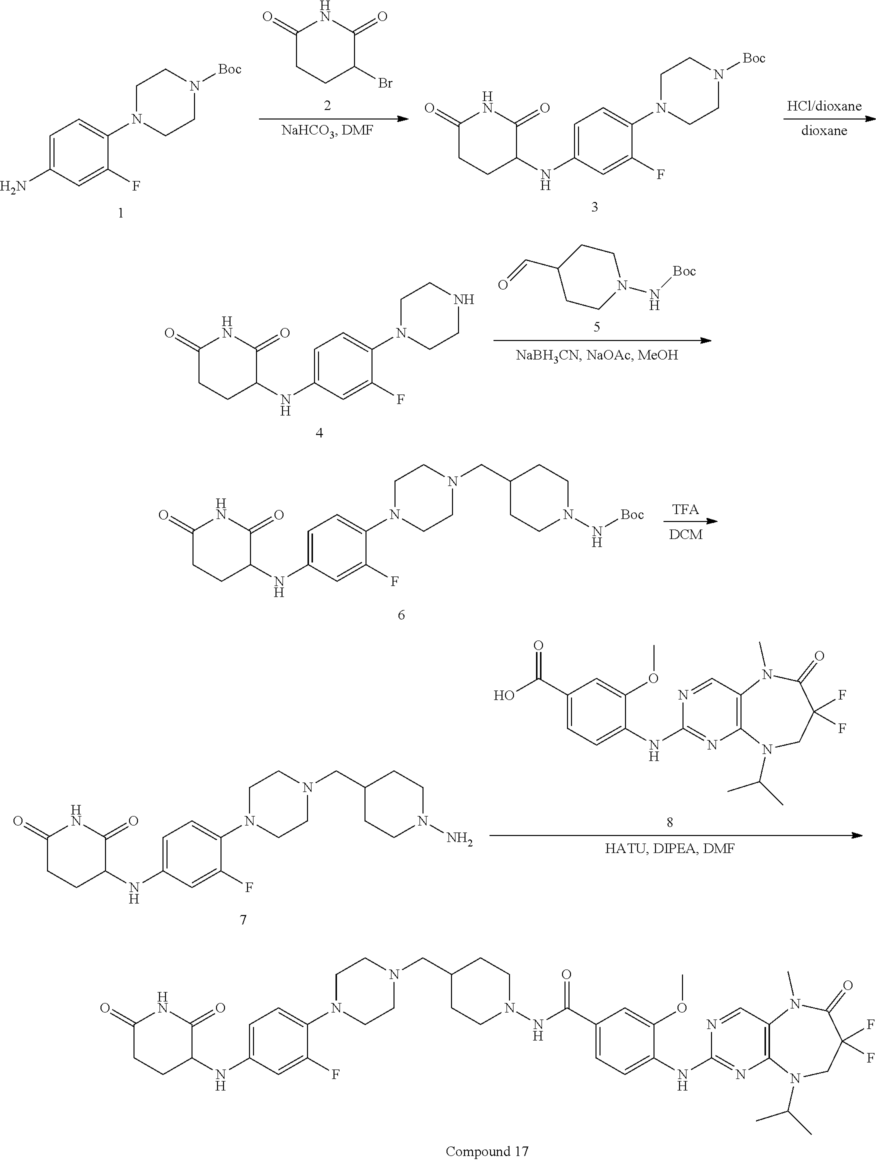

### Step 1. Synthesis of tert-butyl 4-(4-((2,6-dioxopiperidin-3-yl)amino)-2-fluorophenyl)piperazine-1-carboxylate (3)

To a stirred solution of tert-butyl 4-(4-amino-2-fluorophenyl)piperazine-1-carboxylate (2 g, 6.77 mmol) in DMF (40 mL) were added 3-bromopiperidine-2,6-dione (2.60 g, 13.54 mmol) and NaHCO₃ (2.84 g, 33.86 mmol, 1.32 mL), and stirred at 85° C. for 16 h. LCMS showed a main peak with desired mass. The reaction mixture was diluted with brine (120 mL), the aqueous layer was extracted with EtOAc (120 mL×3). The combined organic layers were washed with brine (200 mL×2), dried over Na₂SO₄, filtered. The filtrate was concentrated under reduced pressure to give a residue. The residue was purified by flash silica gel chromatography (Biotage; 20 g SepaFlash® Silica Flash Column, Eluent of 0˜60% EtOAc/Petroleum ether gradient@40 mL/min) to afford tert-butyl 4-(4-((2,6-dioxopiperidin-3-yl)amino)-2-fluorophenyl)piperazine-1-carboxylate (1 g, 2.46 mmol, 36.33% yield, 100% purity) as a blue solid. MS (M+H)⁺=407.0

### Step 2. Synthesis of 3-((3-fluoro-4-(piperazin-1-yl)phenyl)amino)piperidine-2,6-dione (4)

To a solution of tert-butyl 4-(4-((2,6-dioxopiperidin-3-yl)amino)-2-fluorophenyl)piperazine-1-carboxylate (1 g, 2.46 mmol) in dioxane (10 mL) was added HCl/dioxane (4 M, 10 mL), the mixture was stirred at 20° C. for 2 h. LCMS showed a main peak with desired mass. The mixture was concentrated in vacuum to afford 3-((3-fluoro-4-(piperazin-1-yl)phenyl)amino)piperidine-2,6-dione (800 mg, crude, HCl salt) as a blue solid. MS (M+H)⁺=306.9

### Step 3. Synthesis of tert-butyl (4-((4-(4-((2,6-dioxopiperidin-3-yl)amino)-2-fluorophenyl)piperazin-1-yl)methyl)piperidin-1-yl)carbamate (6)

To a solution of 3-((3-fluoro-4-(piperazin-1-yl)phenyl)amino)piperidine-2,6-dione (800 mg, crude, HCl salt) and tert-butyl (4-formylpiperidin-1-yl)carbamate (532.77 mg, 2.33 mmol) in MeOH (20 mL) was added NaOAc (382.89 mg, 4.67 mmol), the mixture was stirred at 20° C. for 0.5 h, NaBH₃CN (439.97 mg, 7.00 mmol) was added, the mixture was stirred at 20° C. for 16 h. LCMS showed a peak (10%) with desired mass. The reaction mixture was diluted with H₂O (50 mL), extracted with EtOAc (50 mL×3). The combined organic layers were washed with brine (50 mL×2), dried over Na₂SO₄, filtered. The filtrate was concentrated under reduced pressure. The residue was purified by flash silica gel chromatography (Biotage; 20 g SepaFlash® Silica Flash Column, Eluent of 50-100% EtOAc/Petroleum ether to 10% Methanol/EtOAc gradient@40 mL/min) twice and re-purified by prep-HPLC (column: Phenomenex luna C18 150*40 mm*15 m; mobile phase: [water (FA)-ACN]; B %: 8%-38%, 10 min), the eluent was lyophilized to afford tert-butyl (4-((4-(4-((2,6-dioxopiperidin-3-yl)amino)-2-fluorophenyl)piperazin-1-yl)methyl)piperidin-1-yl)carbamate (350 mg, 661.37 μmol, 28.34% yield, 98% purity) as a brown solid. MS (M+H)⁺=519.4

### Step 4. Synthesis of 3-((4-(4-((1-aminopiperidin-4-yl)methyl)piperazin-1-yl)-3-fluorophenyl)amino)piperidine-2,6-dione (7)

To a solution of tert-butyl (4-((4-(4-((2,6-dioxopiperidin-3-yl)amino)-2-fluorophenyl)piperazin-1-yl)methyl)piperidin-1-yl)carbamate (350 mg, 674.86 μmol) in DCM (10 mL) was added TFA (3.08 g, 27.01 mmol, 2 mL), the mixture was stirred at 20° C. for 1 h. LCMS showed a main peak with desired mass. The reaction mixture was concentrated under reduced pressure to afford 3-((4-(4-((1-aminopiperidin-4-yl)methyl)piperazin-1-yl)-3-fluorophenyl)amino)piperidine-2,6-dione (350 mg, crude, TFA salt) as a yellow oil. MS (M+H)⁺=419.2

### Step 5. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(4-((4-(4-((2,6-dioxopiperidin-3-yl)amino)-2-fluorophenyl)piperazin-1-yl)methyl)piperidin-1-yl)-3-methoxybenzamide (Compound 17)

To a solution of 4-((7,7-difluoro-9-isopropyl-5-methyl-6-oxo-6,7,8,9-tetrahydro-5H-pyrimido[4,5-b][1,4]diazepin-2-yl)amino)-3-methoxybenzoic acid (276.96 mg, 657.24 μmol) in DMF (10 mL) were added HATU (374.85 mg, 985.86 μmol) and DIPEA (254.83 mg, 1.97 mmol, 343.44 μL), the mixture was stirred at 20° C. for 0.5 h, then 3-((4-(4-((1-aminopiperidin-4-yl)methyl)piperazin-1-yl)-3-fluorophenyl)amino)piperidine-2,6-dione (350 mg, crude, TFA salt) was added and the resulting mixture was stirred at 20° C. for 16 h. LCMS showed a main peak with desired mass. The reaction mixture was diluted with H₂O (20 mL), extracted with EtOAc (20 mL×3). The combined organic layers were washed with brine (20 mL×2), dried over Na₂SO₄, filtered. The filtrate was concentrated under reduced pressure. The residue was purified by flash silica gel chromatography (Biotage; 20 g SepaFlash® Silica Flash Column, Eluent of 20-100% EtOAc/Petroleum ether to 10% Methanol/EtOAc gradient@40 mL/min) and re-purified by prep-HPLC (column: Waters Xbridge C18 150*50 mm*10 μm; mobile phase: [water (NH₄HCO₃)-ACN]; B %: 30%-60%, 10 min). The eluent was lyophilized to afford 4-((7,7-difluoro-9-isopropyl-5-methyl-6-oxo-6,7,8,9-tetrahydro-5H-pyrimido[4,5-b][1,4]diazepin-2-yl)amino)-N-(4-((4-(4-((2,6-dioxopiperidin-3-yl)amino)-2-fluorophenyl)piperazin-1-yl)methyl)piperidin-1-yl)-3-methoxybenzamide (207.1 mg, 249.46 μmol, 37.96% yield, 99% purity) as a yellow solid. MS (M+H)⁺=822.4

¹H NMR (400 MHz, DMSO-d₆) δ=10.77 (s, 1H), 9.27 (s, 1H), 8.33-8.27 (m, 1H), 8.22 (s, 1H), 7.87 (s, 1H), 7.48-7.39 (m, 2H), 6.83 (t, J=9.4 Hz, 1H), 6.54-6.47 (m, 1H), 6.45-6.39 (m, 1H), 5.80 (d, J=7.6 Hz, 1H), 4.93-4.83 (m, 1H), 4.31-4.21 (m, 1H), 4.03 (t, J=13.5 Hz, 2H), 3.93 (s, 3H), 3.30 (s, 3H), 3.05-2.98 (m, 2H), 2.90-2.80 (m, 4H), 2.80-2.68 (m, 3H), 2.60-2.51 (m, 5H), 2.23-2.16 (m, 2H), 2.14-2.03 (m, 1H), 1.92-1.81 (m, 1H), 1.80-1.71 (m, 2H), 1.60-1.46 (m, 1H), 1.31-1.18 (m, 8H).

### Example 18. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(4-((4-(4-((2,6-dioxopiperidin-3-yl)amino)-2,6-difluorophenyl)piperazin-1-yl)methyl)piperidin-1-yl)-3-methoxybenzamide (Compound 18)

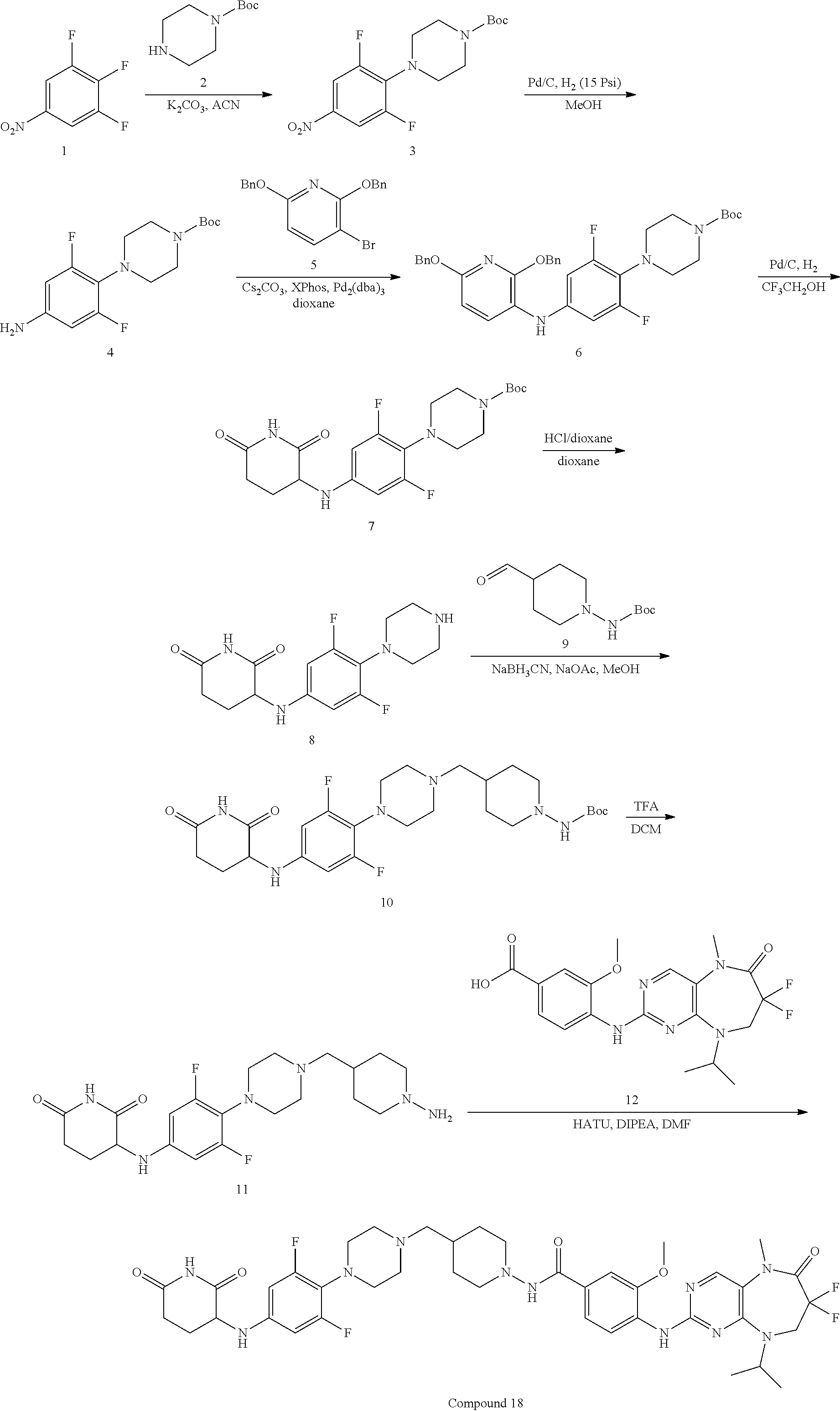

### Step 1. Synthesis of tert-butyl 4-(2,6-difluoro-4-nitrophenyl)piperazine-1-carboxylate (3)

To a solution of tert-butyl piperazine-1-carboxylate (6.29 g, 28.24 mmol) and 1,2,3-trifluoro-5-nitrobenzene (5 g, 28.24 mmol) in ACN (50 mL) was added K₂CO₃ (7.80 g, 56.47 mmol). The resulting mixture was heated to 50° C. for 16 hrs. LCMS showed a main peak with desired mass. The reaction mixture was diluted with H₂O (200 mL), extracted with EtOAc (200 mL×3). The combined organic layers were washed with brine (200 mL×2), dried over Na₂SO₄, filtered. The filtrate was concentrated under reduced pressure. The residue was triturated with MTBE (40 mL) at 20° C. for 10 min to afford tert-butyl 4-(2,6-difluoro-4-nitrophenyl)piperazine-1-carboxylate (9.5 g, 27.67 mmol, 98.00% yield) as a yellow solid. MS (M−56+H)⁺=288.0

### Step 2. Synthesis of tert-butyl 4-(4-amino-2,6-difluorophenyl)piperazine-1-carboxylate (4)

A mixture of tert-butyl 4-(2,6-difluoro-4-nitrophenyl)piperazine-1-carboxylate (9.5 g, 27.67 mmol) and Pd/C (1 g, 10% purity) in MeOH (200 mL) was degassed and purged with H₂ for 3 times. The mixture was stirred at 20° C. for 20 h under H₂ (15 Psi) atmosphere. TLC indicated the starting material was consumed completely, and one major new spot with larger polarity was detected. The mixture was filtered through a pad of celite. The filtrate was concentrated in vacuum to afford tert-butyl 4-(4-amino-2,6-difluorophenyl)piperazine-1-carboxylate (8 g, crude) as a yellow solid. MS (M+H)⁺=314.2

### Step 3. Synthesis of tert-butyl 4-(4-((2,6-bis(benzyloxy)pyridin-3-yl)amino)-2,6-difluorophenyl) piperazine-1-carboxylate (6)

A mixture of tert-butyl 4-(4-amino-2,6-difluorophenyl)piperazine-1-carboxylate (1 g, 3.19 mmol), 2,6-bis(benzyloxy)-3-bromopyridine (1.54 g, 4.15 mmol), Cs₂CO₃ (3.12 g, 9.57 mmol) in dioxane (40 mL) was degassed with nitrogen for 15 minutes. Then XPhos (152.14 mg, 319.14 μmol) and Pd₂(dba)₃ (292.24 mg, 319.14 μmol) were added and the mixture was degassed with nitrogen for 5 minutes. The resulting mixture was heated to 100° C. for 16 h under N₂ atmosphere. LCMS showed a main peak with desired mass. The mixture was filtered through a pad of celite. The filtrate was diluted with H₂O (100 mL), extracted with EtOAc (100 mL×3). The combined organic layers were washed with brine (200 mL×2), dried over Na₂SO₄, filtered. The filtrate was concentrated under reduced pressure. The residue was purified by flash silica gel chromatography (Biotage; 40 g SepaFlash® Silica Flash Column, Eluent of 0˜10% EtOAc/Petroleum ether gradient@40 mL/min) to afford tert-butyl 4-(4-((2,6-bis(benzyloxy)pyridin-3-yl)amino)-2,6-difluorophenyl)piperazine-1-carboxy late (1.9 g, 3.06 mmol, 95.82% yield, 97% purity) as a yellow solid. MS (M+H)⁺=603.2

### Step 4. Synthesis of tert-butyl 4-(4-((2,6-dioxopiperidin-3-yl)amino)-2,6-difluorophenyl)piperazine-1-carboxylate (7)

A mixture of tert-butyl 4-(4-((2,6-bis(benzyloxy)pyridin-3-yl)amino)-2,6-difluorophenyl)piperazine-1-carboxy late (1.9 g, 3.15 mmol) and Pd/C (200 mg, 10% purity) in CF₃CH₂OH (20 mL) was degassed and purged with H₂ for 3 times. The mixture was stirred at 20° C. for 16 h under H₂ (15 Psi) atmosphere. LCMS showed a main peak with desired mass. The mixture was filtered through a pad of celite. The filtrate was concentrated in vacuum to afford tert-butyl 4-(4-((2,6-dioxopiperidin-3-yl)amino)-2,6-difluorophenyl)piperazine-1-carboxylate (1.3 g, crude) as a blue solid. MS (M+H)⁺=425.2

### Step 5. Synthesis of 3-((3,5-difluoro-4-(piperazin-1-yl)phenyl)amino)piperidine-2,6-dione (8)

To a solution of tert-butyl 4-(4-((2,6-dioxopiperidin-3-yl)amino)-2,6-difluorophenyl)piperazine-1-carboxylate (300 mg, 706.81 μmol) in dioxane (3 mL) was added HCl/dioxane (4 M, 3 mL), the mixture was stirred at 20° C. for 1 h. LCMS showed a main peak with desired mass. The mixture was concentrated in vacuum to afford 3-((3,5-difluoro-4-(piperazin-1-yl)phenyl)amino)piperidine-2,6-dione (250 mg, crude, HCl salt) as a blue solid, which was used into the next step directly. MS (M+H)⁺=325.1

### Step 6. Synthesis of tert-butyl (4-((4-(4-((2,6-dioxopiperidin-3-yl)amino)-2,6-difluorophenyl) piperazin-1-yl)methyl)piperidin-1-yl)carbamate (10)

To a solution of 3-((3,5-difluoro-4-(piperazin-1-yl)phenyl)amino)piperidine-2,6-dione (250 mg, 692.93 mol, HCl salt), tert-butyl (4-formylpiperidin-1-yl)carbamate (158.19 mg, 692.93 mol) in MeOH (4 mL) was added NaOAc (113.69 mg, 1.39 mmol), the mixture was stirred at 20° C. for 1 h, then NaBH₃CN (130.64 mg, 2.08 mmol) was added and the resulting mixture was stirred at 20° C. for 16 h. LCMS showed a main peak with desired mass. The reaction mixture was diluted with H₂O (10 mL), extracted with EtOAc (10 mL×3). The combined organic layers were washed with brine (10 mL×2), dried over Na₂SO₄, filtered. The filtrate was concentrated under reduced pressure. The residue was purified by flash silica gel chromatography (Biotage; 20 g SepaFlash® Silica Flash Column, Eluent of 20-100% EtOAc/Petroleum ether to 10% Methanol/EtOAc gradient@40 mL/min) to afford tert-butyl (4-((4-(4-((2,6-dioxopiperidin-3-yl)amino)-2,6-difluorophenyl)piperazin-1-yl)methyl)piperidin-1-yl)carbamate (66 mg, 120.53 μmol, 17.39% yield, 98% purity) as a yellow solid. MS (M+H)⁺=537.3

### Step 7. Synthesis of 3-((4-(4-((1-aminopiperidin-4-yl)methyl)piperazin-1-yl)-3,5-difluorophenyl)amino)piperidine-2,6-dione (11)

To a solution of tert-butyl (4-((4-(4-((2,6-dioxopiperidin-3-yl)amino)-2,6-difluorophenyl)piperazin-1-yl)methyl)piperidin-1-yl)carbamate (66 mg, 122.99 μmol) in DCM (2 mL) was added TFA (0.5 mL), the mixture was stirred at 20° C. for 2 h. LCMS showed a main peak with desired mass. The reaction mixture was concentrated under reduced pressure to afford 3-((4-(4-((1-aminopiperidin-4-yl)methyl)piperazin-1-yl)-3,5-difluorophenyl)amino)piperidine-2,6-dione (65 mg, crude, TFA salt) as a yellow oil, which was used into the next step directly. MS (M+H)⁺=437.3

### Step 8. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(4-((4-(4-((2,6-dioxopiperidin-3-yl)amino)-2,6-difluorophenyl)piperazin-1-yl)methyl)piperidin-1-yl)-3-methoxybenzamide (Compound 18)

To a solution of 4-((7,7-difluoro-9-isopropyl-5-methyl-6-oxo-6,7,8,9-tetrahydro-5H-pyrimido[4,5-b][1,4]diazepin-2-yl)amino)-3-methoxybenzoic acid (49.75 mg, 118.07 μmol) in DMF (2 mL) were added HATU (67.34 mg, 177.11 μmol), DIPEA (45.78 mg, 354.21 μmol, 61.70 μL), the mixture was stirred at 20° C. for 0.5 h, then 3-((4-(4-((1-aminopiperidin-4-yl)methyl)piperazin-1-yl)-3,5-difluorophenyl)amino)piperidine-2,6-dione (65 mg, 118.07 μmol, TFA salt) was added, the mixture was stirred at 20° C. for 16 h. LCMS showed a main peak with desired mass. The reaction mixture was diluted with H₂O (20 mL), extracted with EtOAc (20 mL×3). The combined organic layers were washed with brine (20 mL×2), dried over Na₂SO₄, filtered, and the filtrate was concentrated under reduced pressure. The residue was purified by prep-TLC (SiO₂, DCM:MeOH=10:1) and re-purified by reversed-phase HPLC (column: Waters Xbridge 150×25 mm×5 μm; mobile phase: [water (NH₄HCO₃)-ACN]; B %: 39%-69%, 9 min). The eluent was lyophilized to afford 4-((7,7-difluoro-9-isopropyl-5-methyl-6-oxo-6,7,8,9-tetrahydro-5H-pyrimido[4,5-b][1,4]diazepin-2-yl)amino)-N-(4-((4-(4-((2,6-dioxopiperidin-3-yl)amino)-2,6-difluorophenyl)piperazin-1-yl)methyl)piperidin-1-yl)-3-methoxybenzamide (16.4 mg, 18.94 μmol, 16.04% yield, 97% purity) as a white solid. MS (M+H)⁺=840.4

¹H NMR (400 MHz, CD₃CN) δ=8.73 (s, 1H), 8.47 (d, J=8.3 Hz, 1H), 8.07 (s, 1H), 7.80-7.61 (m, 2H), 7.46-7.30 (m, 2H), 6.33-6.21 (m, 2H), 5.07-4.89 (m, 2H), 4.18-4.09 (m, 1H), 4.02-3.89 (m, 5H), 3.33 (s, 3H), 3.15-3.08 (m, 2H), 3.06-2.99 (m, 4H), 2.77-2.57 (m, 4H), 2.51-2.42 (m, 4H), 2.27-2.22 (m, 3H), 1.90-1.86 (m, 1H), 1.84-1.78 (m, 2H), 1.64-1.49 (m, 1H), 1.36-1.24 (m, 8H).

### Example 19. Synthesis of N-(4-((4-(2-chloro-4-((2,6-dioxopiperidin-3-yl)amino)phenyl)piperazin-1-yl)methyl)piperidin-1-yl)-4-((7,7-difluoro-9-isopropyl-5-methyl-6-oxo-6,7,8,9-tetrahydro-5H-pyrimido[4,5-b][1,4]diazepin-2-yl)amino)-3-methoxybenzamide (Compound 19)

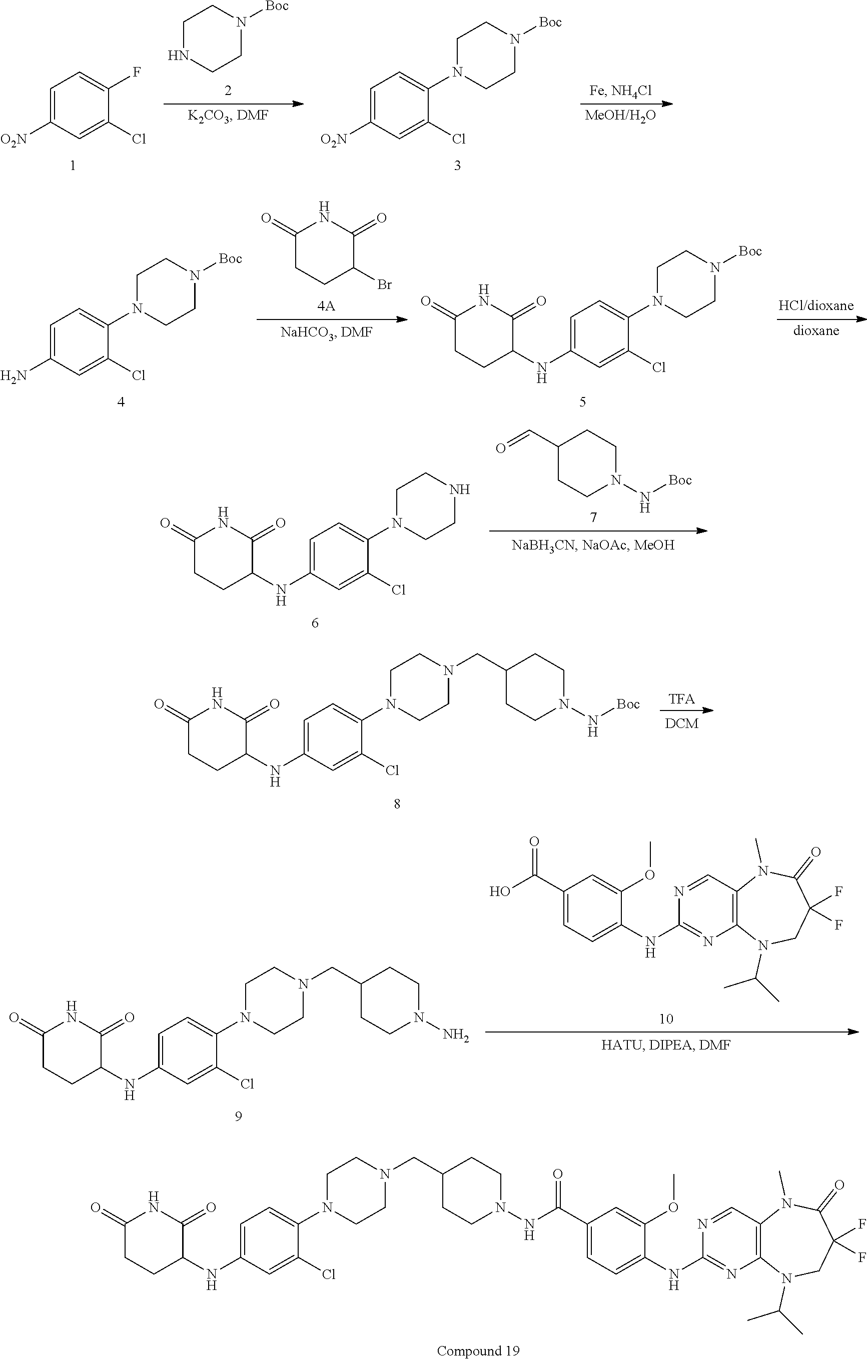

### Step 1. Synthesis of tert-butyl 4-(2-chloro-4-nitrophenyl)piperazine-1-carboxylate (3)

To a solution of 2-chloro-1-fluoro-4-nitrobenzene (5 g, 28.48 mmol) and tert-butyl piperazine-1-carboxylate (5.84 g, 31.33 mmol) in DMF (50 mL) was added K₂CO₃ (7.87 g, 56.97 mmol), the mixture was stirred at 100° C. for 16 hours. LCMS showed 2-chloro-1-fluoro-4-nitrobenzene was consumed completely and 88% of desired mass was detected. The reaction mixture was diluted with H₂O (200 mL) and extracted with EtOAc (150 mL×3). The combined organic layers were washed with brine (200 mL×5), dried over Na₂SO₄, filtered and concentrated in vacuum. The residue was triturated with MTBE (20 mL) for 10 minutes, the suspension was filtered and the filter cake was washed with MTBE (20 mL). The filter cake was collected and dried to afford tert-butyl 4-(2-chloro-4-nitrophenyl)piperazine-1-carboxylate (9.18 g, 26.86 mmol, 94.30% yield) as a gray solid. MS (M−56+H)⁺=286.1

### Step 2. Synthesis of tert-butyl 4-(4-amino-2-chlorophenyl)piperazine-1-carboxylate (4)

To a solution of tert-butyl 4-(2-chloro-4-nitrophenyl)piperazine-1-carboxylate (9.18 g, 26.86 mmol) in MeOH (90 mL) were added Fe (7.50 g, 134.29 mmol), NH₄Cl (7.18 g, 134.29 mmol) and H₂O (9 mL), the mixture was stirred at 70° C. for 16 hours. LCMS showed the starting material was consumed completely and 91% of desired mass. The reaction mixture was poured into HCl solution (1 M, 200 mL), to the resulting mixture was added K₂CO₃ to adjust pH>12, the suspension was filtered and the filter cake was washed with MeOH (50 mL). The filtrate was concentrated in vacuum to remove most of the methanol. The residue was diluted with H₂O (100 mL), and extracted with EtOAc (80 mL×3). The combined organic layers were dried over Na₂SO₄ filtered and concentrated in vacuum to afford tert-butyl 4-(4-amino-2-chlorophenyl)piperazine-1-carboxylate (2.91 g, 9.33 mmol, 34.75% yield) as a black solid, which was used in the next step directly. MS (M+H)⁺=312.1

### Step 3. Synthesis of tert-butyl 4-(2-chloro-4-((2,6-dioxopiperidin-3-yl)amino)phenyl)piperazine-1-carboxylate (5)

To a solution of tert-butyl 4-(4-amino-2-chlorophenyl)piperazine-1-carboxylate (500 mg, 1.60 mmol) and 3-bromopiperidine-2,6-dione (923.70 mg, 4.81 mmol) in DMF (8 mL) was added NaHCO₃ (1.35 g, 16.04 mmol, 623.66 L), the mixture was stirred at 80° C. for 16 hours. LCMS showed 39% of tert-butyl 4-(4-amino-2-chlorophenyl)piperazine-1-carboxylate remained and 59% of desired mass. The reaction mixture was diluted with H₂O (120 mL) and extracted with EtOAc (100 mL×3). The combined organic layers were washed with brine (150 mL×5), dried over Na₂SO₄, filtered and concentrated in vacuum. The residue was purified by flash silica gel chromatography (20 g SepaFlash® Silica Flash Column, Eluent of 12-26% EtOAc/Petroleum ether gradient@100 mL/min) to afford tert-butyl 4-(2-chloro-4-((2,6-dioxopiperidin-3-yl)amino)phenyl)piperazine-1-carboxylate (300 mg, 709.38 μmol, 44.24% yield) as a blue solid. MS (M+H)⁺=423.2

### Step 4. Synthesis of 3-((3-chloro-4-(piperazin-1-yl)phenyl)amino)piperidine-2,6-dione (6)

To a solution of tert-butyl 4-(2-chloro-4-((2,6-dioxopiperidin-3-yl)amino)phenyl)piperazine-1-carboxylate (300 mg, 709.38 μmol) in dioxane (5 mL) was added HCl/dioxane (4 M, 10 mL), the mixture was stirred at 15° C. for 2 hours. LCMS showed 88% of desired mass. The mixture was concentrated in vacuum to afford 3-((3-chloro-4-(piperazin-1-yl)phenyl)amino)piperidine-2,6-dione (420 mg, HCl salt) as a blue solid, MS (M+H)⁺=323.2

### Step 5. Synthesis of tert-butyl (4-((4-(2-chloro-4-((2,6-dioxopiperidin-3-yl)amino)phenyl)piperazin-1-yl)methyl)piperidin-1-yl)carbamate (8)

A mixture of 3-((3-chloro-4-(piperazin-1-yl)phenyl)amino)piperidine-2,6-dione (270 mg, 751.57 μmol, HCl salt), tert-butyl (4-formylpiperidin-1-yl)carbamate (240.20 mg, 1.05 mmol) and NaOAc (184.96 mg, 2.25 mmol) in MeOH (5 mL) was stirred at 15° C. for 30 minutes, then NaBH₃CN (283.38 mg, 4.51 mmol) was added and the resulting mixture was stirred at 15° C. for 12 hours. LCMS showed 76% of desired mass. The reaction mixture was combined with another batch (150 mg scale) and the combined mixture was filtered. The filtrate was concentrated in vacuum. The residue was purified by prep-HPLC (column: Phenomenex luna C18 150×40 mm×15 μm; mobile phase: [water(FA)-ACN]; B %: 10%-40%, 10 min), the eluent was freeze-dried to afford tert-butyl (4-((4-(2-chloro-4-((2,6-dioxopiperidin-3-yl)amino)phenyl)piperazin-1-yl)methyl)piperidin-1-yl)carbamate (55 mg, 71.95 μmol, 9.57% yield, 70% purity) as a brown solid. MS (M+H)⁺=535.3

### Step 6. Synthesis of 3-((4-(4-((1-aminopiperidin-4-yl)methyl)piperazin-1-yl)-3-chlorophenyl)amino)piperidine-2,6-dione (9)

To a solution of tert-butyl (4-((4-(2-chloro-4-((2,6-dioxopiperidin-3-yl)amino)phenyl)piperazin-1-yl)methyl)piperidin-1-yl)carbamate (50.00 mg, 65.41 μmol, 70% purity) in DCM (3 mL) was added TFA (74.58 mg, 654.11 μmol, 48.43 μL) at 0° C., the mixture was stirred at 15° C. for 4 hours. LCMS showed 42% of starting material remained and 32% of desired mass. Additional TFA (74.58 mg, 654.11 μmol, 48.43 μL) was added and the resulting mixture was stirred at 15° C. for further 4 hours, LCMS showed the starting material was consumed completely. The reaction mixture was concentrated in vacuum at 20° C. to afford 3-((4-(4-((1-aminopiperidin-4-yl)methyl)piperazin-1-yl)-3-chlorophenyl)amino)piperidine-2,6-dione (50 mg, TFA salt) as a brown oil. MS (M+H)⁺=435.0

### Step 7. Synthesis of N-(4-((4-(2-chloro-4-((2,6-dioxopiperidin-3-yl)amino)phenyl)piperazin-1-yl)methyl)piperidin-1-yl)-4-((7,7-difluoro-9-isopropyl-5-methyl-6-oxo-6,7,8,9-tetrahydro-5H-pyrimido[4,5-b][1,4]diazepin-2-yl)amino)-3-methoxybenzamide (Compound 19)

To a solution of 4-((7,7-difluoro-9-isopropyl-5-methyl-6-oxo-6,7,8,9-tetrahydro-5H-pyrimido[4,5-b][1,4]diazepin-2-yl)amino)-3-methoxybenzoic acid (25 mg, 59.33 μmol) in DMF (0.5 mL) were added HATU (33.84 mg, 88.99 μmol) and DIPEA (115.01 mg, 889.90 μmol, 155.00 μL), the mixture was stirred at 15° C. for 15 minutes, then a solution of 3-((4-(4-((1-aminopiperidin-4-yl)methyl)piperazin-1-yl)-3-chlorophenyl)amino)piperidine-2,6-dione (50 mg, 91.08 μmol, TFA salt) in DMF (1.5 mL) was added and the resulting mixture was stirred at 15° C. for 1 hour. LCMS showed a peak (90%) with desired mass. The reaction mixture was diluted with H₂O (30 mL) and extracted with EtOAc (20 mL×3). The combined organic layers were washed with brine (40 mL×5), dried over Na₂SO₄, filtered and concentrated in vacuum. The residue was purified by prep-HPLC (column: Waters Xbridge 150×25 mm×5 μm; mobile phase: [water(NH₄HCO₃)-ACN]; B %: 41%-71%, 8 min) and the eluent as freeze-dried to afford N-(4-((4-(2-chloro-4-((2,6-dioxopiperidin-3-yl)amino)phenyl)piperazin-1-yl)methyl)piperidin-1-yl)-4-((7,7-difluoro-9-isopropyl-5-methyl-6-oxo-6,7,8,9-tetrahydro-5H-pyrimido[4,5-b][1,4]diazepin-2-yl)amino)-3-methoxybenzamide (15.0 mg, 16.64 μmol, 28.05% yield, 93% purity) as an off-white solid. MS (M+H)⁺=838.2

¹H NMR (400 MHz, DMSO-d₆) δ=10.77 (s, 1H), 9.34-9.16 (m, 1H), 8.36-8.26 (m, 1H), 8.22 (s, 1H), 7.87 (s, 1H), 7.52-7.38 (m, 2H), 6.96 (d, J=8.8 Hz, 1H), 6.75 (d, J=2.5 Hz, 1H), 6.60 (dd, J=2.5, 8.6 Hz, 1H), 5.86 (d, J=7.9 Hz, 1H), 4.96-4.79 (m, 1H), 4.36-4.19 (m, 1H), 4.03 (br t, J=13.5 Hz, 2H), 3.93 (s, 3H), 3.30 (s, 3H), 3.01 (br d, J=10.4 Hz, 2H), 2.84 (br s, 4H), 2.75 (br dd, J=5.2, 11.8 Hz, 2H), 2.72-2.68 (m, 1H), 2.61-2.51 (m, 5H), 2.21 (br d, J=6.6 Hz, 2H), 2.07 (dt, J=5.0, 8.5 Hz, 1H), 1.93-1.82 (m, 1H), 1.76 (br d, J=11.0 Hz, 2H), 1.57-1.46 (m, 1H), 1.24 (d, J=6.8 Hz, 8H).

### Example 20. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(1-((1-(4-((2,6-dioxopiperidin-3-yl)amino)-2-fluorophenyl)azetidin-3-yl)methyl)piperidin-4-yl)-3-methoxybenzamide (Compound 20)

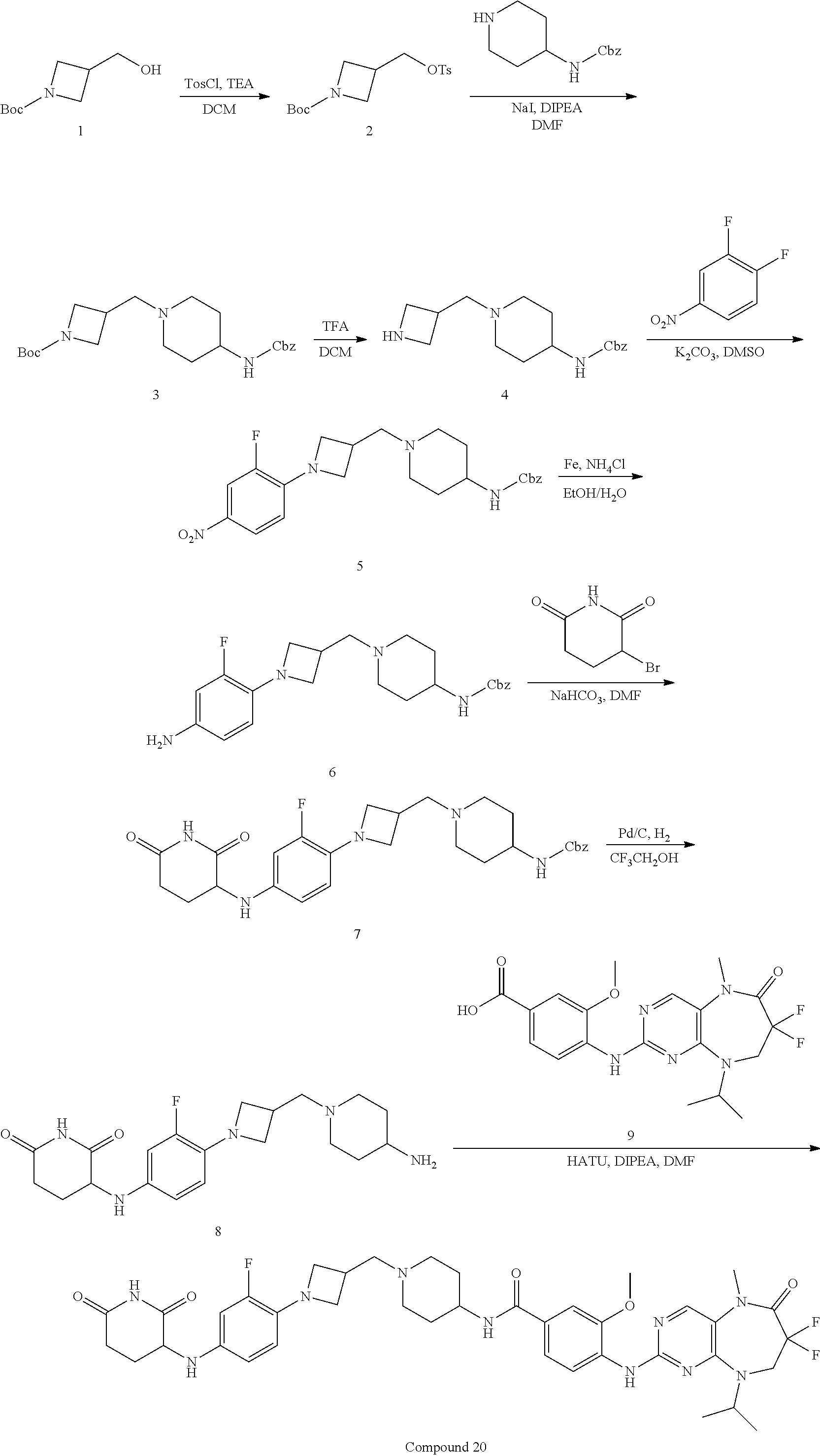

### Step 1. Synthesis of tert-butyl 3-((tosyloxy)methyl)azetidine-1-carboxylate (2)

To a solution of tert-butyl 3-(hydroxymethyl)azetidine-1-carboxylate (5 g, 26.70 mmol) in DCM (50 mL) were added TEA (8.11 g, 80.11 mmol) and TosCl (7.13 g, 37.39 mmol) at 20° C. and the resulting mixture was stirred at 20° C. for 12 h. LCMS showed a peak (76%) with desired mass. The reaction mixture was concentrated in vacuum. The residue was purified by flash silica gel chromatography (80 g SepaFlash® Silica Flash Column, Eluent of 0˜20% EtOAc/Petroleum ether gradient@100 mL/min) to afford tert-butyl 3-((tosyloxy)methyl)azetidine-1-carboxylate (7.5 g, 21.75 mmol, 81.44% yield, 99% purity) as a yellow oil. MS (M−56+H)⁺=286.2

### Step 2. Synthesis of tert-butyl 3-((4-(((benzyloxy)carbonyl)amino)piperidin-1-yl)methyl)azetidine-1-carboxylate (3)

To a solution of tert-butyl 3-((tosyloxy)methyl)azetidine-1-carboxylate (5 g, 14.64 mmol) and benzyl piperidin-4-ylcarbamate (4.46 g, 19.04 mmol) in DMF (50 mL) was added NaI (439.03 mg, 2.93 mmol) and DIPEA (5.68 g, 43.93 mmol, 7.65 mL) at 20° C. and the resulting mixture was stirred at 80° C. for 16 h. LCMS showed starting material was consumed completely and a peak (78%) with desired mass. The reaction mixture was diluted with H₂O (200 mL) and extracted with EtOAc (200 mL×3). The organic layer was washed with brine (200 mL×3), dried over Na₂SO₄, filtered and concentrated. The residue was purified by flash silica gel chromatography (80 g SepaFlash® Silica Flash Column, Eluent of 0˜100% EtOAc/Petroleum ether gradient @100 mL/min) to afford 2 batches of title compound. Batch 1: tert-butyl 3-((4-(((benzyloxy)carbonyl)amino)piperidin-1-yl)methyl)azetidine-1-carboxylate (4.9 g, 12.14 mmol, 82.92% yield) as a yellow oil and Batch 2: tert-butyl 3-((4-(((benzyloxy)carbonyl)amino)piperidin-1-yl)methyl)azetidine-1-carboxylate (1.7 g, 4.21 mmol, 28.77% yield) as a yellow oil. MS (M+H)⁺=404.4

### Step 3. Synthesis of benzyl (1-(azetidin-3-ylmethyl)piperidin-4-yl)carbamate (4)

To a solution of tert-butyl 3-((4-(((benzyloxy)carbonyl)amino)piperidin-1-yl)methyl)azetidine-1-carboxylate (4.9 g, 12.14 mmol) in DCM (10 mL) was added TFA (4.15 g, 36.43 mmol, 2.70 mL) at 20° C. and the resulting mixture was stirred at 20° C. for 1 h. LCMS showed no reaction, additional TFA (5.54 g, 48.57 mmol, 3.60 mL) was added and the reaction mixture was stirred at 20° C. for another 16 h. LCMS showed 56% of starting material remained and 29% peak with desired mass was detected and the reaction mixture was stirred at 20° C. for another 32 h. LCMS showed starting material was consumed. The reaction mixture was combined with another batch (1.7 g scale) for work-up. The combined reaction mixture was concentrated in vacuum to afford benzyl (1-(azetidin-3-ylmethyl)piperidin-4-yl)carbamate (13.9 g, crude, TFA salt) as an orange oil. MS (M+H)⁺=304.4

### Step 4. Synthesis of benzyl (1-((1-(2-fluoro-4-nitrophenyl)azetidin-3-yl)methyl)piperidin-4-yl)carbamate (5)

To a solution of benzyl (1-(azetidin-3-ylmethyl)piperidin-4-yl)carbamate (3.94 g, 9.43 mmol, TFA salt) in DMSO (15 mL) were added K₂CO₃ (2.61 g, 18.86 mmol) and 1,2-difluoro-4-nitrobenzene (1 g, 6.29 mmol, 694.44 L) at 20° C. and the resulting mixture was stirred at 40° C. for 1 h. TLC (SiO₂, Petroleum ether:EtOAc=0:1) indicated 1, 2-difluoro-4-nitro-benzene remained and one new spot was formed. The reaction mixture was diluted with H₂O (50 mL) and extracted with EtOAc (50 mL×3). The combined organic layer was washed with brine (50 mL×3), dried over Na₂SO₄, filtered and concentrated. The residue was purified by flash silica gel chromatography (25 g SepaFlash® Silica Flash Column, Eluent of 0˜100% EtOAc/Petroleum ether gradient@100 mL/min) to afford benzyl (1-((1-(2-fluoro-4-nitrophenyl)azetidin-3-yl)methyl)piperidin-4-yl)carbamate (1.1 g, 2.49 mmol, 39.55% yield) as a yellow solid. MS (M+H)⁺=443.3

### Step 5. Synthesis of benzyl (1-((1-(4-amino-2-fluorophenyl)azetidin-3-yl)methyl)piperidin-4-yl)carbamate (6)

To a solution of benzyl (1-((1-(2-fluoro-4-nitrophenyl)azetidin-3-yl)methyl)piperidin-4-yl)carbamate (1.1 g, 2.49 mmol) in EtOH (10 mL) and H₂O (5 mL) were added Fe (832.98 mg, 14.92 mmol) and NH₄Cl (797.87 mg, 14.92 mmol) at 20° C. and the resulting mixture was stirred at 80° C. for 12 h. LCMS showed the starting material was consumed completely and a peak (70%) with desired mass. The pH of the mixture was adjusted to 10 with saturated NaHCO₃ and the resulting mixture was extracted with EtOAc (15 mL×3). The combined organic layer was dried over Na₂SO₄, filtered and concentrated to afford benzyl (1-((1-(4-amino-2-fluorophenyl)azetidin-3-yl)methyl)piperidin-4-yl)carbamate (926 mg, 2.24 mmol, 90.30% yield) as an orange solid. MS (M+H)⁺=413.2

### Step 6. Synthesis of benzyl (1-((1-(4-((2,6-dioxopiperidin-3-yl)amino)-2-fluorophenyl)azetidin-3-yl)methyl)piperidin-4-yl)carbamate (7)

To a solution of benzyl (1-((1-(4-amino-2-fluorophenyl)azetidin-3-yl)methyl)piperidin-4-yl)carbamate (926 mg, 2.24 mmol) and 3-bromopiperidine-2,6-dione (1.29 g, 6.73 mmol) in DMF (15 mL) was added NaHCO₃ (1.89 g, 22.45 mmol, 873.07 L) at 20° C. and the resulting mixture was stirred at 85° C. for 16 h. LCMS showed starting material was consumed completely and a peak (39%) with desired mass. The reaction mixture was diluted with EtOAc (30 mL) and filtered. The filtrate was diluted with H₂O (100 mL) and extracted with EtOAc (100 mL×3). The combined organic layer was washed with brine (100 mL×3), dried over Na₂SO₄, filtered and concentrated. The residue was purified by flash silica gel chromatography (12 g SepaFlash® Silica Flash Column, Eluent of 0-100% EtOAc/Petroleum ether gradient@100 mL/min) to afford benzyl (1-((1-(4-((2,6-dioxopiperidin-3-yl)amino)-2-fluorophenyl)azetidin-3-yl)methyl)piperidin-4-yl)carbamate (545 mg, 1.04 mmol, 46.37% yield) as a green solid. MS (M+H)⁺=524.2

### Step 7. Synthesis of 3-((4-(3-((4-aminopiperidin-1-yl)methyl)azetidin-1-yl)-3-fluorophenyl) amino)piperidine-2,6-dione (8)

To a solution of benzyl (1-((1-(4-((2,6-dioxopiperidin-3-yl)amino)-2-fluorophenyl)azetidin-3-yl)methyl)piperidin-4-yl)carbamate (200 mg, 381.97 μmol) in CF₃CH₂OH (5 mL) was added Pd/C (0.1 g, 10% purity) under N₂ atmosphere. The suspension was degassed and purged with H₂ for 3 times. The mixture was stirred under H₂ (15 Psi) at 20° C. for 16 h. LCMS showed the starting material was consumed completely and a peak with desired mass was detected. The reaction mixture was diluted with CF₃CH₂OH (20 mL) and filtered. The filtrate was concentrated in vacuum to afford 3-((4-(3-((4-aminopiperidin-1-yl)methyl)azetidin-1-yl)-3-fluorophenyl)amino)piperidine-2,6-dione (201 mg, crude) as a blue solid. MS (M+H)⁺=390.2

### Step 8. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(1-((1-(4-((2,6-dioxopiperidin-3-yl)amino)-2-fluorophenyl)azetidin-3-yl)methyl)piperidin-4-yl)-3-methoxybenzamide (Compound 20)

To a solution of 4-((7,7-difluoro-9-isopropyl-5-methyl-6-oxo-6,7,8,9-tetrahydro-5H-pyrimido[4,5-b][1,4]diazepin-2-yl)amino)-3-methoxybenzoic acid (80 mg, 189.84 μmol) in DMF (2 mL) were added HATU (79.40 mg, 208.83 μmol) and DIPEA (73.61 mg, 569.53 μmol, 99.20 μL). The mixture was stirred at 20° C. for 10 min and a solution of 3-((4-(3-((4-aminopiperidin-1-yl)methyl)azetidin-1-yl)-3-fluorophenyl)amino)piperidine-2,6-dione (147.88 mg, 379.69 μmol) in DMF (2 mL) was added and the resulting mixture was stirred at 20° C. for 1 h. LCMS showed the starting material was consumed completely and a peak with desired mass. The reaction mixture was diluted with H₂O (12 mL) and extracted with EtOAc (12 mL×3). The organic layer was washed with brine (12 mL×3), dried over Na₂SO₄, filtered and concentrated. The residue was purified by prep-TLC (SiO₂, DCM:MeOH=10:1) followed by prep-HPLC (column: Unisil 3-100 C18 Ultra 150×50 mm×3 μm; mobile phase: [water (FA)-ACN]; B %: 10%-40%, 7 min) and the eluent was lyophilized to afford 4-((7,7-difluoro-9-isopropyl-5-methyl-6-oxo-6,7,8,9-tetrahydro-5H-pyrimido[4,5-b][1,4]diazepin-2-yl)amino)-N-(1-((1-(4-((2,6-dioxopiperidin-3-yl)amino)-2-fluorophenyl)azetidin-3-yl)methyl)piperidin-4-yl)-3-methoxybenzamide (32 mg, 34.52 μmol, 18.18% yield, 88% purity, 0.5 FA salt) as a gray solid. MS (M+H)⁺=793.2

¹H NMR (400 MHz, DMSO-d₆) δ=10.75 (s, 1H), 8.34-8.27 (m, 1H), 8.22 (s, 1H), 8.15-8.10 (br, 1H), 7.87 (s, 1H), 7.54-7.45 (m, 2H), 6.52-6.44 (m, 1H), 6.43-6.32 (m, 2H), 5.53 (br d, J=7.1 Hz, 1H), 4.93-4.82 (m, 1H), 4.23-4.14 (m, 1H), 4.04 (br t, J=13.5 Hz, 2H), 3.94 (s, 3H), 3.86 (br t, J=6.7 Hz, 2H), 3.82-3.71 (m, 1H), 3.42-3.34 (m, 5H), 2.91-2.79 (m, 3H), 2.77-2.68 (m, 1H), 2.58 (br d, J=5.1 Hz, 3H), 2.12-2.01 (m, 3H), 1.89-1.74 (m, 3H), 1.65-1.52 (m, 2H), 1.24 (d, J=6.7 Hz, 6H).

### Example 21. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(4-((4-(4-((2,6-dioxopiperidin-3-yl)amino)-2-(trifluoromethyl)phenyl)piperazin-1-yl)methyl)piperidin-1-yl)-3-methoxybenzamide (Compound 21)

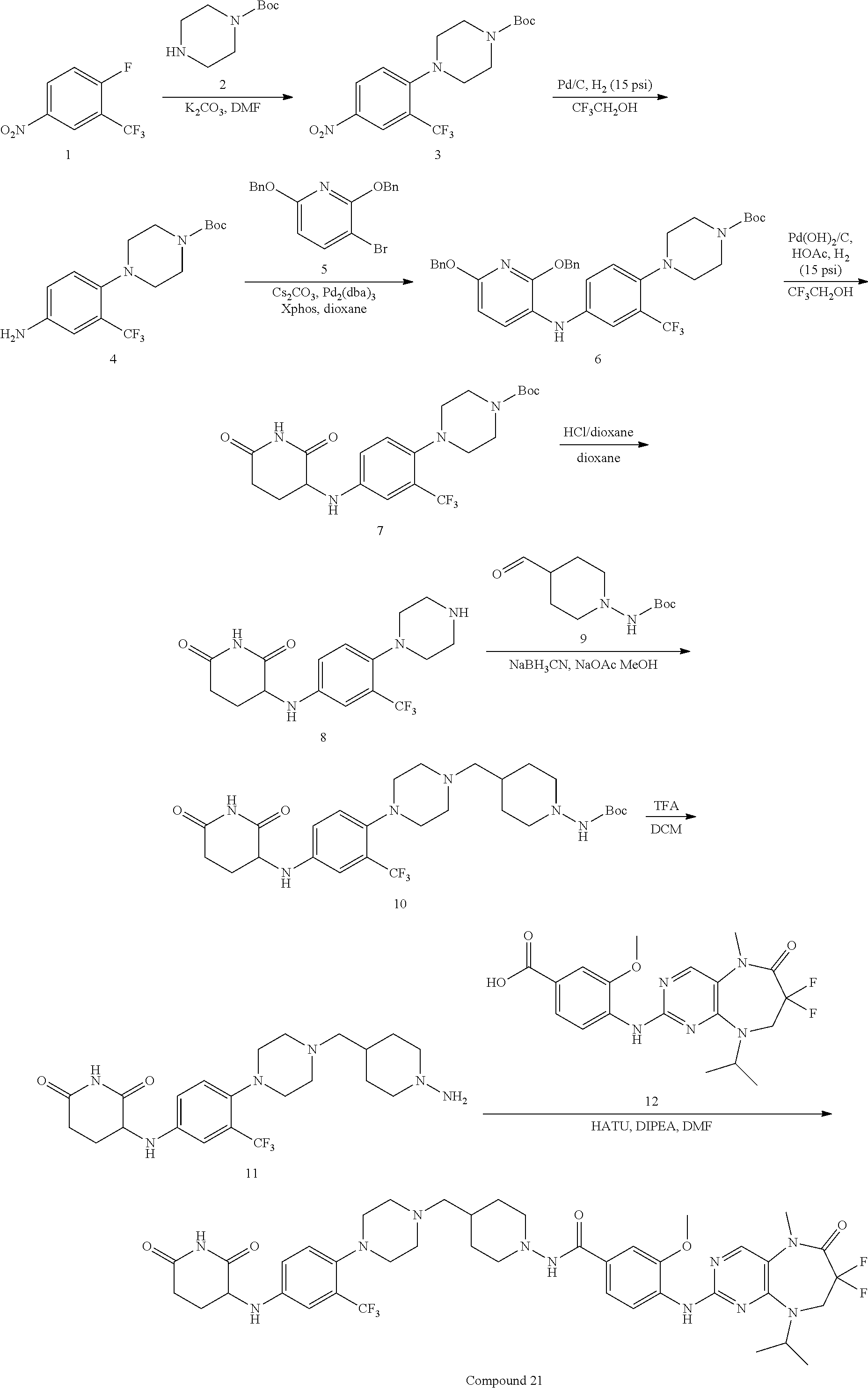

### Step 1. Synthesis of tert-butyl 4-(4-nitro-2-(trifluoromethyl)phenyl)piperazine-1-carboxylate (3)

To a solution of 1-fluoro-4-nitro-2-(trifluoromethyl)benzene (5 g, 23.91 mmol, 3.29 mL) and tert-butyl piperazine-1-carboxylate (4.90 g, 26.30 mmol) in DMF (50 mL) was added K₂CO₃ (6.61 g, 47.82 mmol), the mixture was stirred at 50° C. for 16 hours. LCMS showed a peak (70%) with desired mass. The reaction mixture was diluted with H₂O (250 mL) and extracted with EtOAc (150 mL×3). The combined organic layers were washed with brine (200 mL×5), dried over Na₂SO₄, filtered and concentrated in vacuum. The residue was purified by flash silica gel chromatography (20 g SepaFlash® Silica Flash Column, Eluent of 6% EtOAc/Petroleum ether gradient@100 mL/min) to afford tert-butyl 4-(4-nitro-2-(trifluoromethyl)phenyl)piperazine-1-carboxylate (7.1 g, 18.92 mmol, 79.11% yield) as a yellow solid. MS (M−56+H)⁺=320.1

### Step 2. Synthesis of tert-butyl 4-(4-amino-2-(trifluoromethyl)phenyl)piperazine-1-carboxylate (4)

To a solution of tert-butyl 4-(4-nitro-2-(trifluoromethyl)phenyl)piperazine-1-carboxylate (7.1 g, 18.92 mmol) in CF₃CH₂OH (80 mL) was added Pd/C (1 g, 10% purity) under N₂, the suspension was degassed and purged with H₂ several times. The mixture was stirred at 15° C. for 16 hours under H₂ (15 psi). LCMS showed the starting material was consumed completely and a main peak (99%) with desired mass. The reaction mixture was filtered and the filter cake was washed with CF₃CH₂OH (100 mL), the filtrate was concentrated in vacuum to afford tert-butyl 4-(4-amino-2-(trifluoromethyl)phenyl)piperazine-1-carboxylate (6.08 g, 17.60 mmol, 93.07% yield) as a gray solid. MS (M+H)⁺=346.1

### Step 3. Synthesis of tert-butyl 4-(4-((2,6-bis(benzyloxy)pyridin-3-yl)amino)-2-(trifluoromethyl)phenyl)piperazine-1-carboxylate (6)

To a solution of tert-butyl 4-(4-amino-2-(trifluoromethyl)phenyl)piperazine-1-carboxylate (800 mg, 2.32 mmol) and 2,6-bis(benzyloxy)-3-bromopyridine (1.11 g, 3.01 mmol) in dioxane (20 mL) were added Cs₂CO₃ (2.26 g, 6.95 mmol), Pd₂(dba)₃ (212.12 mg, 231.64 μmol) and XPhos (110.43 mg, 231.64 μmol), the mixture was stirred at 100° C. for 16 hours under N₂. LCMS showed tert-butyl 4-(4-amino-2-(trifluoromethyl)phenyl)piperazine-1-carboxylate was consumed completely and a peak (56%) with desired mass. The reaction mixture was filtered and the filter cake was washed with EtOAc (30 mL), the filtrate was concentrated in vacuum. The residue was purified by flash silica gel chromatography (12 g SepaFlash® Silica Flash Column, Eluent of 0˜6% EtOAc/Petroleum ether gradient@100 mL/min) to afford tert-butyl 4-(4-((2,6-bis(benzyloxy)pyridin-3-yl)amino)-2-(trifluoromethyl)phenyl)piperazine-1-carboxylate (1.43 g, 2.25 mmol, 97.27% yield) as a yellow oil. MS (M+H)⁺=635.2

### Step 4. Synthesis of tert-butyl 4-(4-((2,6-dioxopiperidin-3-yl)amino)-2-(trifluoromethyl)phenyl) piperazine-1-carboxylate (7)

To a solution of tert-butyl 4-(4-((2,6-bis(benzyloxy)pyridin-3-yl)amino)-2-(trifluoromethyl)phenyl)piperazine-1-carboxylate (930 mg, 1.47 mmol) and CH₃COOH (8.80 mg, 146.53 μmol, 8.38 L) in CF₃CH₂OH (30 mL) was added Pd(OH)₂/C (200 mg, 10% purity) under N₂, the suspension was degassed and purged with H₂ several times. The mixture was stirred at 15° C. for 16 hours under H₂ (15 psi). LCMS showed the starting material was consumed completely and a peak (55%) with desired mass. The reaction mixture was filtered, the filter cake was washed with CF₃CH₂OH (50 mL). The filtrate was concentrated in vacuum to afford tert-butyl 4-(4-((2,6-dioxopiperidin-3-yl)amino)-2-(trifluoromethyl)phenyl)piperazine-1-carboxy late (590 mg) as a blue solid. MS (M+H)⁺=457.2

### Step 5. Synthesis of 3-((4-(piperazin-1-yl)-3-(trifluoromethyl)phenyl)amino)piperidine-2,6-dione (8)

To a solution of tert-butyl 4-(4-((2,6-dioxopiperidin-3-yl)amino)-2-(trifluoromethyl)phenyl)piperazine-1-carboxy late (590 mg, 1.29 mmol) in dioxane (6 mL) was added HCl/dioxane (4 M, 12 mL), the mixture was stirred at 15° C. for 2 hours. LCMS showed the starting material was consumed completely and a peak (69%) with desired mass. The residue was concentrated in vacuum. The residue was purified by prep-HPLC (column: Phenomenex luna C18 150×25 mm×10 μm; mobile phase: [water(FA)-ACN]; B %: 1%-23%, 11 min), the eluent was freeze-dried to afford 3-((4-(piperazin-1-yl)-3-(trifluoromethyl)phenyl)amino)piperidine-2,6-dione (240 mg, 610.99 μmol, 47.27% yield, HCl salt) as a light brown solid. MS (M+H)⁺=357.0

¹H NMR (400 MHz, DMSO-d₆) δ=11.22-10.41 (m, 1H), 8.33 (s, 1H), 7.27 (d, J=8.6 Hz, 1H), 7.01-6.79 (m, 2H), 6.24 (br d, J=8.0 Hz, 1H), 4.43-4.34 (m, 1H), 2.93 (br s, 4H), 2.84-2.75 (m, 4H), 2.75-2.68 (m, 1H), 2.58 (td, J=4.0, 17.4 Hz, 1H), 2.13-2.01 (m, 1H), 1.90 (dq, J=4.6, 12.2 Hz, 1H).

### Step 6. Synthesis of tert-butyl (4-((4-(4-((2,6-dioxopiperidin-3-yl)amino)-2-(trifluoromethyl) phenyl)piperazin-1-yl)methyl)piperidin-1-yl)carbamate (10)

To a solution of 3-((4-(piperazin-1-yl)-3-(trifluoromethyl)phenyl)amino)piperidine-2,6-dione (240 mg, 610.99 μmol, HCl salt), tert-butyl (4-formylpiperidin-1-yl)carbamate (488.19 mg, 2.14 mmol) and NaOAc (150.37 mg, 1.83 mmol) in MeOH (8 mL) was stirred at 15° C. for 30 minutes, then NaBH₃CN (153.58 mg, 2.44 mmol) was added and the resulting mixture was stirred at 15° C. for 16 hours. LCMS showed 3-((4-(piperazin-1-yl)-3-(trifluoromethyl)phenyl)amino)piperidine-2,6-dione was consumed completely and desired mass was detected. The reaction mixture was concentrated in vacuum. The residue purified by prep-HPLC (column: Shim-pack C18 150×25×10 μm; mobile phase: [water (TFA)-ACN]; B %: 15%-45%, 10 min), the eluent was freeze-dried to afford tert-butyl (4-((4-(4-((2,6-dioxopiperidin-3-yl)amino)-2-(trifluoromethyl)phenyl)piperazin-1-yl)methyl)piperidin-1-yl)carbamate (160 mg, 234.38 μmol, 38.36% yield, TFA salt) as a brown solid. MS (M+H)⁺=569.3.

### Step 7. Synthesis of 3-((4-(4-((1-aminopiperidin-4-yl)methyl)piperazin-1-yl)-3-(trifluoromethyl)phenyl)amino)piperidine-2,6-dione (11)

To a solution of tert-butyl (4-((4-(4-((2,6-dioxopiperidin-3-yl)amino)-2-(trifluoromethyl) phenyl)piperazin-1-yl)methyl)piperidin-1-yl)carbamate (150 mg, 219.73 μmol, TFA salt) in DCM (2 mL) was added TFA (250.54 mg, 2.20 mmol, 162.69 L), the mixture was stirred at 15° C. for 5 hours. LCMS showed trace of the starting material remained and a peak (53%) with desired mass. The reaction mixture was bubbled with N₂ to remove most of the solvent to afford 3-((4-(4-((1-aminopiperidin-4-yl)methyl)piperazin-1-yl)-3-(trifluoromethyl)phenyl)amino)piperidine-2,6-dione (130 mg, TFA salt) as a brown oil. MS (M+H)⁺=469.2

### Step 8. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(4-((4-(4-((2,6-dioxopiperidin-3-yl)amino)-2-(trifluoromethyl)phenyl)piperazin-1-yl)methyl)piperidin-1-yl)-3-methoxybenzamide (Compound 21)

To a solution of 4-((7,7-difluoro-9-isopropyl-5-methyl-6-oxo-6,7,8,9-tetrahydro-5H-pyrimido[4,5-b][1,4]diazepin-2-yl)amino)-3-methoxybenzoic acid (85 mg, 201.71 μmol) in DMF (2 mL) were added HATU (115.04 mg, 302.56 μmol) and DIPEA (338.90 mg, 2.62 mmol, 456.75 μL), the mixture was stirred at 15° C. for 15 minutes, then 3-((4-(4-((1-aminopiperidin-4-yl)methyl)piperazin-1-yl)-3-(trifluoromethyl)phenyl)amino)piperidine-2,6-dione (129.25 mg, 221.88 μmol, TFA salt) was added and the resulting mixture was stirred at 15° C. for 1 hour. LCMS showed 3-((4-(4-((1-aminopiperidin-4-yl)methyl)piperazin-1-yl)-3-(trifluoromethyl)phenyl)amino)piperidine-2,6-dione was consumed completely and a peak (62%) with desired mass. To the mixture was added CH₃COOH to adjust pH<7 and the resulting mixture was filtered and the filtrate was purified by prep-HPLC (column: Phenomenex C18 75×30 mm×3 μm; mobile phase: [water(FA)-ACN]; B %: 18%-48%, 7 min), the eluent was freeze-dried to afford 4-((7,7-difluoro-9-isopropyl-5-methyl-6-oxo-6,7,8,9-tetrahydro-5H-pyrimido[4,5-b][1,4]diazepin-2-yl)amino)-N-(4-((4-(4-((2,6-dioxopiperidin-3-yl)amino)-2-(trifluoromethyl)phenyl)piperazin-1-yl)methyl)piperidin-1-yl)-3-methoxybenzamide (12.1 mg, 12.77 mol, 6.33% yield, 93% purity, 0.2 FA salt) as a brown solid. MS (M+H)⁺=872.1

¹H NMR (400 MHz, DMSO-d₆) δ=10.80 (s, 1H), 9.29 (s, 1H), 8.34-8.26 (m, 1H), 8.22 (s, 1H), 7.88 (s, 1H), 7.50-7.39 (m, 2H), 7.33 (br d, J=8.8 Hz, 1H), 6.99-6.84 (m, 2H), 6.35-6.12 (m, 1H), 4.96-4.79 (m, 1H), 4.45-4.32 (m, 1H), 4.04 (br t, J=13.5 Hz, 2H), 3.94 (s, 3H), 3.31 (br s, 3H), 3.03 (br d, J=9.9 Hz, 2H), 2.90-2.69 (m, 7H), 2.61 (br d, J=3.8 Hz, 2H), 2.57-2.52 (m, 5H), 2.11-2.05 (m, 1H), 1.97-1.89 (m, 1H), 1.78 (br d, J=10.8 Hz, 2H), 1.67-1.50 (m, 1H), 1.25 (d, J=6.7 Hz, 8H).

### Example 22. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(4-(2-(4-(4-((2,6-dioxopiperidin-3-yl)amino)-2-fluorophenyl)piperazin-1-yl)ethyl)piperidin-1-yl)-3-methoxybenzamide (Compound 22)

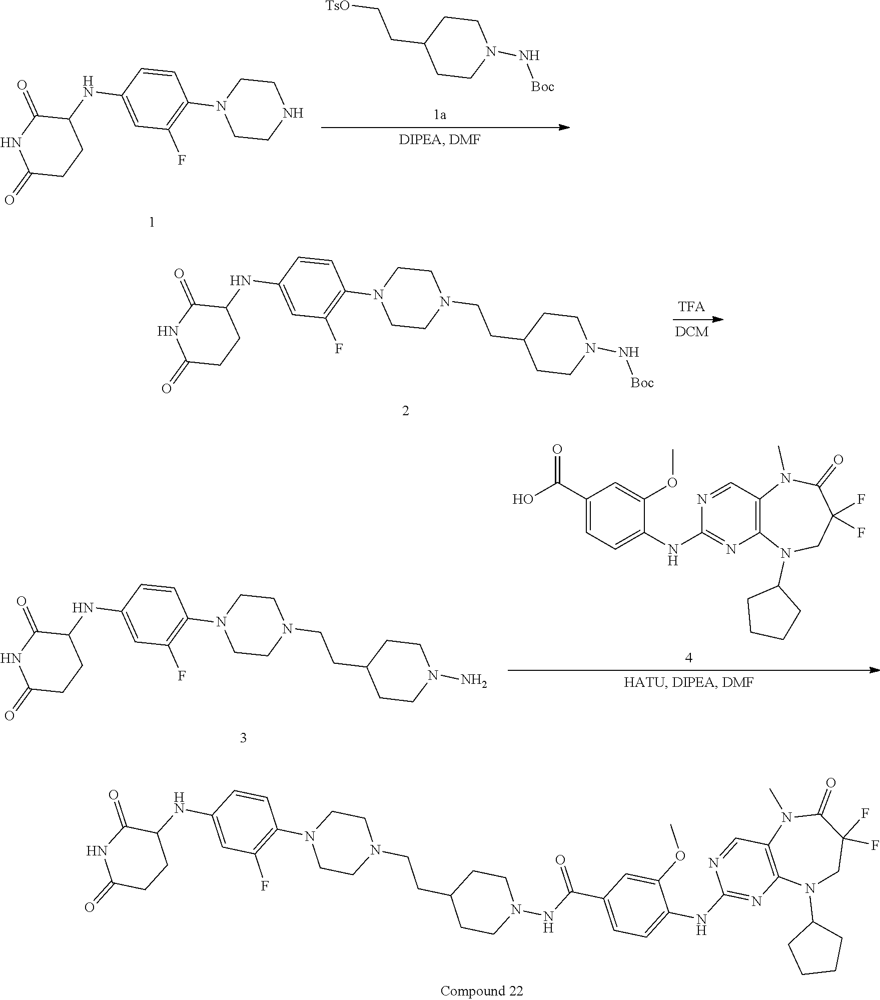 [SMILES: O=C1CCC(Nc2ccc(N3CCNCC3)c(F)c2)C(=O)N1 ; Cc1ccc(S(=O)(=O)OCCC2CCN(NC(=O)OC(C)(C)C)CC2)cc1 ; CC(C)(C)OC(=O)NN1CCC(CCN2CCN(c3ccc(NC4CCC(=O)NC4=O)cc3F)CC2)CC1 ; NN1CCC(CCN2CCN(c3ccc(NC4CCC(=O)NC4=O)cc3F)CC2)CC1 ; COc1cc(C(=O)O)ccc1Nc1ncc2c(n1)N(C1CCCC1)CC(F)(F)C(=O)N2C ; COc1cc(C(=O)NN2CCC(CCN3CCN(c4ccc(NC5CCC(=O)NC5=O)cc4F)CC3)CC2)ccc1Nc1ncc2c(n1)N(C1CCCC1)CC(F)(F)C(=O)N2C]

### Step 1. Synthesis of tert-butyl (4-(2-(4-(4-((2,6-dioxopiperidin-3-yl)amino)-2-fluorophenyl)piperazin-1-yl)ethyl)piperidin-1-yl)carbamate (2)

To a solution of 3-((3-fluoro-4-(piperazin-1-yl)phenyl)amino)piperidine-2,6-dione (0.25 g, 729.30 μmol, HCl salt) and 2-(1-((tert-butoxycarbonyl)amino)piperidin-4-yl)ethyl 4-methylbenzenesulfonate (290.64 mg, 729.30 μmol) in DMF (1 mL) were added DIPEA (282.76 mg, 2.19 mmol, 381.08 μL) and NaI (21.86 mg, 145.86 μmol) at 25° C. The reaction mixture was heated to 60° C. for 2 hours. LCMS showed the starting material was consumed completely and a main peak with desired mass. The crude product was purified by prep-HPLC (column: Phenomenex luna C18 150×40 mmx 15 um; mobile phase: [water (FA)-ACN]; B %: 3%-35%, 9 min), the eluent was and lyophilized to afford tert-butyl (4-(2-(4-(4-((2,6-dioxopiperidin-3-yl)amino)-2-fluorophenyl)piperazin-1-yl)ethyl)piperidin-1-yl)carbamate (0.1 g, crude) as a brown solid. MS (M+H)⁺=533.3

### Step 2. Synthesis of 3-((4-(4-(2-(1-aminopiperidin-4-yl)ethyl)piperazin-1-yl)-3-fluorophenyl)amino)piperidine-2,6-dione (3)

To a solution of tert-butyl (4-(2-(4-(4-((2,6-dioxopiperidin-3-yl)amino)-2-fluorophenyl)piperazin-1-yl)ethyl)piperidin-1-yl)carbamate (0.1 g, 187.74 μmol) in DCM (2 mL) was added TFA (1.26 g, 11.09 mmol, 821.07 μL) at 25° C. The resulting mixture was stirred at 25° C. for 0.5 hr. LCMS showed the starting material was consumed completely and a main peak with desired mass. The reaction mixture was concentrated under reduced pressure to afford 3-((4-(4-(2-(1-aminopiperidin-4-yl)ethyl)piperazin-1-yl)-3-fluorophenyl)amino)piperidine-2,6-dione (0.1 g, crude, TFA salt) as a brown solid. MS (M+H)⁺=433.2

### Step 3. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(4-(2-(4-(4-((2,6-dioxopiperidin-3-yl)amino)-2-fluorophenyl)piperazin-1-yl)ethyl)piperidin-1-yl)-3-methoxybenzamide (Compound 22)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (0.1 g, 223.50 μmol) in DMF (1 mL) were added HATU (101.98 mg, 268.20 μmol) and DIPEA (115.54 mg, 893.99 μmol, 155.71 μL). The mixture was stirred at 25° C. for 10 min. Then 3-((4-(4-(2-(1-aminopiperidin-4-yl)ethyl)piperazin-1-yl)-3-fluorophenyl)amino)piperidine-2,6-dione (97.72 mg, 178.80 μmol, TFA salt) was added and the resulting mixture was stirred at 25° C. for 1 h. LCMS showed the 3-((4-(4-(2-(1-aminopiperidin-4-yl)ethyl)piperazin-1-yl)-3-fluorophenyl)amino)piperidine-2,6-dione was consumed completely and a peak (41%) with desired mass. The reaction mixture was filtered. The filtrate was purified by prep-HPLC (column: Unisil 3-100 C18 Ultra 150×50 mm×3 um; mobile phase: [water (FA)-ACN]; B %: 18%-48%, 7 min) and re-purified by prep-HPLC (column: Waters Xbridge 150×25 mm×5 m; mobile phase: [water (NH₄HCO₃)-ACN]; B %: 37%-67%, 8 min), the eluent was lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-N-(4-(2-(4-(4-((2,6-dioxopiperidin-3-yl)amino)-2-fluorophen yl)piperazin-1-yl)ethyl)piperidin-1-yl)-3-methoxybenzamide (36 mg, 40.51 μmol, 18.13% yield, 97% purity) as a white solid. MS (M+H)⁺=862.5

¹H NMR (400 MHz, DMSO-d₆) δ=10.78 (s, 1H), 9.28 (s, 1H), 8.35-8.20 (m, 2H), 7.96 (s, 1H), 7.51-7.37 (m, 2H), 6.91-6.77 (m, 1H), 6.52 (dd, J=1.8, 15.0 Hz, 1H), 6.43 (dd, J=1.7, 8.8 Hz, 1H), 5.80 (d, J=7.8 Hz, 1H), 4.88-4.70 (m, 1H), 4.26 (td, J=5.5, 11.3 Hz, 1H), 4.05 (br t, J=14.1 Hz, 2H), 3.94 (s, 3H), 3.30 (s, 3H), 3.00 (br d, J=9.6 Hz, 2H), 2.86 (br s, 4H), 2.80-2.66 (m, 3H), 2.59 (br d, J=4.1 Hz, 5H), 2.38-2.32 (m, 2H), 2.14-2.04 (m, 1H), 2.00-1.80 (m, 3H), 1.77-1.54 (m, 8H), 1.48-1.21 (m, 5H).

### Example 23. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(4-(2-(4-(4-((2,6-dioxopiperidin-3-yl)(methyl)amino)-2-fluorophenyl)piperazin-1-yl)ethyl)piperidin-1-yl)-3-methoxybenzamide (Compound 23)

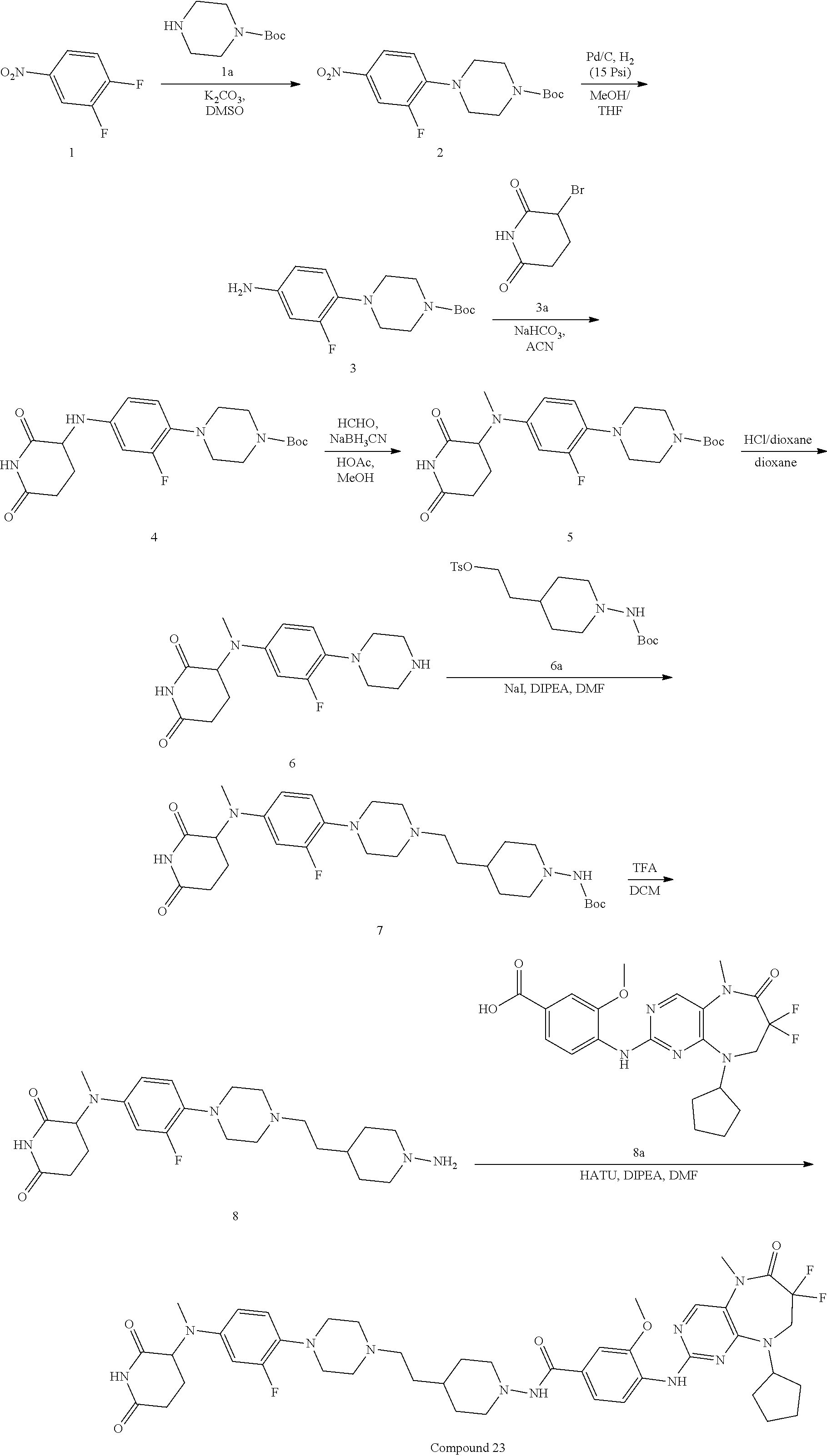

### Step 1. Synthesis of tert-butyl 4-(2-fluoro-4-nitrophenyl)piperazine-1-carboxylate (2)

A mixture of 1,2-difluoro-4-nitrobenzene (6.3 g, 39.60 mmol, 4.37 mL), tert-butyl piperazine-1-carboxylate (7.38 g, 39.60 mmol) and K₂CO₃ (16.42 g, 118.80 mmol) in DMSO (80 mL) was stirred at 60° C. for 4 hr. LCMS showed a main peak with desired mass. The mixture was poured into water (300 mL) and extracted with EtOAc (80 mL×5). The combined organic phase was washed with brine (80 mL×3), dried over Na₂ SO₄, filtered and concentrated to afford tert-butyl 4-(2-fluoro-4-nitrophenyl)piperazine-1-carboxylate (13.7 g, crude) as a yellow solid. MS (M−100+H)⁺=226.0

### Step 2. Synthesis of tert-butyl 4-(4-amino-2-fluorophenyl)piperazine-1-carboxylate (3)

To a solution of tert-butyl 4-(2-fluoro-4-nitrophenyl)piperazine-1-carboxylate (13 g, 39.96 mmol) in THF (300 mL) and MeOH (300 mL) was added Pd/C (1 g, 10% purity) at 25° C. under N₂ atmosphere. The reaction mixture was degassed and purged with H₂ for 3 times. The mixture was stirred at 25° C. for 2 hr under H₂ (15 Psi). LCMS showed the starting material was consumed completely, and a main peak with desired mass. The reaction mixture was filtered through a celite pad and the filtrate was concentrated to afford tert-butyl 4-(4-amino-2-fluorophenyl)piperazine-1-carboxylate (12 g, crude) as a brown solid. MS (M+H)⁺=296.1

### Step 3. Synthesis of tert-butyl 4-(4-((2,6-dioxopiperidin-3-yl)amino)-2-fluorophenyl)piperazine-1-carboxylate (4)

A mixture of tert-butyl 4-(4-amino-2-fluorophenyl)piperazine-1-carboxylate (0.5 g, 1.69 mmol), 3-bromopiperidine-2,6-dione (650.11 mg, 3.39 mmol) and NaHCO₃ (711.07 mg, 8.46 mmol, 329.20 L) in ACN (20 mL) was stirred at 80° C. for 12 hr. LCMS showed most of the starting material was still remained and a peak (14%) with desired mass. Additional 3-bromopiperidine-2,6-dione (325.05 mg, 1.69 mmol) and NaHCO₃ (426.64 mg, 5.08 mmol, 197.52 L) were added at 25° C. and the resulting mixture was stirred at 80° C. for 12 hr. LCMS showed the starting material was still remained and a peak (44%) with desired mass. Another 3-bromopiperidine-2,6-dione (487.58 mg, 2.54 mmol) and NaHCO₃ (426.64 mg, 5.08 mmol, 197.52 μL) were added at 25° C. and the mixture was stirred at 80° C. for another 12 hr. LCMS showed the starting material was still remained and a peak (65%) with desired mass. The reaction mixture was filtered and the filter cake was washed with EtOAc (30 mL). The combined filtrate was concentrated. The residue was purified by flash silica gel chromatography (12 g SepaFlash® Silica Flash Column, Eluent of 0˜35% EtOAc/Petroleum ether gradient@60 mL/min), to afford tert-butyl 4-(4-((2,6-dioxopiperidin-3-yl)amino)-2-fluorophenyl)piperazine-1-carboxylate (0.5 g, crude) as a green solid. MS (M+H)⁺=407.2

### Step 4. Synthesis of tert-butyl 4-(4-((2,6-dioxopiperidin-3-yl)(methyl)amino)-2-fluorophenyl)piperazine-1-carboxylate (5)

A mixture of tert-butyl 4-(4-((2,6-dioxopiperidin-3-yl)amino)-2-fluorophenyl)piperazine-1-carboxylate (0.4 g, 984.13 μmol), HCHO (159.73 mg, 1.97 mmol, 146.54 μL, 37% purity) and AcOH (59.10 mg, 984.13 μmol, 56.28 μL) in MeOH (5 mL) was stirred at 25° C. for 30 min. Then NaBH₃CN (185.53 mg, 2.95 mmol) was added at 25° C., and the resulting mixture was stirred at 25° C. for 1 h. LCMS showed a peak (50%) with mass of the starting material, and a peak (43%) with desired mass. Another portion of HCHO (159.73 mg, 1.97 mmol, 146.54 μL, 37% purity) was added to the reaction mixture at 25° C., after stirring at 25° C. for another 10 min, then NaBH₃CN (123.69 mg, 1.97 mmol) was added at 25° C., and the resulting mixture was stirred at 25° C. for 12 h. LCMS showed a peak (9%) with mass of the starting material, and a peak (86%) with desired mass. The reaction solution was concentrated to remove the organic phase. The crude product was dissolved in EtOAc (50 mL), washed with saturated NaHCO₃ (20 mL), the organic layer was dried over Na₂SO₄, filtered and concentrated under reduced pressure to afford tert-butyl 4-(4-((2,6-dioxopiperidin-3-yl)(methyl)amino)-2-fluorophenyl)piperazine-1-carboxylate (0.6 g, crude) as a blue solid. MS (M+H)⁺=421.0

### Step 5. Synthesis of 3-((3-fluoro-4-(piperazin-1-yl)phenyl)(methyl)amino)piperidine-2,6-dione (6)

To a solution of tert-butyl 4-(4-((2,6-dioxopiperidin-3-yl)(methyl)amino)-2-fluorophenyl)piperazine-1-carboxylate (0.6 g, 1.43 mmol) in dioxane (2 mL) was added HCl/dioxane (4 M, 36.62 mL) at 25° C. The resulting mixture was stirred at 25° C. for 0.5 hr. LCMS showed the starting material was consumed completely. The reaction mixture was concentrated under reduced pressure to afford 3-((3-fluoro-4-(piperazin-1-yl)phenyl)(methyl)amino)piperidine-2,6-dione (0.7 g, crude) as a blue solid. MS (M+H)⁺=321.4

### Step 6. Synthesis of tert-butyl (4-(2-(4-(4-((2,6-dioxopiperidin-3-yl)(methyl)amino)-2-fluorophenyl)piperazin-1-yl)ethyl)piperidin-1-yl)carbamate (7)

To a solution of 3-((3-fluoro-4-(piperazin-1-yl)phenyl)(methyl)amino)piperidine-2,6-dione (200 mg, 560.50 μmol, HCl salt) and 2-(1-((tert-butoxycarbonyl)amino)piperidin-4-yl)ethyl 4-methylbenzenesulfonate (223.37 mg, 560.50 μmol) in DMF (1 mL) were added DIPEA (217.32 mg, 1.68 mmol, 292.88 μL) and NaI (16.80 mg, 112.10 μmol) at 25° C. The reaction mixture was heated to 60° C. for 4 hours. LCMS showed the starting material was consumed completely and a main peak with desired mass. The mixture was poured into water (20 mL) and extracted with EtOAc (15 mL×4). The combined organic phase was washed with brine (15 mL×3), dried over Na₂SO₄, filtered and concentrated under reduced pressure. The crude product was purified by prep-HPLC (column: Phenomenex Synergi Polar-RP 100×25 mm×4 um; mobile phase: [water (TFA)-ACN]; B %: 21%-41%, 7 min) and the eluent was lyophilized to afford tert-butyl (4-(2-(4-(4-((2,6-dioxopiperidin-3-yl)(methyl)amino)-2-fluorophenyl)piperazin-1-yl)ethyl)piperidin-1-yl)carbamate (50 mg, crude) as a brown solid. MS (M+H)⁺=547.4

### Step 7. Synthesis of 3-((4-(4-(2-(1-aminopiperidin-4-yl)ethyl)piperazin-1-yl)-3-fluorophenyl)(methyl)amino)piperidine-2,6-dione (8)

To a solution of tert-butyl (4-(2-(4-(4-((2,6-dioxopiperidin-3-yl)(methyl)amino)-2-fluorophenyl)piperazin-1-yl)ethyl)piperidin-1-yl)carbamate (50 mg, 91.46 μmol) in DCM (2 mL) was added TFA (616.00 mg, 5.40 mmol, 0.4 mL) at 25° C. The resulting mixture was stirred at 25° C. for 0.5 hr. LCMS showed the starting material was consumed completely and a main peak with desired mass. The reaction mixture was concentrated under reduced pressure to afford 3-((4-(4-(2-(1-aminopiperidin-4-yl)ethyl)piperazin-1-yl)-3-fluorophenyl)(methyl)amino)piperidine-2,6-dione (55 mg, crude, TFA salt) as yellow oil. MS (M+H)⁺=447.3

### Step 8. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(4-(2-(4-(4-((2,6-dioxopiperidin-3-yl)(methyl)amino)-2-fluorophenyl)piperazin-1-yl)ethyl)piperidin-1-yl)-3-methoxybenzamide (Compound 23)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (50 mg, 111.75 μmol) in DMF (1 mL) were added HATU (50.99 mg, 134.10 μmol) and DIPEA (57.77 mg, 446.99 μmol, 77.86 μL). The mixture was stirred at 25° C. for 10 min. Then 3-((4-(4-(2-(1-aminopiperidin-4-yl)ethyl)piperazin-1-yl)-3-fluorophenyl)(methyl)amino)piperidine-2,6-dione (50.12 mg, 89.40 μmol, TFA salt) was added and the resulting mixture was stirred at 25° C. for 1 h. LCMS showed the starting material was consumed completely and a peak (34%) with desired mass. The mixture was poured into water (30 mL) and extracted with EtOAc (10 mL×5). The combined organic phase was washed with brine (10 mL×3), dried over Na₂SO₄, filtered. The filtrate was concentrated under reduced pressure. The crude product was purified by prep-HPLC (column: Unisil 3-100 C18 Ultra 150×50 mm×3 μm; mobile phase: [water (FA)-ACN]; B %: 15%-45%, 7 min), the eluent was lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-N-(4-(2-(4-(4-((2,6-dioxopiperidin-3-yl)(methyl)amino)-2-fluorophenyl)piperazin-1-yl)ethyl)piperidin-1-yl)-3-methoxybenzamide (35.6 mg, 37.39 μmol, 33.46% yield, 92% purity) as a white solid. MS (M+H)⁺=876.5

¹H NMR (400 MHz, CD₃CN) δ=8.78-8.65 (m, 1H), 8.46 (d, J=8.4 Hz, 1H), 8.10 (s, 1H), 7.92-7.77 (m, 1H), 7.73 (s, 1H), 7.46-7.30 (m, 2H), 7.01-6.89 (m, 1H), 6.70-6.52 (m, 2H), 4.95-4.81 (m, 1H), 4.60 (dd, J=5.1, 12.8 Hz, 1H), 4.03-3.91 (m, 5H), 3.66-3.49 (m, 2H), 3.44-3.35 (m, 2H), 3.33 (s, 3H), 3.21-3.13 (m, 5H), 3.10-2.99 (m, 2H), 2.77-2.74 (m, 3H), 2.73-2.64 (m, 3H), 2.40-2.26 (m, 2H), 2.02 (dd, J=2.5, 5.0, 10.1 Hz, 3H), 1.83-1.55 (m, 11H), 1.51-1.33 (m, 3H).

### Example 24. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(7-((1-(4-((2,6-dioxopiperidin-3-yl)amino)-2-fluorophenyl)piperidin-4-yl)methyl)-7-azaspiro[3.5]nonan-2-yl)-3-methoxybenzamide (Compound 24)

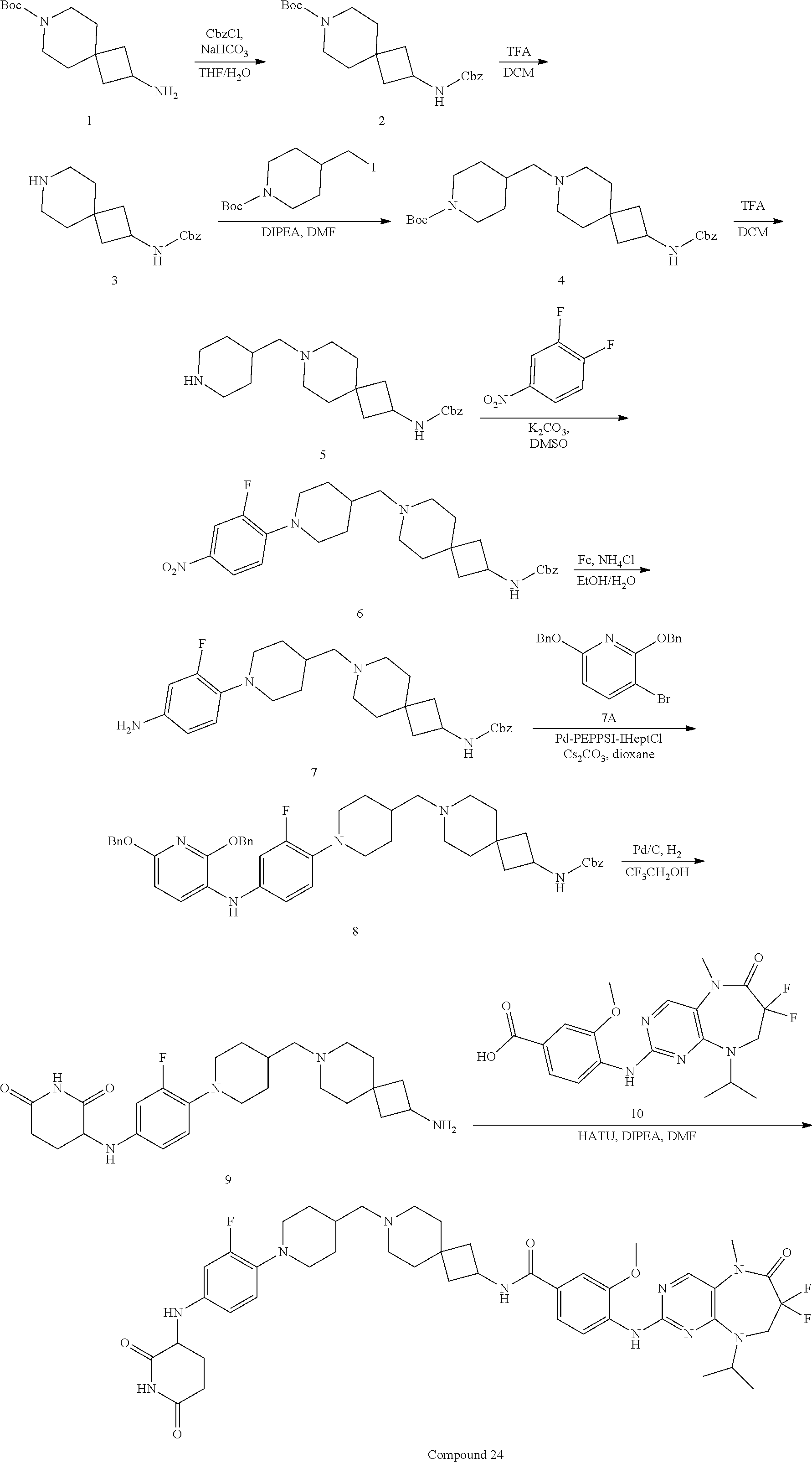

### Step 1. Synthesis of tert-butyl 2-(((benzyloxy)carbonyl)amino)-7-azaspiro[3.5]nonane-7-carboxylate (2)

To a solution of tert-butyl 2-amino-7-azaspiro[3.5]nonane-7-carboxylate (1 g, 4.16 mmol,) in THF (10 mL) and H₂O (5 mL) was added NaHCO₃ (1.05 g, 12.48 mmol, 485.46 L) at 0° C. Then CbzCl (922.73 mg, 5.41 mmol, 768.94 L) was added drop-wise at 0° C. and the resulting mixture was stirred at 20° C. for 2 h. LCMS showed the starting material was consumed completely and a peak (35%) with desired mass. The reaction mixture was diluted with H₂O (20 mL) and extracted with EtOAc (20 mL×3). The combined organic laver was dried over Na₂SO₄, filtered and concentrated. The residue was purified by flash silica gel chromatography (12 g SepaFlash® Silica Flash Column, Eluent of 0˜33% EtOAc/Petroleum ether gradient@100 mL/min) to afford tert-butyl 2-(((benzyloxy)carbonyl)amino)-7-azaspiro[3.5]nonane-7-carboxylate (1.4 g, 3.74 mmol, 89.85% yield) as a white solid. MS (M−100+H)⁺=275.4

### Step 2. Synthesis of benzyl (7-azaspiro[3.5]nonan-2-yl)carbamate (3)

To a solution of tert-butyl 2-(((benzyloxy)carbonyl)amino)-7-azaspiro[3.5]nonane-7-carboxylate (1.4 g, 3.74 mmol) in DCM (5 mL) was added TFA (2.13 g, 18.69 mmol, 1.38 mL) at 20° C. and the mixture was stirred at 20° C. for 12 h. LCMS showed the starting material was consumed completely and a peak (70%) with desired mass. The reaction mixture was concentrated in vacuum to afford benzyl (7-azaspiro[3.5]nonan-2-yl)carbamate (3 g, crude, TFA salt) as a yellow oil. MS (M+H)⁺=275.5

### Step 3. Synthesis of tert-butyl 4-((2-(((benzyloxy)carbonyl)amino)-7-azaspiro[3.5]nonan-7-yl)methyl)piperidine-1-carboxylate (4)

To a solution of benzyl (7-azaspiro[3.5]nonan-2-yl)carbamate (2 g, 5.15 mmol, TFA salt) in DMF (10 mL) were added DIPEA (3.33 g, 25.75 mmol, 4.48 mL) and tert-butyl 4-(iodomethyl) piperidine-1-carboxylate (1.34 g, 4.12 mmol) at 20° C. and the resulting mixture was stirred at 60° C. for 16 h. LCMS showed starting material remained and a peak (21%) with desired mass. The reaction mixture was stirred at 80° C. for 16 h. LCMS showed a little of starting material remained and a peak (28%) with desired mass. The reaction mixture was diluted with H₂O (20 mL) and extracted with EtOAc (20 mL×3). The combined organic layer was washed with brine (20 mL×3), dried over Na₂SO₄, filtered and concentrated. The residue was purified by flash silica gel chromatography (40 g SepaFlash® Silica Flash Column, Eluent of 0˜80% EtOAc/Petroleum ether gradient@100 mL/min) to afford tert-butyl 4-((2-(((benzyloxy)carbonyl)amino)-7-azaspiro[3.5]nonan-7-yl)methyl)piperidine-1-carboxylate (1.6 g, 3.39 mmol, 65.88% yield) as a yellow oil. MS (M+H)⁺=472.3

### Step 4. Synthesis of benzyl (7-(piperidin-4-ylmethyl)-7-azaspiro[3.5]nonan-2-yl)carbamate (5)

To a solution of tert-butyl 4-((2-(((benzyloxy)carbonyl)amino)-7-azaspiro[3.5]nonan-7-yl)methyl)piperidine-1-carboxylate (1.6 g, 3.39 mmol) in DCM (5 mL) was added TFA (1.93 g, 16.96 mmol, 1.26 mL) at 20° C. and the resulting mixture was stirred at 20° C. for 12 h. LCMS showed starting material was consumed completely and a peak (56%) with desired mass. The reaction mixture was concentrated in vacuum to afford benzyl (7-(piperidin-4-ylmethyl)-7-azaspiro[3.5]nonan-2-yl)carbamate (3.1 g, crude, TFA salt) as a yellow oil. MS (M+H)⁺=372.4

### Step 5. Synthesis of benzyl (7-((1-(2-fluoro-4-nitrophenyl)piperidin-4-yl)methyl)-7-azaspiro[3.5]nonan-2-yl)carbamate (6)

To a solution of 1, 2-difluoro-4-nitro-benzene (1 g, 6.29 mmol, 694.44 μL) in DMSO (10 mL) were added K₂CO₃ (2.61 g, 18.86 mmol) and benzyl (7-(piperidin-4-ylmethyl)-7-azaspiro[3.5]nonan-2-yl)carbamate (3.05 g, 6.29 mmol, TFA) at 20° C. and the resulting mixture was stirred at 40° C. for 1 h. LCMS showed 1,2-difluoro-4-nitro-benzene remained and a peak (52%) with desired mass. The reaction mixture was diluted with H₂O (30 mL) and extracted with EtOAc (30 mL×3). The organic layer was washed with brine (30 mL×3), dried over Na₂SO₄, filtered and concentrated. The residue was purified by flash silica gel chromatography (12 g SepaFlash® Silica Flash Column, Eluent of 0˜100% EtOAc/Petroleum ether gradient @100 mL/min) to afford 2 batches of title compound. Batch 1: benzyl (7-((1-(2-fluoro-4-nitrophenyl)piperidin-4-yl)methyl)-7-azaspiro[3.5]nonan-2-yl)carba mate (896 mg, 1.60 mmol, 25.40% yield, 91% purity) was obtained as a yellow solid and Batch 2: benzyl (7-((1-(2-fluoro-4-nitrophenyl)piperidin-4-yl)methyl)-7-azaspiro[3.5]nonan-2-yl)carba mate (490 mg, 921.27 μmol, 14.66% yield, 96% purity) was obtained as a yellow solid. MS (M+H)⁺=511.3

### Step 6. Synthesis of benzyl (7-((1-(4-amino-2-fluorophenyl)piperidin-4-yl)methyl)-7-azaspiro[3.5]nonan-2-yl) carbamate (7)

To a solution of benzyl (7-((1-(2-fluoro-4-nitrophenyl)piperidin-4-yl)methyl)-7-azaspiro[3.5]nonan-2-yl)carba mate (490 mg, 959.66 μmol) in EtOH (6 mL) and H₂O (3 mL) were added Fe (321.55 mg, 5.76 mmol) and NH₄Cl (308.00 mg, 5.76 mmol) at 20° C. and the resulting mixture was stirred at 80° C. for 12 h. LCMS showed the starting material was consumed completely and a peak (91%) with desired mass. The reaction mixture was combined with another batch (896 mg scale) for work-up. Added saturated NaHCO₃ (20 mL) to this reaction mixture to adjust the pH=10 and extracted with EtOAc (40 mL×3). The organic layer was dried over Na₂SO₄, filtered and concentrated to afford benzyl (7-((1-(4-amino-2-fluorophenyl)piperidin-4-yl)methyl)-7-azaspiro[3.5]nonan-2-yl)carbamate (1.1 g, crude) as an orange oil. MS (M+H)⁺=481.4

### Step 7. Synthesis of benzyl (7-((1-(4-((2,6-bis(benzyloxy)pyridin-3-yl)amino)-2-fluorophenyl)piperidin-4-yl)methyl)-7-azaspiro[3.5]nonan-2-yl)carbamate (8)

To a solution of 2,6-dibenzyloxy-3-bromo-pyridine (600 mg, 1.62 mmol) and benzyl (7-((1-(4-amino-2-fluorophenyl)piperidin-4-yl)methyl)-7-azaspiro[3.5]nonan-2-yl)carbamate (934.65 mg, 1.94 mmol) in dioxane (10 mL) were added Pd-PEPPSI-IHeptCl (78.82 mg, 81.03 μmol) and Cs₂CO₃ (1.58 g, 4.86 mmol) at 20° C. under N₂ and the resulting mixture was stirred at 100° C. for 16 h. LCMS showed all starting material was consumed completely and a peak (54%) with desired mass. The reaction mixture was filtered and the filtrate was concentrated in vacuum. The residue was purified by flash silica gel chromatography (25 g SepaFlash® Silica Flash Column, Eluent of 0-80% EtOAc/Petroleum ether gradient@100 mL/min) to afford 2 batches of title compound. Batch 1: benzyl (7-((1-(4-((2,6-bis(benzyloxy)pyridin-3-yl)amino)-2-fluorophenyl)piperidin-4-yl)meth yl)-7-azaspiro[3.5]nonan-2-yl)carbamate (742 mg, 963.71 μmol, 59.47% yield) was obtained as a green oil and Batch 2: benzyl (7-((1-(4-((2,6-bis(benzyloxy)pyridin-3-yl)amino)-2-fluorophenyl)piperidin-4-yl)meth yl)-7-azaspiro[3.5]nonan-2-yl)carbamate (246 mg, 319.50 μmol, 19.72% yield) was obtained as a green oil. MS (M+H)⁺=770.1

### Step 8. Synthesis of 3-((4-(4-((2-amino-7-azaspiro[3.5]nonan-7-yl)methyl)piperidin-1-yl)-3-fluorophen yl)amino)piperidine-2,6-dione (9)

To a solution of benzyl (7-((1-(4-((2,6-bis(benzyloxy)pyridin-3-yl)amino)-2-fluorophenyl)piperidin-4-yl)meth yl)-7-azaspiro[3.5]nonan-2-yl)carbamate (400 mg, 519.52 μmol) in CF₃CH₂OH (10 mL) was added Pd/C (0.1 g, 10% purity) under N₂ atmosphere. The suspension was degassed and purged with H₂ for 3 times. The mixture was stirred at 20° C. for 16 h under H₂ (15 Psi). LCMS showed the starting material was consumed completely and a peak with desired mass. The reaction mixture was combined with another batch (246 mg scale) for work-up. The reaction mixture was diluted with EtOAc (15 mL) and filtered. The filtrate was concentrated in vacuum to afford 3-((4-(4-((2-amino-7-azaspiro[3.5]nonan-7-yl)methyl)piperidin-1-yl)-3-fluorophenyl)amino)piperidine-2,6-dione (219 mg, 478.60 μmol, 92.12% yield) as a green oil. MS (M+H)⁺=458.1

### Step 9. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(7-((1-(4-((2,6-dioxopiperidin-3-yl)amino)-2-fluorophenyl)piperidin-4-yl)methyl)-7-azaspiro[3.5]nonan-2-yl)-3-methoxybenzamide (Compound 24)

To a solution of 4-((7,7-difluoro-9-isopropyl-5-methyl-6-oxo-6,7,8,9-tetrahydro-5H-pyrimido[4,5-b][1,4]diazepin-2-yl)amino)-3-methoxybenzoic acid (100 mg, 237.31 μmol) in DMF (2 mL) were added HATU (99.25 mg, 261.04 μmol) and DIPEA (92.01 mg, 711.92 μmol, 124.00 μL). The mixture was stirred at 20° C. for 10 min and a solution of 3-((4-(4-((2-amino-7-azaspiro[3.5]nonan-7-yl)methyl)piperidin-1-yl)-3-fluorophenyl)amino)piperidine-2,6-dione (217.17 mg, 474.61 μmol) in DMF (2 mL) was added and the resulting mixture was stirred at 20° C. for 1 h. LCMS showed the starting material was consumed completely and a peak (68%) with desired mass. The reaction mixture was diluted with H₂O (15 mL) and extracted with EtAOc (15 mL×3). The organic layer was washed with brine (15 mL×3), dried over Na₂SO₄, filtered and concentrated. The residue was purified by flash silica gel chromatography (12 g SepaFlash® Silica Flash Column, Eluent of 0˜100% EtOAc/Petroleum ether gradient@100 mL/min) followed by prep-HPLC (column: Phenomenex luna C18 150*40 mm*15 m; mobile phase: [water (FA)-ACN]; B %: 13%-43%, 10 min) and the eluent was lyophilized to afford 4-((7,7-difluoro-9-isopropyl-5-methyl-6-oxo-6,7,8,9-tetrahydro-5H-pyrimido[4,5-b][1,4]diazepin-2-yl)amino)-N-(7-((1-(4-((2,6-dioxopiperidin-3-yl)amino)-2-fluorophenyl)piperidin-4-yl)methyl)-7-azaspiro[3.5]nonan-2-yl)-3-methoxybenzamide (119.5 mg, 126.42 μmol, 53.27% yield, 94% purity, 0.6 FA salt) as a gray solid. MS (M+H)⁺=861.1

¹H NMR (400 MHz, DMSO-d₆) δ=10.77 (s, 1H), 8.42 (br d, J=7.2 Hz, 1H), 8.30 (d, J=8.3 Hz, 1H), 8.22 (s, 1H), 8.16 (s, 1H), 7.88 (s, 1H), 7.54-7.45 (m, 2H), 6.82 (br t, J=9.3 Hz, 1H), 6.49 (dd, J=2.0, 15.0 Hz, 1H), 6.41 (br d, J=8.7 Hz, 1H), 5.77 (br d, J=7.3 Hz, 1H), 4.94-4.82 (m, 1H), 4.47-4.34 (m, 1H), 4.30-4.19 (m, 1H), 4.04 (br t, J=13.5 Hz, 2H), 3.94 (s, 3H), 3.32 (s, 3H), 3.10 (br d, J=11.1 Hz, 2H), 2.78-2.67 (m, 1H), 2.61-2.54 (m, 2H), 2.54-2.50 (m, 2H), 2.50-2.23 (m, 2H), 2.22-2.03 (m, 6H), 1.90-1.78 (m, 3H), 1.74 (br d, J=11.7 Hz, 2H), 1.59 (br d, J=19.2 Hz, 5H), 1.27-1.17 (m, 8H).

### Example 25. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(7-((1-(4-((2,6-dioxopiperidin-3-yl)amino)-2,6-difluorophenyl)piperidin-4-yl)methyl)-7-azaspiro[3.5]nonan-2-yl)-3-methoxybenz amide (Compound 25)

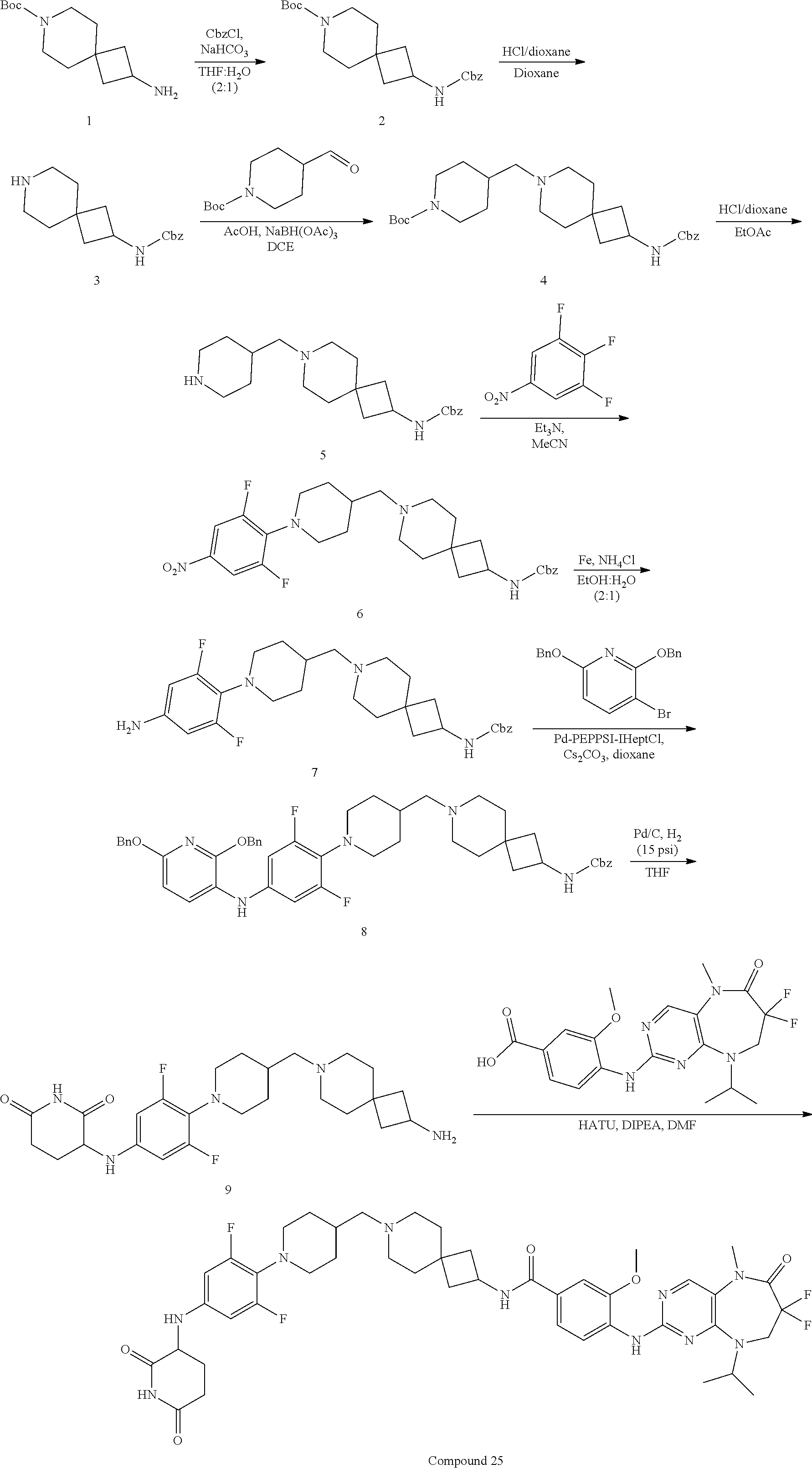

### Step 1. Synthesis of tert-butyl 2-(((benzyloxy)carbonyl)amino)-7-azaspiro[3.5]nonane-7-carboxylate (2)

To a solution of tert-butyl 2-amino-7-azaspiro[3.5]nonane-7-carboxylate (1 g, 4.16 mmol) in THF (10 mL) and H₂O (5 mL) was added NaHCO₃ (1.05 g, 12.48 mmol) at 0° C. Then CbzCl (922.73 mg, 5.41 mmol) was added drop-wise at 0° C. and the resulting mixture was stirred at 20° C. for 2 h. LCMS showed the starting material was consumed completely and a peak (35%) with desired mass. The reaction mixture was diluted with H₂O (20 mL) and extracted with EtOAc (20 mL×3). The organic layer was dried over Na₂SO₄, filtered and concentrated. The residue was purified by flash silica gel chromatography (12 g SepaFlash® Silica Flash Column, Eluent of 0˜33% EtOAc/Petroleum ether gradient@100 mL/min) to afford tert-butyl 2-(((benzyloxy)carbonyl)amino)-7-azaspiro[3.5]nonane-7-carboxylate (1.4 g, 3.74 mmol, 89.85% yield) as a white solid. MS (M−100+H)⁺=275.4

### Step 2. Synthesis of benzyl (7-azaspiro[3.5]nonan-2-yl)carbamate (3)

To a solution of tert-butyl 2-(((benzyloxy)carbonyl)amino)-7-azaspiro[3.5]nonane-7-carboxylate (6 g, 16.02 mmol) in dioxane (30 mL) was added HCl/dioxane (4 M, 30 mL), the mixture was stirred at 20° C. for 2 hrs. LCMS showed the starting material was consumed completely and a peak with desired mass. The mixture was concentrated under vacuum to afford benzyl (7-azaspiro[3.5]nonan-2-yl)carbamate (6 g, crude) as a white solid, which was used for the next step directly. MS (M+H)⁺=275.2

### Step 3. Synthesis of tert-butyl 4-((2-(((benzyloxy)carbonyl)amino)-7-azaspiro[3.5]nonan-7-yl)methyl) piperidine-1-carboxylate (4)

A solution of benzyl (7-azaspiro[3.5]nonan-2-yl)carbamate (5 g, 16.09 mmol, HCl salt), tert-butyl 4-formylpiperidine-1-carboxylate (3.43 g, 16.09 mmol) and AcOH (966.03 mg, 16.09 mmol) in DCE (60 mL) was stirred at 20° C. for 30 mins. Then NaBH(OAc)₃ (6.82 g, 32.17 mmol) was added and the resulting mixture was stirred at 20° C. for 1 h. LCMS showed a main peak with desired mass. The reaction mixture was poured into NaHCO₃ solution (100 mL). The layers were separated. The aqueous layer was extracted with DCM (30 mL×3). The combined organic layers were dried over Na₂SO₄ and concentrated to afford tert-butyl 4-((2-(((benzyloxy)carbonyl)amino)-7-azaspiro[3.5]nonan-7-yl)methyl)piperidine-1-carboxylate (7.5 g, 15.90 mmol, 98.85% yield) as a white solid. MS (M+H)⁺=472.4

### Step 4. Synthesis of benzyl (7-(piperidin-4-ylmethyl)-7-azaspiro[3.5]nonan-2-yl)carbamate (5)

To a solution of tert-butyl 4-((2-(((benzyloxy)carbonyl)amino)-7-azaspiro[3.5]nonan-7-yl)methyl)piperidine-1-carboxylate (6 g, 12.72 mmol) in EtOAc (25 mL) was added HCl/dioxane (4 M, 30.00 mL), the mixture was stirred at 20° C. for 1 h. LCMS showed the starting material was consumed completely and the desired mass. The reaction mixture was concentrated to afford benzyl (7-(piperidin-4-ylmethyl)-7-azaspiro[3.5]nonan-2-yl)carbamate (5 g, crude, 2HCl salt) as a white solid. MS (M+H)⁺=372.2

### Step 5. Synthesis of benzyl (7-((1-(2,6-difluoro-4-nitrophenyl)piperidin-4-yl)methyl)-7-azaspiro[3.5]nonan-2-yl)carbamate (6)

To a solution of benzyl (7-(piperidin-4-ylmethyl)-7-azaspiro[3.5]nonan-2-yl)carbamate (2.5 g, 5.63 mmol, 2HCl salt) and 1,2,3-trifluoro-5-nitro-benzene (996.10 mg, 5.63 mmol) in MeCN (20 mL) was added Et₃N (2.85 g, 28.13 mmol) at 20° C. The resulting mixture was stirred at 80° C. for 1 h. LCMS showed the starting material was consumed completely and the desired mass. The reaction solution was concentrated. The crude product was purified by flash silica gel chromatography (25 g silica gel column, EtOAc/petroleum ether=10-60%, 80 mL/min) to afford benzyl (7-((1-(2,6-difluoro-4-nitrophenyl)piperidin-4-yl)methyl)-7-azaspiro[3.5]nonan-2-yl)carbamate (1 g, 1.89 mmol, 33.63% yield) as a yellow solid. MS (M+H)⁺=529.3

### Step 6. Synthesis of benzyl (7-((1-(4-amino-2,6-difluorophenyl)piperidin-4-yl)methyl)-7-azaspiro[3.5]nonan-2-yl)carbamate (7)

To a mixture of benzyl (7-((1-(2,6-difluoro-4-nitrophenyl)piperidin-4-yl)methyl)-7-azaspiro[3.5]nonan-2-yl)carbamate (1 g, 1.89 mmol) and NH₄Cl (607.18 mg, 11.35 mmol) in EtOH (20 mL) and H₂O (10 mL) was added Fe (633.89 mg, 11.35 mmol) at 20° C. The resulting mixture was stirred at 80° C. for 1 h. LCMS showed the starting material was consumed completely and the desired mass. The reaction mixture was poured into NaHCO₃ solution (200 mL) and extracted with EtOAc (50 mL×4). The combined organic layers were dried over Na₂SO₄ and concentrated to afford benzyl (7-((1-(4-amino-2,6-difluorophenyl)piperidin-4-yl)methyl)-7-azaspiro[3.5]nonan-2-yl)carbamate (900 mg, crude) as a yellow solid, which was used for the next step directly. MS (M+H)⁺=499.5

### Step 7. Synthesis of benzyl (7-((1-(4-((2,6-bis(benzyloxy)pyridin-3-yl)amino)-2,6-difluorophenyl)piperidin-4-yl)methyl)-7-azaspiro[3.5]nonan-2-yl)carbamate (8)

To a mixture of benzyl (7-((1-(4-amino-2,6-difluorophenyl)piperidin-4-yl)methyl)-7-azaspiro[3.5]nonan-2-yl) carbamate (900 mg, 1.81 mmol), 2,6-dibenzyloxy-3-bromo-pyridine (801.95 mg, 2.17 mmol) and Cs₂CO₃ (1.76 g, 5.42 mmol) in dioxane (15 mL) was added PdPEPPSI-IHeptCl (87.79 mg, 90.25 μmol) at 20° C. The resulting mixture was purged and degassed with N₂, heated to 100° C. and stirred for 14 hrs. LCMS showed the starting material was consumed completely and the desired mass. The reaction mixture was diluted with EtOAc (50 mL) and filtered. The filtrate was concentrated. The crude product was purified by flash silica gel chromatography (12 g silica gel column, EtOAc/petroleum ether=10-40%, 80 mL/min) to afford benzyl (7-((1-(4-((2,6-bis(benzyloxy)pyridin-3-yl)amino)-2,6-difluorophenyl)piperidin-4-yl)methyl)-7-azaspiro[3.5]nonan-2-yl)carbamate (900 mg, 959.47 μmol, 53.16% yield, 84% purity) as a brown solid. MS (M+H)⁺=788.8

### Step 8. Synthesis of 3-((4-(4-((2-amino-7-azaspiro[3.5]nonan-7-yl)methyl)piperidin-1-yl)-3,5-difluorophenyl)amino)piperidine-2,6-dione (9)

To a solution of benzyl (7-((1-(4-((2,6-bis(benzyloxy)pyridin-3-yl)amino)-2,6-difluorophenyl) piperidin-4-yl)methyl)-7-azaspiro[3.5]nonan-2-yl)carbamate (800 mg, 1.02 mmol) in THF (15 mL) was added Pd/C (200 mg, 10% purity) at 20° C. under N₂. The mixture was purged and degassed with H₂ for three times, then stirred at 20° C. for 2 hrs under H₂ (15 Psi). LCMS showed most of the starting material remained. The reaction mixture was stirred at 20° C. under H₂ (15 Psi) for another 12 hrs. LCMS showed the starting material remained and the desired mass. The reaction mixture was filtered through celite pad. To the filtrate was added Pd/C (100 mg, 10% purity) at 20° C. under N₂. The mixture was purged and degassed with H₂ for three times and then stirred at 20° C. for 4 hrs under H₂ (15 Psi). LCMS showed the starting material was consumed completely and the desired mass. The reaction mixture was filtered through a celite pad to afford 3-((4-(4-((2-amino-7-azaspiro[3.5]nonan-7-yl)methyl)piperidin-1-yl)-3,5-difluorophenyl)amino)piperidine-2,6-dione (400 mg, crude) as a THF solution which was used for the next step directly. MS (M+H)⁺=476.3

### Step 9. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(7-((1-(4-((2,6-dioxopiperidin-3-yl)amino)-2,6-difluorophenyl)piperidin-4-yl)methyl)-7-azaspiro[3.5]nonan-2-yl)-3-methoxybenz amide (Compound 25)

To a solution of 3-((4-(4-((2-amino-7-azaspiro[3.5]nonan-7-yl)methyl)piperidin-1-yl)-3,5-difluorophenyl)amino)piperidine-2,6-dione (400 mg, 841.09 μmol), 4-[(7,7-difluoro-9-isopropyl-5-methyl-6-oxo-8H-pyrimido[4,5-b][1,4]diazepin-2-yl) amino]-3-methoxy-benzoic acid (248.10 mg, 588.76 μmol) and DIPEA (543.53 mg, 4.21 mmol) in THF (20 mL) was added HATU (319.81 mg, 841.09 μmol) at 20° C. The resulting mixture was stirred at 20° C. for 13 hrs. LCMS showed the starting material was consumed completely and the desired mass. The reaction mixture was concentrated. The crude product was purified by flash silica gel chromatography (4 g silica gel column, EtOAc/petroleum ether=30-100% and then methanol/EtOAc=10-20%, 40 mL/min) to afford the crude product, which was further purified by prep-HPLC (column: Unisil 3-100 C18 Ultra 150*50 mm*3 m; mobile phase: [water (FA)-ACN]; B %: 13%-43%, 7 min) and the eluent was lyophilized to afford 4-((7,7-difluoro-9-isopropyl-5-methyl-6-oxo-6,7,8,9-tetrahydro-5H-pyrimido[4,5-b][1,4]diazepin-2-yl)amino)-N-(7-((1-(4-((2,6-dioxopiperidin-3-yl)amino)-2,6-difluorophenyl)piperidin-4-yl)methyl)-7-azaspiro[3.5]nonan-2-yl)-3-methoxybenzamide (40.6 mg, 45.54 μmol, 5.41% yield, 98.6% purity) as a white solid. MS (M+H)⁺=879.4

¹H NMR (400 MHz, DMSO-d₆) δ=10.82 (s, 1H), 8.45 (br d, J=6.7 Hz, 1H), 8.31 (d, J=8.2 Hz, 1H), 8.22 (s, 1H), 7.89 (s, 1H), 7.53-7.46 (m, 2H), 6.31 (br d, J=12.3 Hz, 2H), 6.24 (br d, J=7.8 Hz, 1H), 4.88 (td, J=6.6, 13.2 Hz, 1H), 4.45-4.37 (m, 1H), 4.35-4.27 (m, 1H), 4.04 (br t, J=13.6 Hz, 2H), 3.94 (s, 3H), 3.32 (br s, 3H), 2.98-2.90 (m, 4H), 2.75-2.71 (m, 1H), 2.40-2.35 (m, 2H), 2.25-2.15 (m, 4H), 2.10-2.01 (m, 2H), 1.90-1.80 (m, 4H), 1.74-1.63 (m, 7H), 1.24 (br d, J=6.7 Hz, 8H).

### Example 26. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(7-((1-(4-((2,6-dioxopiperidin-3-yl)(methyl)amino)-2-fluorophenyl)piperidin-4-yl)methyl)-7-azaspiro[3.5]nonan-2-yl)-3-methoxybenzamide (Compound 26)

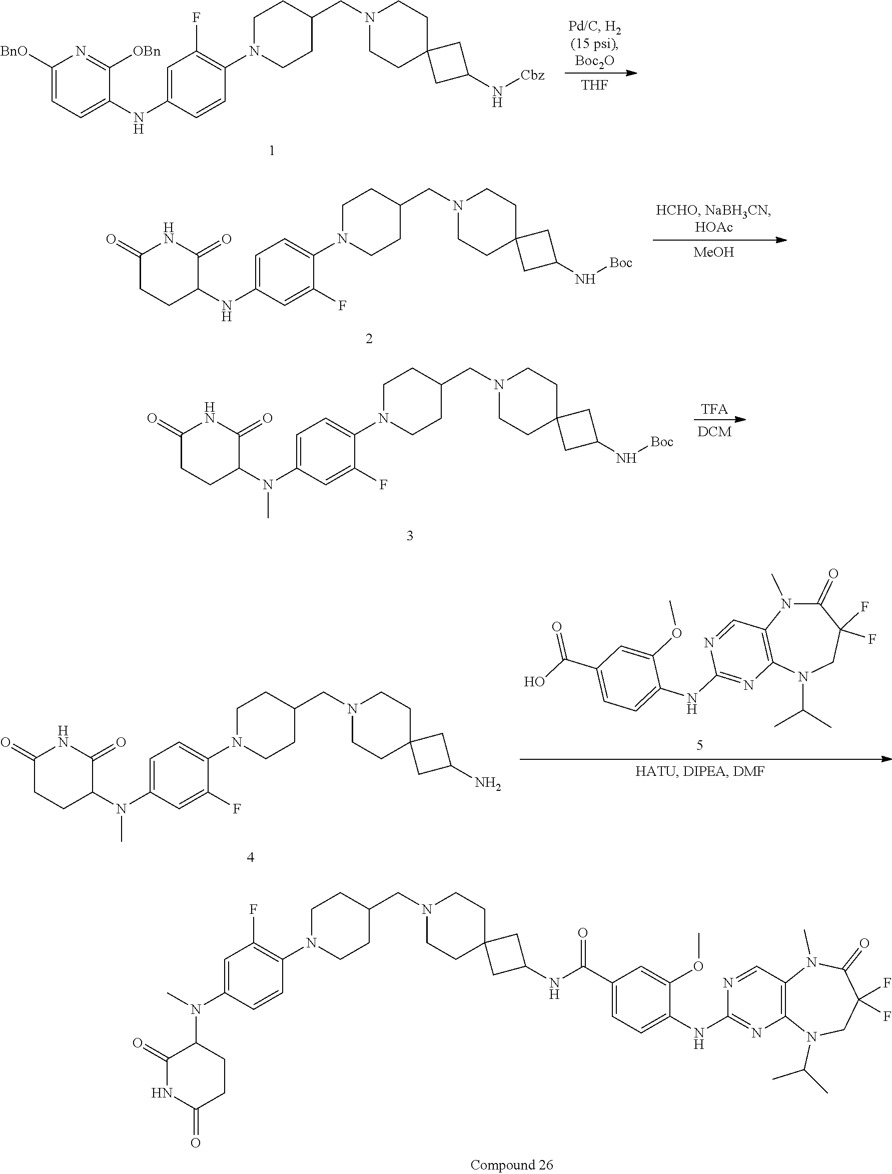

### Step 1. Synthesis of tert-butyl (7-((1-(4-((2,6-dioxopiperidin-3-yl)amino)-2-fluorophenyl)piperidin-4-yl)methyl)-7-azaspiro[3.5]nonan-2-yl)carbamate (2)

To a solution of benzyl (7-((1-(4-((2,6-bis(benzyloxy)pyridin-3-yl)amino)-2-fluorophenyl)piperidin-4-yl)meth yl)-7-azaspiro[3.5]nonan-2-yl)carbamate (500 mg, 649.40 μmol) in THF (20 mL) was added Boc₂O (500 mg, 2.29 mmol, 526.32 μL) and Pd/C (200 mg, 10% purity) under N₂ atmosphere, the suspension was degassed and purged with H₂ for several times, then stirred at 20° C. for 16 hr under H₂ atmosphere (15 Psi). LCMS showed the starting material was consumed completely and one main peak with desired mass. The reaction mixture was filtered and the filtrate was concentrated under reduced pressure. The residue was purified by flash silica gel chromatography (Biotage; 10 g SepaFlash® Silica Flash Column, Eluent of 0˜20% Methanol:Dichloromethane gradient, 60 mL/min) to afford tert-butyl (7-((1-(4-((2,6-dioxopiperidin-3-yl)amino)-2-fluorophenyl)piperidin-4-yl)methyl)-7-azaspiro[3.5]nonan-2-yl)carbamate (300 mg, 537.93 μmol, 82.83% yield) as a brown solid. MS (M+H)⁺=558.3

### Step 2. Synthesis of tert-butyl (7-((1-(4-((2,6-dioxopiperidin-3-yl)(methyl)amino)-2-fluorophenyl)piperidin-4-yl) methyl)-7-azaspiro[3.5]nonan-2-yl)carbamate (3)

To a solution of tert-butyl (7-((1-(4-((2,6-dioxopiperidin-3-yl)amino)-2-fluorophenyl)piperidin-4-yl)methyl)-7-azaspiro[3.5]nonan-2-yl)carbamate (300 mg, 537.93 μmol) in MeOH (20 mL) was added HOAc (32.30 mg, 537.93 μmol, 30.77 L) and HCHO (750.00 mg, 9.24 mmol, 688.07 μL, 37% purity). The mixture was stirred at 20° C. for 1 hr. Then NaBH₃CN (375.00 mg, 5.97 mmol) was added at 0° C., the resulting mixture was stirred at 20° C. for 15 hr. LCMS showed the starting material was consumed completely and one main peak with desired mass. The reaction mixture was diluted with H₂O (15 mL) and concentrated. The residue was dissolved in EtOAc (30 mL), then saturated NaHCO₃ was added to adjust the pH=9, the mixture was extracted with EtOAc (30 mL×2). The combined organic layers were washed with brine (30 mL×2), dried over Na₂SO₄, filtered. The filtrate was concentrated under reduced pressure. The residue was purified by flash silica gel chromatography (Biotage; 10 g SepaFlash® Silica Flash Column, Eluent of 0˜20% Methanol:Dichloromethane gradient, 60 mL/min) to afford tert-butyl (7-((1-(4-((2,6-dioxopiperidin-3-yl)(methyl)amino)-2-fluorophenyl)piperidin-4-yl)methyl)-7-azaspiro[3.5]nonan-2-yl)carbamate (280 mg, 489.75 μmol, 91.04% yield) as a light yellow solid. MS (M+H)⁺=572.3

### Step 3. Synthesis of 3-((4-(4-((2-amino-7-azaspiro[3.5]nonan-7-yl)methyl)piperidin-1-yl)-3-fluorophen yl)(methyl)amino)piperidine-2,6-dione (4)

To a solution of tert-butyl (7-((1-(4-((2,6-dioxopiperidin-3-yl)(methyl)amino)-2-fluorophenyl)piperidin-4-yl)methyl)-7-azaspiro[3.5]nonan-2-yl)carbamate (280 mg, 489.75 μmol) in DCM (5 mL) was added TFA (4.52 g, 39.67 mmol, 2.94 mL) under N₂ atmosphere, the mixture was stirred at 20° C. for 1 hr. LCMS showed the starting material was consumed completely and a peak (65%) with desired mass. The reaction mixture was concentrated to afford 3-((4-(4-((2-amino-7-azaspiro[3.5]nonan-7-yl)methyl)piperidin-1-yl)-3-fluorophenyl)(methyl)amino)piperidine-2,6-dione (286 mg, crude, TFA salt) as a yellow oil. MS (M+H)⁺=472.3

### Step 4. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(7-((1-(4-((2,6-dioxopiperidin-3-yl)(methyl)amino)-2-fluorophenyl)piperidin-4-yl)methyl)-7-azaspiro[3.5]nonan-2-yl)-3-methoxybenzamide (Compound 26)

To a solution of 4-((7,7-difluoro-9-isopropyl-5-methyl-6-oxo-6,7,8,9-tetrahydro-5H-pyrimido[4,5-b][1,4]diazepin-2-yl)amino)-3-methoxybenzoic acid (220 mg, 522.07 μmol) in DMF (4 mL) were added HATU (280 mg, 736.40 μmol), DIPEA (544.12 mg, 4.21 mmol, 733.32 μL) and 3-((4-(4-((2-amino-7-azaspiro[3.5]nonan-7-yl)methyl)piperidin-1-yl)-3-fluorophenyl)(methyl)amino)piperidine-2,6-dione (286 mg, 488.36 μmol, TFA salt) at 20° C. The mixture was stirred at 20° C. for 16 hr under N₂ atmosphere. LCMS showed all starting material was consumed and a peak (40%) with desired mass. The reaction mixture was diluted with H₂O (10 mL), and extracted with EtOAc 40 mL (20 mL×2). The combined organic layers were washed with brine 60 mL (20 mL×3), dried over Na₂ SO₄, filtered. The filtrate was concentrated under reduced pressure. The residue was purified by flash silica gel chromatography (Biotage; 10 g SepaFlash® Silica Flash Column, Eluent of 0˜25% Methanol:Dichloromethane gradient, 60 mL/min) and re-purified by prep-HPLC (column: Unisil 3-100 C18 Ultra 150×50 mm×3 μm; mobile phase: [water (FA)-ACN]; B %: 13%-43%, 7 min; Column Temp: 30° C.), the eluent was lyophilized to afford 4-((7,7-difluoro-9-isopropyl-5-methyl-6-oxo-6,7,8,9-tetrahydro-5H-pyrimido[4,5-b][1,4]diazepin-2-yl)amino)-N-(7-((1-(4-((2,6-dioxopiperidin-3-yl)(methyl)amino)-2-fluoro phenyl)piperidin-4-yl)methyl)-7-azaspiro[3.5]nonan-2-yl)-3-methoxybenzamide (109.6 mg, 124.01 μmol, 23.75% yield, 99% purity) as a white solid. MS (M+H)⁺=875.2

¹H NMR (400 MHz, DMSO-d₆) δ 10.80 (s, 1H), 8.44 (d, J=7.5 Hz, 1H), 8.35-8.28 (m, 1H), 8.22 (s, 1H), 7.89 (s, 1H), 7.55-7.44 (m, 2H), 6.89 (t, J=9.6 Hz, 1H), 6.70-6.62 (m, 1H), 6.56-6.48 (m, 1H), 4.95-4.87 (m, 1H), 4.84-4.76 (m, 1H), 4.45-4.33 (m, 1H), 4.04 (t, J=13.8 Hz, 2H), 3.94 (s, 3H), 3.34 (s, 3H), 3.18-3.10 (m, 2H), 2.89-2.76 (m, 1H), 2.67 (s, 3H), 2.56-2.51 (m, 6H), 2.28-2.13 (m, 6H), 1.87-1.78 (m, 3H), 1.78-1.71 (m, 2H), 1.65-1.53 (m, 5H), 1.29-1.18 (m, 8H).

### Example 27. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(7-((1-(4-((2,6-dioxopiperidin-3-yl)(methyl)amino)phenyl)piperidin-4-yl)methyl)-7-azaspiro[3.5]nonan-2-yl)-3-methoxybenzamide (Compound 27)

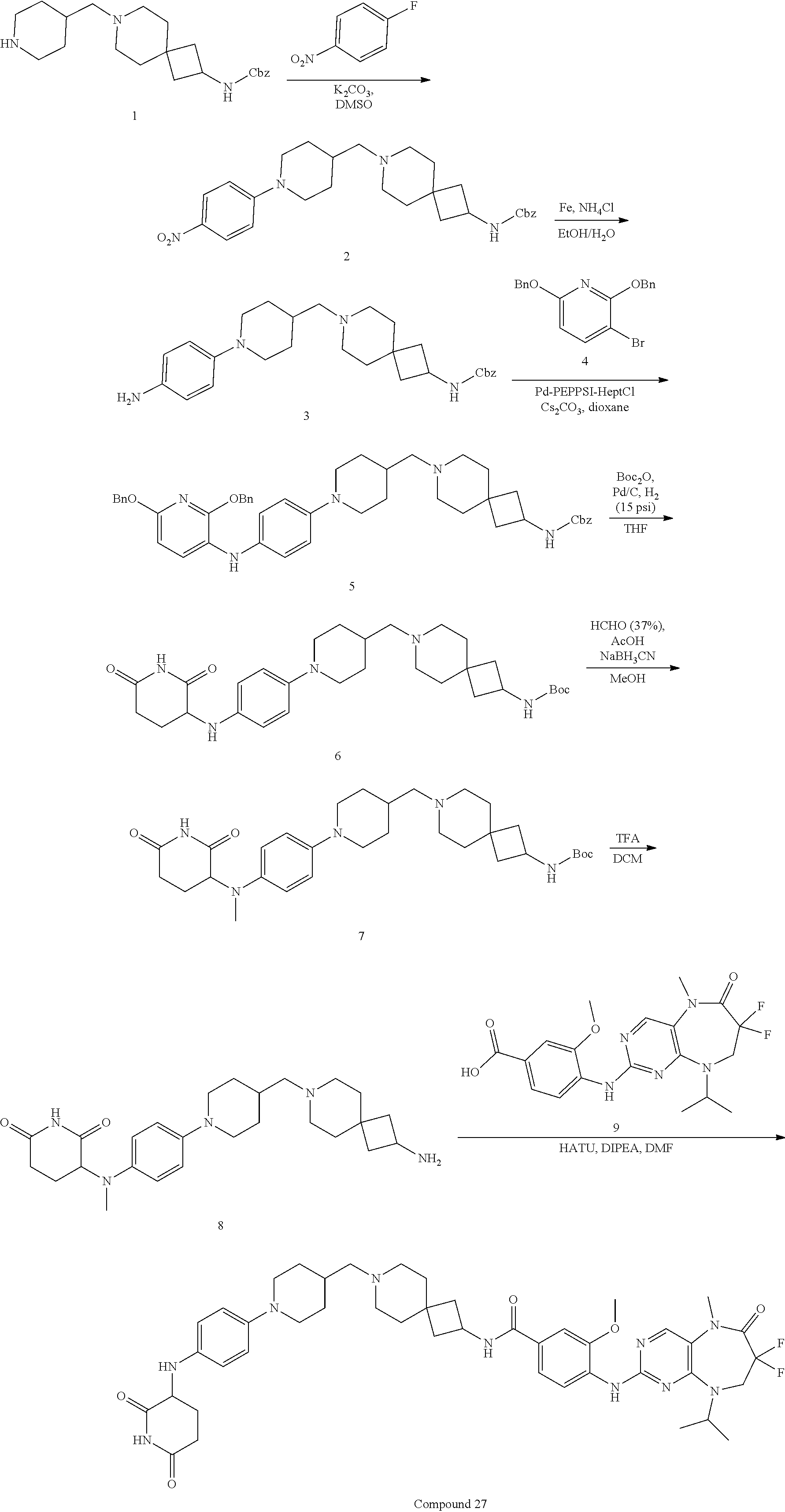

### Step 1. Synthesis of benzyl (7-((1-(4-nitrophenyl)piperidin-4-yl)methyl)-7-azaspiro[3.5]nonan-2-yl)carbamate (2)

To a solution of benzyl (7-(piperidin-4-ylmethyl)-7-azaspiro[3.5]nonan-2-yl)carbamate (2 g, 4.90 mmol, HCl salt) in DMSO (15 mL) were added K₂CO₃ (2.03 g, 14.71 mmol) and 1-fluoro-4-nitrobenzene (1.04 g, 7.35 mmol, 780.12 L). The mixture was stirred at 20° C. for 12 h. LCMS showed ˜45% of desired mass was detected. The reaction mixture was diluted with water (80 mL) and extracted with EtOAc (60 mL×3). The combined organic layers were washed with brine (100 mL), dried over anhydrous sodium sulfate, filtered. The filtrate was concentrated under reduced pressure. The residue was purified by flash silica gel chromatography (ISCO®; 40 g SepaFlash® Silica Flash Column, Eluent of 0˜30% petroleum ether:(EtOAc/ethanol=2/1) gradient@80 mL/min) to afford benzyl (7-((1-(4-nitrophenyl)piperidin-4-yl)methyl)-7-azaspiro[3.5]nonan-2-yl)carbamate (1.2 g, 2.41 mmol, 49.10% yield, 98.8% purity) as a yellow solid. MS (M+H)⁺=493.2

### Step 2. Synthesis of benzyl (7-((1-(4-aminophenyl)piperidin-4-yl)methyl)-7-azaspiro[3.5]nonan-2-yl)carbamate (3)

To a solution of benzyl (7-((1-(4-nitrophenyl)piperidin-4-yl)methyl)-7-azaspiro[3.5]nonan-2-yl)carbamate (1.2 g, 2.44 mmol) in EtOH (8 mL) were added Fe (544.16 mg, 9.74 mmol) and a solution of NH₄Cl (521.22 mg, 9.74 mmol) in H₂O (8 mL). The mixture was stirred at 80° C. for 16 h. LCMS showed a peak (87%) with desired mass. The mixture was diluted with saturated sodium bicarbonate solution (100 mL), then filtered to remove insoluble solid. The filtrate was extracted with EtOAc (50 mL×4). The combined organic layers were washed with brine (150 mL), dried over anhydrous sodium sulfate, filtered. The filtrate was concentrated under reduced pressure to afford benzyl (7-((1-(4-aminophenyl)piperidin-4-yl)methyl)-7-azaspiro[3.5]nonan-2-yl)carbamate (1 g, 1.53 mmol, 62.65% yield, 70.6% purity) as a gray solid. MS (M+H)⁺=463.3

### Step 3. Synthesis of benzyl (7-((1-(4-((2,6-bis(benzyloxy)pyridin-3-yl)amino)phenyl)piperidin-4-yl)methyl)-7-azaspiro[3.5]nonan-2-yl)carbamate (5)

To a solution of benzyl (7-((1-(4-aminophenyl)piperidin-4-yl)methyl)-7-azaspiro[3.5]nonan-2-yl)carbamate (1 g, 2.16 mmol) in dioxane (10 mL) were added 2,6-bis(benzyloxy)-3-bromopyridine (880.33 mg, 2.38 mmol), Cs₂CO₃ (2.11 g, 6.48 mmol) and Pd-PEPPSI-IHeptCl (210.27 mg, 216.16 μmol). The mixture was stirred at 100° C. for 12 h under N₂ atmosphere. LCMS showed a peak (54%) with desired mass. The reaction mixture was diluted with water (100 mL) and extracted with EtOAc (60 mL×3). The combined organic layers were washed with brine (100 mL), dried over anhydrous sodium sulfate, filtered. The filtrate was concentrated under reduced pressure. The residue was purified by flash silica gel chromatography (ISCO®; 20 g SepaFlash® Silica Flash Column, Eluent of 0˜35% petroleum ether:(EtOAc/ethanol=2/1) gradient@80 mL/min) to afford benzyl (7-((1-(4-((2,6-bis(benzyloxy)pyridin-3-yl)amino)phenyl)piperidin-4-yl)methyl)-7-aza spiro[3.5]nonan-2-yl)carbamate (1.45 g, 1.93 mmol, 89.21% yield) as brown oil MS (M+H)⁺=752.4

¹H NMR (400 MHz, DMSO-d₆) δ=7.56-7.49 (m, 1H), 7.42-7.29 (m, 15H), 6.79 (s, 4H), 6.34 (d, J=8.3 Hz, 1H), 5.38 (s, 2H), 5.25 (s, 2H), 4.98 (s, 2H), 3.98-3.89 (m, 1H), 3.45 (d, J=11.9 Hz, 2H), 3.33-3.29 (m, 4H), 2.56-2.52 (m, 4H), 2.0-2.06 (m, 2H), 1.80-1.44 (m, 11H), 1.24-1.21 (m, 2H).

### Step 4. Synthesis of tert-butyl (7-((1-(4-((2,6-dioxopiperidin-3-yl)amino)phenyl)piperidin-4-yl)methyl)-7-azaspiro[3.5]nonan-2-yl)carbamate (6)

To a solution of benzyl (7-((1-(4-((2,6-bis(benzyloxy)pyridin-3-yl)amino)phenyl)piperidin-4-yl)methyl)-7-aza spiro[3.5]nonan-2-yl)carbamate (0.5 g, 664.94 μmol) and Boc₂O (435.36 mg, 1.99 mmol, 458.27 μL) in THF (5 mL) was added Pd/C (50 mg, 66.49 μmol, 10% purity) under N₂ atmosphere. The mixture was stirred at 20° C. for 16 h under H₂ (15 psi). LCMS showed a peak (˜20%) with desired mass. The reaction mixture was filtered. The filtrate was concentrated under reduced pressure. The residue was purified by flash silica gel chromatography (ISCO®; 12 g SepaFlash® Silica Flash Column, Eluent of 0˜40% petroleum ether:(EtOAc/methanol=1/2) gradient@60 mL/min) to afford tert-butyl (7-((1-(4-((2,6-dioxopiperidin-3-yl)amino)phenyl)piperidin-4-yl)methyl)-7-azaspiro[3.5]nonan-2-yl)carbamate (160 mg, 278.67 μmol, 41.91% yield, 94% purity) as a gray solid. MS (M+H)⁺=540.3

### Step 5. Synthesis of tert-butyl (7-((1-(4-((2,6-dioxopiperidin-3-yl)(methyl)amino)phenyl)piperidin-4-yl)methyl)-7-azaspiro[3.5]nonan-2-yl)carbamate (7)

To a solution of tert-butyl (7-((1-(4-((2,6-dioxopiperidin-3-yl)amino)phenyl)piperidin-4-yl)methyl)-7-azaspiro[3.5]nonan-2-yl)carbamate (160 mg, 296.46 μmol) in MeOH (5 mL) were added HCHO (433.04 mg, 5.34 mmol, 397.29 μL, 37% purity), HOAc (8.90 mg, 148.23 μmol, 8.48 μL) at 20° C., after stirring for 1 h, NaBH₃CN (335.34 mg, 5.34 mmol) was added, and the resulting mixture was stirred at 20° C. for 12 h. LCMS showed a main peak (90%) with desired mass. The reaction mixture was diluted with water (50 ml) and extracted with EtOAc (50 mL×3). The combined organic layers were washed with brine (50 mL), dried over anhydrous sodium sulfate, filtered. The filtrate was concentrated under reduced pressure to afford tert-butyl (7-((1-(4-((2,6-dioxopiperidin-3-yl)(methyl)amino)phenyl)piperidin-4-yl)methyl)-7-azaspiro[3.5]nonan-2-yl)carbamate (160 mg, 288.95 μmol, 97.47% yield) as brown oil. MS (M+H)⁺=554.4

### Step 6. Synthesis of 3-((4-(4-((2-amino-7-azaspiro[3.5]nonan-7-yl)methyl)piperidin-1-yl)phenyl)(methyl)amino)piperidine-2,6-dione (8)

To a solution of tert-butyl (7-((1-(4-((2,6-dioxopiperidin-3-yl)(methyl)amino)phenyl)piperidin-4-yl)methyl)-7-azaspiro[3.5]nonan-2-yl)carbamate (160 mg, 288.95 μmol) in DCM (3 mL) was added TFA (1 mL). The mixture was stirred at 20° C. for 1 h. TLC (methanol:dichloromethane=2:1) indicated the starting material was consumed completely. The mixture was concentrated under reduced pressure to afford 3-((4-(4-((2-amino-7-azaspiro[3.5]nonan-7-yl)methyl)piperidin-1-yl)phenyl)(methyl)amino)piperidine-2,6-dione (180 mg, crude, TFA salt) as yellow oil MS (M+H)⁺=454.2

### Step 7. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(7-((1-(4-((2,6-dioxopiperidin-3-yl)(methyl)amino)phenyl)piperidin-4-yl)methyl)-7-azaspiro[3.5]nonan-2-yl)-3-methoxybenzamide (Compound 27)

To a solution of 4-((7,7-difluoro-9-isopropyl-5-methyl-6-oxo-6,7,8,9-tetrahydro-5H-pyrimido[4,5-b][1,4]diazepin-2-yl)amino)-3-methoxybenzoic acid (133.63 mg, 317.10 μmol) in DMF (3 mL) were added HATU (180.86 mg, 475.65 μmol) and DIPEA (122.95 mg, 951.30 μmol, 165.70 L), after stirring for 0.5 h, then 3-((4-(4-((2-amino-7-azaspiro[3.5]nonan-7-yl)methyl)piperidin-1-yl)phenyl)(methyl)amino)piperidine-2,6-dione (180 mg, 317.10 μmol, TFA salt) was added. The mixture was stirred at 20° C. for 12 h. LCMS showed a peak (40%) with desired mass. The reaction mixture was diluted with water (30 mL) and extracted with EtOAc (50 mL×3). The combined organic layers were washed with brine (50 mL), dried over anhydrous sodium sulfate, filtered. The filtrate was concentrated under reduced pressure. The residue was purified by flash silica gel chromatography (ISCO®; 20 g SepaFlash® Silica Flash Column, Eluent of 0˜80% petroleum ether/(EtOAc/ethanol; v/v=1:2) gradient@60 mL/min), then re-purified by prep-HPLC (column: Waters Xbridge 150×50 mm×10 μm; mobile phase: [water (NH₄HCO₃)-ACN]; B %: 50%-80%, 10 min), the eluent was lyophilized to afford 4-((7,7-difluoro-9-isopropyl-5-methyl-6-oxo-6,7,8,9-tetrahydro-5H-pyrimido[4,5-b][1,4]diazepin-2-yl)amino)-N-(7-((1-(4-((2,6-dioxopiperidin-3-yl)(methyl)amino)phenyl)piperidin-4-yl)methyl)-7-azaspiro[3.5]nonan-2-yl)-3-methoxybenzamide (21.4 mg, 24.10 μmol, 7.60% yield, 96.5% purity) as a white solid. MS (M+H)⁺=857.4

¹H NMR (400 MHz, DMSO-d₆) δ=10.72 (s, 1H), 8.41 (d, J=7.6 Hz, 1H), 8.33-8.28 (m, 1H), 8.22 (s, 1H), 7.88 (s, 1H), 7.53-7.46 (m, 2H), 6.83-6.78 (m, 2H), 6.73-6.71 (m, 2H), 4.91-4.84 (m, 1H), 4.71 (dd, J=4.8, 12.3 Hz, 1H), 4.45-4.34 (m, 1H), 4.04 (t, J=13.6 Hz, 2H), 3.94 (s, 3H), 3.42 (d, J=11.6 Hz, 2H), 3.29 (s, 3H), 2.85-2.77 (m, 1H), 2.67 (s, 3H), 2.57-2.52 (m, 3H), 2.29-2.09 (m, 8H), 1.89-1.71 (m, 6H), 1.63-1.51 (m, 5H), 1.24 (d, J=6.8 Hz, 6H), 1.22-1.14 (m, 2H).

### Example 28. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(7-((1-(4-((2,6-dioxopiperidin-3-yl)amino)phenyl)piperidin-4-yl)methyl)-7-azaspiro[3.5]nonan-2-yl)-3-methoxybenzamide (Compound 28)

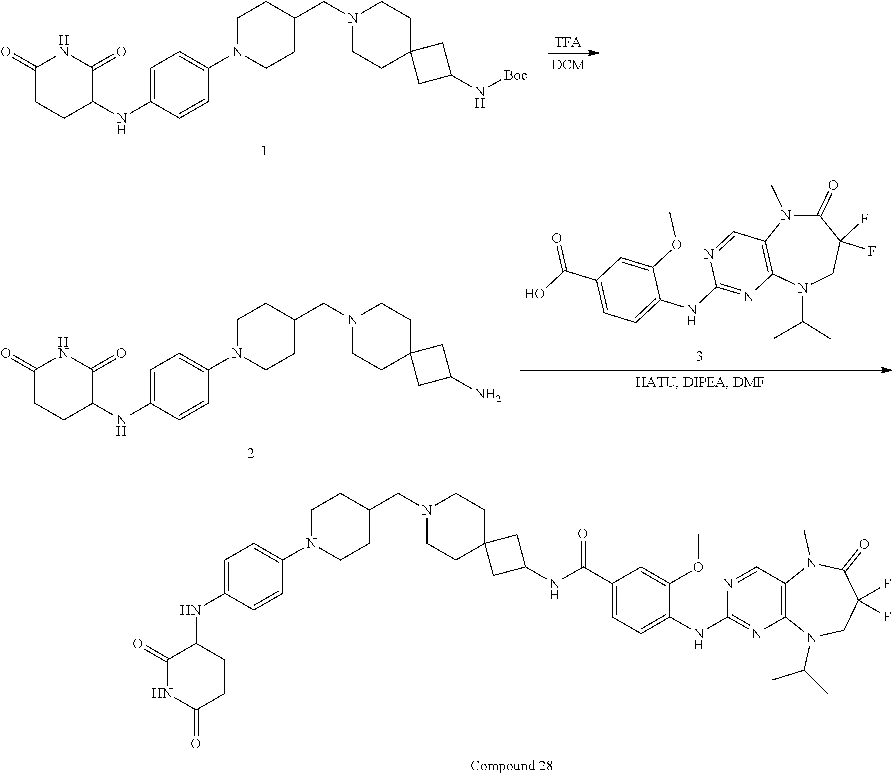

### Step 1. Synthesis of 3-((4-(4-((2-amino-7-azaspiro[3.5]nonan-7-yl)methyl)piperidin-1-yl)phenyl)amino)piperidine-2,6-dione (2)

To a solution of tert-butyl (7-((1-(4-((2,6-dioxopiperidin-3-yl)amino)phenyl)piperidin-4-yl)methyl)-7-azaspiro[3.5]nonan-2-yl)carbamate (160 mg, 296.46 μmol) in DCM (2 mL) was added TFA (33.80 mg, 296.46 μmol, 21.95 μL). The mixture was stirred at 20° C. for 1 h. TLC (methanol:dichloromethane=2:1) indicated one new spot was formed. The mixture was concentrated under reduced pressure to afford 3-((4-(4-((2-amino-7-azaspiro[3.5]nonan-7-yl)methyl)piperidin-1-yl)phenyl)amino)piperidine-2,6-dione (0.2 g, crude, TFA salt) as green oil. MS (M+H)⁺=440.4

### Step 2. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(7-((1-(4-((2,6-dioxopiperidin-3-yl)amino)phenyl)piperidin-4-yl)methyl)-7-azaspiro[3.5]nonan-2-yl)-3-methoxybenzamide (Compound 28)

To a solution of 4-((7,7-difluoro-9-isopropyl-5-methyl-6-oxo-6,7,8,9-tetrahydro-5H-pyrimido[4,5-b][1,4]diazepin-2-yl)amino)-3-methoxybenzoic acid (160 mg, 379.69 μmol) in DMF (3 mL) were added HATU (216.55 mg, 569.53 μmol) and DIPEA (245.36 mg, 1.90 mmol, 330.68 μL), after stirring for 0.5 h. Then 3-((4-(4-((2-amino-7-azaspiro[3.5]nonan-7-yl)methyl)piperidin-1-yl)phenyl)amino) piperidine-2,6-dione (199.69 mg, 360.70 μmol, TFA salt) was added and the resulting mixture was stirred at 20° C. for 12 h. LCMS showed a peak (˜50%) with desired mass. The reaction mixture was diluted with water (30 mL) and extracted with EtOAc (20 mL×3). The combined organic layers were washed with brine (50 mL), dried over anhydrous sodium sulfate, filtered and concentrated under reduced pressure. The residue was purified by prep-HPLC (column: Phenomenex luna C18 150×25 mm×10 m; mobile phase: [water (FA)-ACN]; B %: 3%-33%, 10 min) and then prep-HPCL (column: Waters Xbridge 150×25 mm×5 μm; mobile phase: [water (NH₄HCO₃)-ACN]; B %: 42%-72%, 8 min) followed by lyophilization to afford 4-((7,7-difluoro-9-isopropyl-5-methyl-6-oxo-6,7,8,9-tetrahydro-5H-pyrimido[4,5-b][1,4]diazepin-2-yl)amino)-N-(7-((1-(4-((2,6-dioxopiperidin-3-yl)amino)phenyl)piperidin-4-yl)methyl)-7-azaspiro[3.5]nonan-2-yl)-3-methoxybenzamide (41.5 mg, 43.96 μmol, 11.58% yield, 89.3% purity) as a gray solid. MS (M+H)⁺=843.2

¹H NMR (400 MHz, DMSO-d₆) δ=10.76 (s, 1H), 8.43 (d, J=7.2 Hz, 1H), 8.30 (d, J=8.4 Hz, 1H), 8.22 (s, 1H), 7.88 (s, 1H), 7.53-7.46 (m, 2H), 6.75-6.73 (m, 2H), 6.60-6.57 (m, 2H), 5.36 (d, J=7.2 Hz, 1H), 4.91-4.84 (m, 1H), 4.42-4.35 (m, 1H), 4.21-4.14 (m, 1H), 4.04 (t, J=13.8 Hz, 2H), 3.94 (s, 3H), 3.41-3.40 (m, 2H), 3.32 (s, 3H), 2.75-2.67 (m, 1H), 2.60-2.57 (m, 3H), 2.27-2.06 (m, 8H), 1.91-1.68 (m, 6H), 1.64-1.51 (m, 5H), 1.24 (d, J=6.6 Hz, 6H), 1.21-1.13 (m, 2H).

### Example 29. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(7-((1-(4-((2,6-dioxopiperidin-3-yl)(methyl)amino)-2, 6-difluorophenyl)piperidin-4-yl)methyl)-7-azaspiro[3.5]nonan-2-yl)-3-methoxybenzamide (Compound 29)

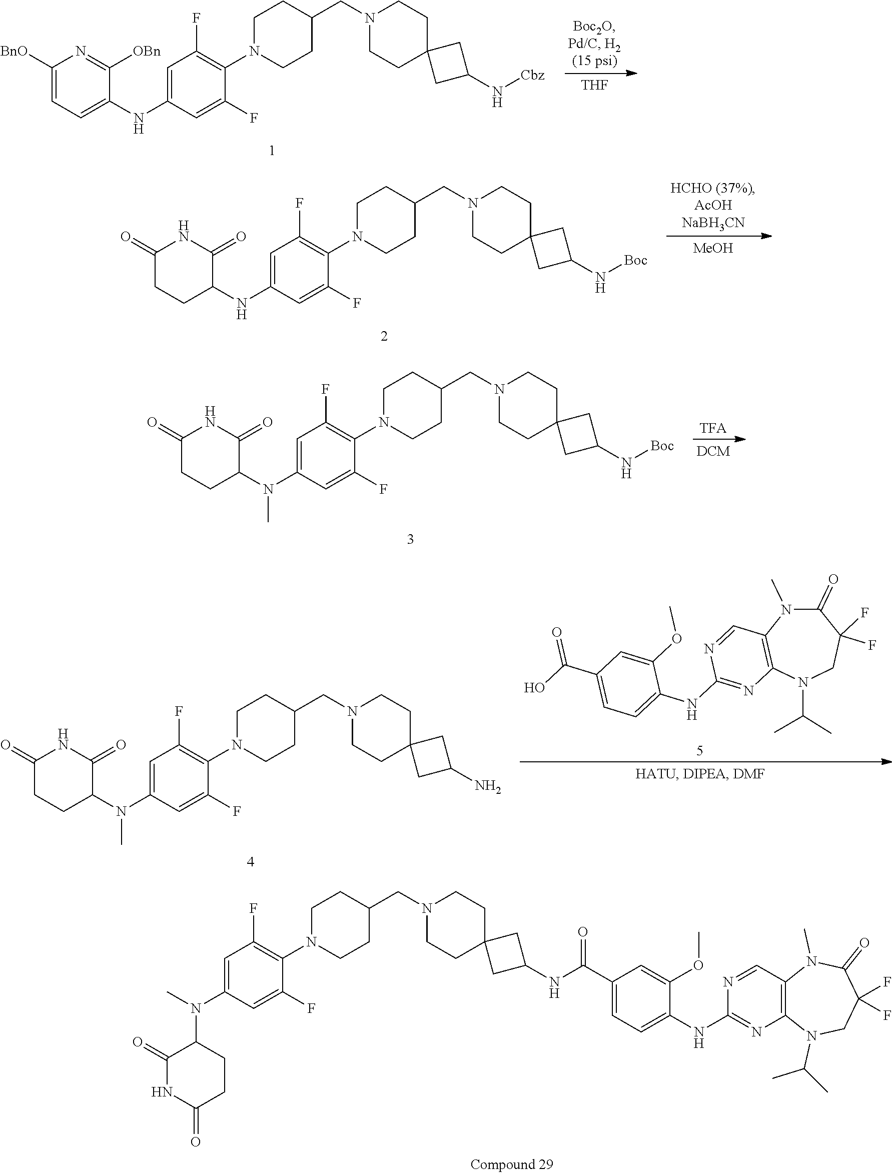

### Step 1. Synthesis of tert-butyl (7-((1-(4-((2,6-dioxopiperidin-3-yl)amino)-2,6-difluorophenyl) piperidin-4-yl)methyl)-7-azaspiro[3.5]nonan-2-yl)carbamate (2)

To a solution of benzyl (7-((1-(4-((2,6-bis(benzyloxy)pyridin-3-yl)amino)-2,6-difluorophenyl)piperidin-4-yl)methyl)-7-azaspiro[3.5]nonan-2-yl)carbamate (500 mg, 634.57 μmol) and Boc₂O (415.48 mg, 1.90 mmol) in THF (20 mL) was added Pd/C (100 mg, 10% purity) under N₂ atmosphere. The resulting mixture was degassed and purged with H₂ for 3 times, then the suspension was stirred at 20° C. for 16 hrs under H₂ (15 Psi). LCMS showed the intermediates and the desired mass. The mixture was filtered. To the filtrate was added Pd/C (200 mg, 10% purity). The resulting mixture was stirred at 20° C. under H₂ (15 Psi) for another 18 hrs. LCMS showed the intermediates were consumed completely and a main peak with desired mass. The reaction mixture was filtered and the filtrate was concentrated to afford tert-butyl (7-((1-(4-((2,6-dioxopiperidin-3-yl)amino)-2,6-difluorophenyl)piperidin-4-yl)methyl)-7-azaspiro[3.5]nonan-2-yl)carbamate (360 mg, crude) as a dark green solid. The crude product was used for the next step directly. MS (M+H)⁺=576.5

### Step 2. Synthesis of tert-butyl (7-((1-(4-((2,6-dioxopiperidin-3-yl)(methyl)amino)-2,6-difluorophenyl)piperidin-4-yl)methyl)-7-azaspiro[3.5]nonan-2-yl)carbamate (3)

A solution of tert-butyl (7-((1-(4-((2,6-dioxopiperidin-3-yl)amino)-2,6-difluorophenyl)piperidin-4-yl)methyl)-7-azaspiro[3.5]nonan-2-yl)carbamate (360 mg, 625.34 μmol), HCHO (1.01 g, 12.51 mmol, 37% purity) and AcOH (37.55 mg, 625.34 μmol) in MeOH (10 mL) was stirred at 20° C. for 1 h. Then NaBH₃CN (785.95 mg, 12.51 mmol) was added, the resulting mixture was stirred at 20° C. for 14 hrs. LCMS showed the starting material remained and the desired mass. Another portion of HCHO (507.47 mg, 6.25 mmol, 465.57 μL, 37% purity) was added to the mixture and the mixture was stirred at 20° C. for 1 h. Then NaBH₃CN (392.97 mg, 6.25 mmol) was added and the resulting mixture was stirred at 20° C. for 15 hrs. LCMS showed the starting material remained and the desired mass. The reaction was concentrated to remove the methanol. The residue was dissolved in EtOAc (20 mL) and washed with NaHCO₃ solution (10 mL), dried over Na₂SO₄, filtered and concentrated. The crude product was purified by prep-TLC (DCM:MeOH=10:1, Rf=0.6) to afford the crude product. The crude product was dissolved in MeOH (6 mL), then HCHO (405.98 mg, 5.00 mmol, 372.46 μL, 37% purity) and AcOH (37.55 mg, 625.34 μmol, 35.80 μL) were added. The mixture was stirred at 20° C. for 1 h. Then NaBH₃CN (314.38 mg, 5.00 mmol) was added. The resulting mixture was stirred at 20° C. for 13 hrs. LCMS showed trace of the starting material remained and the desired mass. The reaction was concentrated to remove the methanol. The residue was dissolved in EtOAc (20 mL) and washed with NaHCO₃ solution (10 mL), dried over Na₂SO₄, filtered and concentrated to afford tert-butyl (7-((1-(4-((2,6-dioxopiperidin-3-yl)(methyl)amino)-2,6-difluorophenyl)piperidin-4-yl) methyl)-7-azaspiro[3.5]nonan-2-yl)carbamate (150 mg, crude) as yellow oil. MS (M+H)⁺=590.5

### Step 3. Synthesis of 3-((4-(4-((2-amino-7-azaspiro[3.5]nonan-7-yl)methyl)piperidin-1-yl)-3,5-difluorophenyl)(methyl)amino)piperidine-2,6-dione (4)

To a solution of tert-butyl (7-((1-(4-((2,6-dioxopiperidin-3-yl)(methyl)amino)-2,6-difluorophenyl)piperidin-4-yl) methyl)-7-azaspiro[3.5]nonan-2-yl)carbamate (150 mg, 254.36 μmol) in DCM (4 mL) was added TFA (1.54 g, 13.51 mmol) at 20° C. The resulting solution was stirred at 20° C. for 0.5 h. LCMS showed the starting material was consumed completely and a main peak with the desired mass. The reaction mixture was concentrated to afford 3-((4-(4-((2-amino-7-azaspiro[3.5]nonan-7-yl)methyl)piperidin-1-yl)-3,5-difluorophenyl)(methyl)amino)piperidine-2,6-dione (150 mg, crude, TFA salt) as brown oil. MS (M+H)⁺=490.4

### Step 4. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(7-((1-(4-((2,6-dioxopiperidin-3-yl)(methyl)amino)-2, 6-difluorophenyl)piperidin-4-yl)methyl)-7-azaspiro[3.5]nonan-2-yl)-3-methoxybenzamide (Compound 29)

To a mixture of 3-((4-(4-((2-amino-7-azaspiro[3.5]nonan-7-yl)methyl)piperidin-1-yl)-3,5-difluorophenyl)(methyl)amino)piperidine-2,6-dione (150 mg, 248.50 μmol, TFA salt), 4-[(7,7-difluoro-9-isopropyl-5-methyl-6-oxo-8H-pyrimido[4,5-b][1,4]diazepin-2-yl)amino]-3-methoxy-benzoic acid (73.30 mg, 173.95 μmol) and DIPEA (192.70 mg, 1.49 mmol) in DMF (3 mL) was added HATU (94.49 mg, 248.50 μmol) at 20° C. The resulting mixture was stirred at 20° C. for 0.5 hr. LCMS showed the starting material was consumed completely and the desired mass. The reaction solution was poured into water (20 mL), and extracted with EtOAc (10 mL×3). The combined organic layers were washed with brine (10 mL), dried over Na₂SO₄, filtered and concentrated. The crude product was purified by prep-HPLC (column: Unisil 3-100 C18 Ultra 150×50 mm×3 μm; mobile phase: [water (FA)-ACN]; B %: 18%-48%, 7 min), the eluent was lyophilized to afford 4-((7,7-difluoro-9-isopropyl-5-methyl-6-oxo-6,7,8,9-tetrahydro-5H-pyrimido[4,5-b][1,4]diazepin-2-yl)amino)-N-(7-((1-(4-((2,6-dioxopiperidin-3-yl)(methyl)amino)-2,6-difluorophenyl)piperidin-4-yl)methyl)-7-azaspiro[3.5]nonan-2-yl)-3-methoxybenzamide (10.3 mg, 9.93 μmol, 3.99% yield, 90.5% purity, FA salt) as a white solid. MS (M+H)⁺=893.5

¹H NMR (400 MHz, DMSO-d₆) δ=10.84 (br s, 1H), 8.44 (br d, J=7.5 Hz, 1H), 8.30 (d, J=8.4 Hz, 1H), 8.26 (s, 1H), 8.22 (s, 1H), 7.89 (s, 1H), 7.53-7.46 (m, 2H), 6.49 (s, 1H), 6.45 (s, 1H), 4.92-4.80 (m, 2H), 4.45-4.35 (m, 1H), 4.04 (br t, J=13.9 Hz, 2H), 3.94 (s, 3H), 3.32 (br s, 3H), 2.98-2.92 (m, 4H), 2.84-2.77 (m, 1H), 2.67 (s, 3H), 2.582.55 (m, 1H), 2.32-2.20 (m, 5H), 2.17-2.10 (m, 4H), 1.87-1.77 (m, 3H), 1.74-1.65 (m, 2H), 1.63-1.51 (m, 5H), 1.24 (d, J=6.6 Hz, 6H), 1.20-1.13 (m, 2H).

### Example 30. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-((7-((1-(4-((2,6-dioxopiperidin-3-yl)amino)-2,6-difluorophenyl)piperidin-4-yl)methyl)-7-azaspiro[3.5]nonan-2-yl)methyl)-3-methoxybenzamide (Compound 30)

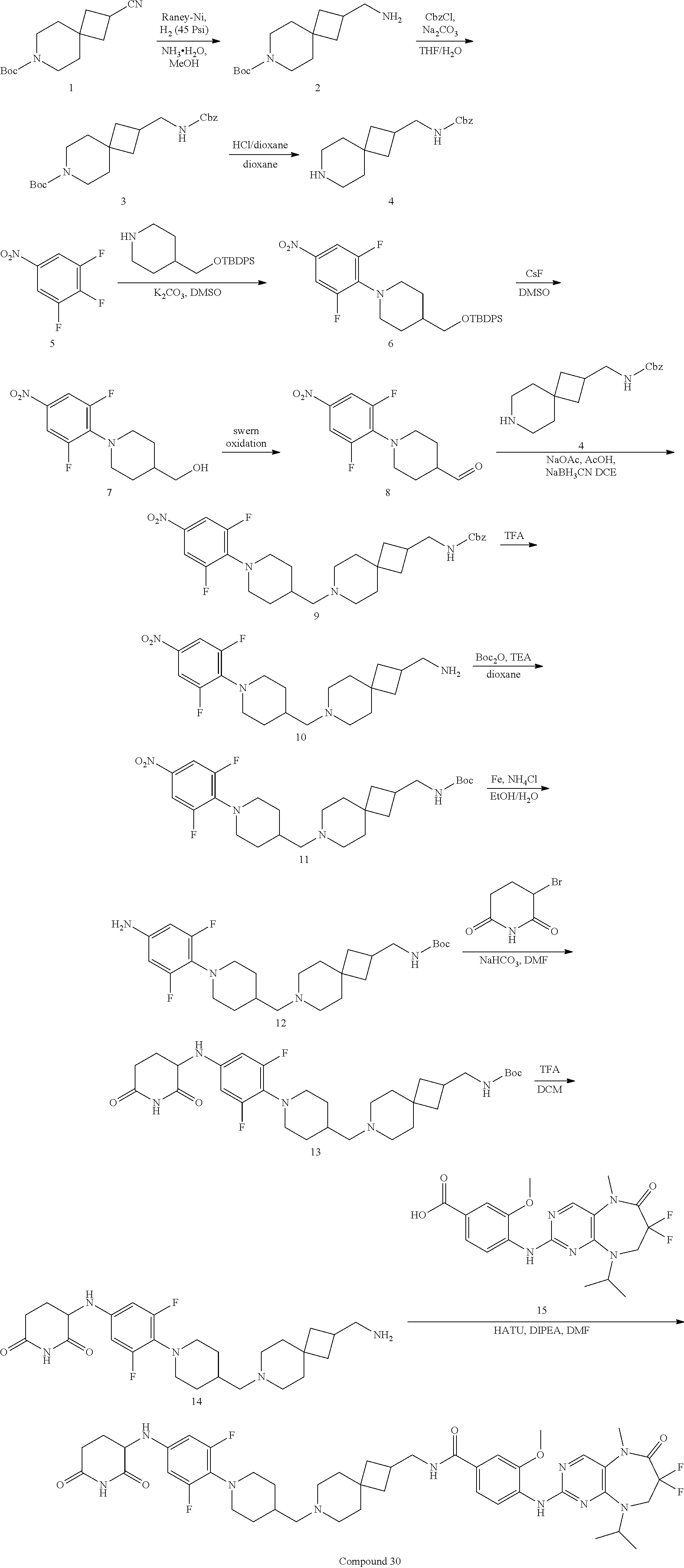

### Step 1. Synthesis of tert-butyl 2-(aminomethyl)-7-azaspiro[3.5]nonane-7-carboxylate (2)

To a solution of tert-butyl 2-cyano-7-azaspiro[3.5]nonane-7-carboxylate (1.5 g, 5.99 mmol) in MeOH (30 mL) were added NH₃·H₂O (2.73 g, 21.81 mmol, 3 mL, 28% purity) and Raney-Ni (300.00 mg, 3.50 mmol) under N₂, then H₂ was bubbled into the mixture, the mixture was stirred under 45 psi of H₂ at 20° C. for 16 h. LCMS showed main peak with desired mass, the mixture was filtered, the filtrate was concentrated under vacuum to afford tert-butyl 2-(aminomethyl)-7-azaspiro[3.5]nonane-7-carboxylate (1.5 g, crude) as yellow oil. MS (M+H)⁺=255.1

### Step 2. Synthesis of tert-butyl 2-((((benzyloxy)carbonyl)amino)methyl)-7-azaspiro[3.5]nonane-7-carboxylate (3)

To a solution of tert-butyl 2-(aminomethyl)-7-azaspiro[3.5]nonane-7-carboxylate (1.4 g, crude) in THF (28 mL) and H₂O (7 mL) were added CbzCl (1.13 g, 6.60 mmol, 938.92 μL) and Na₂CO₃ (1.17 g, 11.01 mmol), the mixture was stirred at 20° C. for 16 hr. LCMS showed main peak with desired mass, the mixture was diluted with water (30 mL), extracted with EtOAc (25 mL×3), the organic layer was dried over Na₂SO₄, filtered and concentrated under vacuum to afford tert-butyl 2-((((benzyloxy)carbonyl)amino)methyl)-7-azaspiro[3.5]nonane-7-carboxylate (2.6 g, crude) as white powder. MS (M+H)⁺=389.1

### Step 3. Synthesis of benzyl ((7-azaspiro[3.5]nonan-2-yl)methyl)carbamate (4)

To a solution of tert-butyl 2-((((benzyloxy)carbonyl)amino)methyl)-7-azaspiro[3.5]nonane-7-carboxylate (100 mg, crude) in dioxane (2 mL) was added HCl/dioxane (4 M, 1.00 mL), the mixture was stirred at 20° C. for 1 h. LCMS showed desired mass and the starting material consumed up. The mixture was concentrated under vacuum to afford benzyl ((7-azaspiro[3.5]nonan-2-yl)methyl)carbamate (110 mg, crude) as yellow oil. MS (M+H)⁺=289.1

### Step 4. Synthesis of 4-(((tert-butyldiphenylsilyl)oxy)methyl)-1-(2,6-difluoro-4-nitrophenyl)piperidine (6)

To a solution of 4-(((tert-butyldiphenylsilyl)oxy)methyl)piperidine (505.16 mg, 1.43 mmol) in DMSO (10 mL) were added 1,2,3-trifluoro-5-nitrobenzene (230 mg, 1.30 mmol) and K₂CO₃ (538.52 mg, 3.90 mmol), the mixture was stirred at 20° C. for 16 h. LCMS showed desired mass, the mixture was diluted with water (15 mL), extracted with EtOAc (15 mL×3), the organic layer was dried over Na₂SO₄, filtered and concentrated under vacuum. The crude product was purified by flash silica gel chromatography (Biotage, 4 g SepaFlash® Silica Flash Column, Eluent of 4-60% EtOAc/Petroleum ether gradient@15 mL/min) to afford 4-(((tert-butyldiphenylsilyl)oxy)methyl)-1-(2,6-difluoro-4-nitrophenyl)piperidine (420 mg, 822.49 μmol, 63.32% yield, 100% purity) as yellow oil. MS (M+H)⁺=511.2.

### Step 5. Synthesis of (1-(2,6-difluoro-4-nitrophenyl)piperidin-4-yl)methanol (7)

To a solution of 4-(((tert-butyldiphenylsilyl)oxy)methyl)-1-(2,6-difluoro-4-nitrophenyl)piperidine (420 mg, 822.49 μmol) in DMSO (10 mL) was added CsF (187.40 mg, 1.23 mmol), the mixture was stirred at 20° C. for 16 h. LCMS showed main peak with desired mass, the mixture was diluted with water (3 mL), extracted with EtOAc (5 mL×3), the organic layer was dried over Na₂SO₄, filtered and concentrated under vacuum to afford (1-(2,6-difluoro-4-nitrophenyl)piperidin-4-yl)methanol (450 mg, crude) as yellow oil. MS (M+H)⁺=273.0

### Step 6. Synthesis of 1-(2,6-difluoro-4-nitrophenyl)piperidine-4-carbaldehyde (8)

To a solution of DMSO (717.45 mg, 9.18 mmol, 717.45 L) in DCM (6 mL) was added oxalyl chloride (233.12 mg, 1.84 mmol, 160.77 L) at −70° C. and it was stirred for 10 min, then a solution of (1-(2,6-difluoro-4-nitrophenyl)piperidin-4-yl)methanol (250 mg, crude) in DCM (2 mL) was added at −65° C. over 20 min, TEA (464.60 mg, 4.59 mmol, 639.07 μL) was dropwise added at −65° C., then the mixture was warmed to 20° C. and stirred for 0.5 h, TLC (EtOAc/Petroleum ether=1/1) showed the starting material consumed up. The mixture was diluted with water (3 mL), extracted with EtOAc (5 mL×3), the organic layer was dried over Na₂SO₄, filtered and concentrated under vacuum to afford 1-(2,6-difluoro-4-nitrophenyl)piperidine-4-carbaldehyde (200 mg, crude) as yellow oil, used directly. MS (M+H)⁺=271.2

### Step 7. Synthesis of benzyl ((7-((1-(2,6-difluoro-4-nitrophenyl)piperidin-4-yl)methyl)-7-azaspiro[3.5]nonan-2-yl)methyl)carbamate (9)

To a mixture of benzyl ((7-azaspiro[3.5]nonan-2-yl)methyl)carbamate (200 mg, crude) in DCE (2 mL) were added 1-(2,6-difluoro-4-nitrophenyl)piperidine-4-carbaldehyde (96.17 mg, 296.04 μmol, HCl), NaOAc (60.71 mg, 740.11 μmol) and AcOH (4.44 mg, 74.01 μmol, 4.23 μL), the mixture stirred at 20° C. for 0.5 h, then NaBH(OAc)₃ (188.23 mg, 888.13 μmol) was added, the mixture was stirred at 20° C. for 15.5 h. LCMS showed desired mass, the mixture was diluted with water (3 mL), extracted with EtOAc (5 mL×3), the organic layer was dried over sodium sulfate, filtered and concentrated under vacuum. The crude product was purified by flash silica gel chromatography (Biotage, 4 g SepaFlash® Silica Flash Column, Eluent of 4-98% EtOAc/Petroleum ether gradient@20 mL/min) to afford benzyl ((7-((1-(2,6-difluoro-4-nitrophenyl)piperidin-4-yl)methyl)-7-azaspiro[3.5]nonan-2-yl) methyl)carbamate (700 mg, crude) as yellow oil. MS (M+H)⁺=543.3

### Step 8. Synthesis of (7-((1-(2,6-difluoro-4-nitrophenyl)piperidin-4-yl)methyl)-7-azaspiro[3.5]nonan-2-yl)methanamine (10)

A solution of benzyl ((7-((1-(2,6-difluoro-4-nitrophenyl)piperidin-4-yl)methyl)-7-azaspiro[3.5]nonan-2-yl) methyl)carbamate (700 mg, crude) in TFA (3 mL), the mixture was stirred at 40° C. for 16 h. LCMS showed desired mass, the mixture was concentrated under vacuum to afford (7-((1-(2,6-difluoro-4-nitrophenyl)piperidin-4-yl)methyl)-7-azaspiro[3.5]nonan-2-yl)methanamine (500 mg, crude) as brown oil. MS (M+H)⁺=409.2

### Step 9. Synthesis of tert-butyl ((7-((1-(2,6-difluoro-4-nitrophenyl)piperidin-4-yl)methyl)-7-azaspiro[3.5]nonan-2-yl)methyl)carbamate (11)

To a solution of (7-((1-(2,6-difluoro-4-nitrophenyl)piperidin-4-yl)methyl)-7-azaspiro[3.5]nonan-2-yl)methanamine (500 mg, crude) in dioxane (10 mL) was added TEA (1.24 g, 12.24 mmol, 1.70 mL) to adjust pH to 8, then Boc₂O (534.29 mg, 2.45 mmol, 562.41 μL) was added, the mixture was stirred at 20° C. for 16 hr. LCMS showed desired mass, the mixture was diluted with water (3 mL), extracted with EtOAc (5 mL×3), the organic layer was dried over sodium sulfate, filtered and concentrated under vacuum. The crude product was purified by flash silica gel chromatography (Biotage, 4 g SepaFlash® Silica Flash Column, Eluent of 4˜77% EtOAc/Petroleum ether gradient@20 mL/min) to afford tert-butyl ((7-((1-(2,6-difluoro-4-nitrophenyl)piperidin-4-yl)methyl)-7-azaspiro[3.5]nonan-2-yl) methyl)carbamate (140 mg, 261.50 μmol, 21.36% yield, 95% purity) as yellow oil. MS (M+H)⁺=509.2

### Step 10. Synthesis of tert-butyl ((7-((1-(4-amino-2,6-difluorophenyl)piperidin-4-yl)methyl)-7-azaspiro[3.5]nonan-2-yl)methyl)carbamate (12)

To a solution of tert-butyl ((7-((1-(2,6-difluoro-4-nitrophenyl)piperidin-4-yl)methyl)-7-azaspiro[3.5]nonan-2-yl) methyl)carbamate (140 mg, 275.27 μmol) in EtOH (5 mL) and H₂O (5 mL) were added Fe (153.72 mg, 2.75 mmol) and NH₄Cl (147.24 mg, 2.75 mmol) at 80° C., the mixture was stirred at 80° C. for 2 hr. LCMS showed main peak with desired mass, the mixture was diluted with the aqueous solution of NaHCO₃ (10 mL), extracted with EtOAc (10 mL×3), the organic layer was dried over sodium sulfate, filtered and concentrated under vacuum to afford tert-butyl ((7-((1-(4-amino-2,6-difluorophenyl)piperidin-4-yl)methyl)-7-azaspiro[3.5]nonan-2-yl) methyl)carbamate (140 mg, crude) as yellow oil, used directly. MS (M+H)⁺=479.2

### Step 11. Synthesis of tert-butyl ((7-((1-(4-((2,6-dioxopiperidin-3-yl)amino)-2,6-difluorophenyl)piperidin-4-yl)meth yl)-7-azaspiro[3.5]nonan-2-yl)methyl)carbamate (13)

To a solution of tert-butyl ((7-((1-(4-amino-2,6-difluorophenyl)piperidin-4-yl)methyl)-7-azaspiro[3.5]nonan-2-yl) methyl)carbamate (120 mg, crude) in DMF (0.2 mL) were added 3-bromopiperidine-2,6-dione (481.41 mg, 2.51 mmol) and NaHCO₃ (421.25 mg, 5.01 mmol, 195.02 μL), the mixture was stirred at 80° C. for 72 h. LCMS showed desired mass, the mixture was diluted with water (10 mL), extracted with EtOAc (10 mL×3), the organic layer was dried over sodium sulfate, filtered and concentrated under vacuum. The crude product was purified by flash silica gel chromatography (Biotage, 4 g SepaFlash® Silica Flash Column, Eluent of 4-70% EtOAc/Petroleum ether gradient @20 mL/min) to afford tert-butyl ((7-((1-(4-((2,6-dioxopiperidin-3-yl)amino)-2,6-difluorophenyl)piperidin-4-yl)methyl)-7-azaspiro[3.5]nonan-2-yl)methyl)carbamate (80 mg, 135.66 μmol, 54.11% yield, 100% purity) as yellow oil. MS (M+H)⁺=590.1

### Step 12. Synthesis of 3-((4-(4-((2-(aminomethyl)-7-azaspiro[3.5]nonan-7-yl)methyl)piperidin-1-yl)-3,5-difluorophenyl)amino)piperidine-2,6-dione (14)

To a solution of tert-butyl ((7-((1-(4-((2,6-dioxopiperidin-3-yl)amino)-2,6-difluorophenyl)piperidin-4-yl)methyl)-7-azaspiro[3.5]nonan-2-yl)methyl)carbamate (70 mg, 118.70 μmol) in DCM (2.5 mL) was added TFA (770.00 mg, 6.75 mmol, 0.5 mL), the mixture was stirred at 20° C. for 1 h. LCMS showed desired mass, the mixture was concentrated under vacuum to afford 3-((4-(4-((2-(aminomethyl)-7-azaspiro[3.5]nonan-7-yl)methyl)piperidin-1-yl)-3,5-difluorophenyl)amino)piperidine-2,6-dione (100 mg, crude, TFA) as brown oil, used directly. MS (M+H)⁺=490.3

### Step 13. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-((7-((1-(4-((2,6-dioxopiperidin-3-yl)amino)-2,6-difluorophenyl)piperidin-4-yl)methyl)-7-azaspiro[3.5]nonan-2-yl)methyl)-3-methoxybenzamide (Compound 30)

To a solution of 4-((7,7-difluoro-9-isopropyl-5-methyl-6-oxo-6,7,8,9-tetrahydro-5H-pyrimido[4,5-b][1,4]diazepin-2-yl)amino)-3-methoxybenzoic acid (50 mg, 118.65 μmol) in DMF (2 mL) were added HATU (67.67 mg, 177.98 μmol) and DIPEA (46.01 mg, 355.96 μmol, 62.00 μL), then 3-((4-(4-((2-(aminomethyl)-7-azaspiro[3.5]nonan-7-yl)methyl)piperidin-1-yl)-3,5-difluorophenyl)amino)piperidine-2,6-dione (71.62 mg, crude, TFA) was added, the mixture was stirred at 25° C. for 16 h. LCMS showed a peak (46%) with desired mass, the mixture was diluted with water (3 mL), extracted with EtOAc (5 mL×3), the organic layer was dried over sodium sulfate, filtered and concentrated under vacuum. The crude product was purified by flash silica gel chromatography (Biotage, 4 g SepaFlash® Silica Flash Column, Eluent of 4-15% EtOAc/MeOH gradient@20 mL/min) and re-purified by Prep-HPLC (column: Waters Xbridge 150*25 mm*5 μm; mobile phase: [water(NH₄HCO₃)-ACN]; B %: 50%-80%, 8 min) which dried by freeze drying to afford 4-((7,7-difluoro-9-isopropyl-5-methyl-6-oxo-6,7,8,9-tetrahydro-5H-pyrimido[4,5-b][1,4]diazepin-2-yl)amino)-N-((7-((1-(4-((2,6-dioxopiperidin-3-yl)amino)-2,6-difluorophenyl)piperidin-4-yl)methyl)-7-azaspiro[3.5]nonan-2-yl)methyl)-3-methoxybenzamide (9.3 mg, 9.48 μmol, 7.99% yield, 91.0% purity) as white powder. MS (M+H)⁺=893.5

¹H NMR (400 MHz, DMSO-d₆) δ=10.81 (s, 1H), 8.38-8.28 (m, 2H), 8.22 (s, 1H), 7.89 (s, 1H), 7.54-7.47 (m, 2H), 6.31 (d, J=12.2 Hz, 2H), 6.22 (d, J=7.8 Hz, 1H), 4.88 (td, J=6.6, 13.5 Hz, 1H), 4.35-4.27 (m, 1H), 4.04 (t, J=13.6 Hz, 2H), 3.94 (s, 3H), 3.51-3.46 (m, 3H), 3.38 (s, 3H), 2.92 (d, J=6.7 Hz, 4H), 2.82-2.72 (m, 2H), 2.30-2.02 (m, 7H), 1.88-1.76 (m, 3H), 1.68 (d, J=11.7 Hz, 2H), 1.58-1.43 (m, 7H), 1.25 (d, J=6.7 Hz, 6H), 1.20-1.10 (m, 2H).

### Example 31. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(4-(7-(4-((2,6-dioxopiperidin-3-yl)(methyl)amino)phenyl)-2,7-diazaspiro[3.5]nonan-2-yl)piperidin-1-yl)-3-methoxybenzamide (Compound 31)

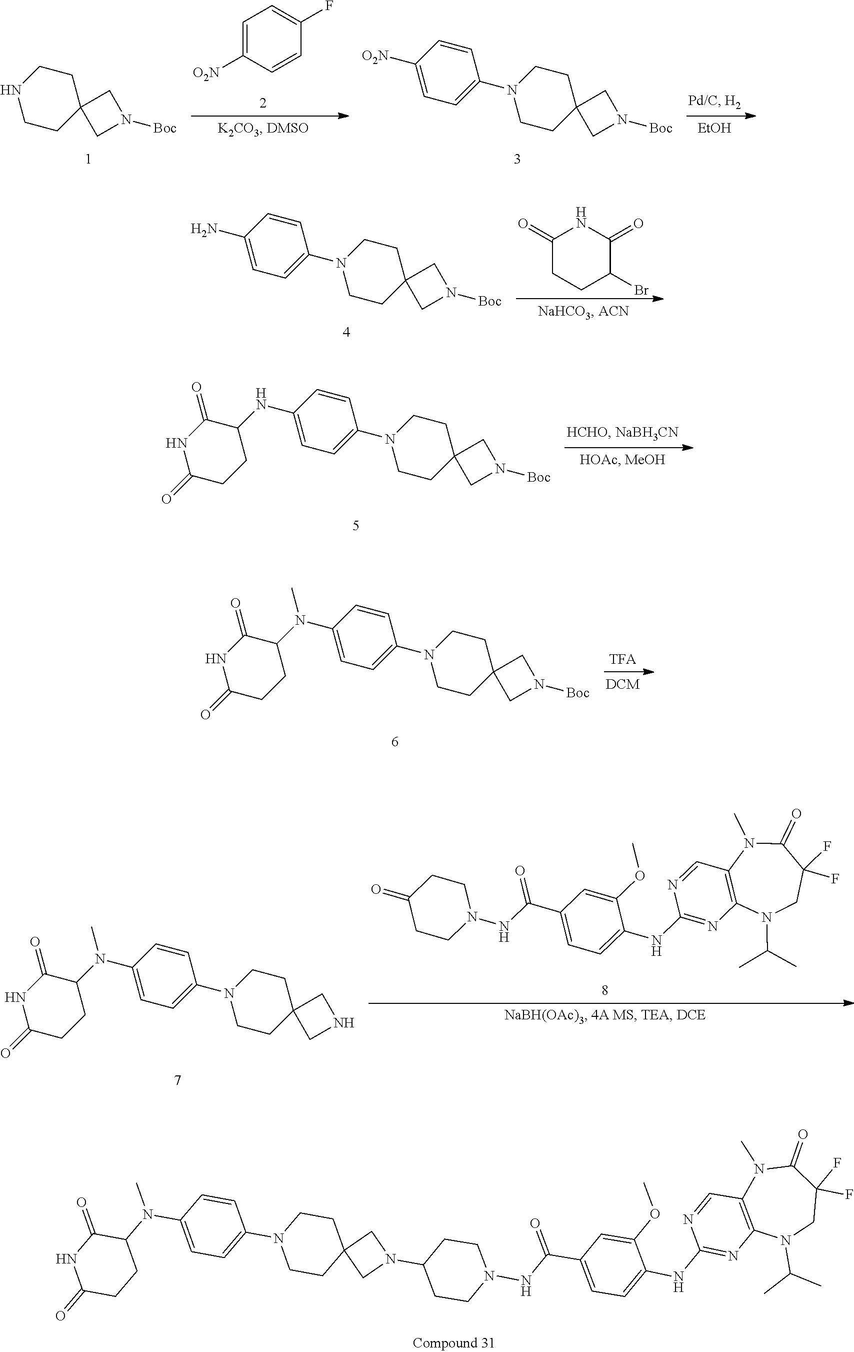

### Step 1. Synthesis of tert-butyl 7-(4-nitrophenyl)-2,7-diazaspiro[3.5]nonane-2-carboxylate (3)

To a solution of 1-fluoro-4-nitrobenzene (1 g, 7.09 mmol, 751.88 L) and tert-butyl 2,7-diazaspiro[3.5]nonane-2-carboxylate (1.5 g, 6.63 mmol) in DMSO (10 mL) was added K₂CO₃ (2.75 g, 19.88 mmol) and the mixture was stirred at 60° C. for 5 h. LCMS showed a major peak (100%) with desired mass. The mixture was diluted with water (30 mL) and filtered. The filter cake was washed with water (50 mL), collected and dried under reduced pressure to afford tert-butyl 7-(4-nitrophenyl)-2,7-diazaspiro[3.5]nonane-2-carboxylate (2.1 g, crude) as a yellow solid. MS (M+H)⁺=348.1

### Step 2. Synthesis of tert-butyl 7-(4-aminophenyl)-2,7-diazaspiro[3.5]nonane-2-carboxylate (4)

To a solution of tert-butyl 7-(4-nitrophenyl)-2,7-diazaspiro[3.5]nonane-2-carboxylate (2.1 g, 6.04 mmol) in EtOH (40 mL) was added Pd/C (0.2 g, 10% purity) under N₂ atmosphere, the mixture was degassed and purged with H₂ for 3 times, then the mixture was stirred at 20° C. for 14 h under H₂ (15 Psi). LCMS showed a main peak (100%) with desired mass. The mixture was filtered. The filter cake was washed with MeOH (100 mL). The filtrate was concentrated under reduced pressure to afford tert-butyl 7-(4-aminophenyl)-2,7-diazaspiro[3.5]nonane-2-carboxylate (1.9 g, crude) as a gray solid. MS (M+H)⁺=318.2

### Step 3. Synthesis of tert-butyl 7-(4-((2,6-dioxopiperidin-3-yl)amino)phenyl)-2,7 diazaspiro[3.5]nonane-2-carboxylate (5)

To a solution of tert-butyl 7-(4-aminophenyl)-2,7-diazaspiro[3.5]nonane-2-carboxylate (1 g, 3.15 mmol) and 3-bromopiperidine-2,6-dione (1.31 g, 6.84 mmol) in MeCN (10 mL) was added NaHCO₃ (1.44 g, 17.11 mmol, 665.33 μL) and the mixture was stirred at 80° C. for 14 h. HPLC showed a peak (92%) with desired mass. The mixture was filtered and the filter cake was washed with EtOAc (50 mL) and THF (50 mL). The filtrate was concentrated under reduced pressure. The residue was purified by flash silica gel chromatography (12 g SepaFlash® Silica Flash Column, Eluent of 70-100% EtOAc/Petroleum ether gradient@80 mL/min) to afford tert-butyl 7-(4-((2,6-dioxopiperidin-3-yl)amino)phenyl)-2,7-diazaspiro[3.5]nonane-2-carboxylate (1 g, 1.87 mmol, 59.26% yield, 80% purity) as a black brown solid. MS (M+H)⁺=429.2

### Step 4. Synthesis of tert-butyl 7-(4-((2,6-dioxopiperidin-3-yl)(methyl)amino)phenyl)-2,7-diazaspiro[3.5]nonane-2-carboxylate (6)

To a solution of tert-butyl 7-(4-((2,6-dioxopiperidin-3-yl)amino)phenyl)-2,7-diazaspiro[3.5]nonane-2-carboxylate (1 g, 1.87 mmol, 80% purity) in MeOH (20 mL) were added HCHO (196.20 mg, 2.42 mmol, 180 μL, 37% purity) and AcOH (105.00 mg, 1.75 mmol, 0.1 mL) and the mixture was stirred at 20° C. for 0.5 h. NaBH₃CN (352 mg, 5.60 mmol) was added and the mixture was stirred at 20° C. for another 3 h. LCMS showed a peak (70%) with desired mass. Additional HCHO (109.00 mg, 1.34 mmol, 0.1 mL, 37% purity) was added and the mixture was stirred at 20° C. for 0.5 h. Then NaBH₃CN (351.95 mg, 5.60 mmol) was added and the mixture was stirred at 20° C. for 1 h. LCMS showed the desired mass and the mixture was stirred at 20° C. for 2 h. The mixture was concentrated under reduced pressure. The crude was diluted with H₂O (30 mL) and extracted with EtOAc (10 mL×3), the combined organic layer was washed with water (10 mL×2), dried over Na₂SO₄ and filtered. The filtrate was concentrated under reduced pressure to afford tert-butyl 7-(4-((2,6-dioxopiperidin-3-yl)(methyl)amino)phenyl)-2,7-diazaspiro[3.5]nonane-2-carboxylate (1 g, crude) as a gray solid. MS (M+H)⁺=443.4

### Step 5. Synthesis of 3-((4-(2,7-diazaspiro[3.5]nonan-7-yl)phenyl)(methyl)amino)piperidine-2,6-dione (7)

To a solution of tert-butyl 7-(4-((2,6-dioxopiperidin-3-yl)(methyl)amino)phenyl)-2,7-diazaspiro[3.5]nonane-2-carboxylate (0.1 g, 225.96 μmol) in DCM (0.5 mL) was added TFA (246.40 mg, 2.16 mmol, 160 μL) at 0° C. and the mixture was stirred at 20° C. for 1 h. LCMS showed trace of the starting material remained and the desired mass. The mixture was concentrated under reduced pressure to afford 3-((4-(2,7-diazaspiro[3.5]nonan-7-yl)phenyl)(methyl)amino)piperidine-2,6-dione (0.1 g, crude, TFA salt) as a black brown oil. MS (M+H)⁺=343.4

### Step 6. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(4-(7-(4-((2,6-dioxopiperidin-3-yl)(methyl)amino)phenyl)-2,7-diazaspiro[3.5]nonan-2-yl)piperidin-1-yl)-3-methoxybenzamide (Compound 31)

To a solution of 3-((4-(2,7-diazaspiro[3.5]nonan-7-yl)phenyl)(methyl)amino)piperidine-2,6-dione (0.3 g, 657.24 μmol, TFA salt) in DCE (10 mL) were added TEA (392.58 mg, 3.88 mmol, 540 μL) and 4 A MS (0.3 g) at 0° C. followed by 4-((7,7-difluoro-9-isopropyl-5-methyl-6-oxo-6,7,8,9-tetrahydro-5H-pyrimido[4,5-b][1,4]diazepin-2-yl)amino)-3-methoxy-N-(4-oxopiperidin-1-yl)benzamide (450 mg, 869.52 μmol). The mixture was stirred at 0° C. for 0.5 h. Then NaBH(OAc)₃ (417.59 mg, 1.97 mmol) was added and the mixture was stirred at 20° C. for 3 h. LCMS showed the desired mass. The mixture was filtered and the filter cake was washed with DCM (20 mL). The filtrate was concentrated under reduced pressure. The residue was purified by flash silica gel chromatography (12 g SepaFlash® Silica Flash Column, Eluent of 25-100% MeOH/EtOAc gradient@80 mL/min). The crude was triturated with MTBE (10 mL) at 20° C. for 30 min. The mixture was filtered and the filter cake was washed with MTBE (20 mL). The filtrate was diluted with DCM/MeOH=10/1 (20 mL) and H₂O (10 mL) and extracted with DCM/MeOH=10/1 (10 mL×2), the combined organic layer was washed with water (10 mL×2), dried over Na₂SO₄ and filtered. The filtrate was concentrated under reduced pressure. The product was diluted with ACN (6 mL), MeOH (2 mL) and deionized water (40 mL) and lyophilized to afford the product (214.6 mg, 221.22 μmol, 33.66% yield, 87% purity) as a blue solid. 80 mg of the product (87% purity) was purified by prep-HPLC (column: Phenomenex Synergi Polar-RP 100*25 mm*4 m; mobile phase: [water (TFA)-ACN]; B %: 19%-39%, 7 min) and the eluent was lyophilized. 70 mg of the product (87% purity) was purified by prep-HPLC (column: Phenomenex Synergi Polar-RP 100*25 mm*4 m; mobile phase: [water (TFA)-ACN]; B %: 20%-40%, 7 min). The eluent was combined and lyophilized to afford 4-((7,7-difluoro-9-isopropyl-5-methyl-6-oxo-6,7,8,9-tetrahydro-5H-pyrimido[4,5-b][1,4]diazepin-2-yl)amino)-N-(4-(7-(4-((2,6-dioxopiperidin-3-yl)(methyl)amino)phenyl)-2,7-diazaspiro[3.5]nonan-2-yl)piperidin-1-yl)-3-methoxybenzamide (140.4 mg, 133.37 mol, 75.04% yield, 91% purity, 2TFA) as a gray solid. MS (M+H)⁺=844.2

¹H NMR (400 MHz, DMSO-d₆) δ=10.85-10.80 (m, 1H), 9.55-9.48 (m, 1H), 8.31-8.20 (m, 2H), 8.14-8.02 (m, 1H), 7.46-7.40 (m, 2H), 7.39-7.15 (m, 2H), 6.94-6.83 (m, 2H), 4.95-4.83 (m, 2H), 4.11-4.04 (m, 6H), 3.93 (s, 3H), 3.38-3.31 (m, 6H), 3.15-3.07 (m, 2H), 2.93-2.70 (m, 5H), 2.62-2.52 (m, 4H), 2.20-2.04 (m, 4H), 2.00-1.83 (m, 4H), 1.56-1.42 (m, 2H), 1.24 (d, J=6.7 Hz, 6H).

### Example 32. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(4-(7-(4-((2,6-dioxopiperidin-3-yl)amino)-2-fluorophenyl)-2,7-diazaspiro[3.5]nonan-2-yl)piperidin-1-yl)-3-methoxybenzamide (Compound 32)

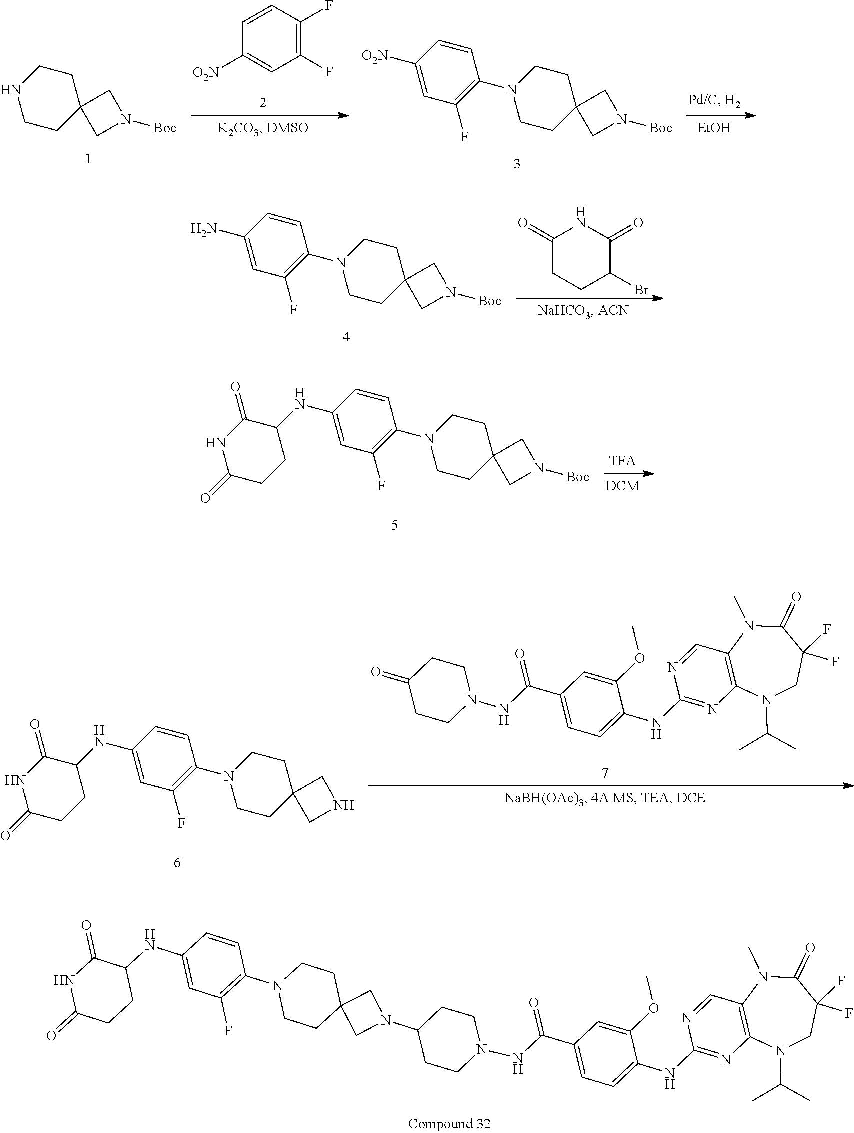

### Step 1. Synthesis of tert-butyl 7-(2-fluoro-4-nitrophenyl)-2,7-diazaspiro[3.5]nonane-2-carboxylate (3)

To a solution of 1,2-difluoro-4-nitrobenzene (1.5 g, 9.43 mmol, 1.04 mL) and tert-butyl 2,7-diazaspiro[3.5]nonane-2-carboxylate (2 g, 8.84 mmol) in DMSO (20 mL) was added K₂CO₃ (3.66 g, 26.51 mmol) and the mixture was stirred at 60° C. for 5 h. TLC (Petroleum ether:EtOAc=5:1) showed new spots were formed. The mixture was diluted with H₂O (50 mL) and extracted with EtOAc (20 mL×3). The combined organic layer was washed with brine (10 mL×2), dried over Na₂SO₄ and filtered. The filtrate was concentrated under reduced pressure. The residue was purified by column chromatography (SiO₂, Petroleum ether/EtOAc=10/1 to 0/1) to afford tert-butyl 7-(2-fluoro-4-nitrophenyl)-2,7-diazaspiro[3.5]nonane-2-carboxylate (2.93 g, 7.30 mmol, 82.57% yield, 91% purity) as a yellow solid. MS (M+H)⁺=366.2

### Step 2. Synthesis of tert-butyl 7-(4-amino-2-fluorophenyl)-2,7-diazaspiro[3.5]nonane-2-carboxylate (4)

To a solution of tert-butyl 7-(2-fluoro-4-nitrophenyl)-2,7-diazaspiro[3.5]nonane-2-carboxylate (2.93 g, 8.02 mmol) in EtOH (50 mL) was added Pd/C (300 mg, 10% purity) under N₂ atmosphere, the reaction mixture was degassed and purged with H₂ for 3 times, then the mixture was stirred at 20° C. for 14 h under H₂ (15 Psi). LCMS showed a major peak (99%) with desired mass. The mixture was filtered and the filter cake was washed with MeOH (100 mL) and THF (30 mL). The filtrate was concentrated under reduced pressure to afford tert-butyl 7-(4-amino-2-fluorophenyl)-2,7-diazaspiro[3.5]nonane-2-carboxylate (2.6 g, crude) as a gray solid. MS (M+H)⁺=336.1

### Step 3. Synthesis of tert-butyl 7-(4-((2,6-dioxopiperidin-3-yl)amino)-2-fluorophenyl)-2,7-diazaspiro[3.5]nonane-2-carboxylate (5)

To a solution of tert-butyl 7-(4-amino-2-fluorophenyl)-2,7-diazaspiro[3.5]nonane-2-carboxylate (1 g, 2.98 mmol) and 3-bromopiperidine-2,6-dione (1.24 g, 6.47 mmol) in MeCN (15 mL) was added NaHCO₃ (1.36 g, 16.19 mmol, 629.64 μL) and the mixture was stirred at 80° C. for 14 h. LCMS showed the starting material remained and the desired mass was detected. The mixture was concentrated under reduced pressure. The residue was purified by flash silica gel chromatography (12 g SepaFlash® Silica Flash Column, Eluent of 40-60% EtOAc/Petroleum ether gradient@50 mL/min) to afford tert-butyl 7-(4-((2,6-dioxopiperidin-3-yl)amino)-2-fluorophenyl)-2,7-diazaspiro[3.5]nonane-2-carboxylate (610 mg, 1.35 mmol, 45.36% yield, 99% purity) as a gray solid. MS (M+H)⁺=447.2

### Step 4. Synthesis of 3-((3-fluoro-4-(2,7-diazaspiro[3.5]nonan-7-yl)phenyl)amino)piperidine-2,6-dione (6)

To a solution of tert-butyl 7-(4-((2,6-dioxopiperidin-3-yl)amino)-2-fluorophenyl)-2,7-diazaspiro[3.5]nonane-2-carboxylate (0.3 g, 671.87 μmol) in DCM (1.5 mL) was added TFA (770.00 mg, 6.75 mmol, 0.5 mL) at 0° C. and the mixture was stirred at 0° C. for 2 h. LCMS showed 26% of the starting material remained and 59% of the desired mass. The mixture was stirred at 0° C. for 0.5 h. The mixture was concentrated under reduced pressure to afford 3-((3-fluoro-4-(2,7-diazaspiro[3.5]nonan-7-yl)phenyl)amino)piperidine-2,6-dione (0.3 g, crude, TFA salt) as a black brown oil. MS (M+H)⁺=347.3

### Step 5. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(4-(7-(4-((2,6-dioxopiperidin-3-yl)amino)-2-fluorophenyl)-2,7-diazaspiro[3.5]nonan-2-yl)piperidin-1-yl)-3-methoxybenzamide (Compound 32)

To a solution of 3-((3-fluoro-4-(2,7-diazaspiro[3.5]nonan-7-yl)phenyl)amino)piperidine-2,6-dione (0.3 g, 651.58 μmol, TFA salt) in DCE (10 mL) was added TEA (399.85 mg, 3.95 mmol, 550 μL) and 4 A MS (0.5 g) at 0° C. followed by 4-((7,7-difluoro-9-isopropyl-5-methyl-6-oxo-6,7,8,9-tetrahydro-5H-pyrimido[4,5-b][1,4]diazepin-2-yl)amino)-3-methoxy-N-(4-oxopiperidin-1-yl)benzamide (0.5 g, 966.13 mol). The mixture was stirred at 0° C. for 0.5 h. Then NaBH(OAc)₃ (414 mg, 1.95 mmol) was added and the mixture was stirred at 20° C. for 3 h. LCMS showed a peak (73%) with desired mass. The mixture was filtered and the filter cake was washed with DCM (20 mL). The filtrate was concentrated under reduced pressure. The residue was purified by flash silica gel chromatography (12 g SepaFlash® Silica Flash Column, Eluent of 30% MeOH/EtOAc gradient@80 mL/min). The product was diluted with MTBE (20 mL) and stirred at 20° C. for 0.5 h. The mixture was filtered and the filter cake was washed with MTBE (30 mL). The filtrate was collected and dried under reduced pressure. The product was diluted with ACN (5 mL) and deionized water (40 mL), then lyophilized to afford 4-((7,7-difluoro-9-isopropyl-5-methyl-6-oxo-6,7,8,9-tetrahydro-5H-pyrimido[4,5-b][1,4]diazepin-2-yl)amino)-N-(4-(7-(4-((2,6-dioxopiperidin-3-yl)amino)-2-fluorophenyl)-2,7-diazaspiro[3.5]nonan-2-yl)piperidin-1-yl)-3-methoxybenzamide (406.1 mg, 464.57 mol, 71.30% yield, 97% purity) as a gray solid. MS (M+H)⁺=848.2

¹H NMR (400 MHz, DMSO-d₆) δ=10.79 (s, 1H), 9.43 (br s, 1H), 8.31 (br d, J=8.2 Hz, 1H), 8.22 (s, 1H), 7.89 (s, 1H), 7.46-7.40 (m, 2H), 6.83 (t, J=9.3 Hz, 1H), 6.54-6.48 (m, 1H), 6.44-6.39 (m, 1H), 5.83 (br d, J=7.7 Hz, 1H), 4.92-4.84 (m, 1H), 4.31-4.22 (m, 1H), 4.04 (br t, J=13.6 Hz, 2H), 3.94 (s, 3H), 3.33-3.29 (m, 8H), 3.12-3.02 (m, 3H), 2.88-2.66 (m, 7H), 2.12-2.04 (m, 1H), 1.97-1.79 (m, 7H), 1.52-1.37 (m, 2H), 1.25 (d, J=6.7 Hz, 6H).

### Example 33. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(4-(7-(4-((2,6-dioxopiperidin-3-yl)(methyl)amino)-2-fluorophenyl)-2,7-diazaspiro[3.5]nonan-2-yl)piperidin-1-yl)-3-methoxybenzamide (Compound 33)

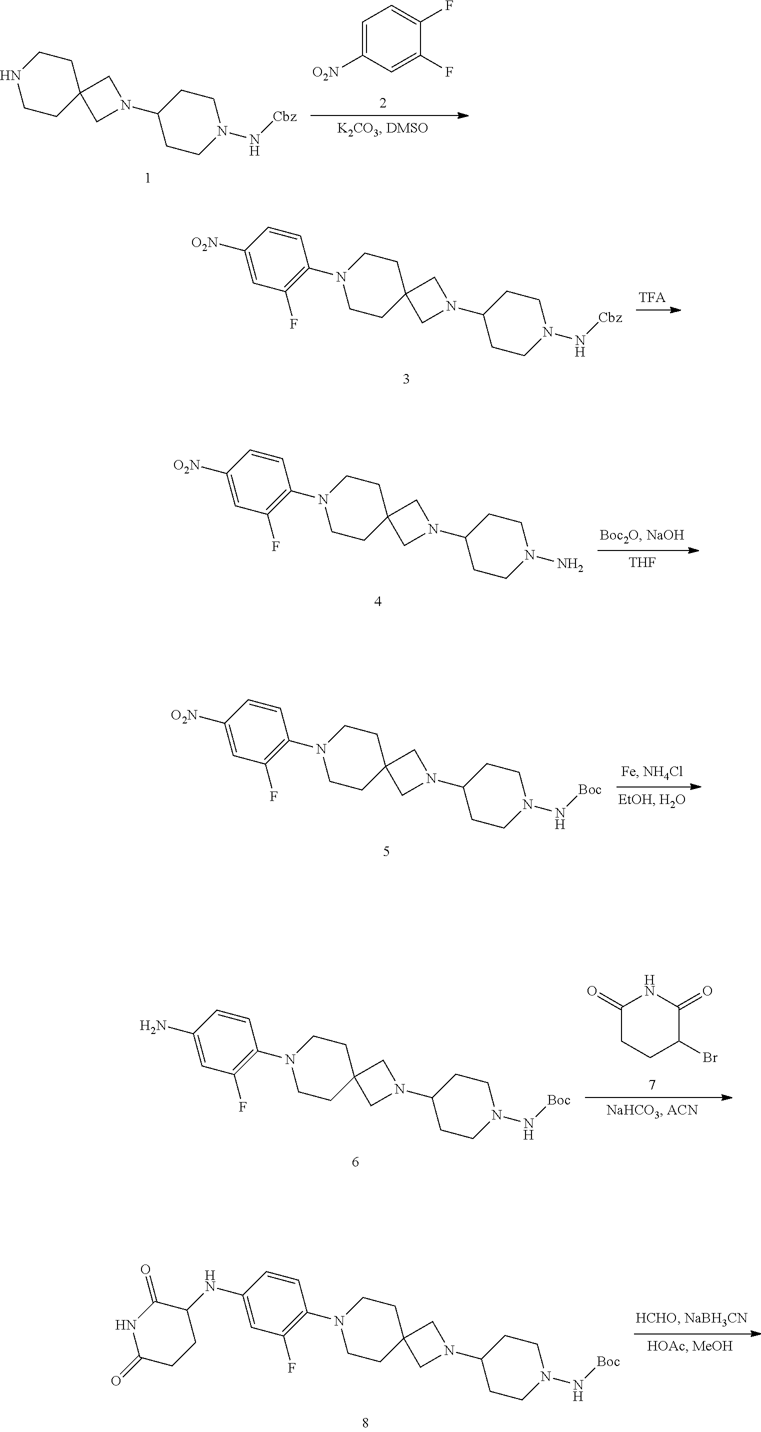

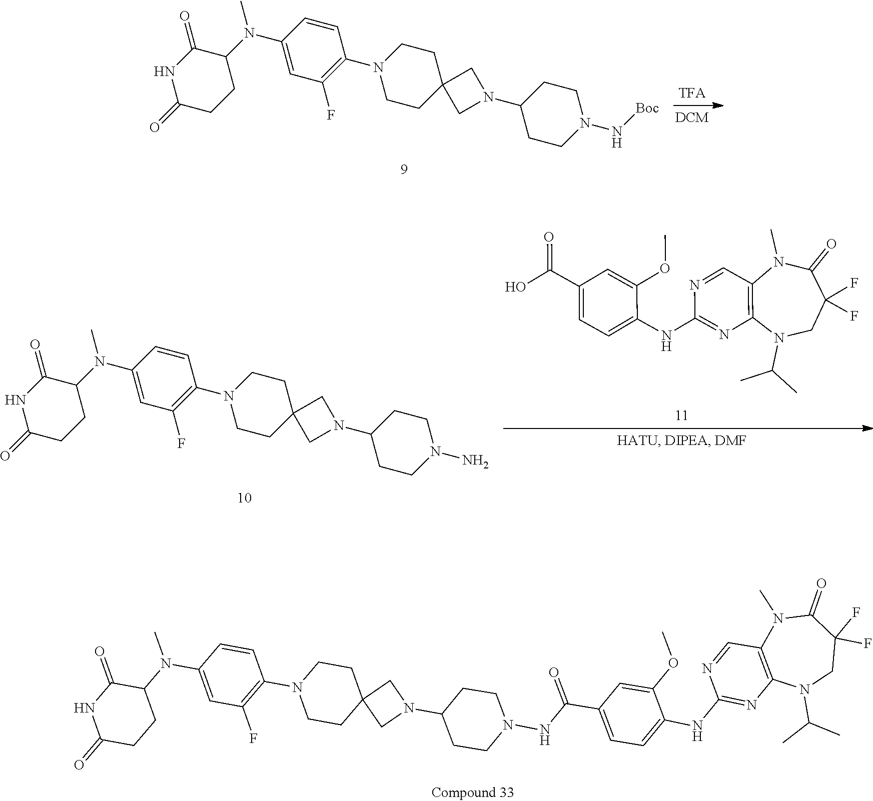 [SMILES: CN(c1ccc(N2CCC3(CC2)CN(C2CCN(NC(=O)OC(C)(C)C)CC2)C3)c(F)c1)C1CCC(=O)NC1=O ; CN(c1ccc(N2CCC3(CC2)CN(C2CCN(N)CC2)C3)c(F)c1)C1CCC(=O)NC1=O ; COc1cc(C(=O)O)ccc1Nc1ncc2c(n1)N(C(C)C)CC(F)(F)C(=O)N2C ; COc1cc(C(=O)NN2CCC(N3CC4(CCN(c5ccc(N(C)C6CCC(=O)NC6=O)cc5F)CC4)C3)CC2)ccc1Nc1ncc2c(n1)N(C(C)C)CC(F)(F)C(=O)N2C]

### Step 1. Synthesis of benzyl (4-(7-(2-fluoro-4-nitrophenyl)-2,7-diazaspiro[3.5]nonan-2-yl)piperidin-1-yl)carba mate (3)

To a solution of benzyl (4-(2,7-diazaspiro[3.5]nonan-2-yl)piperidin-1-yl)carbamate (1.6 g, 4.05 mmol, HCl salt) and 1,2-difluoro-4-nitrobenzene (800.00 mg, 5.03 mmol, 555.56 μL) in DMSO (15 mL) was added K₂CO₃ (1.68 g, 12.15 mmol) and the mixture was stirred at 20° C. for 14 h. LCMS showed the desired mass was detected. The mixture was diluted with H₂O (50 mL) and extracted with EtOAc (20 mL×3). The combined organic layer was washed with brine (20 mL×2), dried over Na₂SO₄ and filtered. The filtrate was concentrated under reduced pressure. The residue was purified by flash silica gel chromatography (12 g SepaFlash® Silica Flash Column, Eluent of 0-30% MeOH/EtOAc gradient@70 mL/min) to afford benzyl (4-(7-(2-fluoro-4-nitrophenyl)-2,7-diazaspiro[3.5]nonan-2-yl)piperidin-1-yl)carbamate (1.19 g, 2.39 mmol, 59.03% yield) as yellow oil. MS (M+H)⁺=498.3

### Step 2. Synthesis of 4-(7-(2-fluoro-4-nitrophenyl)-2,7-diazaspiro[3.5]nonan-2-yl)piperidin-1-amine (4)

A solution of benzyl (4-(7-(2-fluoro-4-nitrophenyl)-2,7-diazaspiro[3.5]nonan-2-yl)piperidin-1-yl)carbamate (2.28 g, 4.58 mmol) in TFA (20 mL) was stirred at 60° C. for 3 h. LCMS showed 13% of the starting material remained and 62% of the desired mass. The mixture was stirred at 60° C. for 2 h. LCMS showed 6% of the starting material remained and 67% of the desired mass. The mixture was concentrated under reduced pressure to afford 4-(7-(2-fluoro-4-nitrophenyl)-2,7-diazaspiro[3.5]nonan-2-yl)piperidin-1-amine (2.2 g, crude, TFA salt) as yellow oil. MS (M+H)⁺=364.0

### Step 3. Synthesis of tert-butyl (4-(7-(2-fluoro-4-nitrophenyl)-2,7-diazaspiro[3.5]nonan-2-yl)piperidin-1-yl)carba mate (5)

To a solution of 4-(7-(2-fluoro-4-nitrophenyl)-2,7-diazaspiro[3.5]nonan-2-yl)piperidin-1-amine (2.2 g, 4.61 mmol, TFA salt) in THF (20 mL) were added NaOH (1 M, 7 mL) and Boc₂O (2.01 g, 9.22 mmol, 2.12 mL) at 0° C. and the mixture was stirred at 20° C. for 14 h. LCMS showed the desired mass was detected. The mixture was diluted with H₂O (50 mL) and extracted with EtOAc (20 mL×3). The combined organic layer was washed with H₂O (10 mL×2), dried over Na₂SO₄ and filtered. The filtrate was concentrated under reduced pressure to give residue A. The aqueous phase was concentrated under reduced pressure. The crude was diluted with saturated NaHCO₃ (10 mL) and THF (10 mL), then Boc₂O (1.01 g, 4.61 mmol, 1.06 mL) was added and the mixture was stirred at 20° C. for 14 h. The mixture was extracted with EtOAc (20 mL×3), the combined organic layer was washed with H₂O (10 mL×2), dried over Na₂SO₄ and filtered, the filtrate was concentrated in vacuo to give residue B. The residue A and B was combined and purified by flash silica gel chromatography (5 g SepaFlash® Silica Flash Column, Eluent of 20% MeOH/EtOAc gradient@50 mL/min) to afford tert-butyl (4-(7-(2-fluoro-4-nitrophenyl)-2,7-diazaspiro[3.5]nonan-2-yl)piperidin-1-yl)carbamate (1.17 g, 2.27 mmol, 49.30% yield, 90% purity) as yellow oil. MS (M−100+H)⁺=364.3

### Step 4. Synthesis of tert-butyl (4-(7-(4-amino-2-fluorophenyl)-2,7-diazaspiro[3.5]nonan-2-yl)piperidin-1-yl)carbamate (6)

To a solution of tert-butyl (4-(7-(2-fluoro-4-nitrophenyl)-2,7-diazaspiro[3.5]nonan-2-yl)piperidin-1-yl)carbamate (1.17 g, 2.52 mmol) in EtOH (20 mL) and H₂O (20 mL) were added Fe (845.80 mg, 15.14 mmol) and NH₄Cl (810.06 mg, 15.14 mmol) and the mixture was stirred at 80° C. for 2 h. TLC (Dichloromethane:Methanol=10:1) showed new spot was detected. The mixture was filtered and the filtrate cake was washed with DMF (20 mL) and EtOH (20 mL). The filtrate was concentrated under reduced pressure. The crude was diluted with H₂O (20 mL) and extracted with EtOAc (10 mL×3). The combined organic layer was washed with water (10 mL×2), dried over Na₂SO₄ and filtered. The filtrate was concentrated under reduced pressure to afford tert-butyl (4-(7-(4-amino-2-fluorophenyl)-2,7-diazaspiro[3.5]nonan-2-yl)piperidin-1-yl)carbamate (460 mg, 1.06 mmol, 42.04% yield) as brown oil. MS (M+H)⁺=434.3

### Step 5. Synthesis of tert-butyl (4-(7-(4-((2,6-dioxopiperidin-3-yl)amino)-2-fluorophenyl)-2,7-diazaspiro[3.5]nonan-2-yl)piperidin-1-yl)carbamate (8)

To a solution of tert-butyl (4-(7-(4-amino-2-fluorophenyl)-2,7-diazaspiro[3.5]nonan-2-yl)piperidin-1-yl)carbamate (460 mg, 1.06 mmol) and 3-bromopiperidine-2,6-dione (442.07 mg, 2.30 mmol) in ACN (10 mL) was added NaHCO₃ (483.71 mg, 5.76 mmol, 223.94 μL) and the mixture was stirred at 80° C. for 14 h. LCMS showed tert-butyl (4-(7-(4-amino-2-fluorophenyl)-2,7-diazaspiro[3.5]nonan-2-yl)piperidin-1-yl)carbamate remained and the desired mass was detected. Additional 3-bromopiperidine-2,6-dione (407.44 mg, 2.12 mmol) and NaHCO₃ (445.65 mg, 5.30 mmol, 206.32 μL) was added and the mixture was stirred at 80° C. for 14 h. LCMS showed tert-butyl (4-(7-(4-amino-2-fluorophenyl)-2,7-diazaspiro[3.5]nonan-2-yl)piperidin-1-yl)carbamate was consumed completely. The mixture was filtered and the filter cake was washed with DCM (20 mL) and THF (20 mL). The filter cake was collected and dried in vacuo. The residue was purified by flash silica gel chromatography (5 g SepaFlash® Silica Flash Column, Eluent of 30-50% MeOH/EtOAc gradient@50 mL/min) to afford tert-butyl (4-(7-(4-((2,6-dioxopiperidin-3-yl)amino)-2-fluorophenyl)-2,7-diazaspiro[3.5]nonan-2-yl)piperidin-1-yl)carbamate (190 mg, 289.54 μmol, 27.29% yield, 83% purity) as a brown solid. MS (M+H)⁺=545.2

### Step 6. Synthesis of tert-butyl (4-(7-(4-((2,6-dioxopiperidin-3-yl)(methyl)amino)-2-fluorophenyl)-2,7-diazaspiro[3.5]nonan-2-yl)piperidin-1-yl)carbamate (9)

To a solution of tert-butyl (4-(7-(4-((2,6-dioxopiperidin-3-yl)amino)-2-fluorophenyl)-2,7-diazaspiro[3.5]nonan-2-yl)piperidin-1-yl)carbamate (100.29 mg, 184.14 μmol) and formaldehyde (21.80 mg, 268.63 μmol, 20 μL, 37% purity) in MeOH (2 mL) was added AcOH (10.41 mg, 173.31 μmol, 9.91 L) and the mixture was stirred at 20° C. for 0.5 h. NaBH₃CN (32.67 mg, 519.94 μmol) was added and the mixture was stirred at 20° C. for 3 h. LCMS showed the desired mass was detected. The mixture was concentrated under reduced pressure. The residue was purified by flash silica gel chromatography (5 g SepaFlash® Silica Flash Column, Eluent of 30% MeOH/EtOAc gradient@50 mL/min) to afford tert-butyl (4-(7-(4-((2,6-dioxopiperidin-3-yl)(methyl)amino)-2-fluorophenyl)-2,7-diazaspiro[3.5]nonan-2-yl)piperidin-1-yl)carbamate (80 mg, 143.19 μmol, 82.62% yield) as a brown solid. MS (M+H)⁺=559.3

### Step 7. Synthesis of 3-((4-(2-(1-aminopiperidin-4-yl)-2,7-diazaspiro[3.5]nonan-7-yl)-3-fluorophenyl)(m ethyl)amino)piperidine-2,6-dione (10)

To a solution of tert-butyl (4-(7-(4-((2,6-dioxopiperidin-3-yl)(methyl)amino)-2-fluorophenyl)-2,7-diazaspiro[3.5]nonan-2-yl)piperidin-1-yl)carbamate (80 mg, 143.19 μmol) in DCM (1 mL) was added TFA (246.40 mg, 2.16 mmol, 160 L) and the mixture was stirred at 20° C. for 1 h. LCMS showed 16% of the starting material remained and 54% of the desired mass after work-up. The mixture was concentrated under reduced pressure to afford 3-((4-(2-(1-aminopiperidin-4-yl)-2,7-diazaspiro[3.5]nonan-7-yl)-3-fluorophenyl)(methyl)amino)piperidine-2,6-dione (80 mg, crude, TFA salt) as brown oil. MS (M+H)⁺=459.1

### Step 8. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(4-(7-(4-((2,6-dioxopiperidin-3-yl)(methyl)amino)-2-fluorophenyl)-2,7-diazaspiro[3.5]nonan-2-yl)piperidin-1-yl)-3-methoxybenzamide (Compound 33)

To a solution of 4-((7,7-difluoro-9-isopropyl-5-methyl-6-oxo-6,7,8,9-tetrahydro-5H-pyrimido[4,5-b][1,4]diazepin-2-yl)amino)-3-methoxybenzoic acid (45 mg, 106.79 μmol) and HATU (50.60 mg, 133.07 μmol) in DMF (1 mL) was added DIPEA (29.79 mg, 230.53 μmol, 40.15 μL) and the mixture was stirred at 20° C. for 15 min. Then a solution of 3-((4-(2-(1-aminopiperidin-4-yl)-2,7-diazaspiro[3.5]nonan-7-yl)-3-fluorophenyl)(methyl)amino)piperidine-2,6-dione (80.00 mg, 139.72 μmol, TFA salt) and DIPEA (148.97 mg, 1.15 mmol, 200.77 L) in DMF (1 mL) was added and the mixture was stirred at 20° C. for 1 h. TLC (Dichloromethane:Methanol=10:1) showed the new spots were formed. The reaction mixture was diluted with H₂O (10 mL) and extracted with EtOAc (10 mL×3). The combined organic layer was washed with brine (10 mL×2), dried over Na₂SO₄ and filtered. The filtrate was concentrated under reduced pressure. The crude was purified by prep-TLC (Dichloromethane:Methanol=10:1), then re-purified by prep-HPLC (column: Phenomenex Synergi Polar-RP 100×25 mm×4 μm; mobile phase: [water (TFA)-ACN]; B %: 28%-48%, 7 min) and the eluent was lyophilized to afford 4-((7,7-difluoro-9-isopropyl-5-methyl-6-oxo-6,7,8,9-tetrahydro-5H-pyrimido[4,5-b][1,4]diazepin-2-yl)amino)-N-(4-(7-(4-((2,6-dioxopiperidin-3-yl)(methyl)amino)-2-fluoro phenyl)-2,7-diazaspiro[3.5]nonan-2-yl)piperidin-1-yl)-3-methoxybenzamide (31.6 mg, 25.41 μmol, 22.91% yield, 96.8% purity, 3TFA) as a blue solid. MS (M+H)⁺=862.4

¹H NMR (400 MHz, DMSO-d₆) δ=10.77-10.75 (m, 1H), 10.01-9.63 (m, 1H), 9.54-9.42 (m, 1H), 8.31-8.17 (m, 2H), 7.47-7.41 (m, 2H), 6.73-6.62 (m, 2H), 6.57-6.50 (m, 1H), 4.92-4.84 (m, 1H), 4.77-4.70 (m, 1H), 4.13-4.02 (m, 2H), 3.92 (s, 3H), 3.69-3.56 (m, 2H), 3.40-3.18 (m, 9H), 3.13-3.05 (m, 2H), 2.90-2.77 (m, 2H), 2.66 (s, 3H), 2.57-2.50 (m, 3H), 2.30-2.20 (m, 1H), 2.17-2.07 (m, 3H), 2.04-1.90 (m, 3H), 1.89-1.82 (m, 1H), 1.79-1.65 (m, 2H), 1.24 (d, J=6.7 Hz, 6H)

### Example 34. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-((1r,4r)-4-(((1-(4-((2,6-dioxopiperidin-3-yl)amino)-2-fluorophenyl)piperidin-4-yl)methyl)(methyl)amino)cyclohexyl)-3-methoxybenzamide (Compound 34)

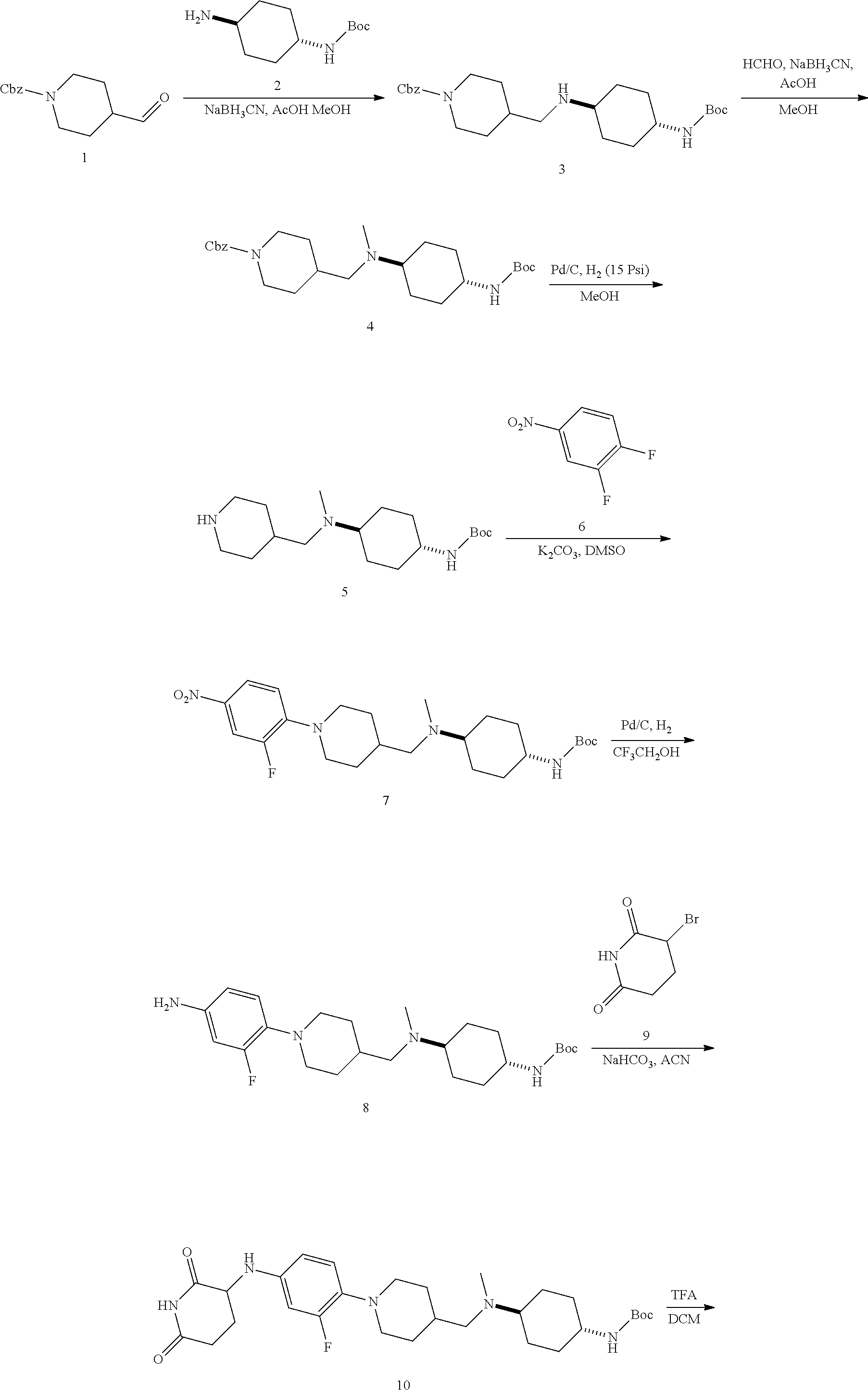

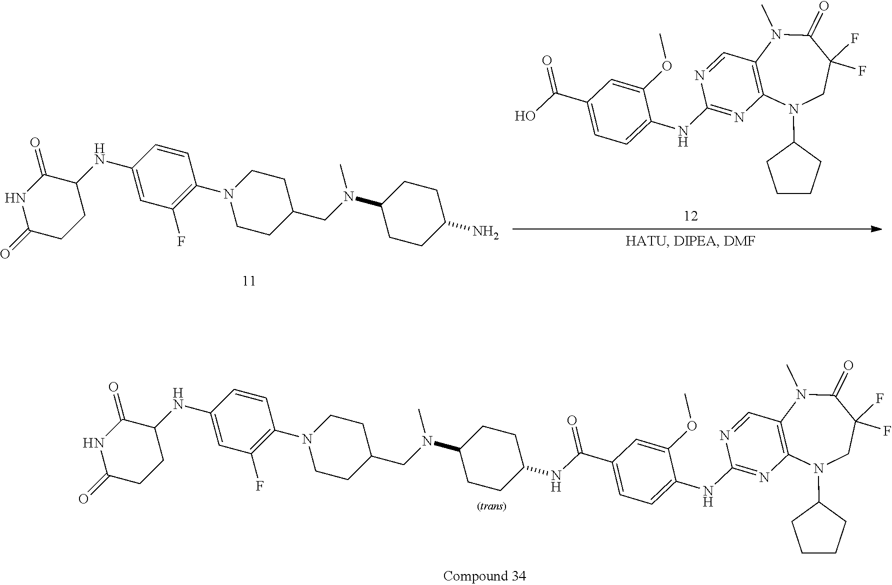 [SMILES: CN(CC1CCN(c2ccc(NC3CCC(=O)NC3=O)cc2F)CC1)[C@H]1CC[C@H](N)CC1 ; COc1cc(C(=O)O)ccc1Nc1ncc2c(n1)N(C1CCCC1)CC(F)(F)C(=O)N2C ; COc1cc(C(=O)N[C@H]2CC[C@H](N(C)CC3CCN(c4ccc(NC5CCC(=O)NC5=O)cc4F)CC3)CC2)ccc1Nc1ncc2c(n1)N(C1CCCC1)CC(F)(F)C(=O)N2C]

### Step 1. Synthesis of benzyl 4-((((1r,4r)-4-((tert-butoxycarbonyl)amino)cyclohexyl)amino)methyl)piperidine-1-carboxylate (3)

A mixture of benzyl 4-formylpiperidine-1-carboxylate (2 g, 8.09 mmol), tert-butyl ((1r,4r)-4-aminocyclohexyl)carbamate (1.73 g, 8.09 mmol) and AcOH (485.68 mg, 8.09 mmol, 462.55 μL) in MeOH (20 mL) was stirred at 20° C. for 0.5 h, NaBH₃CN (1.52 g, 24.26 mmol) was added, the mixture was stirred at 20° C. for 16 h. LCMS showed a main peak with desired mass. The reaction mixture was concentrated in vacuum to remove most of solvent. The mixture was quenched with NaHCO₃ (20 mL), extracted with EtOAc (20 mL×3). The combined organic layers were dried over Na₂ SO₄, filtered. The filtrate was concentrated in vacuum to give a residue. The residue was purified by flash silica gel chromatography (Biotage; 20 g SepaFlash® Silica Flash Column, Eluent of 50-100% EtOAc/Petroleum ether to 10% Methanol/EtOAc gradient@100 mL/min) to afford benzyl 4-((((1r,4r)-4-((tert-butoxycarbonyl)amino)cyclohexyl)amino)methyl)piperidine-1-carboxylate (3.6 g, 8.08 mmol, 99.89% yield) as a colorless oil. MS (M+H)⁺=446.3

### Step 2. Synthesis of benzyl 4-((((1r,4r)-4-((tert-butoxycarbonyl)amino)cyclohexyl)(methyl)amino)methyl)piperidine-1-carboxylate (4)

A mixture of benzyl 4-((((1r,4r)-4-((tert-butoxycarbonyl)amino)cyclohexyl)amino)methyl)piperidine-1-carboxylate (3.6 g, 8.08 mmol), formaldehyde (1.31 g, 16.16 mmol, 1.20 mL, 37% in H₂O) and AcOH (485.15 mg, 8.08 mmol, 462.05 μL) in MeOH (30 mL) was stirred at 20° C. for 0.5 h. NaBH₃CN (1.52 g, 24.24 mmol) was added, the mixture was stirred at 20° C. for 16 h. LCMS showed a main peak with desired mass. The reaction mixture was concentrated in vacuum to remove most of solvent. The mixture was quenched with NaHCO₃ (30 mL), extracted with EtOAc (40 mL×3). The combined organic layers were washed with brine (50 mL×2), dried over Na₂SO₄, filtered. The filtrate was concentrated in vacuum. The residue was purified by flash silica gel chromatography (Biotage; 20 g SepaFlash® Silica Flash Column, Eluent of 50-100% EtOAc/Petroleum ether to 10% Methanol/EtOAc gradient@100 mL/min) to afford benzyl 4-((((1r,4r)-4-((tert-butoxycarbonyl)amino)cyclohexyl)(methyl)amino)methyl)piperidine-1-carboxylate (3.7 g, 8.05 mmol, 99.64% yield) as a colorless oil. MS (M+H)⁺=460.3

### Step 3. Synthesis of tert-butyl ((1r,4r)-4-(methyl(piperidin-4-ylmethyl)amino)cyclohexyl)carbamate (5)

To a mixture of benzyl 4-((((1r,4r)-4-((tert-butoxycarbonyl)amino)cyclohexyl)(methyl)amino)methyl)piperidine-1-carboxylate (3.7 g, 8.05 mmol) in MeOH (40 mL) was added Pd/C (370 mg, 8.05 mmol, 10% purity) under N₂ and degassed and purged with H₂ for 3 times, the mixture was stirred at 20° C. for 44 h. LCMS showed a main peak with desired mass. The mixture was filtered through a pad of celite. The filtrate was concentrated in vacuum to afford tert-butyl ((1r,4r)-4-(methyl(piperidin-4-ylmethyl)amino)cyclohexyl)carbamate (2.6 g, crude) as a yellow oil. MS (M+H)⁺=326.2

### Step 4. Synthesis of tert-butyl ((1r,4r)-4-(((1-(2-fluoro-4-nitrophenyl)piperidin-4-yl)methyl)(methyl)amino)cyclo hexyl)carbamate (7)

To a solution of tert-butyl ((1r,4r)-4-(methyl(piperidin-4-ylmethyl)amino)cyclohexyl)carbamate (500 mg, crude), 1,2-difluoro-4-nitrobenzene (244.39 mg, 1.54 mmol, 169.71 L) in DMSO (5 mL) was added K₂CO₃ (636.92 mg, 4.61 mmol), the mixture was stirred at 20° C. for 16 h. LCMS showed a main peak with desired mass. The mixture was diluted with brine (15 mL) and extracted with EtOAc (15 mL×3). The combined organic layers washed with brine (40 mL×2), dried over Na₂SO₄, filtered. The filtrate was concentrated in vacuum to give a residue. The residue was purified by flash silica gel chromatography (Biotage; 20 g SepaFlash® Silica Flash Column, Eluent of 50-100% EtOAc/Petroleum ether to 20% Methanol/EtOAc gradient@100 mL/min) to afford tert-butyl ((1r,4r)-4-(((1-(2-fluoro-4-nitrophenyl)piperidin-4-yl)methyl)(methyl)amino)cyclohex yl)carbamate (500 mg, 1.08 mmol, 70.06% yield) as yellow solid. MS (M+H)⁺=465.3

### Step 5. Synthesis of tert-butyl ((1r,4r)-4-(((1-(4-amino-2-fluorophenyl)piperidin-4-yl)methyl)(methyl)amino)cyclohexyl)carbamate (8)

To a mixture of tert-butyl ((1r,4r)-4-(((1-(2-fluoro-4-nitrophenyl)piperidin-4-yl)methyl)(methyl)amino)cyclohex yl)carbamate (500 mg, 1.08 mmol) in CF₃CH₂OH (10 mL) was added Pd/C (100 mg, 1.08 mmol, 10% purity) under N₂ and degassed and purged with H₂ for 3 times, the mixture was stirred at 20° C. for 12 h under H₂ (15 Psi) atmosphere. LCMS showed a main peak with desired mass. The mixture was filtered through a pad of celite. The filtrate was concentrated in vacuum to afford tert-butyl ((1r,4r)-4-(((1-(4-amino-2-fluorophenyl)piperidin-4-yl)methyl)(methyl)amino)cyclohexyl)carbamate (450 mg, crude) as a brown oil. MS (M+H)⁺=435.3

### Step 6. Synthesis of tert-butyl ((1r,4r)-4-(((1-(4-((2,6-dioxopiperidin-3-yl)amino)-2-fluorophenyl)piperidin-4-yl) methyl)(methyl)amino)cyclohexyl)carbamate (10)

To a solution of 3-bromopiperidine-2,6-dione (309.27 mg, 1.61 mmol), tert-butyl ((1r,4r)-4-(((1-(4-amino-2-fluorophenyl)piperidin-4-yl)methyl)(methyl)amino)cyclohexyl)carbamate (350 mg, crude) in ACN (5 mL) was added NaHCO₃ (338.28 mg, 4.03 mmol, 156.61 μL), the mixture was stirred at 80° C. for 16 h. LCMS showed a main peak with desired mass. The mixture was diluted with water (10 mL) and extracted with EtOAc (10 mL×3). The combined organic layers was dried over Na₂SO₄, filtered. The filtrate was concentrated in vacuum to give a residue. The residue was purified by flash silica gel chromatography (Biotage; 20 g SepaFlash® Silica Flash Column, Eluent of 20-100% EtOAc/Petroleum ether to 10% Methanol/EtOAc gradient @100 mL/min) to afford tert-butyl ((1r,4r)-4-(((1-(4-((2,6-dioxopiperidin-3-yl)amino)-2-fluorophenyl)piperidin-4-yl)meth yl)(methyl)amino)cyclohexyl)carbamate (400 mg, crude) as a gray solid. MS (M+H)⁺=546.4

### Step 7. Synthesis of 3-((4-(4-((((1r,4r)-4-aminocyclohexyl)(methyl)amino)methyl)piperidin-1-yl)-3-fluorophenyl)amino)piperidine-2,6-dione (11)

To a solution of tert-butyl ((1r,4r)-4-(((1-(4-((2,6-dioxopiperidin-3-yl)amino)-2-fluorophenyl)piperidin-4-yl)meth yl)(methyl)amino)cyclohexyl)carbamate (100 mg, 183.26 μmol) in DCM (1 mL) was added TFA (770.00 mg, 6.75 mmol, 0.5 mL), the mixture was stirred at 20° C. for 1 h. LCMS showed a main peak with desired mass. The mixture was concentrated in vacuum to afford 3-((4-(4-((((1r,4r)-4-aminocyclohexyl)(methyl)amino)methyl)piperidin-1-yl)-3-fluorophenyl)amino)piperidine-2,6-dione (100 mg, crude, TFA) as a brown oil. MS (M+H)⁺=446.3

### Step 8. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-((1r,4r)-4-(((1-(4-((2,6-dioxopiperidin-3-yl)amino)-2-fluorophenyl)piperidin-4-yl)methyl)(methyl)amino)cyclohexyl)-3-methoxybenzamide (Compound 34)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (79.96 mg, 178.70 μmol) in DMF (2 mL) were added HATU (101.92 mg, 268.05 μmol), DIPEA (115.48 mg, 893.50 μmol, 155.63 μL), the mixture was stirred at 20° C. for 1 h. 3-((4-(4-((((1r,4r)-4-aminocyclohexyl)(methyl)amino)methyl)piperidin-1-yl)-3-fluorophenyl)amino)piperidine-2,6-dione (100 mg, crude, TFA) was added, the resulting mixture was stirred at 20° C. for 15 h. LCMS showed a main peak with desired mass. The reaction mixture was diluted with brine (30 mL), extracted with EtOAc (20 mL×3). The combined organic layers were washed with brine (40 mL×2), dried over Na₂ SO₄, filtered. The filtrate was concentrated in vacuum to give a residue. The residue was purified by prep-TLC (SiO₂, DCM:MeOH=10:1) and re-purified by reversed-phase HPLC (column: Waters Xbridge 150*25 mm*5 m; mobile phase: [water (NH₄HCO₃)-ACN]; B %: 46%-76%, 8 min). The eluent was lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-N-((1r,4r)-4-(((1-(4-((2,6-dioxopiperidin-3-yl)amino)-2-fluorophenyl)piperidin-4-yl)methyl)(methyl)amino)cyclohexyl)-3-methoxybenzamide (39.9 mg, 44.23 μmol, 24.75% yield, 97% purity) as a white solid. MS (M+H)⁺=875.4

¹H NMR (400 MHz, DMSO-d₆) δ=10.77 (s, 1H), 8.30-8.21 (m, 2H), 8.06 (d, J=7.6 Hz, 1H), 7.95 (s, 1H), 7.52-7.44 (m, 2H), 6.83 (t, J=9.3 Hz, 1H), 6.54-6.45 (m, 1H), 6.44-6.37 (m, 1H), 5.77 (d, J=7.6 Hz, 1H), 4.82-4.70 (m, 1H), 4.30-4.20 (m, 1H), 4.04 (t, J=14.1 Hz, 2H), 3.93 (s, 3H), 3.78-3.67 (m, 1H), 3.30 (s, 3H), 3.16-3.05 (m, 2H), 2.77-2.67 (m, 1H), 2.61-2.53 (m, 3H), 2.35-2.32 (m, 1H), 2.23-2.24 (m, 2H), 2.20 (s, 3H), 2.13-2.04 (m, 1H), 1.98-1.81 (m, 5H), 1.80-1.67 (m, 6H), 1.66-1.54 (m, 4H), 1.51-1.42 (m, 1H), 1.41-1.29 (m, 4H), 1.27-1.15 (m, 2H).

### Example 35. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(7-((1-(4-((2,6-dioxopiperidin-3-yl)oxy)-2-fluorophen yl)piperidin-4-yl)methyl)-7-azaspiro[3.5]nonan-2-yl)-3-methoxybenzamide (Compound 35)

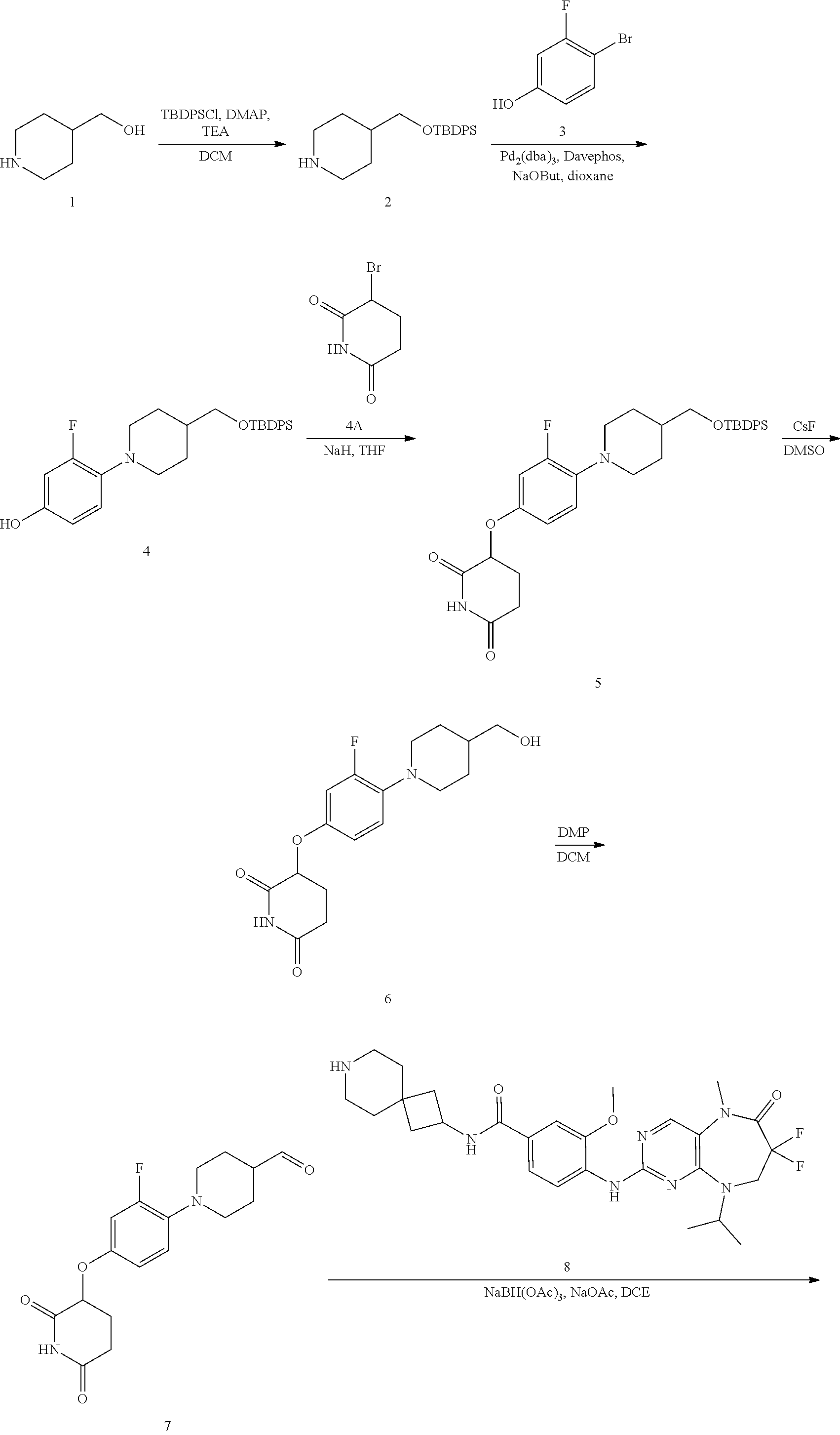

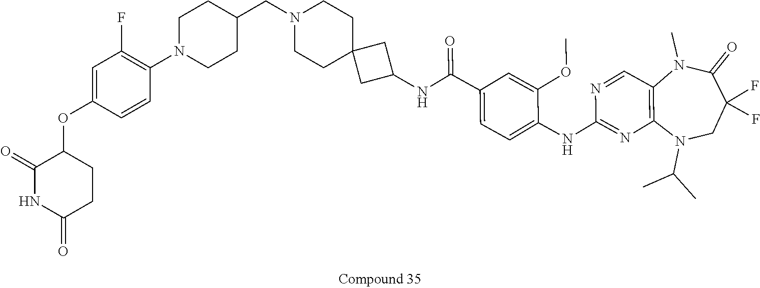 [SMILES: COc1cc(C(=O)NC2CC3(CCN(CC4CCN(c5ccc(OC6CCC(=O)NC6=O)cc5F)CC4)CC3)C2)ccc1Nc1ncc2c(n1)N(C(C)C)CC(F)(F)C(=O)N2C]

### Step 1. Synthesis of 4-(((tert-butyldiphenylsilyl)oxy)methyl)piperidine (2)

To a solution of piperidin-4-ylmethanol (10 g, 86.83 mmol) in DCM (150 mL) was added TEA (13.18 g, 130.24 mmol, 18.13 mL) and DMAP (530.37 mg, 4.34 mmol), then tert-butylchlorodiphenylsilane (35.80 g, 130.24 mmol, 33.46 mL) was added dropwise. The mixture was stirred at 20° C. for 16 hr. TLC (Dichloromethane:Methanol=10:1) indicated piperidin-4-ylmethanol was consumed completely and one main new spot was formed. The reaction mixture was diluted with water (100 mL) at 0° C., and then extracted with dichloromethane (80 mL×2). The combined organic layers were washed with brine (30 mL×3), dried over Na₂SO₄, filtered and concentrated under reduced pressure to give a residue, which was purified by flash silica gel chromatography (ISCO®; 80 g SepaFlash® Silica Flash Column, Eluent of 0-100% EtOAc/Petroleum ether gradient@80 mL/min) followed by trituration with MTBE to afford 4-(((tert-butyldiphenylsilyl)oxy)methyl)piperidine (9.6 g, 27.15 mmol, 31.27% yield) as a white solid. MS (M+H)⁺=354.6

### Step 2. Synthesis of 4-(4-(((tert-butyldiphenylsilyl)oxy)methyl)piperidin-1-yl)-3-fluorophenol (4)

A mixture of 4-bromo-3-fluorophenol (500 mg, 2.62 mmol), 4-(((tert-butyldiphenylsilyl)oxy)methyl)piperidine (1.02 g, 2.88 mmol), DavePhos (164.84 mg, 418.85 μmol), Pd₂(dba)₃ (143.83 mg, 157.07 μmol) and NaOBu-t (2 M, 3.93 mL) in dioxane (12 mL) was degassed and purged with N₂ for 3 times, then the mixture was stirred at 100° C. for 16 hr under N₂ atmosphere. LCMS showed 38% of 4-(((tert-butyldiphenylsilyl)oxy)methyl)piperidine remained. Several new peaks were showed on LCMS and a peak (31%) with desired compound. The reaction mixture was diluted with saturated ammonium chloride aqueous solution (20 mL) at 0° C., then extracted with EtOAc (30 mL×2). The combined organic layers were washed with brine (20 mL), dried over Na₂SO₄, filtered and concentrated under reduced pressure to give a residue, which was purified by flash silica gel chromatography (ISCO®; 20 g SepaFlash® Silica Flash Column, Eluent of 0˜16% EtOAc/Petroleum ether gradient@50 mL/min) to afford 4-(4-(((tert-butyldiphenylsilyl)oxy)methyl)piperidin-1-yl)-3-fluorophenol (400 mg, 819.57 μmol, 31.31% yield, 95% purity) as a yellow solid. MS (M+H)⁺=464.2

### Step 3. Synthesis of 3-(4-(4-(((tert-butyldiphenylsilyl)oxy)methyl)piperidin-1-yl)-3-fluorophenoxy)piperidine-2,6-dione (5)

To a solution of 4-(4-(((tert-butyldiphenylsilyl)oxy)methyl)piperidin-1-yl)-3-fluorophenol (200 mg, 431.35 μmol) in THF (5 mL) was added NaH (43.14 mg, 1.08 mmol, 60% purity) at 0° C., the mixture was stirred at 0° C. for 1 hr, then a solution of 3-bromopiperidine-2,6-dione (91.11 mg, 474.49 μmol) in THF (2 mL) was added dropwise at 0° C. and the resulting mixture was stirred at 20° C. for 2 hr. LCMS showed 40% of 4-(4-(((tert-butyldiphenylsilyl)oxy)methyl)piperidin-1-yl)-3-fluorophenol remained, and a peak (52%) with desired mass. Then the mixture was stirred for another 2 hours. TLC (Petroleum ether:EtOAc=3:1, Rf=0.23) indicated one major new spot with larger polarity was detected. The reaction mixture was quenched by addition of saturated ammonium chloride aqueous solution (15 mL) at 0° C., then extracted with EtOAc (30 mL×2). The combined organic layers were washed with brine (20 mL), dried over Na₂SO₄, filtered and concentrated under reduced pressure to give a residue, which was purified by flash silica gel chromatography (ISCO®; 12 g SepaFlash® Silica Flash Column, Eluent of 0˜45% EtOAc/Petroleum ether gradient@50 mL/min) to afford 3-(4-(4-(((tert-butyldiphenylsilyl)oxy)methyl)piperidin-1-yl)-3-fluorophenoxy)piperidine-2,6-dione (190 mg, 320.66 μmol, 74.34% yield, 97% purity) as a yellow solid. MS (M+H)⁺=575.4

### Step 4. Synthesis of 3-(3-fluoro-4-(4-(hydroxymethyl)piperidin-1-yl)phenoxy)piperidine-2,6-dione (6)

To a solution of 3-(4-(4-(((tert-butyldiphenylsilyl)oxy)methyl)piperidin-1-yl)-3-fluorophenoxy)piperidine-2,6-dione (140 mg, 243.58 μmol) in DMSO (1 mL) was added CsF (74.00 mg, 487.16 μmol, 17.96 L). The mixture was stirred at 30° C. for 2.5 hour. LCMS showed 9% of 3-(4-(4-(((tert-butyldiphenylsilyl)oxy)methyl)piperidin-1-yl)-3-fluorophenoxy)piperidine-2,6-dione remained and a peak (44%) with desired mass. The reaction mixture was diluted with saturated ammonium chloride aqueous solution (20 mL) at 0° C., then extracted with EtOAc (20 mL×2). The combined organic layers were washed with brine (10 mL), dried over Na₂SO₄, filtered and concentrated under reduced pressure to give a residue, which was purified by flash silica gel chromatography (ISCO®; 12 g SepaFlash® Silica Flash Column, Eluent of 0˜100% EtOAc/Petroleum ether gradient @50 mL/min) to afford 3-(3-fluoro-4-(4-(hydroxymethyl)piperidin-1-yl)phenoxy)piperidine-2,6-dione (50 mg, 148.65 μmol, 61.03% yield) as a yellow solid. MS (M+H)⁺=337.4

¹H NMR (400 MHz, CDCl₃) δ=7.76 (s, 1H), 6.99-6.87 (m, 1H), 6.85-6.74 (m, 2H), 4.77 (dd, J=4.4, 7.5 Hz, 1H), 3.58 (d, J=6.0 Hz, 2H), 3.44-3.33 (m, 2H), 3.02-2.90 (m, 1H), 2.74-2.58 (m, 3H), 2.38-2.23 (m, 2H), 1.91-1.78 (m, 2H), 1.69-1.59 (m, 1H), 1.51-1.41 (m, 2H), 1.37-1.29 (m, 1H).

### Step 5. Synthesis of 1-(4-((2,6-dioxopiperidin-3-yl)oxy)-2-fluorophenyl)piperidine-4-carbaldehyde (7)

To a solution of 3-(3-fluoro-4-(4-(hydroxymethyl)piperidin-1-yl)phenoxy)piperidine-2,6-dione (50 mg, 148.65 μmol) in DCM (1 mL) was added DMP (75.66 mg, 178.38 μmol, 55.23 μL). The mixture was stirred at 20° C. for 2 hour. LCMS showed some of 3-(3-fluoro-4-(4-(hydroxymethyl)piperidin-1-yl)phenoxy)piperidine-2,6-dione remained and hydrate mass. Then the mixture was stirred for another 2 hours, LCMS showed 3-(3-fluoro-4-(4-(hydroxymethyl)piperidin-1-yl)phenoxy)piperidine-2,6-dione was consumed completely. The reaction mixture was filtered and the filtrate was concentrated to afford 1-(4-((2,6-dioxopiperidin-3-yl)oxy)-2-fluorophenyl)piperidine-4-carbaldehyde (49 mg, crude) as a yellow solid, which was used into the next step without further purification. MS (M+H+18)⁺=353.1

### Step 6. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(7-((1-(4-((2,6-dioxopiperidin-3-yl)oxy)-2-fluorophen yl)piperidin-4-yl)methyl)-7-azaspiro[3.5]nonan-2-yl)-3-methoxybenzamide (Compound 35)

To a solution of 1-(4-((2,6-dioxopiperidin-3-yl)oxy)-2-fluorophenyl)piperidine-4-carbaldehyde (49 mg, 146.56 μmol) and 4-((7,7-difluoro-9-isopropyl-5-methyl-6-oxo-6,7,8,9-tetrahydro-5H-pyrimido[4,5-b][1,4]diazepin-2-yl)amino)-3-methoxy-N-(7-azaspiro[3.5]nonan-2-yl)benzamide (76.51 mg, 131.90 μmol, HCl salt) in DCE (0.5 mL) was added NaOAc (18.03 mg, 219.84 μmol) at 20° C. After addition, the mixture was stirred at 20° C. for 1 hr, then NaBH(OAc)₃ (155.31 mg, 732.78 μmol) was added and the resulting mixture was stirred at 20° C. for 16 hr. LCMS showed 1-(4-((2,6-dioxopiperidin-3-yl)oxy)-2-fluorophenyl)piperidine-4-carbaldehyde was consumed completely and one peak (52%) with desired mass. The reaction mixture was quenched by addition of saturated sodium bicarbonate aqueous solution (10 mL) at 0° C., then extracted with EtOAc (20 mL×2). The combined organic layers were washed with brine (15 mL), dried over Na₂SO₄, filtered and concentrated under reduced pressure to give a residue, which was purified by prep-TLC (SiO₂, DCM:MeOH=5:1) followed by prep-HPLC (column: Phenomenex luna C18 150×25 mm×10 m; mobile phase: [water (FA)-ACN]; B %: 15%-45%, 10 min) and prep-HPLC (column: Waters Xbridge 150×25 mm×5 μm; mobile phase: [water (NH₄HCO₃)-ACN]; B %: 44%-74%, 8 min), the eluent was lyophilized to afford 4-((7,7-difluoro-9-isopropyl-5-methyl-6-oxo-6,7,8,9-tetrahydro-5H-pyrimido[4,5-b][1,4]diazepin-2-yl)amino)-N-(7-((1-(4-((2,6-dioxopiperidin-3-yl)oxy)-2-fluorophenyl)piperidin-4-yl)methyl)-7-azaspiro[3.5]nonan-2-yl)-3-methoxybenzamide (10.2 mg, 11.36 mol, 7.75% yield, 96% purity) as a white solid. MS (M+H)⁺=862.2

¹H NMR (400 MHz, DMSO-d₆) δ=10.92 (s, 1H), 8.42 (d, J=7.5 Hz, 1H), 8.35-8.27 (m, 1H), 8.22 (s, 1H), 7.88 (s, 1H), 7.54-7.46 (m, 2H), 6.96 (t, J=9.4 Hz, 1H), 6.88 (dd, J=2.7, 13.9 Hz, 1H), 6.77-6.74 (m, 1H), 5.13-5.09 (m 1H), 4.93-4.83 (m, 1H), 4.46-4.32 (m, 1H), 4.04 (t, J=13.6 Hz, 2H), 3.94 (s, 3H), 3.30 (s, 3H), 3.21-3.18 (m, 2H), 2.66-2.59 (m, 2H), 2.57-2.56 (m, 2H), 2.32-2.26 (m, 2H), 2.26-2.19 (m, 2H), 2.19-2.08 (m, 6H), 1.85-1.72 (m, 4H), 1.64-1.52 (m, 5H), 1.24 (d, J=6.6 Hz, 8H).

### Example 36. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(7-((1-(4-((2,6-dioxopiperidin-3-yl)oxy)phenyl)piperidin-4-yl)methyl)-7-azaspiro[3.5]nonan-2-yl)-3-methoxybenzamide (Compound 36)

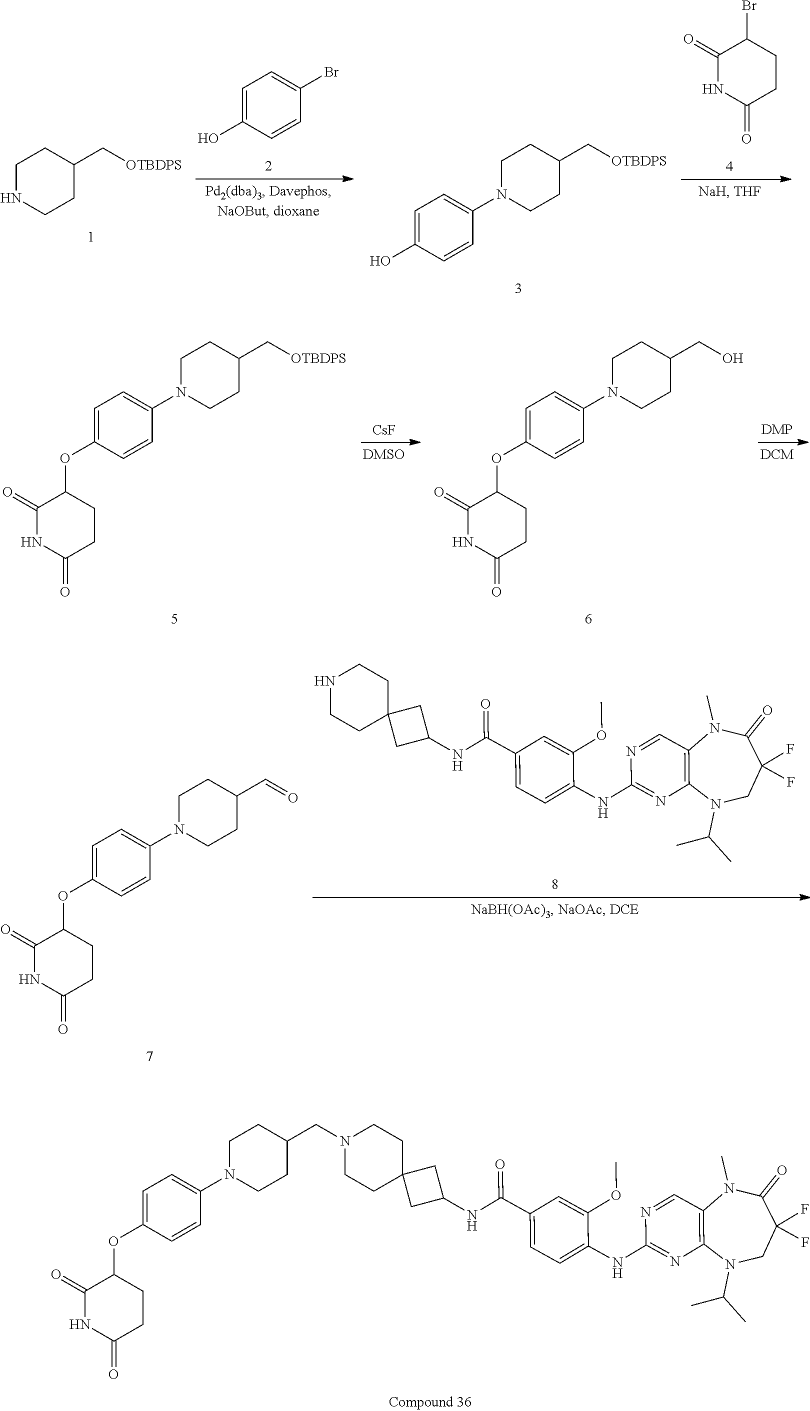

### Step 1. Synthesis of 4-(4-(((tert-butyldiphenylsilyl)oxy)methyl)piperidin-1-yl)phenol (3)

A mixture of 4-bromophenol (450 mg, 2.60 mmol), 4-(((tert-butyldiphenylsilyl)oxy)methyl)piperidine (1.01 g, 2.86 mmol), DavePhos (204.73 mg, 520.21 μmol), Pd₂(dba)₃ (142.91 mg, 156.06 μmol) and NaOBu-t (2 M, 3.90 mL) in dioxane (10 mL) was degassed and purged with N₂ for 3 times, then the mixture was stirred at 100° C. for 16 hr under N₂ atmosphere. LCMS showed 12% of 4-(((tert-butyldiphenylsilyl)oxy)methyl)piperidine remained. and a peak (54%) with desired compound. The reaction mixture was diluted with saturated ammonium chloride aqueous solution (20 mL) at 0° C., then extracted with EtOAc (30 mL×2). The combined organic layers were washed with brine (20 mL), dried over Na₂SO₄, filtered and concentrated under reduced pressure to give a residue, which was purified by flash silica gel chromatography (ISCO®; 20 g SepaFlash® Silica Flash Column, Eluent of 0˜18% EtOAc/Petroleum ether gradient@50 mL/min) to afford 4-(4-(((tert-butyldiphenylsilyl)oxy)methyl)piperidin-1-yl)phenol (650 mg, 1.40 mmol, 53.83% yield, 96% purity) as a yellow solid. MS (M+H)⁺=446.2

### Step 2. Synthesis of 3-(4-(4-(((tert-butyldiphenylsilyl)oxy)methyl)piperidin-1-yl)phenoxy)piperidine-2, 6-dione (5)

To a solution of 4-(4-(((tert-butyldiphenylsilyl)oxy)methyl)piperidin-1-yl)phenol (320 mg, 718.02 μmol) in THF (10 mL) was added NaH (71.80 mg, 1.80 mmol, 60% purity) at 0° C. After addition, the mixture was stirred at 0° C. for 1 hr, then a solution of 3-bromopiperidine-2,6-dione (165.44 mg, 861.63 μmol) in THF (2 mL) was added dropwise at 0° C. and the resulting mixture was stirred at 20° C. for 3 hr. LCMS showed 4-(4-(((tert-butyldiphenylsilyl)oxy)methyl)piperidin-1-yl)phenol was consumed completely and desired mass was detected. The reaction mixture was quenched by addition of saturated ammonium chloride aqueous solution (20 mL) at 0° C., then extracted with EtOAc (30 mL×2). The combined organic layers were washed with brine (20 mL), dried over Na₂SO₄, filtered and concentrated under reduced pressure to give a residue, which was purified by flash silica gel chromatography (ISCO®; 12 g SepaFlash® Silica Flash Column, Eluent of 0˜50% EtOAc/Petroleum ether gradient@50 mL/min) to afford 3-(4-(4-(((tert-butyldiphenylsilyl)oxy)methyl)piperidin-1-yl)phenoxy)piperidine-2,6-di one (380 mg, 662.04 μmol, 92.20% yield, 97% purity) as a yellow solid. MS (M+H)⁺=557.3

### Step 3. Synthesis of 3-(4-(4-(hydroxymethyl)piperidin-1-yl)phenoxy)piperidine-2,6-dione (6)

To a solution of 3-(4-(4-(((tert-butyldiphenylsilyl)oxy)methyl)piperidin-1-yl)phenoxy)piperidine-2,6-di one (380 mg, 682.51 μmol) in DMSO (2 mL) was added CsF (207.35 mg, 1.37 mmol, 50.33 μL). The mixture was stirred at 30° C. for 4 hour. LCMS showed 3-(4-(4-(((tert-butyldiphenylsilyl)oxy)methyl)piperidin-1-yl)phenoxy)piperidine-2,6-di one was consumed completely and desired mass. The reaction mixture was diluted with saturated ammonium chloride aqueous solution (10 mL) at 0° C., then extracted with EtOAc (30 mL×2). The combined organic layers were washed with brine (20 mL), dried over Na₂SO₄, filtered and concentrated under reduced pressure to give a residue, which was purified by flash silica gel chromatography (ISCO®; 20 g SepaFlash® Silica Flash Column, Eluent of 0˜100% EtOAc/Petroleum ether gradient @50 mL/min) to afford 3-(4-(4-(hydroxymethyl)piperidin-1-yl)phenoxy)piperidine-2,6-dione (120 mg, 376.92 mol, 55.23% yield) as a yellow solid. MS (M+H)⁺=319.1

### Step 4. Synthesis of 1-(4-((2,6-dioxopiperidin-3-yl)oxy)phenyl)piperidine-4-carbaldehyde (7)

To a solution of 3-(4-(4-(hydroxymethyl)piperidin-1-yl)phenoxy)piperidine-2,6-dione (60 mg, 188.46 mol) in DCM (2 mL) was added DMP (95.92 mg, 226.15 μmol, 70.02 μL). The mixture was stirred at 20° C. for 5 hr. TLC (Petroleum ether:EtOAc=0:1, Rf=0.7) indicated 3-(4-(4-(hydroxymethyl)piperidin-1-yl)phenoxy)piperidine-2,6-dione was consumed completely, and new spots with lower polarity was detected. The reaction mixture was filtered and the filtrate was concentrated to afford 1-(4-((2,6-dioxopiperidin-3-yl)oxy)phenyl)piperidine-4-carbaldehyde (59 mg, crude) as a red solid, which was used into the next step without further purification. MS (M+H)⁺=317.4

### Step 5. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(7-((1-(4-((2,6-dioxopiperidin-3-yl)oxy)phenyl)piperidin-4-yl)methyl)-7-azaspiro[3.5]nonan-2-yl)-3-methoxybenzamide (Compound 36)

To a solution of 1-(4-((2,6-dioxopiperidin-3-yl)oxy)phenyl)piperidine-4-carbaldehyde (59 mg, 186.50 μmol) and 4-((7,7-difluoro-9-isopropyl-5-methyl-6-oxo-6,7,8,9-tetrahydro-5H-pyrimido[4,5-b][1,4]diazepin-2-yl)amino)-3-methoxy-N-(7-azaspiro[3.5]nonan-2-yl)benzamide (97.37 mg, 167.85 μmol, HCl salt) in DCE (2 mL) was added NaOAc (22.95 mg, 279.75 μmol) at 20° C. After addition, the mixture was stirred at 20° C. for 1 hr, then NaBH(OAc)₃ (237.16 mg, 1.12 mmol) was added and the resulting mixture was stirred at 20° C. for 16 hr. TLC (Dichloromethane:Methanol=5:1) indicated 4-((7,7-difluoro-9-isopropyl-5-methyl-6-oxo-6,7,8,9-tetrahydro-5H-pyrimido[4,5-b][1,4]diazepin-2-yl)amino)-3-methoxy-N-(7-azaspiro[3.5]nonan-2-yl)benzamide remained, and some new spots with lower polarity were formed. The reaction mixture was diluted with saturated sodium bicarbonate aqueous solution (15 mL) at 0° C., then extracted with EtOAc (40 mL×2). The combined organic layers were washed with brine (20 mL), dried over Na₂SO₄, filtered and concentrated under reduced pressure to give a residue, which was purified by prep-TLC (SiO₂, DCM:MeOH=5:1) followed by (column: Waters Xbridge 150×25 mm×5 μm; mobile phase: [water (NH₄HCO₃)-ACN]; B %: 38%-68%, 9 min) and the eluent was lyophilized to afford 4-((7,7-difluoro-9-isopropyl-5-methyl-6-oxo-6,7,8,9-tetrahydro-5H-pyrimido[4,5-b][1,4]diazepin-2-yl)amino)-N-(7-((1-(4-((2,6-dioxopiperidin-3-yl)oxy)phenyl)piperidin-4-yl)methyl)-7-azaspiro[3.5]nonan-2-yl)-3-methoxybenzamide (10.0 mg, 10.90 μmol, 5.84% yield, 92% purity) as a white solid. MS (M+H)⁺=844.4

¹H NMR (400 MHz, DMSO-d₆) δ=10.88 (s, 1H), 8.42-8.40 (m, 1H), 8.35-8.28 (m, 1H), 8.22 (s, 1H), 7.92-7.84 (m, 1H), 7.56-7.45 (m, 2H), 6.99-6.78 (m, 4H), 5.01-5.0 (m, 1H), 4.94-4.83 (m, 1H), 4.45-4.34 (m, 1H), 4.10-3.99 (m, 2H), 3.94 (s, 3H), 3.50 (d, J=12.0 Hz, 2H), 3.29 (s, 3H), 2.73-2.63 (m, 2H), 2.62-2.58 (m, 2H), 2.32-2.24 (m, 2H), 2.24-2.18 (m, 2H), 2.18-2.01 (m, 6H), 1.86-1.72 (m, 4H), 1.65-1.50 (m, 5H), 1.30-1.13 (m, 8H).

### Example 37. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-(1-(1-(2,6-dioxopiperidin-3-yl)-3-methyl-2-oxo-2,3-dihydro-1H-benzo[d]imidazol-4-yl)piperidin-4-yl)ethyl)piperidin-4-yl)-3-methoxybenzamide

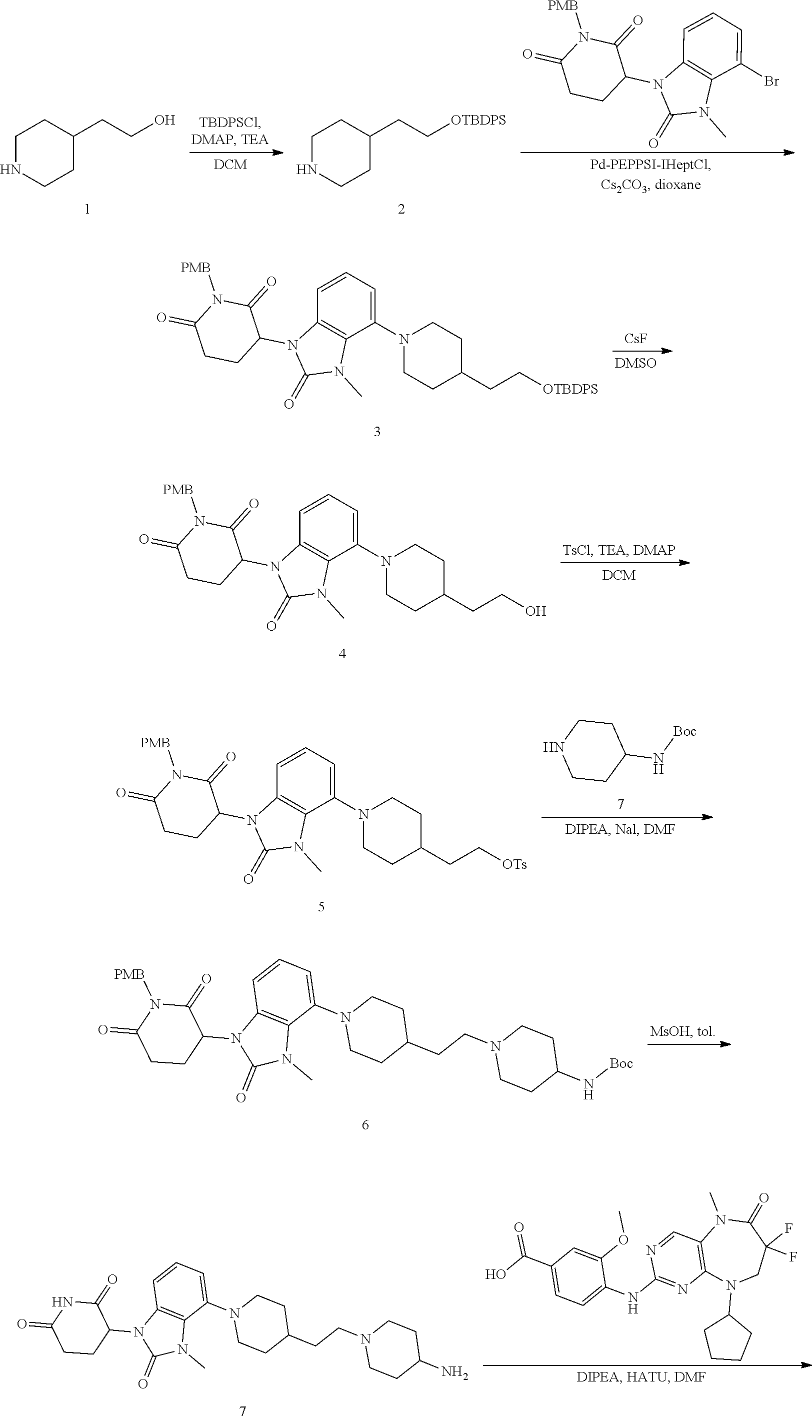

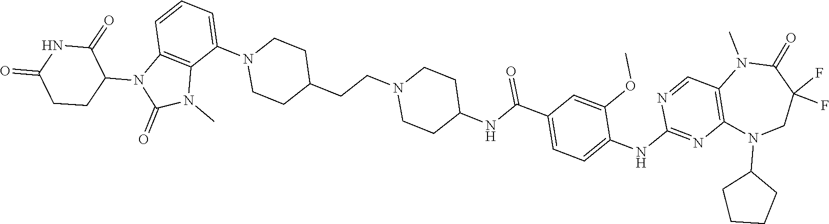 [SMILES: COc1cc(C(=O)NC2CCN(CCC3CCN(c4cccc5c4n(C)c(=O)n5C4CCC(=O)NC4=O)CC3)CC2)ccc1Nc1ncc2c(n1)N(C1CCCC1)CC(F)(F)C(=O)N2C]

### Step 1. Synthesis of 4-(2-((tert-butyldiphenylsilyl)oxy)ethyl)piperidine (2)

To a solution of 2-(piperidin-4-yl)ethan-1-ol (10 g, 77.40 mmol) in DCM (150 mL) were added TEA (15.66 g, 154.80 mmol, 21.55 mL) and DMAP (472.79 mg, 3.87 mmol), followed by tert-butyl-chloro-diphenyl-silane (31.91 g, 116.10 mmol, 29.82 mL) was added slowly, the mixture was stirred at 20° C. for 16 hr. LCMS showed a peak (33%) with desired mass. The mixture was diluted with water (30 mL), extracted with EtOAc (50 mL×3). The organic layer was dried over Na₂SO₄, filtered and concentrated under vacuum. The crude product was purified by flash silica gel chromatography (Biotage, 4 g SepaFlash® Silica Flash Column, Eluent of 4-100% EtOAc/Petroleum ether gradient@100 mL/min) to afford the product, which was triturated with MTBE (20 mL) at 20° C. for 30 min and filtered to afford 4-(2-((tert-butyldiphenylsilyl)oxy)ethyl)piperidine (2.54 g, 6.56 mmol, 8.48% yield, 95%) as a white solid. MS (M+H)⁺=368.2

### Step 2. Synthesis of 3-(4-(4-(2-((tert-butyldiphenylsilyl)oxy)ethyl)piperidin-1-yl)-3-methyl-2-oxo-2,3-di hydro-1H-benzo[d]imidazol-1-yl)-1-(4-methoxybenzyl)piperidine-2,6-dione (3)

To a solution of 3-(4-bromo-3-methyl-2-oxo-2,3-dihydro-1H-benzo[d]imidazol-1-yl)-1-(4-methoxybenzyl)piperidine-2,6-dione (450 mg, 981.88 μmol) in dioxane (10 mL) were added 4-(2-((tert-butyldiphenylsilyl)oxy)ethyl)piperidine (360.94 mg, 981.88 μmol), Cs₂CO₃ (639.83 mg, 1.96 mmol) and Pd-PEPPSI-IHeptCl (47.76 mg, 49.09 μmol), the mixture was stirred at 100° C. for 16 h under N₂ atmosphere. LCMS showed desired mass, the mixture was diluted with water (10 mL), extracted with EtOAc (10 mL×3). The combined organic layer was dried over Na₂SO₄, filtered and concentrated under vacuum. The crude product was purified by flash silica gel chromatography (Biotage, 4 g SepaFlash® Silica Flash Column, Eluent of 4-70% EtOAc/Petroleum ether gradient @20 mL/min) to afford 3-(4-(4-(2-((tert-butyldiphenylsilyl)oxy)ethyl)piperidin-1-yl)-3-methyl-2-oxo-2,3-dihydro-1H-benzo[d]imidazol-1-yl)-1-(4-methoxybenzyl)piperidine-2,6-dione (730 mg, crude) as yellow oil. MS (M+H)⁺=745.3

### Step 3. Synthesis of 3-(4-(4-(2-hydroxyethyl)piperidin-1-yl)-3-methyl-2-oxo-2,3-dihydro-1H-benzo[d]imidazol-1-yl)-1-(4-methoxybenzyl)piperidine-2,6-dione (4)

To a solution of 3-(4-(4-(2-((tert-butyldiphenylsilyl)oxy)ethyl)piperidin-1-yl)-3-methyl-2-oxo-2,3-dihydro-1H-benzo[d]imidazol-1-yl)-1-(4-methoxybenzyl)piperidine-2,6-dione (730 mg, 979.88 μmol) in DMSO (15 mL) was added CsF (223.27 mg, 1.47 mmol, 54.19 μL), the mixture was stirred at 20° C. for 16 hr. LCMS showed desired mass, the mixture was diluted with water (3 mL), extracted with EtOAc (5 mL×3). The combined organic layer was dried over Na₂SO₄, filtered and concentrated under vacuum, the crude product was purified by flash silica gel chromatography (Biotage, 4 g SepaFlash® Silica Flash Column, Eluent of 4-65% EtOAc/Petroleum ether gradient@20 mL/min) to afford 3-(4-(4-(2-hydroxyethyl)piperidin-1-yl)-3-methyl-2-oxo-2,3-dihydro-1H-benzo[d]imidazol-1-yl)-1-(4-methoxybenzyl)piperidine-2,6-dione (200 mg, 363.21 μmol, 37.07% yield, 92% purity) as yellow oil. MS (M+H)⁺=507.3

### Step 4. Synthesis of 2-(1-(1-(1-(4-methoxybenzyl)-2,6-dioxopiperidin-3-yl)-3-methyl-2-oxo-2,3-dihydro-1H-benzo[d]imidazol-4-yl)piperidin-4-yl)ethyl 4-methylbenzenesulfonate (5)

To a solution of 3-(4-(4-(2-hydroxyethyl)piperidin-1-yl)-3-methyl-2-oxo-2,3-dihydro-1H-benzo[d]imidazol-1-yl)-1-(4-methoxybenzyl)piperidine-2,6-dione (200 mg, 394.80 μmol) in DCM (2 mL) were added TEA (119.85 mg, 1.18 mmol, 164.85 L), TosCl (112.90 mg, 592.19 μmol) and DMAP (4.82 mg, 39.48 μmol), the mixture was stirred at 20° C. for 16 hr. LCMS showed the desired mass, the mixture was diluted with water (3 mL), extracted with EtOAc (5 mL×3). The organic layer was dried over Na₂SO₄, filtered and concentrated under vacuum, the crude product was purified by flash silica gel chromatography (Biotage, 4 g SepaFlash® Silica Flash Column, Eluent of 4-98% EtOAc/Petroleum ether gradient@20 mL/min) to afford 2-(1-(1-(1-(4-methoxybenzyl)-2,6-dioxopiperidin-3-yl)-3-methyl-2-oxo-2,3-dihydro-1H-benzo[d]imidazol-4-yl)piperidin-4-yl)ethyl 4-methylbenzenesulfonate (200 mg, 236.09 μmol, 59.80% yield, 78% purity) as yellow oil. MS (M+H)⁺=661.2

### Step 5. Synthesis of tert-butyl (1-(2-(1-(1-(1-(4-methoxybenzyl)-2,6-dioxopiperidin-3-yl)-3-methyl-2-oxo-2,3-dihydro-1H-benzo[d]imidazol-4-yl)piperidin-4-yl)ethyl)piperidin-4-yl)carbamate (6)

To a solution of 2-(1-(1-(1-(4-methoxybenzyl)-2,6-dioxopiperidin-3-yl)-3-methyl-2-oxo-2,3-dihydro-1H-benzo[d]imidazol-4-yl)piperidin-4-yl)ethyl 4-methylbenzenesulfonate (200 mg, 302.67 μmol) and tert-butyl N-(4-piperidyl) carbamate (90.93 mg, 454.01 μmol) in DMF (8 mL) were added DIEA (117.35 mg, 908.02 μmol) and NaI (4.54 mg, 30.27 mol) at 20° C. The resulting mixture was stirred at 80° C. for 2 hrs. LCMS showed a main peak with desired mass. The reaction mixture was poured into water (20 mL) and extracted with EtOAc (10 mL×3). The combined organic layers were washed with brine (10 mL×3), dried over Na₂SO₄ and concentrated to afford tert-butyl (1-(2-(1-(1-(1-(4-methoxybenzyl)-2,6-dioxopiperidin-3-yl)-3-methyl-2-oxo-2,3-dihydro-1H-benzo[d]imidazol-4-yl)piperidin-4-yl)ethyl)piperidin-4-yl)carbamate (210 mg, crude) as a brown solid. The crude product was used for the next step directly. MS (M+H)⁺=689.4.

### Step 6. Synthesis of 3-(4-(4-(2-(4-aminopiperidin-1-yl)ethyl)piperidin-1-yl)-3-methyl-2-oxo-2,3-dihydro-1H-benzo[d]imidazol-1-yl)piperidine-2,6-dione (7)

To a solution of tert-butyl (1-(2-(1-(1-(1-(4-methoxybenzyl)-2,6-dioxopiperidin-3-yl)-3-methyl-2-oxo-2,3-dihydro-1H-benzo[d]imidazol-4-yl)piperidin-4-yl)ethyl)piperidin-4-yl)carbamate (200 mg, crude) in Toluene (9 mL) were added MsOH (4.05 g, 42.14 mmol, 3 mL), the mixture was stirred at 100° C. for 3 hr. LCMS showed desired mass, the mixture was diluted with the aqueous of NaHCO₃ (20 mL), extracted with EtOAc (20 mL×3), the aqueous was concentrated by freeze drying to afford the white solid, then the solid was triturated with THF (50 mL) and stirred at 20° C. for 1 h, filtered, the filtrate was concentrated under vacuum to afford 3-(4-(4-(2-(4-aminopiperidin-1-yl)ethyl)piperidin-1-yl)-3-methyl-2-oxo-2,3-dihydro-1H-benzo[d]imidazol-1-yl)piperidine-2,6-dione (48 mg, crude) as yellow oil. MS (M+H)+=469.3

### Step 7. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-(1-(1-(2,6-dioxopiperidin-3-yl)-3-methyl-2-oxo-2,3-dihydro-1H-benzo[d]imidazol-4-yl)piperidin-4-yl)ethyl)piperidin-4-yl)-3-methoxybenzamide (Compound 37)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (30 mg, 67.05 μmol) in DMF (1 mL) were added HATU (38.24 mg, 100.57 μmol) and DIPEA (26.00 mg, 201.15 μmol, 35.04 μL), then 3-(4-(4-(2-(4-aminopiperidin-1-yl)ethyl)piperidin-1-yl)-3-methyl-2-oxo-2,3-dihydro-1H-benzo[d]imidazol-1-yl)piperidine-2,6-dione (38.79 mg, crude) was added, the mixture was stirred at 20° C. for 16 hr. LCMS showed a major peak with desired mass, the mixture was diluted with water (3 mL), extracted with EtOAc (5 mL×3), the organic layer was dried over Na₂SO₄, filtered and concentrated under vacuum, the crude product was purified by Prep-HPLC (column: Waters Xbridge 150*25 mm*5 m; mobile phase: [water(NH₄HCO₃)-ACN]; B %: 48%-78%, 8 min) and dried by lyophilization to afford 4-[(9-cyclopentyl-7,7-difluoro-5-methyl-6-oxo-8H-pyrimido[4,5-b][1,4]diazepin-2-yl) amino]-N-[1-[2-[1-[1-(2,6-dioxo-3-piperidyl)-3-methyl-2-oxo-benzimidazol-4-yl]-4-piperidyl]ethyl]-4-piperidyl]-3-methoxy-benzamide (8.5 mg, 8.99 μmol, 13.41% yield, 95% purity) as a white powder. MS (M+H)⁺=898.4

¹H NMR (400 MHz, DMSO-d₆) δ=11.10 (s, 1H), 8.32-8.24 (m, 2H), 8.12 (d, J=7.6 Hz, 1H), 7.97 (s, 1H), 7.53-7.46 (m, 2H), 7.02-6.83 (m, 3H), 5.36 (dd, J=5.3, 12.5 Hz, 1H), 4.77 (t, J=8.3 Hz, 1H), 4.05 (t, J=14.2 Hz, 2H), 3.94 (s, 3H), 3.84-3.71 (m, 1H), 3.63 (s, 3H), 3.34 (s, 3H), 3.10 (d, J=9.8 Hz, 2H), 2.92 (d, J=9.7 Hz, 2H), 2.74-2.60 (m, 4H), 2.37-2.35 (m, 2H), 2.07-1.87 (m, 6H), 1.90-1.78 (m, 4H), 1.76-1.75 (m, 2H), 1.68-1.52 (m, 6H), 1.49-1.34 (m, 5H).

### Example 38. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(7-((1-(1-(2,6-dioxopiperidin-3-yl)-3-methyl-2-oxo-2, 3-dihydro-1H-benzo[d]imidazol-4-yl)piperidin-4-yl)methyl)-7-azaspiro[3.5]nonan-2-yl)-3-methoxybenzamide (Compound 38)

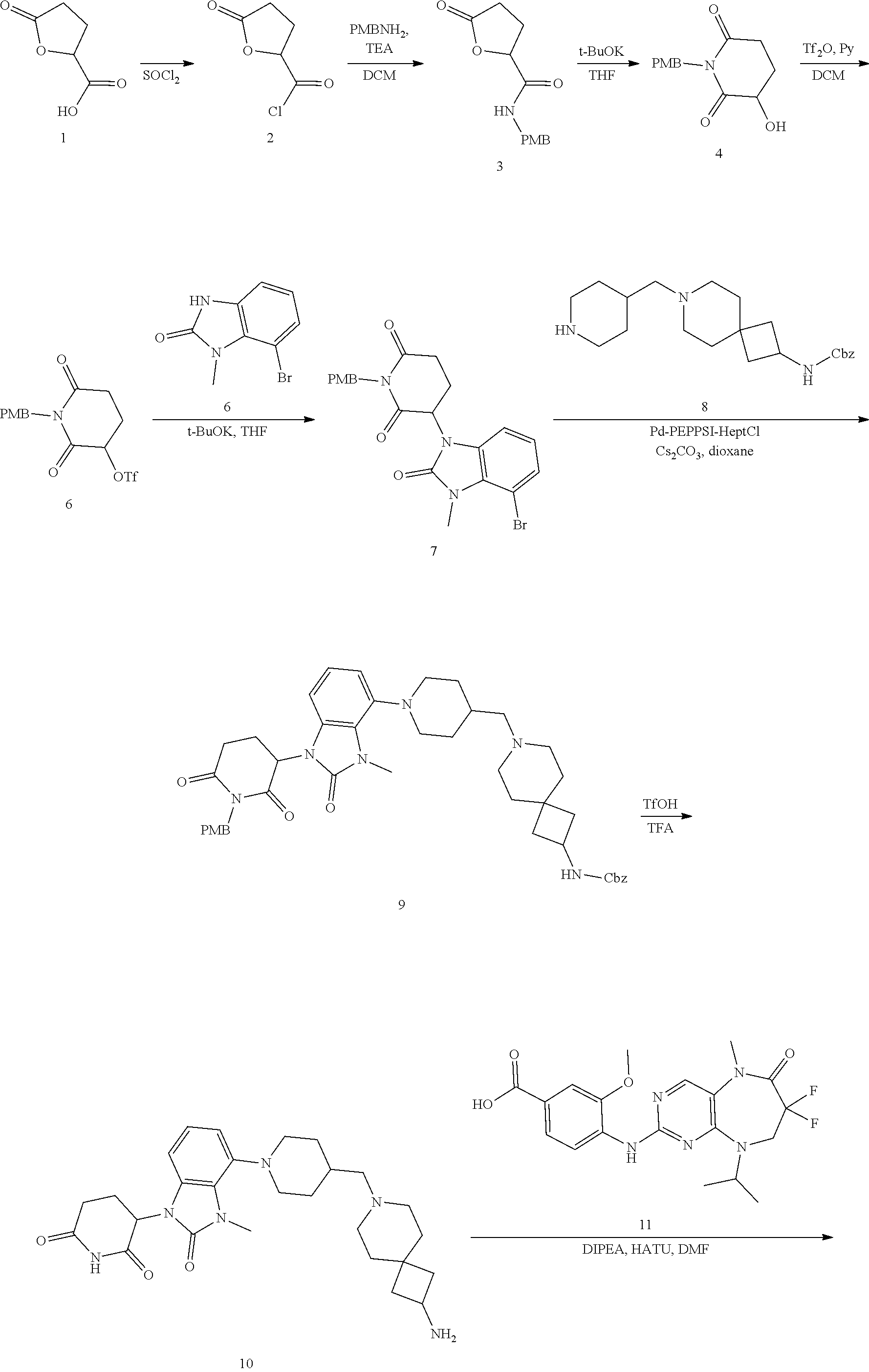

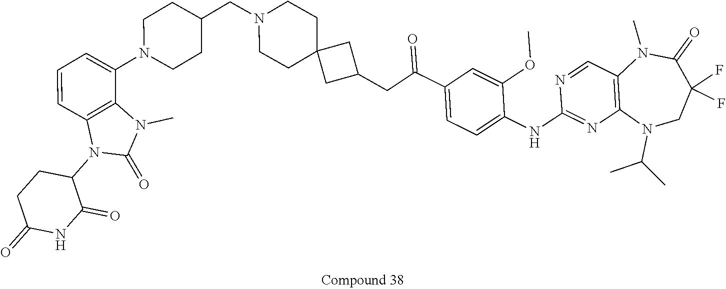 [SMILES: COc1cc(C(=O)CC2CC3(CCN(CC4CCN(c5cccc6c5n(C)c(=O)n6C5CCC(=O)NC5=O)CC4)CC3)C2)ccc1Nc1ncc2c(n1)N(C(C)C)CC(F)(F)C(=O)N2C]

### Step 1. Synthesis of 5-oxotetrahydrofuran-2-carbonyl chloride (2)

To 5-oxotetrahydrofuran-2-carboxylic acid (10 g, 76.86 mmol) was added SOCl₂ (20 mL) at 0° C. slowly, the mixture was stirred at 85° C. for 2 h, and then the mixture was stirred at 15° C. for 4 h. TLC (Dichloromethane:Methanol=10:1) indicated the starting material was consumed completely, and one major new spot with lower polarity was detected. The mixture was concentrated in vacuum to afford 5-oxotetrahydrofuran-2-carbonyl chloride (11 g, crude) as a brown oil, which was used into the next step directly. MS (M+H)⁺=149.5

### Step 2. Synthesis of N-(4-methoxybenzyl)-5-oxotetrahydrofuran-2-carboxamide (3)

5-oxotetrahydrofuran-2-carbonyl chloride (11 g, 74.05 mmol) was dissolved in dry DCM (100 mL) at 0° C. under N₂. Then a solution of TEA (14.99 g, 148.10 mmol, 20.61 mL) and PMBNH₂ (8.13 g, 59.24 mmol, 7.67 mL) in DCM (30 mL) was added and the mixture was stirred at 15° C. for 3 hrs. LCMS showed a main peak with desired mass. H₂O (100 mL) was added and the organic phase was separated, the aqueous phase was extracted with EtOAc (100 mL×3). The combined organic phase was washed with 0.5 M HCl (50 mL) and brine (50 mL), dried over with anhydrous sodium sulfate and filtered. The filtrate was concentrated in vacuo. The residue was purified by flash silica gel chromatography (Biotage; 20 g SepaFlash® Silica Flash Column, Eluent of 50-100% EtOAc/Petroleum ether gradient@40 mL/min) to afford N-(4-methoxybenzyl)-5-oxotetrahydrofuran-2-carboxamide (10 g, 40.12 mmol, 54.18% yield) as a yellow solid. MS (M+H)⁺=250.0

### Step 3. Synthesis of 3-hydroxy-1-(4-methoxybenzyl)piperidine-2,6-dione (4)

A solution of N-(4-methoxybenzyl)-5-oxotetrahydrofuran-2-carboxamide (1.5 g, 6.02 mmol) in anhydrous THF (15 mL) was cooled to −78° C. Then t-BuOK (1 M, 6.62 mL) (1 N in THF) in a solution of anhydrous THF (10 mL) was added dropwise at −78° C. under nitrogen atmosphere. The resulting reaction mixture was stirred at −40° C. for 1 hr. TLC (EtOAc) indicated the starting material was consumed completely, and two new spots were formed. The reaction mixture was quenched by addition of NH₄Cl (20 mL), extracted with EtOAc (20 mL×3). The combined organic layers were washed with brine (20 mL×2), dried over Na₂SO₄, filtered. The filtrate was concentrated in vacuo. The residue was purified by flash silica gel chromatography (Biotage; 20 g SepaFlash® Silica Flash Column, Eluent of 0˜60% EtOAc/Petroleum ether gradient@40 mL/min) to afford 3-hydroxy-1-(4-methoxybenzyl)piperidine-2,6-dione (900 mg, crude) as a white solid. MS (M+H)⁺=250.3

### Step 4. Synthesis of 1-(4-methoxybenzyl)-2,6-dioxopiperidin-3-yl trifluoromethanesulfonate (5)

To a solution of 3-hydroxy-1-(4-methoxybenzyl)piperidine-2,6-dione (500 mg, 2.01 mmol) and Py (317.44 mg, 4.01 mmol, 323.92 μL) in DCM (5 mL) was added Tf₂O (848.84 mg, 3.01 mmol, 496.40 μL) dropwise at 0° C. The mixture was stirred at −10° C. for 1.5 hours under N₂. TLC (Petroleum ether:EtOAc=3:1) indicated the starting material was consumed completely, and one major new spot with lower polarity was detected. The mixture was concentrated in vacuum. The residue was purified by flash silica gel chromatography (Biotage; 20 g SepaFlash® Silica Flash Column, Eluent of 0-50% EtOAc/Petroleum ether gradient@40 mL/min) to afford 1-(4-methoxybenzyl)-2,6-dioxopiperidin-3-yl trifluoromethanesulfonate (740 mg, 1.94 mmol, 96.74% yield) as a yellow oil. MS (M+H)⁺=381.3

### Step 5. Synthesis of 3-(4-bromo-3-methyl-2-oxo-2,3-dihydro-1H-benzo[d]imidazole-1-yl)-1-(4-methoxy benzyl)piperidine-2,6-dione (7)

To a solution of 7-bromo-1-methyl-1,3-dihydro-2H-benzo[d]imidazol-2-one (396.57 mg, 1.75 mmol) in THF (10 mL) was added t-BuOK (1 M, 2.33 mL). The reaction mixture was stirred at 0° C. for 0.5 hr. Subsequently, 1-(4-methoxybenzyl)-2,6-dioxopiperidin-3-yl trifluoromethanesulfonate (740 mg, 1.94 mmol) in THF (4 mL) was added dropwise. The resulting reaction mixture was stirred at 20° C. for 0.5 h under N₂. LCMS showed a main peak with desired mass. The reaction mixture was diluted with water 20 mL, extracted with EtOAc (20 mL×3). The combined organic layers were washed with brine (20 mL×2), dried over Na₂SO₄, filtered. The filtrate was concentrated in vacuo. The residue was purified by flash silica gel chromatography (Biotage; 20 g SepaFlash® Silica Flash Column, Eluent of 20-100% EtOAc/Petroleum ether to 10% Methanol/EtOAc gradient@40 mL/min) and re-purified by prep-TLC (SiO₂, Petroleum ether:EtOAc=1:1) to afford 3-(4-bromo-3-methyl-2-oxo-2,3-dihydro-1H-benzo[d]imidazol-1-yl)-1-(4-methoxybenzyl)piperidine-2,6-dione (600 mg, 1.17 mmol, 60.04% yield, 89% purity) as a yellow solid. MS (M+H)⁺=458.0

### Step 6. Synthesis of benzyl (7-((1-(1-(1-(4-methoxybenzyl)-2,6-dioxopiperidin-3-yl)-3-methyl-2-oxo-2,3-dihydro-1H-benzo[d]imidazol-4-yl)piperidin-4-yl)methyl)-7-azaspiro[3.5]nonan-2-yl)carbamate (9)

To a solution of 3-(4-bromo-3-methyl-2-oxo-2,3-dihydro-1H-benzo[d]imidazol-1-yl)-1-(4-methoxybenzyl)piperidine-2,6-dione (300 mg, 654.59 μmol) and benzyl (7-(piperidin-4-ylmethyl)-7-azaspiro[3.5]nonan-2-yl)carbamate (291.09 mg, 654.97 mol, 2HCl) in dioxane (3 mL) was added Pd-PEPPSI-IHeptCl (63.68 mg, 65.46 mol) and Cs₂CO₃ (1.07 g, 3.27 mmol), the mixture was stirred at 100° C. for 16 h. LCMS showed a peak (20%) with desired mass. The mixture was filtered through a pad of celite and filtrate was concentrated in vacuum. The residue was purified by flash silica gel chromatography (Biotage; 20 g SepaFlash® Silica Flash Column, Eluent of 50-100% EtOAc/Petroleum ether to 10% Methanol/EtOAc gradient@40 mL/min) and re-purified by prep-TLC (SiO₂, DCM:MeOH=10:1) to afford benzyl (7-((1-(1-(1-(4-methoxybenzyl)-2,6-dioxopiperidin-3-yl)-3-methyl-2-oxo-2,3-dihydro-1H-benzo[d]imidazol-4-yl)piperidin-4-yl)methyl)-7-azaspiro[3.5]nonan-2-yl)carbamate (70 mg, 93.47 μmol, 14.28% yield, N/A purity) as a yellow solid. MS (M+H)⁺=749.4

### Step 7. Synthesis of 3-(4-(4-((2-amino-7-azaspiro[3.5]nonan-7-yl)methyl)piperidin-1-yl)-3-methyl-2-oxo-2,3-dihydro-1H-benzo[d]imidazol-1-yl)piperidine-2,6-dione (10)

To a solution of benzyl (7-((1-(1-(1-(4-methoxybenzyl)-2,6-dioxopiperidin-3-yl)-3-methyl-2-oxo-2,3-dihydro-1H-benzo[d]imidazol-4-yl)piperidin-4-yl)methyl)-7-azaspiro[3.5]nonan-2-yl)carbamate (70 mg, 93.47 μmol) in TFA (0.5 mL) was added TfOH (1.19 g, 7.93 mmol, 700.01 μL), the mixture was stirred at 100° C. for 2 h. LCMS showed a peak (33%) with desired mass. The mixture was concentrated in vacuum to afford 3-(4-(4-((2-amino-7-azaspiro[3.5]nonan-7-yl)methyl)piperidin-1-yl)-3-methyl-2-oxo-2, 3-dihydro-1H-benzo[d]imidazol-1-yl)piperidine-2,6-dione (50 mg, crude, TFA) as a brown oil, which was used into the next step directly. MS (M+H)⁺=495.3

### Step 8. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(7-((1-(1-(2,6-dioxopiperidin-3-yl)-3-methyl-2-oxo-2, 3-dihydro-1H-benzo[d]imidazol-4-yl)piperidin-4-yl)methyl)-7-azaspiro[3.5]nonan-2-yl)-3-methoxybenzamide (Compound 38)

To a solution of 4-((7,7-difluoro-9-isopropyl-5-methyl-6-oxo-6,7,8,9-tetrahydro-5H-pyrimido[4,5-b][1,4]diazepin-2-yl)amino)-3-methoxybenzoic acid (35.55 mg, 84.37 μmol) in DMF (1 mL) were added HATU (46.85 mg, 123.22 μmol) and DIPEA (31.85 mg, 246.45 μmol, 42.93 μL) the mixture was stirred at 20° C. for 1 h. 3-(4-(4-((2-amino-7-azaspiro[3.5]nonan-7-yl)methyl)piperidin-1-yl)-3-methyl-2-oxo-2, 3-dihydro-1H-benzo[d]imidazol-1-yl)piperidine-2,6-dione (50 mg, crude, TFA) was added. The mixture was stirred at 20° C. for 16 h. LCMS showed a peak (37%) with desired mass. The reaction mixture was diluted with water 5 mL, extracted with EtOAc (10 mL×5). The combined organic layers were washed with brine (10 mL×2), dried over Na₂SO₄, filtered and concentrated under reduced pressure to give a residue. The residue was purified by prep-TLC (SiO₂, DCM:MeOH=9:1) and re-purified by reversed-phase HPLC (column: Waters Xbridge 150×25 mm×5 μm; mobile phase: [water (NH₄HCO₃)-ACN]; B %: 43%-73%, 9 min). The eluent was lyophilized to afford 4-((7,7-difluoro-9-isopropyl-5-methyl-6-oxo-6,7,8,9-tetrahydro-5H-pyrimido[4,5-b][1,4]diazepin-2-yl)amino)-N-(7-((1-(1-(2,6-dioxopiperidin-3-yl)-3-methyl-2-oxo-2,3-dihydro-1H-benzo[d]imidazol-4-yl)piperidin-4-yl)methyl)-7-azaspiro[3.5]nonan-2-yl)-3-m ethoxybenzamide (8.7 mg, 9.30 μmol, 11.32% yield, 96% purity) as a white solid. MS (M+H)⁺=898.4

¹H NMR (400 MHz, DMSO-d₆) δ=11.08 (s, 1H), 8.42 (d, J=7.5 Hz, 1H), 8.33-8.28 (m, 1H), 8.22 (s, 1H), 7.88 (s, 1H), 7.53-7.44 (m, 2H), 7.00-6.93 (m, 1H), 6.92-6.83 (m, 2H), 5.41-5.29 (m, 1H), 4.94-4.82 (m, 1H), 4.46-4.35 (m, 1H), 4.04 (t, J=13.6 Hz, 2H), 3.94 (s, 3H), 3.62 (s, 3H), 3.29 (s, 3H), 3.15-3.06 (m, 2H), 2.92-2.84 (m, 1H), 2.72-2.69 (m, 1H), 2.65-2.57 (m, 3H), 2.29-2.11 (m, 7H), 2.03-1.97 (m, 1H), 1.86-1.76 (m, 4H), 1.66-1.51 (m, 5H), 1.37-1.17 (m, 9H).

### Example 39. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(7-((1-(3-(2,6-dioxopiperidin-3-yl)-1-methyl-1H-indazol-6-yl)piperidin-4-yl)methyl)-7-azaspiro[3.5]nonan-2-yl)-3-methoxybenzamide (Compound 39)

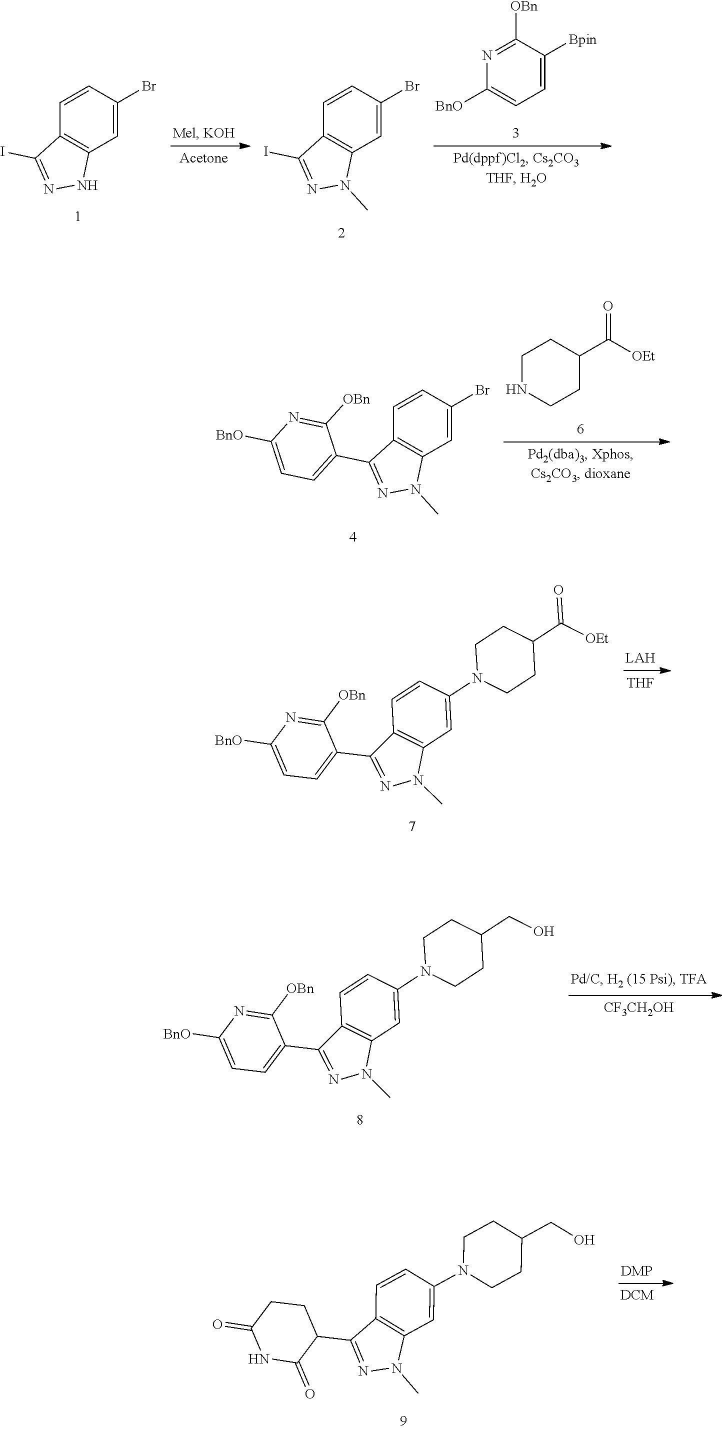

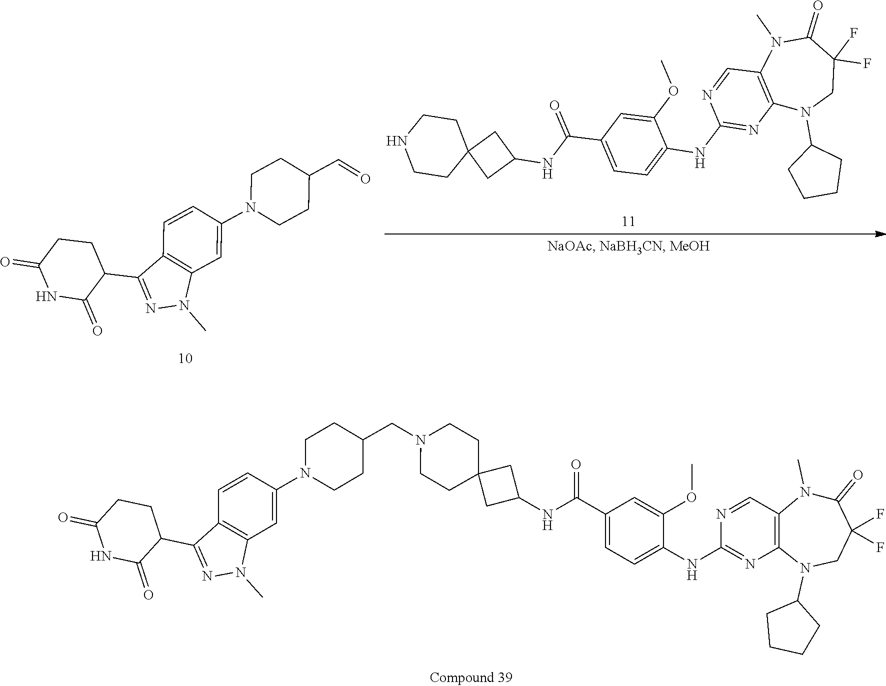 [SMILES: Cn1nc(C2CCC(=O)NC2=O)c2ccc(N3CCC(C=O)CC3)cc21 ; COc1cc(C(=O)NC2CC3(CCNCC3)C2)ccc1Nc1ncc2c(n1)N(C1CCCC1)CC(F)(F)C(=O)N2C ; COc1cc(C(=O)NC2CC3(CCN(CC4CCN(c5ccc6c(C7CCC(=O)NC7=O)nn(C)c6c5)CC4)CC3)C2)ccc1Nc1ncc2c(n1)N(C1CCCC1)CC(F)(F)C(=O)N2C]

### Step 1. Synthesis of 6-bromo-3-iodo-1-methyl-1H-indazole (2)

To a solution of 6-bromo-3-iodo-1H-indazole (5 g, 15.48 mmol) and KOH (2.17 g, 38.71 mmol) in acetone (30 mL) was added Mel (3.30 g, 23.22 mmol, 1.45 mL) dropwise, the mixture was stirred at 20° C. for 16 h. TLC (SiO₂, Petroleum ether:EtOAc=5:1) indicated trace of the starting material remained and one major new spot with lower polarity was formed. The mixture was filtered and the filter cake was washed with acetone (50 mL), the filtrate was concentrated in vacuum. The residue was diluted with EtOAc (30 mL), washed with water (30 mL) and brine (30 mL). The organic layer was dried over Na₂SO₄, filtered and concentrated in vacuum. The residue was purified by flash silica gel chromatography (20 g SepaFlash® Silica Flash Column, Eluent of 4% EtOAc/Petroleum ether gradient@200 mL/min) to afford 6-bromo-3-iodo-1-methyl-1H-indazole (5.04 g, 14.96 mmol, 96.60% yield) as a light yellow solid. MS (M+H)⁺=336.6

### Step 2. Synthesis of 3-(2,6-bis(benzyloxy)pyridin-3-yl)-6-bromo-1-methyl-1H-indazole (4)

To a solution of 6-bromo-3-iodo-1-methyl-1H-indazole (1.3 g, 3.86 mmol) and 2,6-bis(benzyloxy)-3-(4,4,5,5-tetramethyl-1,3,2-dioxaborolan-2-yl)pyridine (1.8 g, 3.45 mmol, 80% purity) in THF (36 mL) and H₂O (6 mL) were added Cs₂CO₃ (2.25 g, 6.90 mmol) and Pd(dppf)Cl₂ (75.75 mg, 103.52 μmol) and the mixture was stirred at 80° C. for 14 h under N₂ atmosphere. LCMS showed a peak (70%) with desired mass. The mixture was diluted with water (60 mL) and extracted with EtOAc (30 mL×3). The combined organic layer was washed with water (30 mL), dried over Na₂SO₄ and filtered. The filtrate was concentrated under reduced pressure. The residue was purified by flash silica gel chromatography (12 g SepaFlash® Silica Flash Column, Eluent of 15% EtOAc/Petroleum ether gradient@80 mL/min) to afford 3-(2,6-bis(benzyloxy)pyridin-3-yl)-6-bromo-1-methyl-1H-indazole (1.6 g, 3.01 mmol, 87.10% yield, 94% purity) as a yellow solid. MS (M+H)⁺=500.3

### Step 3. Synthesis of ethyl 1-(3-(2,6-bis(benzyloxy)pyridin-3-yl)-1-methyl-1H-indazol-6-yl)piperidine-4-carboxylate (7)

To a solution of 3-(2,6-bis(benzyloxy)pyridin-3-yl)-6-bromo-1-methyl-1H-indazole (860 mg, 1.72 mmol) and ethyl piperidine-4-carboxylate (367.20 mg, 2.34 mmol, 360 μL) in dioxane (10 mL) were added Cs₂CO₃ (1.68 g, 5.16 mmol), Pd₂(dba)₃ (31.48 mg, 34.37 μmol) and XPhos (24.58 mg, 51.56 μmol) at 20° C. and the mixture was stirred at 100° C. for 14 h under N₂ atmosphere. LCMS showed a peak (87%) with desired mass. The mixture was filtered and the filter cake was washed with EtOAc (20 mL) and THF (20 mL). The filtrate was concentrated under reduced pressure. The residue was purified by flash silica gel chromatography (12 g SepaFlash® Silica Flash Column, Eluent of 40% EtOAc/Petroleum ether gradient@80 mL/min) to afford ethyl 1-(3-(2,6-bis(benzyloxy)pyridin-3-yl)-1-methyl-1H-indazol-6-yl)piperidine-4-carboxylate (870 mg, 1.51 mmol, 87.78% yield) as a yellow oil. MS (M+H)⁺=577.4

### Step 4. Synthesis of (1-(3-(2,6-bis(benzyloxy)pyridin-3-yl)-1-methyl-1H-indazol-6-yl)piperidin-4-yl)methanol (8)

To a solution of ethyl 1-(3-(2,6-bis(benzyloxy)pyridin-3-yl)-1-methyl-1H-indazol-6-yl)piperidine-4-carboxylate (870 mg, 1.51 mmol) in THF (15 mL) was added LAH (87.00 mg, 2.29 mmol) slowly and the mixture was stirred at 20° C. for 1 h. LCMS showed a main peak (95%) with desired mass. The mixture was quenched with water (5 mL) and stirred at 20° C. for 5 min. The mixture was diluted with EtOAc (30 mL), dried over Na₂SO₄, filtered and concentrated under reduced pressure to afford (1-(3-(2,6-bis(benzyloxy)pyridin-3-yl)-1-methyl-1H-indazol-6-yl)piperidin-4-yl)methanol (0.8 g, crude) as a yellow oil. MS (M+H)⁺=535.3

### Step 5. Synthesis of 3-(6-(4-(hydroxymethyl)piperidin-1-yl)-1-methyl-1H-indazol-3-yl)piperidine-2,6-dione (9)

To a solution of (1-(3-(2,6-bis(benzyloxy)pyridin-3-yl)-1-methyl-1H-indazol-6-yl)piperidin-4-yl)methanol (0.8 g, 1.50 mmol) in CF₃CH₂OH (30 mL) were added TFA (184.80 mg, 1.62 mmol, 120 μL) followed by Pd/C (100 mg, 10% purity) under N₂ atmosphere, the reaction mixture was degassed and purged with H₂ for 3 times and the mixture was stirred at 20° C. for 14 h under H₂ (15 Psi). LCMS showed a peak (85%) with desired mass. The mixture was filtered and the filter cake was washed with EtOH (100 mL) and THF (20 mL). The filtrate was concentrated under reduced pressure. The crude was diluted with THF (10 mL) and adjusted the pH=7 with DIPEA. The mixture was concentrated and then purified by flash silica gel chromatography (12 g SepaFlash® Silica Flash Column, Eluent of 20-30% MeOH/EtOAc gradient@80 mL/min). The product was diluted with EtOH (2 mL) and Petroleum ether (10 mL). The mixture was filtered. The filter cake was collected and dried under reduced pressure to afford 3-(6-(4-(hydroxymethyl)piperidin-1-yl)-1-methyl-1H-indazol-3-yl)piperidine-2,6-dion e (370 mg, 1.01 mmol, 67.30% yield, 97% purity) as a white solid. MS (M+H)⁺=357.4

### Step 6. Synthesis of 1-(3-(2,6-dioxopiperidin-3-yl)-1-methyl-1H-indazol-6-yl)piperidine-4-carbaldehyde (10)

To a solution of 3-(6-(4-(hydroxymethyl)piperidin-1-yl)-1-methyl-1H-indazol-3-yl)piperidine-2,6-dion e (100 mg, 280.57 μmol) in DCM (2 mL) was added DMP (179 mg, 422.03 μmol, 130.66 μL) and the mixture was stirred at 20° C. for 3 h. The mixture was concentrated under reduced pressure. The crude was diluted with MeOH (1 mL) and filtered. The filter cake was washed with MeOH (1 mL) and the filtrate was concentrated under reduced pressure to afford 1-(3-(2,6-dioxopiperidin-3-yl)-1-methyl-1H-indazol-6-yl)piperidine-4-carbaldehyde (0.1 g, crude) as a yellow oil. MS (M+H)⁺=355.1

### Step 7. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(7-((1-(3-(2,6-dioxopiperidin-3-yl)-1-methyl-1H-indazol-6-yl)piperidin-4-yl)methyl)-7-azaspiro[3.5]nonan-2-yl)-3-methoxybenzamide (Compound 39)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-methoxy-N-(7-azaspiro[3.5]nonan-2-yl)benzamide (70 mg, 115.49 μmol, HCl salt) in MeOH (1 mL) was added NaOAc (9.5 mg, 115.81 μmol) followed by 1-(3-(2,6-dioxopiperidin-3-yl)-1-methyl-1H-indazol-6-yl)piperidine-4-carbaldehyde (100 mg, 282.17 μmol) in MeOH (1 mL) and the mixture was stirred at 20° C. for 0.5 h. Then NaBH₃CN (21.77 mg, 346.47 μmol) was added and the mixture was stirred at 20° C. for 2 h. LCMS showed a peak (19%) with desired mass. The mixture was stirred at 20° C. for 12 h. LCMS showed a peak (18%) with desired mass. The mixture was concentrated under reduced pressure. The crude was purified by prep-TLC (Dichloromethane:Methanol=9/1) followed by prep-HPLC (column: Phenomenex Synergi Polar-RP 100*25 mm*4 um; mobile phase: [water (TFA)-ACN]; B %: 25%-55%, 9 min), the eluent was lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-N-(7-((1-(3-(2,6-dioxopiperidin-3-yl)-1-methyl-1H-indazol-6-yl)piperidin-4-yl)methyl)-7-azaspiro[3.5]nonan-2-yl)-3-methoxybenzamide (14.0 mg, 14.65 μmol, 12.68% yield, 95% purity) as a white solid. MS (M+H)⁺=907.9

¹H NMR (400 MHz, DMSO-d₆) δ=10.86 (s, 1H), 8.99-8.80 (m, 1H), 8.53-8.45 (m, 1H), 8.29-8.22 (m, 2H), 8.16-8.10 (m, 1H), 7.54-7.47 (m, 3H), 6.95 (br d, J=7.7 Hz, 1H), 6.91-6.87 (m, 1H), 4.84-4.73 (m, 1H), 4.49-4.38 (m, 1H), 4.29-4.24 (m, 1H), 4.08 (br t, J=13.9 Hz, 2H), 3.95 (s, 3H), 3.90 (s, 3H), 3.87-3.81 (m, 2H), 3.42-3.38 (m, 2H), 3.34 (s, 3H), 3.08-2.94 (m, 3H), 2.88-2.77 (m, 3H), 2.64-2.61 (m, 1H), 2.41-2.36 (m, 1H), 2.30-2.26 (m, 1H), 2.23-2.12 (m, 2H), 2.03-1.69 (m, 14H), 1.67-1.52 (m, 4H), 1.43-1.32 (m, 2H).

### Example 40. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(7-((1-(3-(2,4-dioxotetrahydropyrimidin-1 (2H)-yl)-1-methyl-1H-indazol-6-yl)piperidin-4-yl)methyl)-7-azaspiro[3.5]nonan-2-yl)-3-methoxybenzamide (Compound 40)

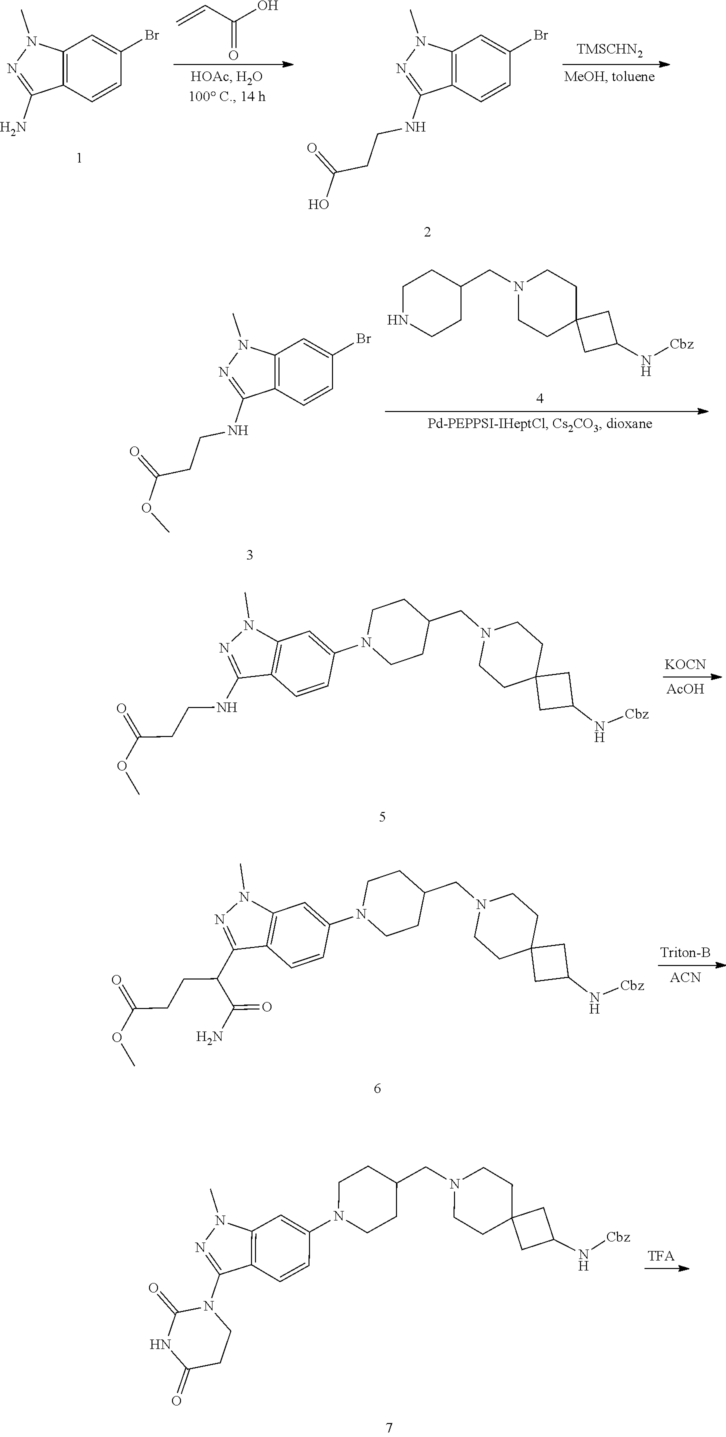

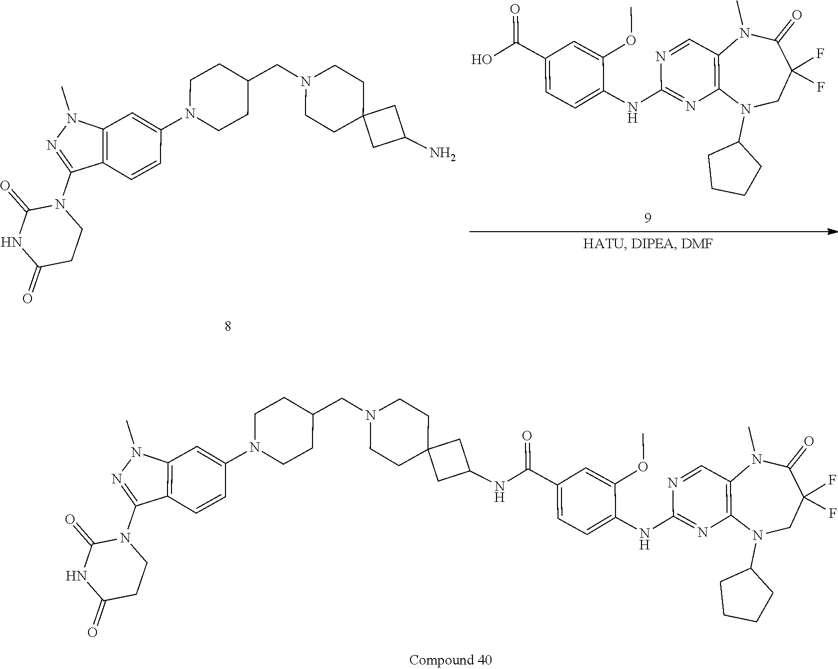 [SMILES: Cn1nc(N2CCC(=O)NC2=O)c2ccc(N3CCC(CN4CCC5(CC4)CC(N)C5)CC3)cc21 ; COc1cc(C(=O)O)ccc1Nc1ncc2c(n1)N(C1CCCC1)CC(F)(F)C(=O)N2C ; COc1cc(C(=O)NC2CC3(CCN(CC4CCN(c5ccc6c(N7CCC(=O)NC7=O)nn(C)c6c5)CC4)CC3)C2)ccc1Nc1ncc2c(n1)N(C1CCCC1)CC(F)(F)C(=O)N2C]

### Step 1. Synthesis of 3-((6-bromo-1-methyl-1H-indazol-3-yl)amino)propanoic acid (2)

To a solution of acrylic acid (956.27 mg, 13.27 mmol, 910.73 μL) and 6-bromo-1-methyl-1H-indazol-3-amine (3 g, 13.27 mmol) in H₂O (3 mL) was added HOAc (1.89 g, 31.47 mmol, 1.8 mL). The mixture was stirred at 100° C. for 14 h. LCMS showed the desired mass. The mixture was diluted with EtOAc (5 mL), MTBE (5 mL) and H₂O (10 mL), then filtered. The filter cake was washed with EtOAc (10 mL) and water (10 mL). The filter cake was collected and dried under reduced pressure. The filtrate was adjusted the pH=9 with Na₂CO₃ saturation solution and then washed with EtOAc (10 mL×3), the organic layer was discarded. The aqueous phase was adjusted the pH=3 with 1 N HCl, and extracted with EtOAc (10 mL×3). The combined organic layer was dried over Na₂SO₄ and filtered and concentrated under reduced pressure to afford 3-((6-bromo-1-methyl-1H-indazol-3-yl)amino)propanoic acid (1.67 g, crude) as a yellow solid. MS (M+H)⁺=298.2

### Step 2. Synthesis of methyl 3-((6-bromo-1-methyl-1H-indazol-3-yl)amino)propanoate (3)

To a solution of 3-((6-bromo-1-methyl-1H-indazol-3-yl)amino)propanoic acid (1.67 g, 5.60 mmol) in toluene (2 mL) and MeOH (2 mL) was added TMSCHN₂ (2 M, 8.40 mL) and the mixture was stirred at 20° C. for 14 h. LCMS showed the desired mass. The mixture was concentrated under reduced pressure. The crude combined with another batch (1 g scale), and diluted with MTBE (10 mL) and the mixture was stirred at 20° C. for 30 min. The mixture was filtered and the filter cake was washed with MTBE (10 mL). The combined filtrates was concentrated under reduced pressure. The residue was purified by flash silica gel chromatography (12 g SepaFlash® Silica Flash Column, Eluent of 40% EtOAc/Petroleum ether gradient@80 mL/min) to afford methyl 3-((6-bromo-1-methyl-1H-indazol-3-yl)amino)propanoate (1.3 g, 3.87 mmol, 69.09% yield, 93% purity) as a yellow oil. MS (M+H)⁺=312.1

### Step 3. Synthesis of methyl 3-((6-(4-((2-(((benzyloxy)carbonyl)amino)-7-azaspiro[3.5]nonan-7-yl)methyl)piperidin-1-yl)-1-methyl-1H-indazol-3-yl)amino)propanoate (5)

To a solution of methyl 3-((6-bromo-1-methyl-1H-indazol-3-yl)amino)propanoate (0.5 g, 1.60 mmol) and benzyl (7-(piperidin-4-ylmethyl)-7-azaspiro[3.5]nonan-2-yl)carbamate (734 mg, 1.80 mmol, HCl salt) in dioxane (13 mL) were added Cs₂CO₃ (1.57 g, 4.81 mmol) and PdPEPPSI-IHeptCl (31.16 mg, 32.03 μmol) and the mixture was stirred at 100° C. for 28 h. LCMS showed methyl 3-((6-bromo-1-methyl-1H-indazol-3-yl)amino)propanoate remained and additional Pd-PEPPSI-IHeptCl (31.16 mg, 32.03 μmol) was added at 20° C. and the mixture was stirred at 100° C. for another 48 h. LCMS showed a peak (28%) with desired mass. The mixture was filtered and the filter cake was washed with EtOAc (50 mL). The filtrate was concentrated under reduced pressure. The residue was purified by flash silica gel chromatography (12 g SepaFlash® Silica Flash Column, Eluent of 100-10% MeOH/EtOAc gradient@80 mL/min) followed by flash silica gel chromatography (12 g SepaFlash® Silica Flash Column, Eluent of 100% EtOAc/Petroleum ether gradient@80 mL/min) to afford methyl 3-((6-(4-((2-(((benzyloxy)carbonyl)amino)-7-azaspiro[3.5]nonan-7-yl)methyl)piperidin-1-yl)-1-methyl-1H-indazol-3-yl)amino)propanoate (190 mg, 277.39 μmol, 17.32% yield, 88% purity) as a yellow oil. MS (M+H)⁺=603.6

### Step 4. Synthesis of methyl 3-(1-(6-(4-((2-(((benzyloxy)carbonyl)amino)-7-azaspiro[3.5]nonan-7-yl)methyl)piperidin-1-yl)-1-methyl-1H-indazol-3-yl)ureido)propanoate (6)

To a solution of methyl 3-((6-(4-((2-(((benzyloxy)carbonyl)amino)-7-azaspiro[3.5]nonan-7-yl)methyl)piperidin-1-yl)-1-methyl-H-indazol-3-yl)amino)propanoate (190 mg, 277.39 μmol, 88% purity) in AcOH (4 mL) was added KOCN (35 mg, 411.06 μmol) and the mixture was stirred at 20° C. for 14 h. LCMS showed a peak (67%) with desired mass. The mixture was concentrated under reduced pressure. The crude was diluted with MeCN (3 mL) and water (30 mL) and lyophilized to afford methyl 3-(1-(6-(4-((2-(((benzyloxy)carbonyl)amino)-7-azaspiro[3.5]nonan-7-yl)methyl)piperidin-1-yl)-1-methyl-1H-indazol-3-yl)ureido)propanoate (0.2 g, crude) as a yellow oil. MS (M+H)⁺=646.5

### Step 5. Synthesis of benzyl (7-((1-(3-(2,4-dioxotetrahydropyrimidin-1 (2H)-yl)-1-methyl-1H-indazol-6-yl)piperidin-4-yl)methyl)-7-azaspiro[3.5]nonan-2-yl)carbamate (7)

To a solution of methyl 3-(1-(6-(4-((2-(((benzyloxy)carbonyl)amino)-7-azaspiro[3.5]nonan-7-yl)methyl)piperidin-1-yl)-1-methyl-1H-indazol-3-yl)ureido)propanoate (190 mg, 294.21 μmol) in MeCN (4 mL) was added Triton B (828.00 mg, 1.98 mmol, 0.9 mL, 40% purity) slowly and the mixture was stirred at 20° C. for 1 h. LCMS showed a main peak (90%) with desired mass. The mixture was diluted with water (10 mL) and extracted with EtOAc/MeOH=10/1 (10 mL×3). The combined organic layer was washed with water (10 mL×3), dried over Na₂SO₄ and filtered. The filtrate was concentrated under reduced pressure. The residue was purified by flash silica gel chromatography (5 g SepaFlash® Silica Flash Column, Eluent of 0˜30% MeOH/EtOAc gradient@80 mL/min) to afford benzyl (7-((1-(3-(2,4-dioxotetrahydropyrimidin-1 (2H)-yl)-1-methyl-1H-indazol-6-yl)piperidin-4-yl)methyl)-7-azaspiro[3.5]nonan-2-yl)carbamate (110 mg, 161.30 μmol, 54.83% yield, 90% purity) as a yellow solid. MS (M+H)⁺=614.6

### Step 6. Synthesis of 1-(6-(4-((2-amino-7-azaspiro[3.5]nonan-7-yl)methyl)piperidin-1-yl)-1-methyl-1H-indazol-3-yl)dihydropyrimidine-2,4 (1H,3H)-dione (8)

A solution of benzyl (7-((1-(3-(2,4-dioxotetrahydropyrimidin-1 (2H)-yl)-1-methyl-1H-indazol-6-yl)piperidin-4-yl)methyl)-7-azaspiro[3.5]nonan-2-yl)carbamate (95 mg, 154.79 μmol) in TFA (1.5 mL) was stirred at 60° C. for 2 h. LCMS showed a main peak (95%) with desired mass. The mixture was concentrated under reduced pressure to afford 1-(6-(4-((2-amino-7-azaspiro[3.5]nonan-7-yl)methyl)piperidin-1-yl)-1-methyl-1H-indazol-3-yl)dihydropyrimidine-2,4 (1H,3H)-dione (90 mg, crude, TFA salt) as a yellow oil. MS (M+H)⁺=480.5

### Step 7. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(7-((1-(3-(2,4-dioxotetrahydropyrimidin-1 (2H)-yl)-1-methyl-1H-indazol-6-yl)piperidin-4-yl)methyl)-7-azaspiro[3.5]nonan-2-yl)-3-methoxybenzamide (Compound 40)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (60 mg, 134.10 μmol) in DMF (0.7 mL) were added HATU (56.09 mg, 147.51 μmol) and DIPEA (29.68 mg, 229.64 μmol, 40 μL) and the mixture was stirred at 20° C. for 15 min. Then a solution of 1-(6-(4-((2-amino-7-azaspiro[3.5]nonan-7-yl)methyl)piperidin-1-yl)-1-methyl-1H-indazol-3-yl)dihydropyrimidine-2,4 (1H,3H)-dione (90 mg, 151.61 μmol, TFA salt) and DIPEA (89.04 mg, 688.93 μmol, 120.00 L) in DMF (0.8 mL) was added and the mixture was stirred at 20° C. for 1 h. LCMS showed a peak (65%) with desired mass. The mixture was diluted with water (20 mL) and extracted with EtOAc (10 mL×3). The combined organic layer was washed with brine (10 mL×3), dried over Na₂SO₄ and filtered. The filtrate was concentrated under reduced pressure. The crude was purified by prep-TLC (Dichloromethane:Methanol=10:1). The product was diluted with MeCN (3 mL), MeOH (2 mL) and deionized water (30 mL), then the mixture was lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-N-(7-((1-(3-(2,4-dioxotetrahydropyrimidin-1 (2H)-yl)-1-meth yl-1H-indazol-6-yl)piperidin-4-yl)methyl)-7-azaspiro[3.5]nonan-2-yl)-3-methoxybenz amide (61.1 mg, 63.18 μmol, 47.12% yield, 94% purity) as a yellow solid. MS (M+H)⁺=908.9

¹H NMR (400 MHz, DMSO-d₆) δ=10.50 (s, 1H), 8.48-8.41 (m, 1H), 8.30-8.25 (m, 2H), 7.97 (s, 1H), 7.50-7.46 (m, 2H), 7.43 (d, J=9.2 Hz, 1H), 6.90 (br d, J=9.0 Hz, 1H), 6.81 (s, 1H), 4.81-4.72 (m, 1H), 4.45-4.36 (m, 1H), 4.05 (br t, J=14.2 Hz, 2H), 3.94 (s, 3H), 3.91-3.86 (m, 5H), 3.84-3.76 (m, 2H), 3.30 (s, 3H), 2.79-2.70 (m, 4H), 2.63-2.55 (m, 3H), 2.25-2.10 (m, 4H), 1.98-1.91 (m, 3H), 1.86-1.77 (m, 4H), 1.75-1.52 (m, 11H), 1.34-1.18 (m, 2H).

### Example 41. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(4-(7-(1-(2,6-dioxopiperidin-3-yl)-3-methyl-2-oxo-2,3-dihydro-1H-benzo[d]imidazol-4-yl)-2,7-diazaspiro[3.5]nonan-2-yl)piperidin-1-yl)-3-methoxybenzamide (Compound 41)

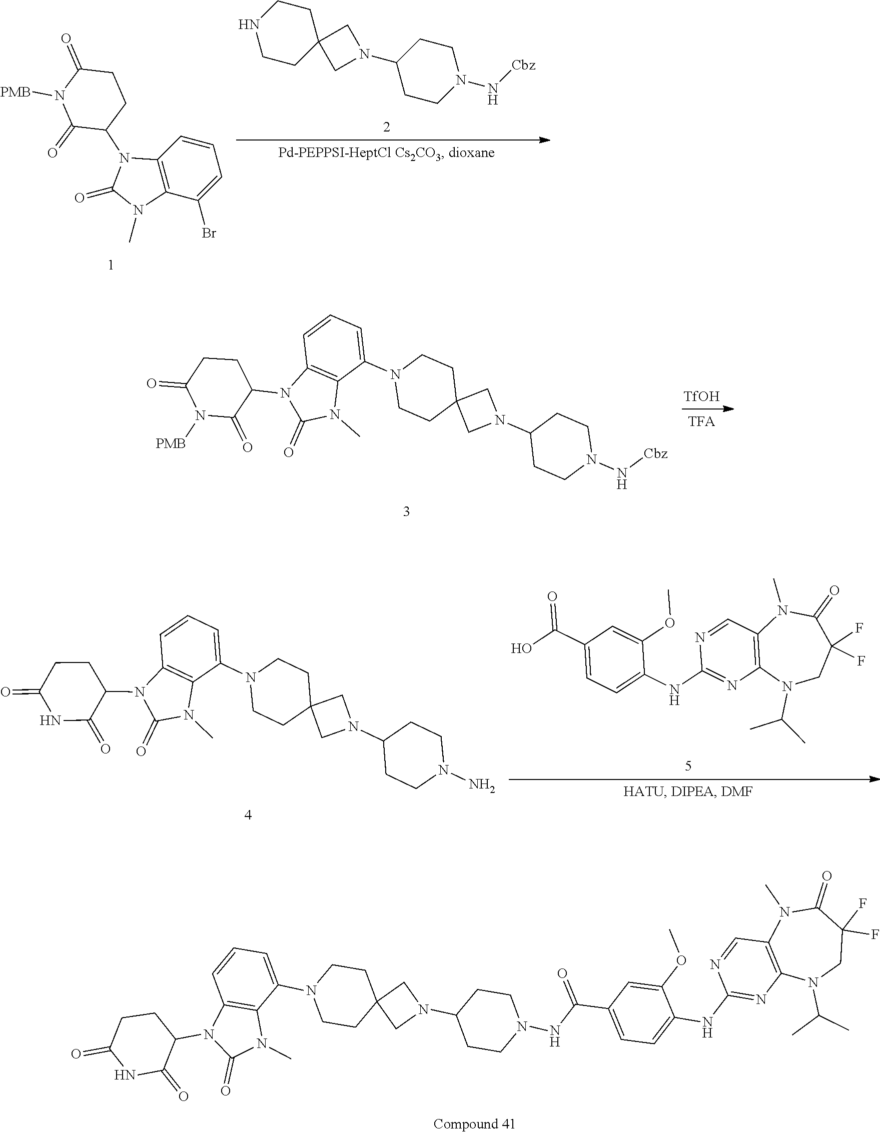

### Step 1. Synthesis of benzyl (4-(7-(1-(1-(4-methoxybenzyl)-2,6-dioxopiperidin-3-yl)-3-methyl-2-oxo-2,3-dihydro-1H-benzo[d]imidazol-4-yl)-2,7-diazaspiro[3.5]nonan-2-yl)piperidin-1-yl)carbamate (3)

To a solution of 3-(4-bromo-3-methyl-2-oxo-2,3-dihydro-1H-benzo[d]imidazol-1-yl)-1-(4-methoxybenzyl)piperidine-2,6-dione (360 mg, 785.50 μmol) and benzyl (4-(2,7-diazaspiro[3.5]nonan-2-yl)piperidin-1-yl)carbamate (310.23 mg, 785.50 μmol, HCl) in dioxane (15 mL) was added Pd-PEPPSI-IHeptCl (76.41 mg, 78.55 μmol) and Cs₂CO₃ (1.28 g, 3.93 mmol), the mixture was stirred at 100° C. for 16 h. LCMS showed a peak (19%) with desired mass. The mixture was filtered through a pad of celite, the filtrate was concentrated in vacuum. The residue was purified by flash silica gel chromatography (Biotage; 20 g SepaFlash® Silica Flash Column, Eluent of 20-100% EtOAc/Petroleum ether to 10% Methanol/EtOAc gradient@40 mL/min) and re-purified by prep-TLC (SiO₂, DCM:MeOH=9:1) to afford benzyl (4-(7-(1-(1-(4-methoxybenzyl)-2,6-dioxopiperidin-3-yl)-3-methyl-2-oxo-2,3-dihydro-1H-benzo[d]imidazol-4-yl)-2,7-diazaspiro[3.5]nonan-2-yl)piperidin-1-yl)carbamate (110 mg, 136.03 μmol, 17.32% yield, 91% purity) as a yellow solid. MS (M+H)⁺=736.4

### Step 2. Synthesis of 3-(4-(2-(1-aminopiperidin-4-yl)-2,7-diazaspiro[3.5]nonan-7-yl)-3-methyl-2-oxo-2,3-dihydro-1H-benzo[d]imidazol-1-yl)piperidine-2,6-dione (4)

To a solution of benzyl (4-(7-(1-(1-(4-methoxybenzyl)-2,6-dioxopiperidin-3-yl)-3-methyl-2-oxo-2,3-dihydro-1H-benzo[d]imidazol-4-yl)-2,7-diazaspiro[3.5]nonan-2-yl)piperidin-1-yl)carbamate (100 mg, 135.89 μmol) in TFA (1 mL) was added TfOH (340.00 mg, 2.27 mmol, 0.2 mL), the mixture was stirred at 100° C. for 3 h. LCMS showed a peak (32%) with desired mass. The mixture was concentrated in vacuum to afford 3-(4-(2-(1-aminopiperidin-4-yl)-2,7-diazaspiro[3.5]nonan-7-yl)-3-methyl-2-oxo-2,3-di hydro-1H-benzo[d]imidazol-1-yl)piperidine-2,6-dione (70 mg, crude, TFA) as a brown oil, which was used into the next step directly. MS (M+H)⁺=482.3.

### Step 3. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(4-(7-(1-(2,6-dioxopiperidin-3-yl)-3-methyl-2-oxo-2,3-dihydro-1H-benzo[d]imidazol-4-yl)-2,7-diazaspiro[3.5]nonan-2-yl)piperidin-1-yl)-3-methoxybenzamide (Compound 41)

To a solution of 4-((7,7-difluoro-9-isopropyl-5-methyl-6-oxo-6,7,8,9-tetrahydro-5H-pyrimido[4,5-b][1,4]diazepin-2-yl)amino)-3-methoxybenzoic acid (56.00 mg, 132.89 μmol) in DMF (2 mL) were added HATU (67.03 mg, 176.29 μmol) and DIPEA (45.57 mg, 352.58 μmol, 61.41 μL), the mixture was stirred at 20° C. for 0.5 h, 3-(4-(2-(1-aminopiperidin-4-yl)-2,7-diazaspiro[3.5]nonan-7-yl)-3-methyl-2-oxo-2,3-di hydro-1H-benzo[d]imidazol-1-yl)piperidine-2,6-dione (70 mg, 117.53 μmol, TFA) was added, the mixture was stirred at 20° C. for 2 h. LCMS showed a peak (36%) with desired mass. The reaction mixture was diluted with water 5 mL, extracted with EtOAc (10 mL×5). The combined organic layers were washed with brine (10 mL×2), dried over Na₂SO₄, filtered and concentrated under reduced pressure to give a residue. The residue was purified by prep-TLC (SiO₂, DCM:MeOH=9:1) and re-purified by reversed-phase HPLC (column: Waters Xbridge 150×25 mm×5 μm; mobile phase: [water (NH₄HCO₃)-ACN]; B %: 33%-63%, 9 min). The eluent was lyophilized to afford 4-((7,7-difluoro-9-isopropyl-5-methyl-6-oxo-6,7,8,9-tetrahydro-5H-pyrimido[4,5-b][1,4]diazepin-2-yl)amino)-N-(4-(7-(1-(2,6-dioxopiperidin-3-yl)-3-methyl-2-oxo-2,3-dihydro-1H-benzo[d]imidazol-4-yl)-2,7-diazaspiro[3.5]nonan-2-yl)piperidin-1-yl)-3-methoxybenzamide (10.2 mg, 11.18 μmol, 9.51% yield, 97% purity) as a white solid. MS (M+H)⁺=885.5.

¹H NMR (400 MHz, DMSO-d₆) δ=11.14-11.04 (m, 1H), 9.28 (s, 1H), 8.30 (d, J=5.9 Hz, 1H), 8.22 (s, 1H), 7.87 (s, 1H), 7.47-7.40 (m, 2H), 7.00-6.93 (m, 1H), 6.90 685 (m, 2H), 5.41-5.29 (m, 1H), 4.94-4.81 (m, 1H), 4.08-3.99 (m, 2H), 3.93 (s, 3H), 3.62 (s, 3H), 3.30 (s, 3H), 3.06-2.92 (m, 9H), 2.81-2.73 (m, 3H), 2.70-2.67 (m, 2H), 2.17-2.09 (m, 1H), 2.03-1.97 (m, 1H), 1.91-1.79 (m, 4H), 1.75-1.67 (m, 2H), 1.37-1.30 (m, 2H), 1.27-1.21 (m, 7H).

### Example 42. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(7-((1-(6-(6,8-dioxo-2,7-diazaspiro[4.5]decan-2-yl) pyridin-2-yl)piperidin-4-yl)methyl)-7-azaspiro[3.5]nonan-2-yl)-3-methoxybenzamide (Compound 42)

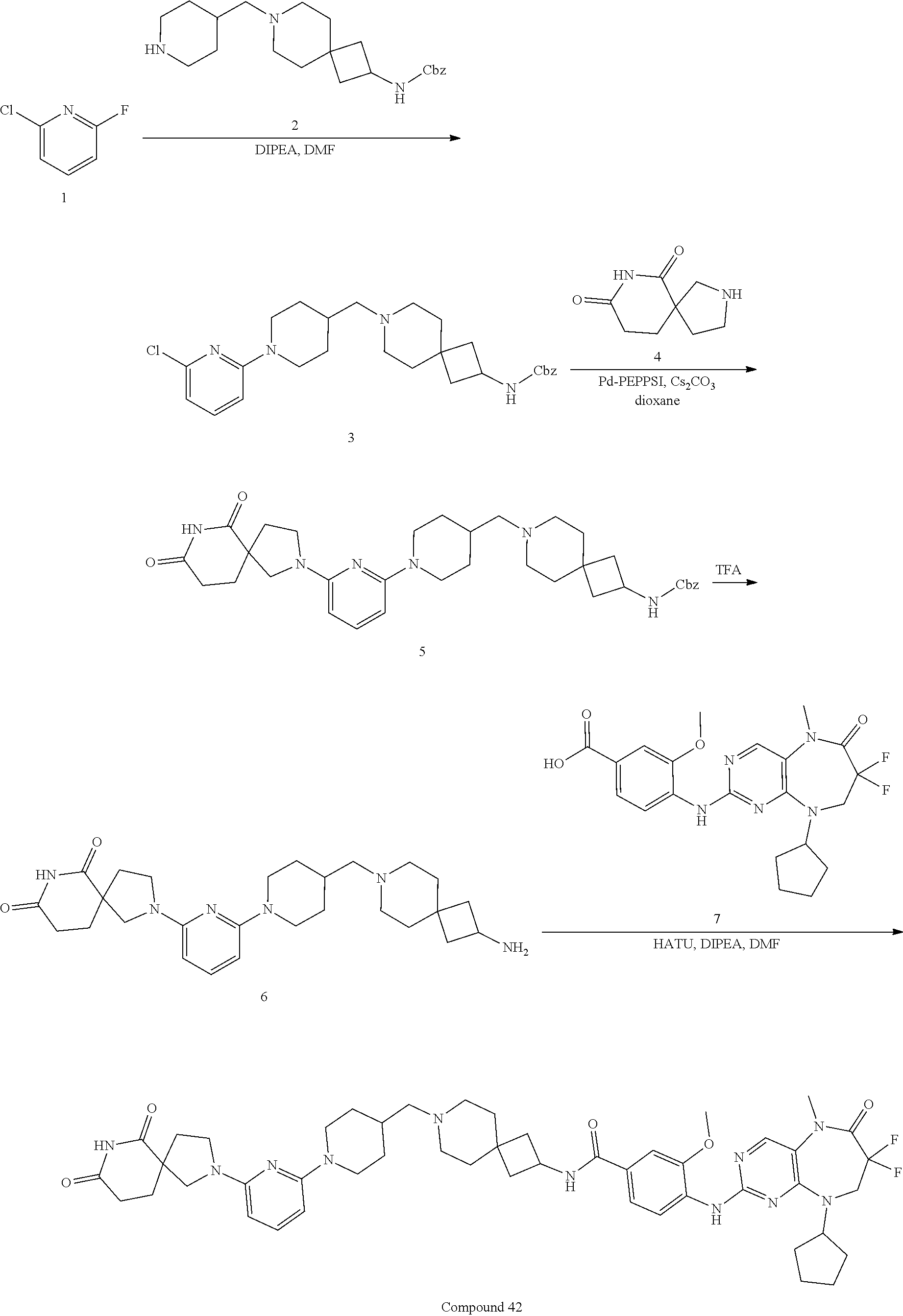

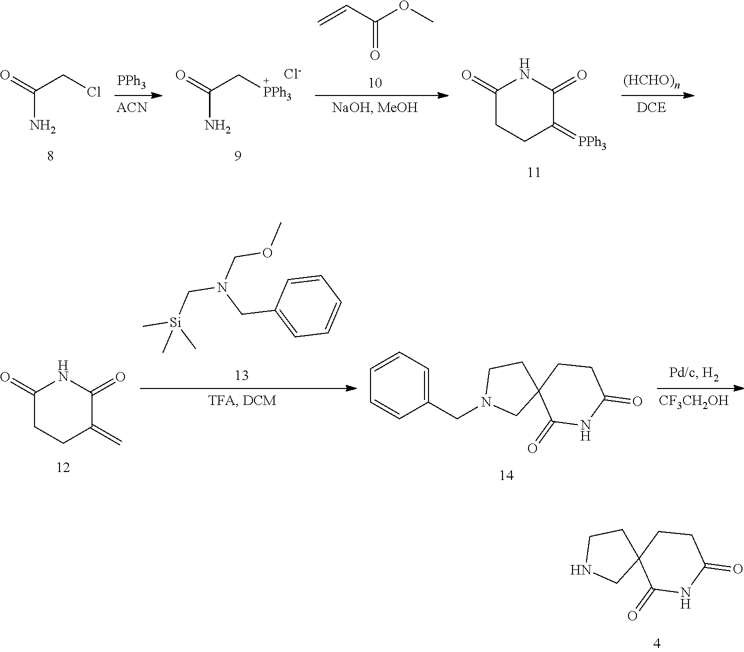

### Step 1. Synthesis of benzyl (7-((1-(6-chloropyridin-2-yl)piperidin-4-yl)methyl)-7-azaspiro[3.5]nonan-2-yl)carbamate (3)

To a solution of 2-chloro-6-fluoro-pyridine (300 mg, 2.28 mmol) and benzyl (7-(piperidin-4-ylmethyl)-7-azaspiro[3.5]nonan-2-yl)carbamate (600 mg, 1.47 mmol, HCl) in DMF (6 mL) was added DIPEA (884.32 mg, 6.84 mmol, 1.19 mL) at 20° C. and the resulting mixture was stirred at 60° C. for 12 h. LCMS showed starting material was consumed completely and 22% peak with desired mass was detected. The reaction mixture was diluted with H₂O (15 mL) and extracted with EtOAc (15 mL×3). The organic layer was washed with brine (15 mL×3), dried over Na₂SO₄, filtered and concentrated. The residue was purified by flash silica gel chromatography (10 g SepaFlash® Silica Flash Column, Eluent of 0˜100% EtOAc/Petroleum ether gradient @100 mL/min) to afford benzyl (7-((1-(6-chloropyridin-2-yl)piperidin-4-yl)methyl)-7-azaspiro[3.5]nonan-2-yl)carbamate (430 mg, 863.48 μmol, 37.86% yield, 97% purity) as a white solid. MS (M+H)⁺=483.4

### Step 2. Synthesis of (2-amino-2-oxoethyl)triphenylphosphonium chloride (9)

To a solution of 2-chloroacetamide (10 g, 106.94 mmol) in ACN (100 mL) was added PPh₃ (29.45 g, 112.28 mmol) at 20° C. and the resulting mixture was stirred at 80° C. for 16 h. LCMS showed starting material was consumed completely and a peak (54%) with desired mass. The reaction mixture was filtered and the filter cake was dried in vacuum. The crude product was triturated with EtOAc (100 mL) at 20° C. for 15 min and filtered. The filter cake was dried in vacuum to afford (2-amino-2-oxoethyl)triphenylphosphonium chloride (27.2 g, 84.91 mmol, 79.40% yield, 100% purity) as a white solid. MS (M+H)⁺=320.4

### Step 3. Synthesis of 3-(triphenyl-15-phosphaneylidene)piperidine-2,6-dione (11)

To a solution of (2-amino-2-oxoethyl)triphenylphosphonium chloride (20 g, 62.43 mmol) in MeOH (200 mL) was added NaOH (2.50 g, 62.43 mmol) and methyl prop-2-enoate (6.45 g, 74.92 mmol, 6.75 mL) at 20° C. and the resulting mixture was stirred at 20 for 12 h. LCMS showed starting material was consumed completely and a peak (33%) with desired mass. The reaction mixture was concentrated in vacuum to afford 3-(triphenyl-15-phosphaneylidene)piperidine-2,6-dione (20 g, crude) as a white solid. MS (M+H)⁺=374.3

### Step 4. Synthesis of 3-methylenepiperidine-2,6-dione (12)

To a solution of 3-(triphenyl-15-phosphaneylidene)piperidine-2,6-dione (20 g, 53.56 mmol) in DCE (200 mL) was added paraformaldehyde (1.93 g) at 20° C. and the resulting mixture was stirred at 80° C. for 1 h. LCMS showed starting material was consumed completely and a peak with desired mass. The reaction mixture was concentrated in vacuum to afford 3-methylenepiperidine-2,6-dione (6.7 g, crude) as a white solid. MS (M+H)⁺=126.1

### Step 5. Synthesis of 2-benzyl-2,7-diazaspiro[4.5]decane-6,8-dione (14)

To a solution of 3-methylenepiperidine-2,6-dione (6.7 g, 53.55 mmol) and N-benzyl-1-methoxy-N-((trimethylsilyl)methyl)methanamine (10.17 g, 42.84 mmol) in DCM (100 mL) was added TFA (3.97 g, 34.81 mmol, 2.58 mL) at 20° C. and the resulting mixture was stirred at 20° C. for 16 h. LCMS showed starting material was consumed completely and a peak with desired mass. The reaction mixture was concentrated in vacuum. The residue was purified by flash silica gel chromatography (80 g SepaFlash® Silica Flash Column, Eluent of 0˜100% EtOAc/Petroleum ether gradient @100 mL/min) to afford impure product, which was triturated with mix solution (20 mL, MTBE:EtOAc=5:1) at 20° C. for 15 min and filtered. The filtrate was dried in vacuum to afford impure product B, which was re-purified by flash silica gel chromatography (80 g SepaFlash® Silica Flash Column, Eluent of 0˜100% EtOAc/Petroleum ether gradient@100 mL/min) and flash silica gel chromatography (40 g SepaFlash® Silica Flash Column, Eluent of 0˜100% EtOAc/Petroleum ether gradient@100 mL/min) to afford 2-benzyl-2,7-diazaspiro[4.5]decane-6,8-dione (976 mg, 3.78 mmol, 32.53% yield) as a light yellow solid. MS (M+H)⁺=259.1

### Step 6. Synthesis of 2,7-diazaspiro[4.5]decane-6,8-dione (4)

To a solution of 2-benzyl-2,7-diazaspiro[4.5]decane-6,8-dione (976 mg, 3.78 mmol) in CF₃CH₂OH (10 mL) was added Pd/C (100 mg, 10% purity) under N₂ atmosphere. The suspension was degassed and purged with H₂ for 3 times. The mixture was stirred at 20° C. for 16 h under H₂ (15 Psi). LCMS showed starting material was consumed completely and a peak with desired mass. The reaction mixture was diluted with CF₃ CH₂OH (30 mL) and filtered. The filtrate was concentrated in vacuum to afford 2,7-diazaspiro[4.5]decane-6,8-dione (742 mg, crude) as a yellow oil. MS (M+H)⁺=169.0

### Step 7. Synthesis of benzyl (7-((1-(6-(6,8-dioxo-2,7-diazaspiro[4.5]decan-2-yl)pyridin-2-yl)piperidin-4-yl)meth yl)-7-azaspiro[3.5]nonan-2-yl)carbamate (5)

To a solution of benzyl (7-((1-(6-chloropyridin-2-yl)piperidin-4-yl)methyl)-7-azaspiro[3.5]nonan-2-yl)carbamate (100 mg, 207.02 μmol) and 2,7-diazaspiro[4.5]decane-6,8-dione (104.46 mg, 621.06 μmol) in dioxane (3 mL) were added Pd-PEPPSI-IHeptCl (10.07 mg, 10.35 mol) and Cs₂CO₃ (202.35 mg, 621.06 μmol) at 20° C. under N₂ and the resulting reaction mixture was stirred at 100° C. for 12 h under N₂. LCMS showed 12% of benzyl (7-((1-(6-chloropyridin-2-yl)piperidin-4-yl)methyl)-7-azaspiro[3.5]nonan-2-yl)carbamate remained and a peak (70%) with desired mass. The reaction mixture was concentrated in vacuum. The residue was purified by prep-TLC (SiO₂, EtOAc:Methanol=10:1) to afford benzyl (7-((1-(6-(6,8-dioxo-2,7-diazaspiro[4.5]decan-2-yl)pyridin-2-yl)piperidin-4-yl)methyl)-7-azaspiro[3.5]nonan-2-yl)carbamate (66 mg, 101.99 μmol, 49.26% yield, 95% purity) as a white solid. MS (M+H)⁺=615.3

### Step 8. Synthesis of 2-(6-(4-((2-amino-7-azaspiro[3.5]nonan-7-yl)methyl)piperidin-1-yl)pyridin-2-yl)-2, 7-diazaspiro[4.5]decane-6,8-dione (6)

A mixture of benzyl (7-((1-(6-(6,8-dioxo-2,7-diazaspiro[4.5]decan-2-yl)pyridin-2-yl)piperidin-4-yl)methyl)-7-azaspiro[3.5]nonan-2-yl)carbamate (66 mg, 107.36 μmol) in TFA (2 mL) at 20° C. and the resulting reaction mixture was stirred at 40° C. for 13 h. LCMS showed starting material was consumed completely and a peak (77%) with desired mass. The reaction mixture was concentrated in vacuum to afford 2-(6-(4-((2-amino-7-azaspiro[3.5]nonan-7-yl)methyl)piperidin-1-yl)pyridin-2-yl)-2,7-diazaspiro[4.5]decane-6,8-dione (64 mg, crude, TFA) as a yellow oil. MS (M+H)⁺=481.3

### Step 9. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(7-((1-(6-(6,8-dioxo-2,7-diazaspiro[4.5]decan-2-yl) pyridin-2-yl)piperidin-4-yl)methyl)-7-azaspiro[3.5]nonan-2-yl)-3-methoxybenzamide (Compound 42)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (50 mg, 111.75 μmol) in DMF (1 mL) were added HATU (46.74 mg, 122.92 μmol) and DIPEA (28.89 mg, 223.50 μmol, 38.93 μL). The mixture was stirred at 20° C. for 10 min and a solution of 2-(6-(4-((2-amino-7-azaspiro[3.5]nonan-7-yl)methyl)piperidin-1-yl)pyridin-2-yl)-2,7-diazaspiro[4.5]decane-6,8-dione (64 mg, 107.62 μmol, TFA) in DMF (1 mL) with DIPEA (43.33 mg, 335.24 μmol, 58.39 L) was added and the resulting mixture was stirred at 20° C. for 1 h. LCMS showed all starting material was consumed completely and a peak (28%) with desired mass. The reaction mixture was diluted with H₂O (10 mL) and extracted with EtOAc (10 mL×3). The organic layer was washed with brine (10 mL×3), dried over Na₂SO₄, filtered and concentrated. The residue was purified prep-TLC (SiO₂, DCM:MeOH=10:1) and lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-N-(7-((1-(6-(6,8-dioxo-2,7-diazaspiro[4.5]decan-2-yl)pyridin-2-yl)piperidin-4-yl)methyl)-7-azaspiro[3.5]nonan-2-yl)-3-methoxybenzamide (16.1 mg, 15.92 μmol, 14.25% yield, 90% purity) as a light yellow solid. MS (M+H)⁺=910.2

¹H NMR (400 MHz, DMSO-d₆) δ=10.73 (s, 1H), 8.43 (br d, J=7.1 Hz, 1H), 8.31-8.21 (m, 2H), 7.96 (s, 1H), 7.53-7.44 (m, 2H), 7.23 (t, J=8.1 Hz, 1H), 5.97 (d, J=8.2 Hz, 1H), 5.68 (d, J=7.9 Hz, 1H), 4.83-4.71 (m, 1H), 4.46-4.32 (m, 1H), 4.28-4.15 (m, 2H), 4.04 (br t, J=14.1 Hz, 2H), 3.94 (s, 3H), 3.72 (br d, J=10.9 Hz, 1H), 3.52-3.41 (m, 3H), 3.31 (br s, 3H), 2.66-2.56 (m, 4H), 2.31-2.27 (m, 2H), 2.21 (br dd, J=4.5, 6.4 Hz, 2H), 2.18-2.12 (m, 2H), 2.09 (br d, J=5.5 Hz, 2H), 2.04-1.98 (m, 2H), 1.97-1.90 (m, 3H), 1.81 (br t, J=10.0 Hz, 2H), 1.73-1.68 (m, 4H), 1.63-1.52 (m, 8H), 1.30-1.19 (m, 2H), 1.11-0.96 (m, 2H).

### Example 43. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(7-((1-(6-(6,8-dioxo-2,7-diazaspiro[4.5]decan-2-yl) pyridin-3-yl)piperidin-4-yl)methyl)-7-azaspiro[3.5]nonan-2-yl)-3-methoxybenzamide (Compound 43)

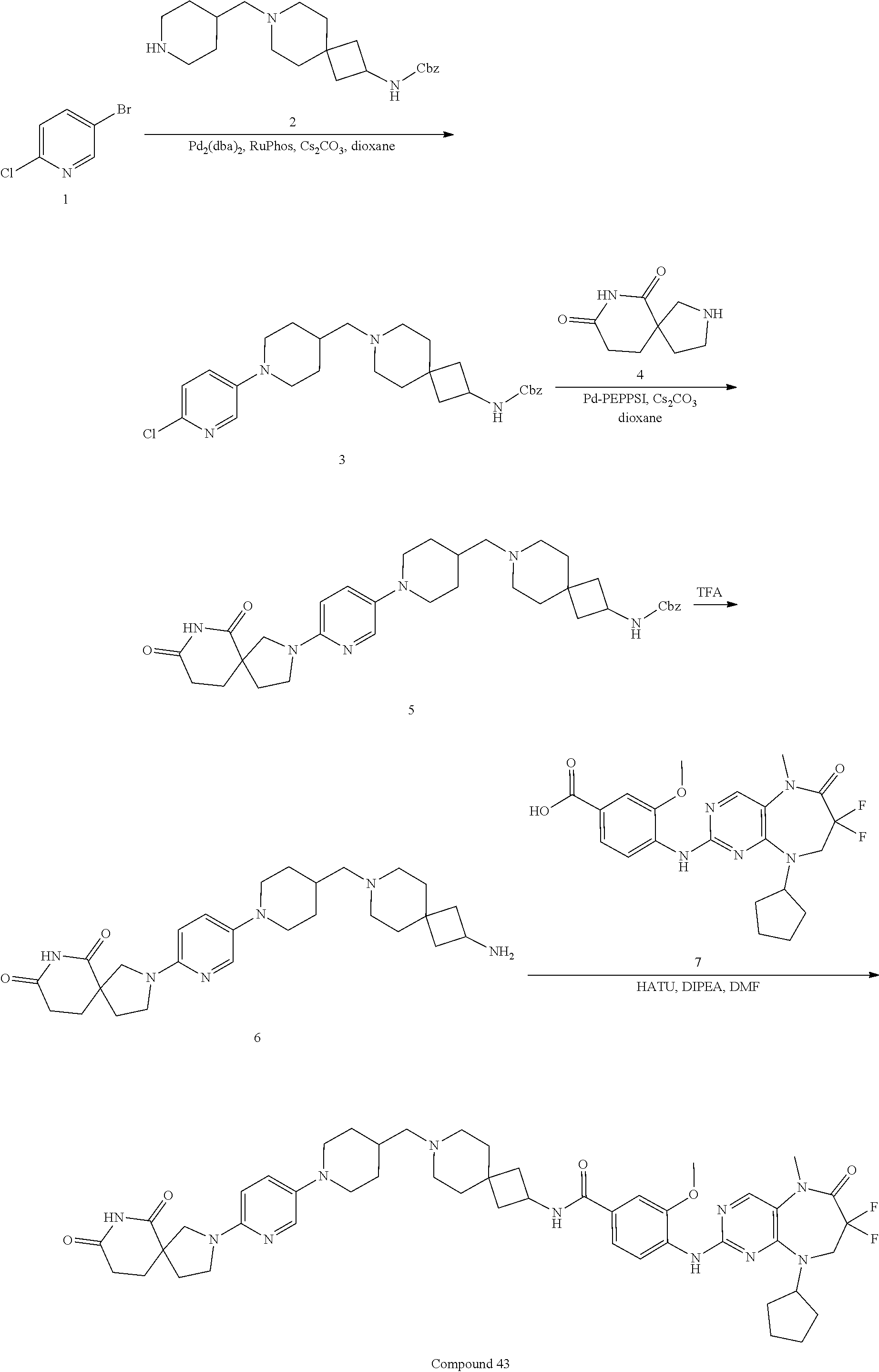

MS (M+H)⁺=911.1, ¹H NMR (400 MHz, DMSO-d₆) δ=10.72 (s, 1H), 8.46 (br d, J=7.6 Hz, 1H), 8.29-8.24 (m, 2H), 7.96 (s, 1H), 7.78 (d, J=2.9 Hz, 1H), 7.52-7.46 (m, 2H), 7.27 (dd, J=2.7, 9.2 Hz, 1H), 6.40 (d, J=9.1 Hz, 1H), 4.83-4.71 (m, 1H), 4.46-4.32 (m, 1H), 4.04 (t, J=14.0 Hz, 2H), 3.94 (s, 3H), 3.71 (d, J=10.6 Hz, 1H), 3.51-3.38 (m, 5H), 3.31 (s, 3H), 2.61-2.57 (m, 2H), 2.29-2.18 (m, 3H), 2.17 (br s, 1H), 2.15 (br d, J=1.4 Hz, 1H), 2.12 (br d, J=5.5 Hz, 2H), 2.05-1.98 (m, 3H), 1.96-1.90 (m, 3H), 1.85-1.79 (m, 2H), 1.78-1.69 (m, 4H), 1.62-1.54 (m, 8H), 1.28-1.17 (m, 6H).

### Example 44. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(7-((1-(1-(2,6-dioxopiperidin-3-yl)-3-methyl-2-oxo-2, 3-dihydro-1H-benzo[d]imidazol-5-yl)piperidin-4-yl)methyl)-7-azaspiro[3.5]nonan-2-yl)-3-methoxybenzamide (Compound 44)

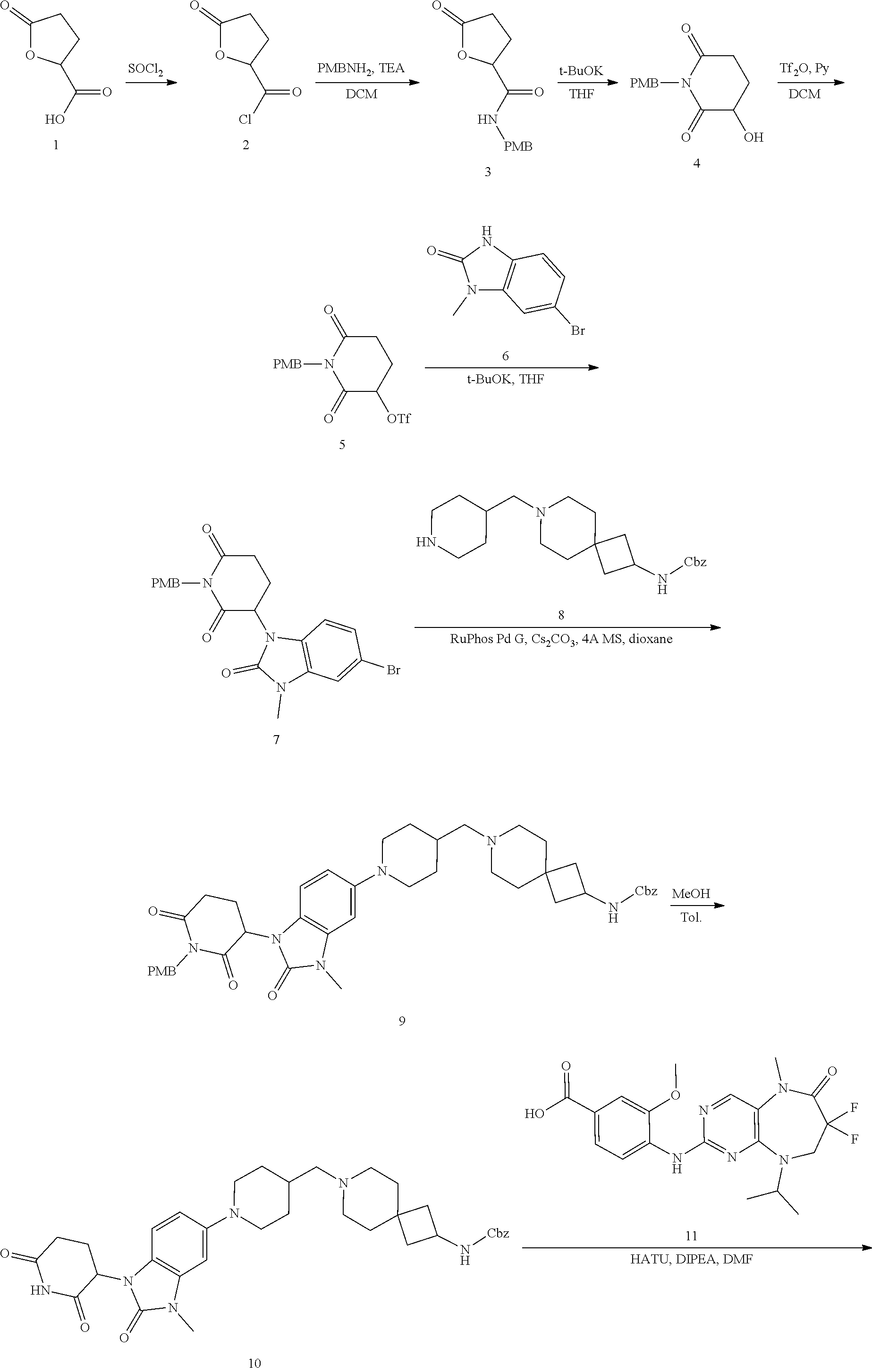

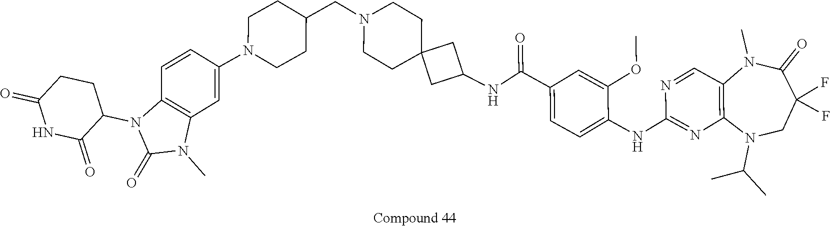 [SMILES: COc1cc(C(=O)NC2CC3(CCN(CC4CCN(c5ccc6c(c5)n(C)c(=O)n6C5CCC(=O)NC5=O)CC4)CC3)C2)ccc1Nc1ncc2c(n1)N(C(C)C)CC(F)(F)C(=O)N2C]

MS (M+H)⁺=899.0, ¹H NMR (400 MHz, DMSO-d₆) δ=11.06 (s, 1H), 8.52-8.39 (m, 1H), 8.34-8.27 (m, 1H), 8.22 (s, 1H), 7.89 (s, 1H), 7.54-7.46 (m, 2H), 6.96-6.91 (m, 1H), 6.86-6.78 (m, 1H), 6.69-6.58 (m, 1H), 5.34-5.25 (m, 1H), 4.93-4.82 (m, 1H), 4.49-4.35 (m, 1H), 4.10-3.99 (m, 2H), 3.94 (s, 3H), 3.69-3.54 (m, 2H), 3.31-3.28 (m, 6H), 3.09-2.83 (m, 3H), 2.72-2.57 (m, 4H), 2.22-2.09 (m, 3H), 2.03-1.95 (m, 2H), 1.86-1.76 (m, 4H), 1.65-1.52 (m, 2H), 1.41-1.13 (m, 13H).

### Experimental Examples

### 1. Western Blot Assay for PLK1

### (1) Culture of HeLa Cell Line

The HeLa cell line was purchased from Korea Cell Line Bank (KCLB), Seoul, Korea. The passage in cell culture was maintained at P115 to P125.

For cell counting, cell counter (Thermo Fisher Scientific Inc., Catalog #AMQAX1000) and 0.4% trypan blue solution were used.

For cell culture, DMEM (Gibco, Cat. No. 1195-65; Lot. No. 2085318), FBS (Gibco, Cat. No. 16000-044; Lot. No. 2097593), Penicillin/Streptomycin (PS) (Gibco, Cat. No. 15140-122; Lot. No. 2058855), 100 mm² cell culture dish (SPL, Cat. No. 20100), 150 mm² cell culture dish (SPL, Cat. No. 20150), 12-well culture plate (SPL, Cat. No. 30012), PBS pH 7.4 (Gibco, Cat. No. 10010-023; Lot. No. 2085080), TrypLE™ Express (Gibco, Cat. No. 12605-010; Lot No. 2070638), Counting Chamber (Hematocytometer) (Hirschmann, Cat. No. 8100204), and 0.4% Trypan Blue Solution (DYNEBIO, Cat. No. CBT3710; Lot. No. 20190723) were used.

### (2) Treatment of Compounds of the Present Invention

2×10⁵ cells were seeded for each well of a 12-well plate (SPL), and the cells were cultured in the culture medium in a total volume of 2 mL.

The compounds of Examples were completely dissolved in DMSO and used in the experiment, and thymidine was completely dissolved in DW and used in the experiment. For thymidine block, the products were treated with 2 mM of thymidine (Sigma-Aldrich Cat. No. T9250-5G) and then incubated for 24 hours.

For release and chemical treatment, the medium was suctioned and washed 3 times with 1×PBS. Complete media was added, followed by incubation for 4 hours in a CO₂ incubator. Each compound was diluted three folds from the highest concentration of 3 μM to the lowest concentration and then incubated for 6 hours again.

### (3) Western Blotting

For SDS-PAGE and Western blotting, 1×RIPA lysis buffer (Rockland, Cat. No. MB-030-0050; Lot no. 39751), 100× Protease Inhibitor Cocktail (Quartett, Cat. No. PPI1015; Lot no. PC050038424), Pierce™ BCA protein assay kit (ThermoScientific, Cat. No. 23225; Lot no. UC276876), albumin standard (ThermoScientific, Cat. No. 23209; Lot no. UB269561), 4-15% Mini-PROTEAN TGX stain-free gel (Bio-rad, Cat. No. 4568085; Lot no. L007041B), 10×Tris/Glycine/SDS buffer (Bio-rad, Cat. No. 1610732; Lot no. 10000044375B); 10×TBS (Bio-rad, Cat. No. 1706435; Lot no. 1000045140B), 10% Tween 20 (Cat. No. 1610781; Lot no. L004152B), Color protein standard broad range (NEB, Cat. No. P7719S; Lot no. 10040349), 4× Laemmli sample buffer (Bio-rad, Cat. No. 1610747; Lot no. L004133B), β-mercaptoethanol (Sigma-Aldrich, Cat. No. M3148; Lot no. 60-24-2), SuperBlock™ T20 (TBS) blocking buffer (ThermoScientific, Cat. No. 37536; Lot no. UC282578), 1 M sodium azide solution (Sigma-Aldrich, Cat. No. 08591-1 mL-F; Lot no. BCBV4989), α-Rabbit pAb to Ms IgG (abcam, Cat. No. ab97046; Lot no. GR3252115-1), α-Goat pAb to Rb IgG (CST, Cat. No. 7074S; Lot no. 28), α-GAPDH (abeam, Cat. No. ab8245; Lot no. GR3275542-2), α-PLK1 (CST, Cat. No. 208G4), α-BRD4 (CST, Cat. No. 13440S), ECL™ Prime western blotting reagents (GE Healthcare, Cat. No. RPN2232; Lot no. 17001655), Ponceau S solution (Sigma-Aldrich, Cat. No. P7170; Lot no. SLBV4112), Difco™ Skim milk (BD, Cat. No. 232100; Lot no. 8346795), and iBlot© 2 NC Regular stacks (Invitrogen, Cat. No. IB23001; Lot no. 2NR110619-02) were used.

For cell harvesting, the cells were first separated from the plate using trypsin and then washed with the medium and PBS. Specifically, the medium was suctioned off and washed with 1 mL of PBS, and PBS was suctioned off. The cells were treated with 0.5 mL TrypLE™ Express at 37° C. for 7 minutes to separate the cells, and then 0.5 mL of complete medium was added to collect 1 mL of cell culture solution. Then, 1 mL of the cell collection solution was centrifuged at 8,000 rpm for 120 seconds, and the supernatant was removed. After washing with 0.2 mL of PBS, the PBS was removed.

For cell lysis, a lysis buffer was added and cell debris was removed to obtain a cell lysate. Specifically, the cells were treated with 70 μL of 1×RIPA buffer containing a protease inhibitor and incubated for 30 minutes on ice. Then, the cells were centrifuged at 4° C. and 15,000 rpm for 10 minutes to obtain a cell lysate.

Then, a standard curve was obtained using the BCA assay, and the protein mass in the lysate was quantified by substituting the curve equation. The mixture was incubated at 37° C. for 30 minutes using 20 μL of standard or sample solution, and 200 μL of BCA or Bradford solution, and measured at 562 nm absorbance. Samples were prepared by adding 4× sample buffer so that the quantity of protein added to each well was 15 g.

Sodium dodecyl sulfate polyacrylamide gel electrophoresis (SDS-PAGE) was performed by setting a running time of 100 minutes at 120 V on a 4-15% Mini-PROTEAN TGX stain-free gel (15 well). Transferring was performed on iBlot© 2 NC Mini stacks at P0 mode of the dry blotting system. After staining using Ponceau S solution, blocking was performed for 1 hour with a blocking buffer (Thermo). After washing with 1×TBS containing 0.05% Tween 20, the product was reacted at 4° C. for 16 hours with anti-PLK1 (CST) antibody (1:500), anti-BRD4 (Cell signaling) antibody (1:1000) or anti-GAPDH (abeam) antibody (1:10,000) in 1×TBS-T as a primary antibody. After washing three times for 10 minutes with 1×TBS containing 0.05% Tween20, the product was reacted at room temperature for 1 hour with anti-mouse antibody (abcam) (1:10000) or anti-rabbit antibody (CST) (1:5000) in 1×TBS-T as a secondary antibody. Then, after washing three times for 10 minutes with 1×TBS containing 0.05% Tween 20, the product was detected with an ECL working solution (1:1).

To analyze the results, an image analyzer (GE) was used to obtain final blot data. As a result, it was confirmed that all of the compounds of the present invention degraded PLK1 protein significantly.

### 2. Luciferase Assay for PLK1

### (1) Preparation and Culture of HeLa LgBit (Plk1-HiBit KI) Cell Line

A HeLa cell line in which the LgBit vector was transfected and expressed stably was prepared. Then, after constructing gRNA and donor to express the HiBit amino acid sequence behind the C-terminal of the Plk1 gene, which was inherent in the cell, it was inserted into the cell together with a vector capable of expressing CRISPR/Cas9. Only the cells in which the insertion was completed and knock-in had progressed were selected, sub-cultured and used.

For cell culture, DMEM (Gibco, Cat. No. 11995-065; Lot. No. 2467189), FBS (Gibco, Cat. No. 16000-044; Lot. No. 2420173P), Penicillin/Streptomycin (PS)(Gibco, Cat. No. 15140-122; Lot. No. 2321114), 100 mm′ cell culture dish (SPL, Cat. No. 20100), 150 mm′ cell culture dish (SPL, Cat. No. 20150), 96-well culture plate (SPL, Cat. No. 30196), PBS pH 7.4 (Gibco, Cat. No. 10010-023; Lot. No. 2085080), TrypLE™ Express (Gibco, Cat. No. 12605-010; Lot. No. 2323417), Counting Chamber (Hematocytometer)(Marienfeld Superior, Cat. No. 0650010) and 0.4% Trypan Blue Solution (DYNEBIO, Cat. No. CBT3710; Lot. No. 20211201) were used.

### (2) Treatment of Compounds of the Present Invention and Method of Luciferase Assay

The compounds of Examples were completely dissolved in DMSO (Sigma-Aldrich Cat. No. D2438, Lot. No. RNBJ9566) and used in the experiment.

In the case of HeLa LgBit (Plk1-HiBit KI), the compounds were treated after being released after thymidine block, and the process was as follows. Thymidine (Sigma-Aldrich Cat. No. T9250-5G) was completely dissolved in DW and used in the experiment. For thymidine block, the products were treated with 2 mM of thymidine and then incubated for 24 hours. For release and chemical treatment, the medium was suctioned and washed with 1×PBS. TrypLE™ was added and incubated in 37° C. CO₂ incubator for 5 min. Cells neutralized by adding complete media were counted through a counter. For each well of a 96-well culture plate (SPL), 3.3×10⁴ cells and a total medium volume of 150 μL were seeded and incubated in a CO₂ incubator.

Each cell line was incubated in a CO₂ incubator for 18 hours, and Endurazine was added to each well to make up 4% of the total volume. After adding the compound of the present invention in a 96-well white plate (SPL) to a concentration of 300 nM, the wavelength of the plate reader (BMG Labtech, CLARIOstar Plus) was set to 470-480 nM, and then the luminescence was tracked in real time. After 9 hours, the luminescence value was obtained and displayed as a bar graph through an Excel program.

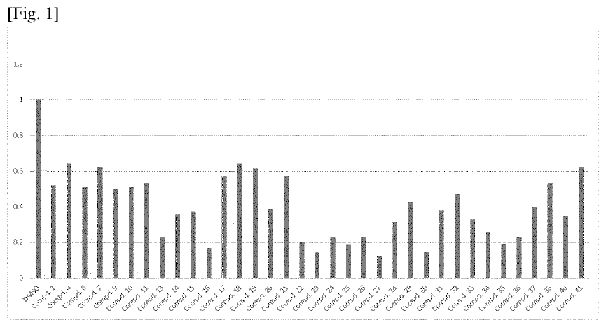

The results are shown in Table 2 below and FIG. 1.

TABLE 2

| Examplary Compound | Activity |
|---|---|
| Compound 1 | ++ |
| Compound 4 | + |
| Compound 6 | ++ |
| Compound 7 | + |
| Compound 9 | ++ |
| Compound 10 | ++ |
| Compound 11 | ++ |
| Compound 13 | +++ |
| Compound 14 | ++ |
| Compound 15 | ++ |
| Compound 16 | +++ |
| Compound 17 | ++ |
| Compound 18 | + |
| Compound 19 | + |
| Compound 20 | ++ |
| Compound 21 | ++ |
| Compound 22 | +++ |
| Compound 23 | +++ |
| Compound 24 | +++ |
| Compound 25 | +++ |
| Compound 26 | +++ |
| Compound 27 | +++ |
| Compound 28 | ++ |
| Compound 29 | ++ |
| Compound 30 | +++ |
| Compound 31 | ++ |
| Compound 32 | ++ |
| Compound 33 | ++ |
| Compound 34 | +++ |
| Compound 35 | +++ |
| Compound 36 | +++ |
| Compound 37 | ++ |
| Compound 38 | ++ |
| Compound 40 | ++ |
| Compound 41 | + |

In Table 2, Activity represents the ratio of the luminescence value of each Exemplary Compound treatment group to DMSO treatment group (+++: <0.3, ++<0.6, +<0.7).

### 3. Cell Viability Assay

### (1) Culture of NCI-H526 Cell Line

The NCI-H526 (hereafter H526) cell line was purchased from Korea Cell Line Bank (KCLB, Seoul, Korea). For cell culture, RPMI 1640 (Gibco, Cat. No. 22400-089; Lot. No. 2277021), FBS (Gibco, Cat. No. 16000-044; Lot. No. 2351176P), Penicillin/Streptomycin (PS)(Gibco, Cat. No. 15140-122; Lot. No. 2321114), 75T cell culture flask (SPL, Cat. No. 71075), 175T cell culture flask (SPL, Cat. No. 71175), 96-well cell culture plate (SPL, Cat. No. 30096), PBS pH 7.4 (Gibco, Cat. No. 10010-023; Lot. No. 2085080), TrypLE™ Express (Gibco, Cat. No. 12605-010; Lot. No. 2323417), Counting Chamber (Hematocytometer)(Marienfeld Superior, Cat. No. 0650010), and 0.4% Trypan Blue Solution (DYNEBIO, Cat. No. CBT3710; Lot. No. 20211201) were used.

### (2) Treatment of Compounds of the Present Invention and Method of Cell Viability Assay

The compounds of Examples were completely dissolved in DMSO (Sigma-Aldrich Cat. No. D2438, Lot. No. RNBJ9566) and used in the experiment.

3×10⁴ cells were seeded for each well of a 96-well plate (SPL), and the cells were cultured in total volume of 150 μL.

Each compound was diluted 3-folds from the highest concentration of 3000 nM to the lowest concentration of 0.46 nM. After treating the compound to each well to make the total volume of 200 μL, it was cultured in a CO₂ incubator (Thermo Fisher Science, Cat. No. 4111) for 5 days.

Then, after treating EZ-Cytox (DOGEN, Cat. NO. EZ-3000, Lot. No. DLS2109) 20 μL in each well, it was cultured in CO₂ incubator for 4 hours. The absorbance of the completely cultured sample was measured by setting the wavelength of a plate reader (BMG Labtech, CLARIOstar Plus) to 450 nM, and was measured after shaking for 3 minutes in a plate reader before measurement. The final measured value was arranged with Excel program, a graph was displayed through Prism-GraphPad program, and the IC₅₀ value was measured.

The results are shown in Table 3 below.

TABLE 3
Cell Viability Assay for H526 cell line

| Examplary Compound | Activity |
|---|---|
| Compound 1 | B |
| Compound 6 | B |
| Compound 9 | B |
| Compound 10 | A |
| Compound 11 | A |
| Compound 13 | A |
| Compound 14 | A |
| Compound 15 | B |
| Compound 16 | A |
| Compound 17 | B |
| Compound 18 | A |
| Compound 19 | B |
| Compound 20 | B |
| Compound 21 | C |
| Compound 22 | A |
| Compound 23 | A |
| Compound 24 | A |
| Compound 25 | A |
| Compound 26 | B |
| Compound 27 | A |
| Compound 28 | A |
| Compound 29 | B |
| Compound 30 | A |
| Compound 31 | C |
| Compound 32 | B |
| Compound 33 | B |
| Compound 34 | A |
| Compound 35 | B |
| Compound 36 | A |
| Compound 37 | B |
| Compound 38 | A |
| Compound 40 | A |

In Table 3, Activity represents IC₅₀ value of each Exemplary Compound treatment group to H526 cell line (A: <30 nM, B: <50 nM, C: <100 nM, D: <200 nM, E: <400 nM).

### 4. Cell Viability Assay for MRC-5 Cell Line

### (1) Culture of MRC-5 Cell Line

The MRC-5 cell line was purchased from Korea Cell Line Bank (KCLB), Seoul, Korea. Passage of cultured cells was maintained within P15.

For cell culture, MEM/EBSS (Hyclone, Cat. No. SH30024.01; Lot. No. AG29697698), FBS (Gibco, Cat. No. 16000-044; Lot. No. 2234018P), Penicillin/Streptomycin (PS)(Gibco, Cat. No. 15140-122; Lot. No. 2211099), 175T cell culture flask (SPL, Cat. No. 71175), 96-well cell culture plate (SPL, Cat. No. 30096), PBS pH 7.4 (Gibco, Cat. No. 10010-023; Lot. No. 2085080), TrypLE™ Express (Gibco, Cat. No. 12605-010; Lot. No. 2070638), Counting Chamber (Hematocytometer)(Hirschmann, Cat. No. 8100204), and 0.4% Trypan Blue Solution (DYNEBIO, Cat. No. CBT3710; Lot. No. 20190723) were used.

### (2) Treatment of Compounds of the Present Invention

MRC-5 cell line cultured in 175T cell culture flask was isolated using TrypLE™ Express. 6×10³ cells were seeded for each well of a 96-well plate (SPL), and the cells were cultured in total volume of 150 μL.

The compounds of Examples were completely dissolved in DMSO (Sigma-Aldrich, Cat. No. D2438-50ML, Lot. No. RNBK6387) and used in the experiment. Each compound was diluted 3-folds from the highest concentration of 10000 nM to the lowest concentration of 1.52 nM. Each well was mixed with a medium and treated, and the volume was set to 50 μL, so that the total volume of each well was 200 μL. Then, it was cultured in 37° C. CO₂ incubator (Thermo Fisher Science, Cat. No. 4111, Lot. No. 300512709) for 5 days.

The following compounds were used as comparative examples, and the cell viability assay was performed in the same manner as in the compounds of Examples.

### Comparative Example 1. Exemplary Compound Described in Mu et al. BBRC, 2020, 521(4): 833 (Comparative Compound 1)

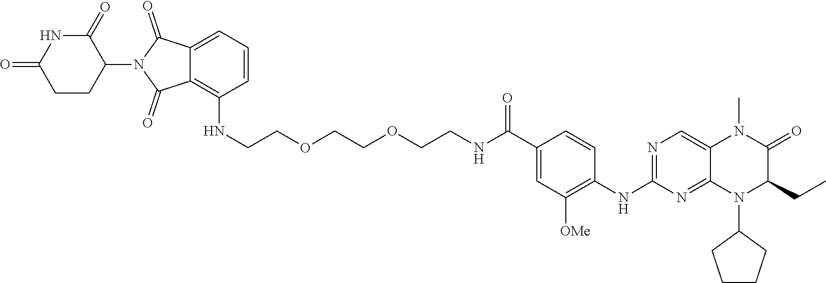 [SMILES: CC[C@@H]1C(=O)N(C)c2cnc(Nc3ccc(C(=O)NCCOCCOCCNc4cccc5c4C(=O)N(C4CCC(=O)NC4=O)C5=O)cc3OC)nc2N1C1CCCC1]

### Comparative Example 2. B12536 (Comparative Compound 2)

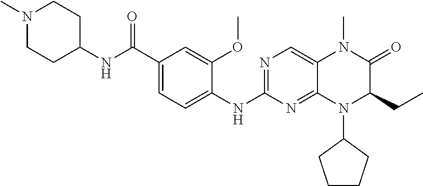 [SMILES: CC[C@@H]1C(=O)N(C)c2cnc(Nc3ccc(C(=O)NC4CCN(C)CC4)cc3OC)nc2N1C1CCCC1]

### Comparative Example 3. Volasertib (Comparative Compound 3)

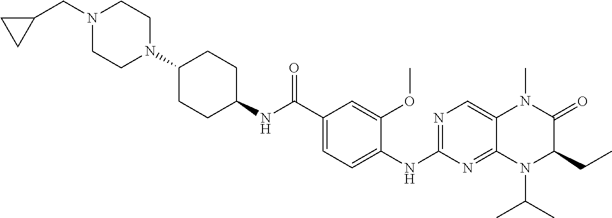 [SMILES: CC[C@@H]1C(=O)N(C)c2cnc(Nc3ccc(C(=O)N[C@H]4CC[C@H](N5CCN(CC6CC6)CC5)CC4)cc3OC)nc2N1C(C)C]

### Comparative Example 4. TAK960 (Comparative Compound 4)

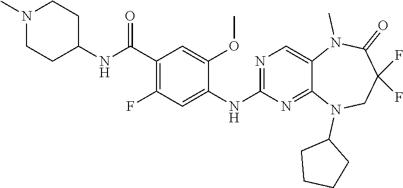 [SMILES: COc1cc(C(=O)NC2CCN(C)CC2)c(F)cc1Nc1ncc2c(n1)N(C1CCCC1)CC(F)(F)C(=O)N2C]

### (3) Cytotoxicity Experiment

After treating EZ-Cytox (DOGEN, Cat. NO. EZ-3000, Lot. No. DLS2112) 20 μL in each well of completely cultured plate, it was cultured in 37° C. CO₂ incubator for 4 hours. The 96-well plate was placed in a plate reader (BMG Labtech, Clariostar Plus), mixed for 2 minutes, and absorbance was measured at 450 nM wavelength. The data were converted into graphs using the Prism (ver. 9) program.

The results are shown in Table 4 and Table 5 below.

TABLE 4
Cell Viability Assay for MRC-5 cell line

| Examplary Compound | Activity |
|---|---|
| Compound 1 | N.D. |
| Compound 6 | N.D. |
| Compound 9 | N.D. |
| Compound 10 | N.D. |
| Compound 11 | N.D. |
| Compound 13 | N.D. |
| Compound 14 | N.D. |
| Compound 15 | N.D. |
| Compound 16 | N.D. |
| Compound 17 | N.D. |
| Compound 18 | N.D. |
| Compound 19 | N.D. |
| Compound 20 | N.D. |
| Compound 21 | N.D. |
| Compound 22 | N.D. |
| Compound 23 | N.D. |
| Compound 24 | 23219 |
| Compound 25 | 9241 |
| Compound 26 | 6553 |
| Compound 28 | 10213 |
| Compound 29 | 18491 |
| Compound 30 | 7869 |
| Compound 32 | N.D. |
| Compound 33 | 25034 |
| Compound 34 | N.D. |
| Compound 35 | 16877 |
| Compound 36 | 9802 |
| Compound 37 | N.D. |
| Compound 38 | 3963 |

In Table 4, Activity represents IC₅₀ value (nM) of each Exemplary Compound treatment group to MRC-5 cell line. N.D. (not determined) means that cytotoxicity did not appear until 10 μM. As a result, it was confirmed that all of the compounds of the present invention specifically exhibited a high level of cytotoxicity in cancer cell lines rather than normal cell lines.

TABLE 5
Cell Viability Assay for MRC-5 cell line

| Comparative Compound | Activity |
|---|---|
| Comparative Compound 1 | 106.6 |
| Comparative Compound 2 | 3085.4 |
| Comparative Compound 3 | 2939.3 |
| Comparative Compound 4 | 9152.5 |

In Table 5, Activity represents IC₅₀ value (nM) of each Comparative Compound treatment group to MRC-5 cell line. In particular, it was found that Comparative Compound 1, a known PROTAC compound, exhibited a high level of cytotoxicity in normal cell line, unlike the Exemplary Compounds of the present invention.

1. A compound represented by the following Formula I, a stereoisomer thereof or a pharmaceutically acceptable salt thereof:
ULM-Linker-PTM [Formula 1]
in the Formula I above,
ULM is a moiety represented by the following Formula 1; 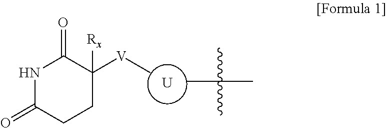
PTM is a moiety represented by the following Formula 2; 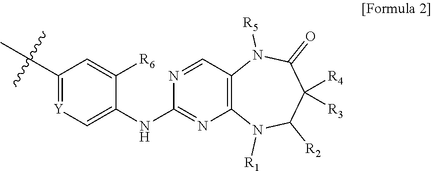
Linker is a group that chemically links ULM and PTM;
Rx is —H or —C₁₋₄alkyl;
V is —NH—C(═O)—, —(CH₂)v-NH—, —(CH₂)v-N—C₁₋₄alkyl-, —O—, —C(═O)— or —C(═NH)— {wherein the N atom of —(CH₂)v-NH— in the V may be linked with the Rx to form a 5- to 6-membered ring, and the v is 0, 1, 2, 3 or 4};
ring U is phenyl, pyridinyl or pyrimidinyl {wherein at least one H of the phenyl, pyridinyl or pyrimidinyl ring may be substituted with R_{U}};
R_{U} is —C₁₋₄alkyl, —C₁₋₄hydroxyalkyl, —C₁₋₄aminoalkyl, —C₁₋₄haloalkyl, —C₁₋₄alkoxy, —NH₂, —OH or -halo {wherein the R_{U} may be linked with the N atom of —(CH₂)v-NH— in the V to form 5- to 6-membered ring [wherein at least one H of the 5- to 6-membered ring may be substituted with —C₁₋₄alkyl or ═O], and the R_{U} may be linked with the N atom of —C(═NH)— in the V to form 5- to 6-membered ring [wherein at least one H of the 5- to 6-membered ring may be substituted with —C₁₋₄alkyl]};
Y is CR₇;
R₁ is —C₁₋₄alkyl or 3- to 7-membered cycloalkyl;
R₂ is —H;
R₃ and R₄ are each independently —H, —C₁₋₄alkyl or -halo;
R₅ is —C₁₋₄alkyl;
R₆ is —C₁₋₄alkyl or —C₁₋₄alkoxy; and
R₇ is —H or -halo.

2. The compound, the stereoisomer thereof or the pharmaceutically acceptable salt thereof according to claim 1,
ULM is a moiety represented by following Formula 1-1 or Formula 1-2; 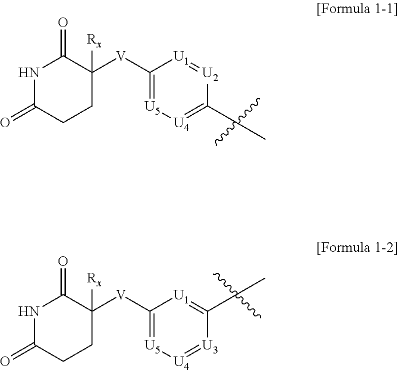
Rx is —H or —C₁₋₄alkyl;
V is —NH—C(═O)—, —(CH₂)v-NH—, —(CH₂)v-N—C₁₋₄alkyl-, —O— or —C(═NH)— {wherein the N atom of —(CH₂)v-NH— in the V may be linked with the Rx to form a 5- to 6-membered ring, and the v is 0, 1 or 2};
U₁ to U₅ are each independently CR_{U} or N {wherein R_{U} of the U₁ may be linked with the N atom of —(CH₂)v-NH— in the V to form a 5- to 6-membered ring [wherein at least one H of the 5- to 6-membered ring may be substituted with —C₁₋₄alkyl or ═O], and R_{U} of the U₁ may be linked with the N atom of —C(═NH)— in the V to form a 5- to 6-membered ring [wherein at least one H of the 5- to 6-membered ring may be substituted with —C₁₋₄alkyl]}; and
R_{U} is —C₁₋₄alkyl, —C₁₋₄haloalkyl, —NH₂ or -halo.

3. The compound, the stereoisomer thereof or the pharmaceutically acceptable salt thereof according to claim 1, 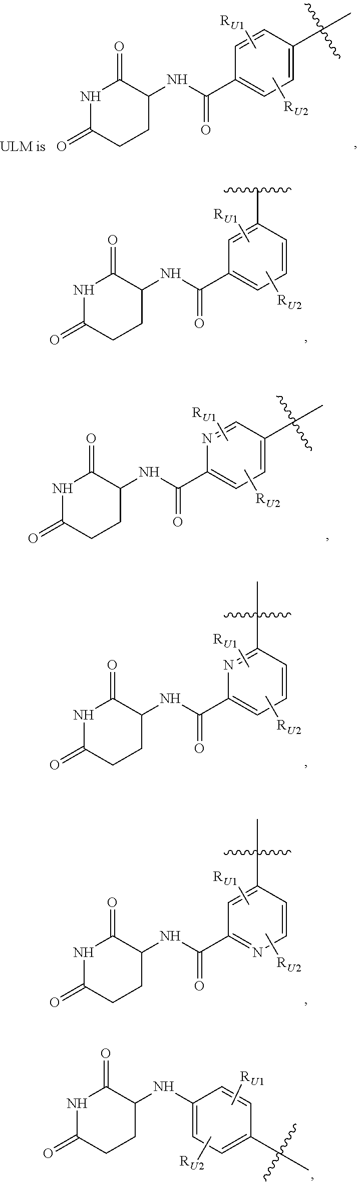 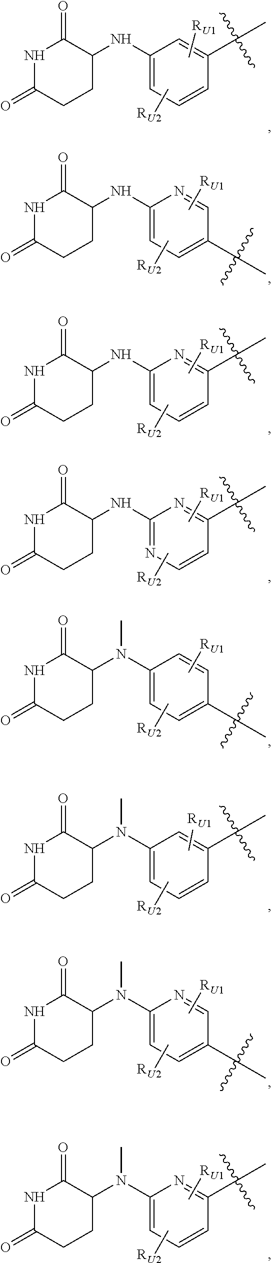 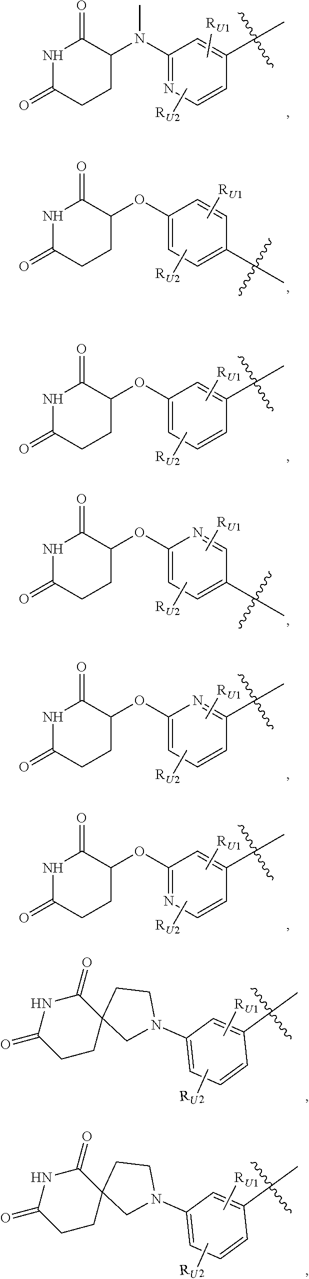 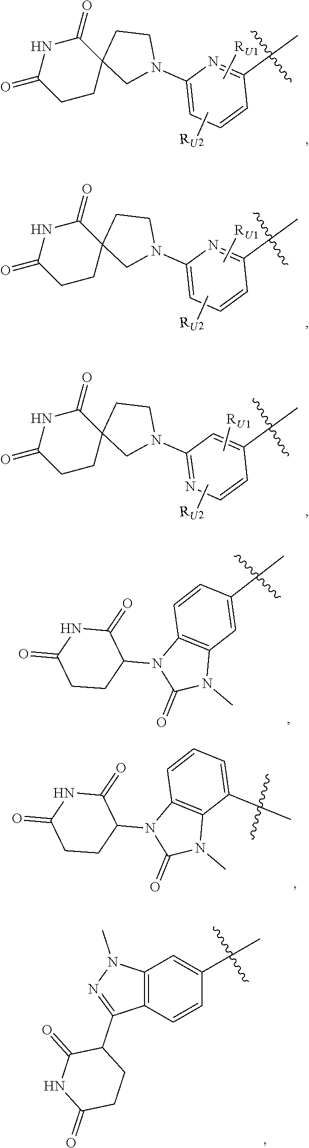 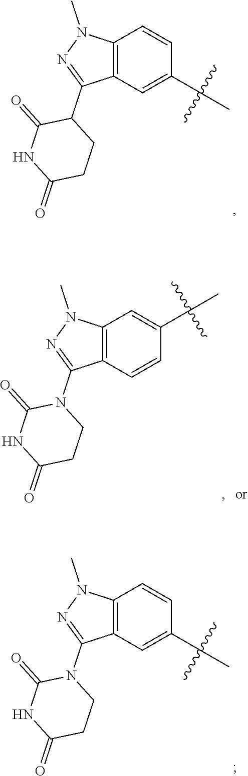 [SMILES: *c1ccc2c(c1)c(C1CCC(=O)NC1=O)nn2C ; *c1ccc2c(N3CCC(=O)NC3=O)nn(C)c2c1 ; *c1ccc2c(c1)c(N1CCC(=O)NC1=O)nn2C]
and
R_{U1} and R_{U2} are each independently —C₁₋₄alkyl, —C₁₋₄haloalkyl or -halo.

4. The compound, the stereoisomer thereof or the pharmaceutically acceptable salt thereof according to claim 3, 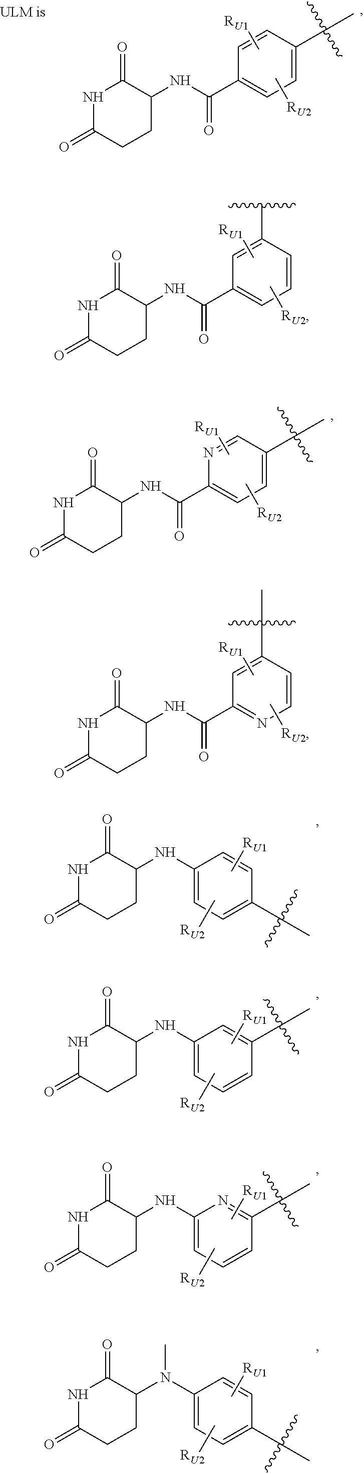 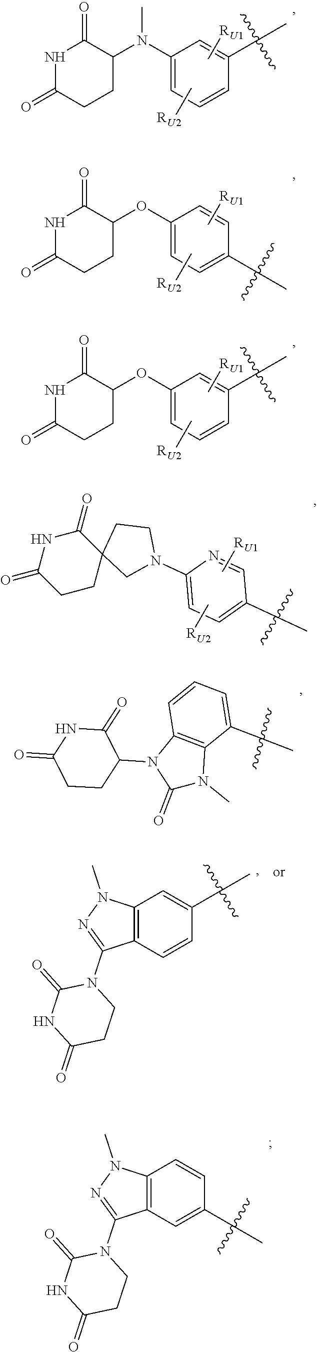
R_{U1} and R_{U2} are each independently —C₁₋₄haloalkyl or -halo.

5. The compound, the stereoisomer thereof or the pharmaceutically acceptable salt thereof according to claim 1,
PTM is 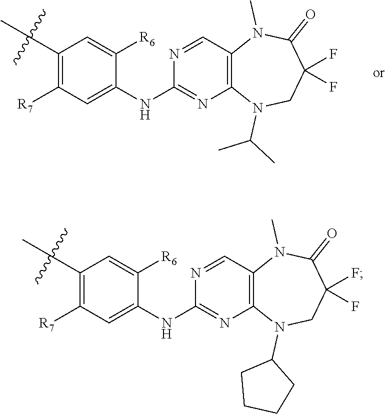
R₆ is —C₁₋₄alkoxy; and
R₇ is —H or -halo.

6. The compound, the stereoisomer thereof or the pharmaceutically acceptable salt thereof according to claim 1,
Linker is -L_{U}-L₁-L₂-L₃-L_{P}-;
L_{U} is —(CH₂)x-, —(CH₂)x-NH—, —(CH₂)x-O—, —C(═O)—, phenyl or nothing (null) {wherein L_{U} is linked with ULM [wherein, when the L_{U} is nothing (null), L₁ is directly linked with ULM], and the x is 0, 1, 2, 3 or 4};
L₁ is heterocycloalkyl or nothing (null) {wherein, when the L₁ is nothing (null), L_{U} and L₂ are directly linked, the heterocycloalkyl contains at least one N atom in the ring, and at least one H of the heterocycloalkyl ring may be substituted with —C₁₋₄alkyl, —C₁₋₄haloalkyl, —C₁₋₄alkoxy, —OH, -halo or ═O};
L₂ is —(CH₂)y₁-, -(CD₂)y₁-, —(CH₂)y₂-C(═O)—(CH₂)y₃-, —(CH₂)y₂-NH—(CH₂)y₃- or —(CH₂)y₂-N(C₁₋₄alkyl)-(CH₂)y₃-{wherein the y₁ to y₃ are each independently 0, 1, 2, 3, 4, 5 or 6};
L₃ is cycloalkyl, heterocycloalkyl or nothing (null) {wherein, when the L₃ is nothing (null), L₂ and Lₚ are directly linked, the heterocycloalkyl contains at least one N atom in the ring, and at least one H of the cycloalkyl or heterocycloalkyl ring may be substituted with —C₁₋₄alkyl, —C₁₋₄haloalkyl or -halo}; and
L_{P} is —(CH₂)p-NH—C(═O)— or —(CH₂)p-O—{wherein —(C═O)— or —O— of the L_{P} is linked with PTM, and
p is 0, 1 or 2}.

7. The compound, the stereoisomer thereof or the pharmaceutically acceptable salt thereof according to claim 6,
L_{U} is —(CH₂)x- or —(CH₂)x-NH—{wherein L_{U} is linked with ULM, and the x is 0 or 1};
L₁ is 4- to 12-membered heterocycloalkyl or nothing (null) {wherein, when the L₁ is nothing (null), L_{U} and L₂ are directly linked, the 4- to 12-membered heterocycloalkyl is single ring, bridged bicyclic ring or spiro ring, the 4- to 12-membered heterocycloalkyl contains at least one N atom in the ring, the N atom is directly linked with L_{U} or ULM, and at least one H of the 4- to 12-membered heterocycloalkyl ring may be substituted with —C₁₋₄alkyl, —OH or -halo};
L₂ is —(CH₂)y₁-, —(CH₂)y₂-C(═O)—(CH₂)y₃-, —(CH₂)y₂-NH—(CH₂)y₃- or —(CH₂)y₂-N(C₁₋₄alkyl)-(CH₂)y₃-{wherein the y₁ to y₃ are each independently 0, 1, 2 or 3};
L₃ is 4- to 6-membered cycloalkyl or 4- to 12-membered heterocycloalkyl {wherein the 4- to 12-membered heterocycloalkyl is single ring, bridged bicyclic ring or spiro ring, the 4- to 12-membered heterocycloalkyl contains at least one N atom in the ring, and at least one H of the 4- to 6-membered cycloalkyl or 4- to 12-membered heterocycloalkyl ring may be substituted with —C₁₋₄alkyl, —C₁₋₄haloalkyl or -halo}; and
L_{P} is —(CH₂)p-NH—C(═O)—{wherein —(C═O)— of the L_{P} is linked with PTM, and p is 0 or 1}.

8. The compound, the stereoisomer thereof or the pharmaceutically acceptable salt thereof according to claim 1, wherein the compound represented by the Formula I is selected from:

| Com- | |
|---|---|
| pound | Structure |
| 1 | 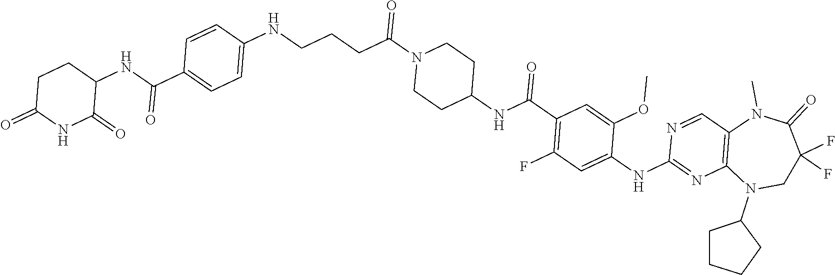 [SMILES: COc1cc(C(=O)NC2CCN(C(=O)CCCNc3ccc(C(=O)NC4CCC(=O)NC4=O)cc3)CC2)c(F)cc1Nc1ncc2c(n1)N(C1CCCC1)CC(F)(F)C(=O)N2C] |
| 2 | 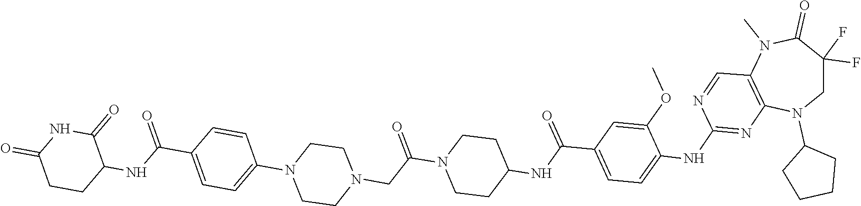 [SMILES: COc1cc(C(=O)NC2CCN(C(=O)CN3CCN(c4ccc(C(=O)NC5CCC(=O)NC5=O)cc4)CC3)CC2)ccc1Nc1ncc2c(n1)N(C1CCCC1)CC(F)(F)C(=O)N2C] |
| 3 | 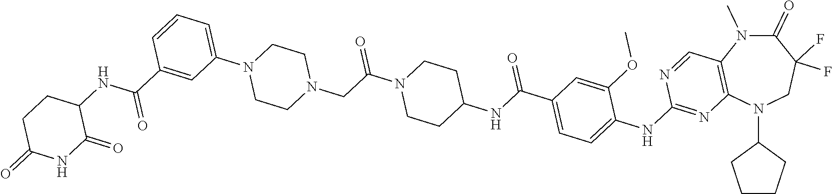 [SMILES: COc1cc(C(=O)NC2CCN(C(=O)CN3CCN(c4cccc(C(=O)NC5CCC(=O)NC5=O)c4)CC3)CC2)ccc1Nc1ncc2c(n1)N(C1CCCC1)CC(F)(F)C(=O)N2C] |
| 4 | 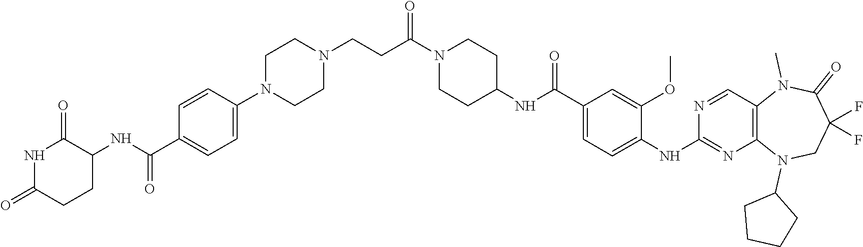 [SMILES: COc1cc(C(=O)NC2CCN(C(=O)CCN3CCN(c4ccc(C(=O)NC5CCC(=O)NC5=O)cc4)CC3)CC2)ccc1Nc1ncc2c(n1)N(C1CCCC1)CC(F)(F)C(=O)N2C] |
| 5 | 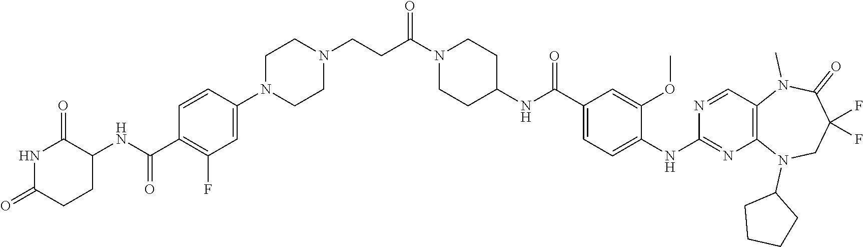 [SMILES: COc1cc(C(=O)NC2CCN(C(=O)CCN3CCN(c4ccc(C(=O)NC5CCC(=O)NC5=O)c(F)c4)CC3)CC2)ccc1Nc1ncc2c(n1)N(C1CCCC1)CC(F)(F)C(=O)N2C] |
| 6 | 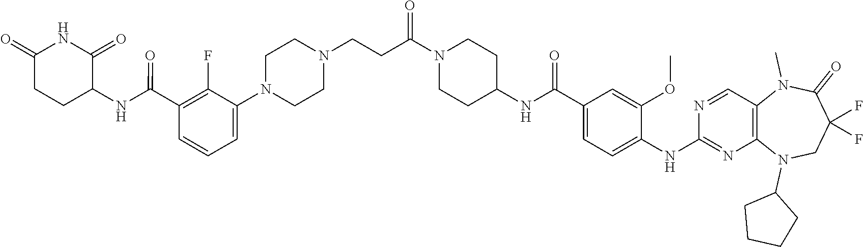 [SMILES: COc1cc(C(=O)NC2CCN(C(=O)CCN3CCN(c4cccc(C(=O)NC5CCC(=O)NC5=O)c4F)CC3)CC2)ccc1Nc1ncc2c(n1)N(C1CCCC1)CC(F)(F)C(=O)N2C] |
| 7 | 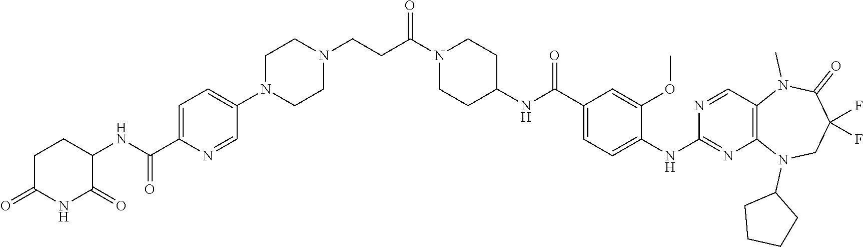 [SMILES: COc1cc(C(=O)NC2CCN(C(=O)CCN3CCN(c4ccc(C(=O)NC5CCC(=O)NC5=O)nc4)CC3)CC2)ccc1Nc1ncc2c(n1)N(C1CCCC1)CC(F)(F)C(=O)N2C] |
| 8 | 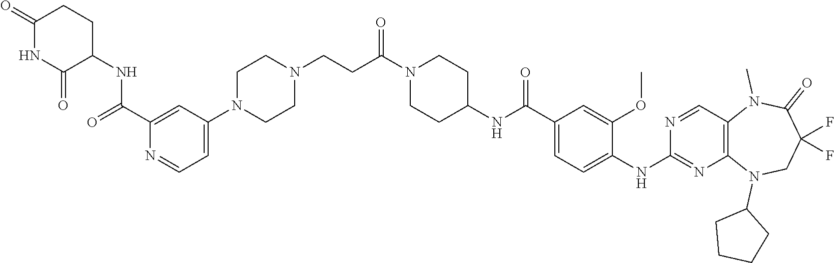 [SMILES: COc1cc(C(=O)NC2CCN(C(=O)CCN3CCN(c4ccnc(C(=O)NC5CCC(=O)NC5=O)c4)CC3)CC2)ccc1Nc1ncc2c(n1)N(C1CCCC1)CC(F)(F)C(=O)N2C] |
| 9 | 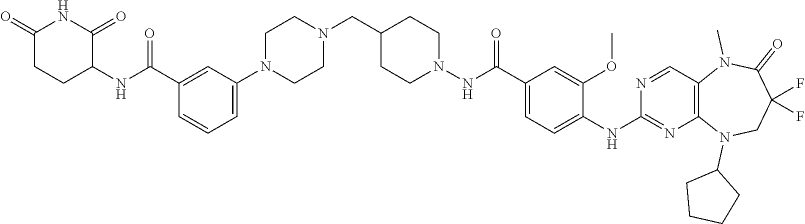 [SMILES: COc1cc(C(=O)NN2CCC(CN3CCN(c4cccc(C(=O)NC5CCC(=O)NC5=O)c4)CC3)CC2)ccc1Nc1ncc2c(n1)N(C1CCCC1)CC(F)(F)C(=O)N2C] |
| 10 | 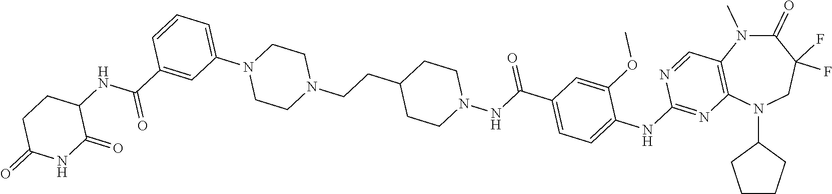 [SMILES: COc1cc(C(=O)NN2CCC(CCN3CCN(c4cccc(C(=O)NC5CCC(=O)NC5=O)c4)CC3)CC2)ccc1Nc1ncc2c(n1)N(C1CCCC1)CC(F)(F)C(=O)N2C] |
| 11 | 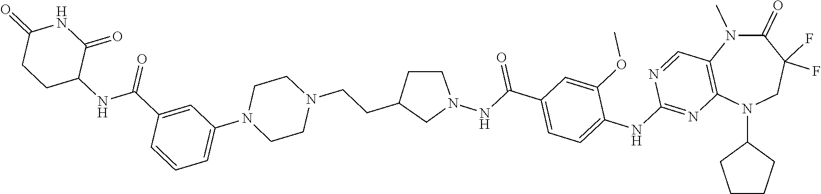 [SMILES: COc1cc(C(=O)NN2CCC(CCN3CCN(c4cccc(C(=O)NC5CCC(=O)NC5=O)c4)CC3)C2)ccc1Nc1ncc2c(n1)N(C1CCCC1)CC(F)(F)C(=O)N2C] |
| 12 | 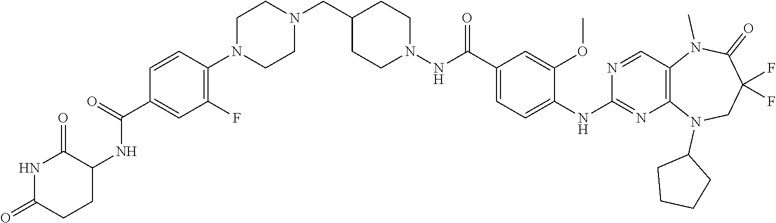 [SMILES: COc1cc(C(=O)NN2CCC(CN3CCN(c4ccc(C(=O)NC5CCC(=O)NC5=O)cc4F)CC3)CC2)ccc1Nc1ncc2c(n1)N(C1CCCC1)CC(F)(F)C(=O)N2C] |
| 13 | 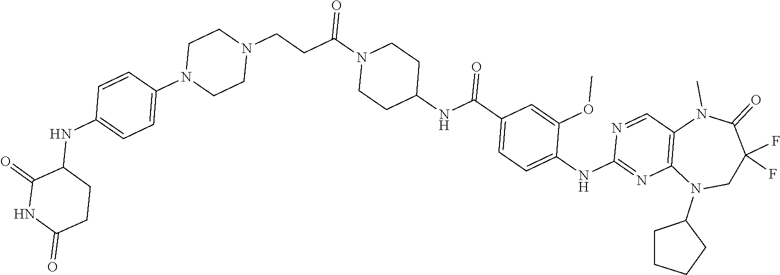 [SMILES: COc1cc(C(=O)NC2CCN(C(=O)CCN3CCN(c4ccc(NC5CCC(=O)NC5=O)cc4)CC3)CC2)ccc1Nc1ncc2c(n1)N(C1CCCC1)CC(F)(F)C(=O)N2C] |
| 14 | 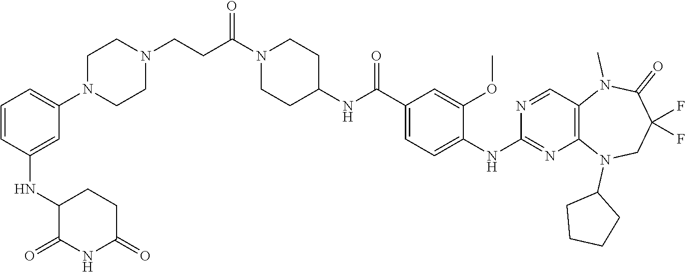 [SMILES: COc1cc(C(=O)NC2CCN(C(=O)CCN3CCN(c4cccc(NC5CCC(=O)NC5=O)c4)CC3)CC2)ccc1Nc1ncc2c(n1)N(C1CCCC1)CC(F)(F)C(=O)N2C] |
| 15 | 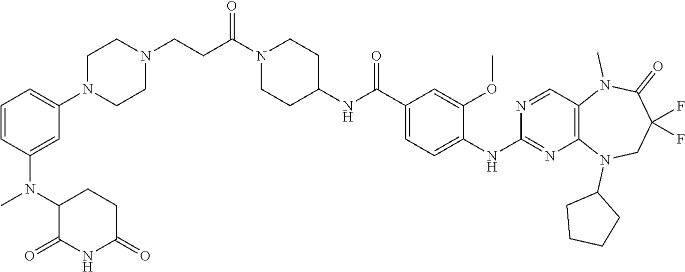 [SMILES: COc1cc(C(=O)NC2CCN(C(=O)CCN3CCN(c4cccc(N(C)C5CCC(=O)NC5=O)c4)CC3)CC2)ccc1Nc1ncc2c(n1)N(C1CCCC1)CC(F)(F)C(=O)N2C] |
| 16 | 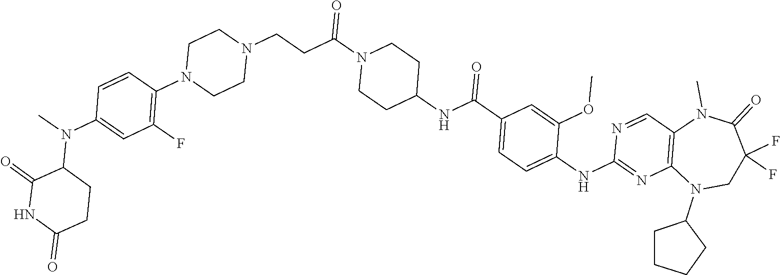 [SMILES: COc1cc(C(=O)NC2CCN(C(=O)CCN3CCN(c4ccc(N(C)C5CCC(=O)NC5=O)cc4F)CC3)CC2)ccc1Nc1ncc2c(n1)N(C1CCCC1)CC(F)(F)C(=O)N2C] |
| 17 | 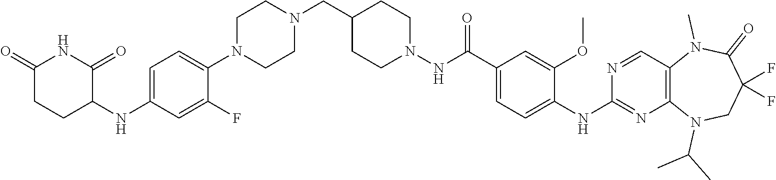 [SMILES: COc1cc(C(=O)NN2CCC(CN3CCN(c4ccc(NC5CCC(=O)NC5=O)cc4F)CC3)CC2)ccc1Nc1ncc2c(n1)N(C(C)C)CC(F)(F)C(=O)N2C] |
| 18 | 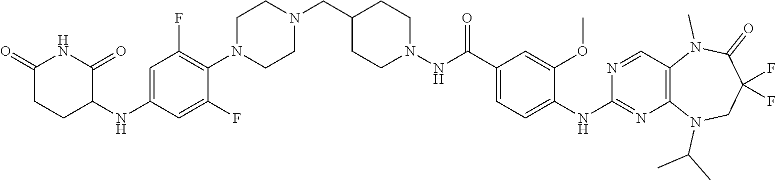 [SMILES: COc1cc(C(=O)NN2CCC(CN3CCN(c4c(F)cc(NC5CCC(=O)NC5=O)cc4F)CC3)CC2)ccc1Nc1ncc2c(n1)N(C(C)C)CC(F)(F)C(=O)N2C] |
| 19 | 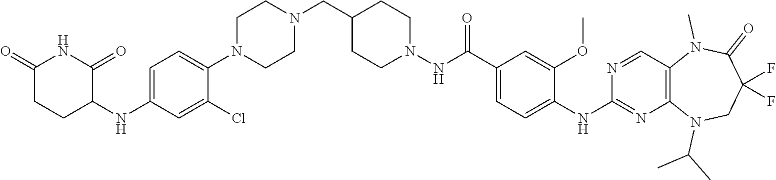 [SMILES: COc1cc(C(=O)NN2CCC(CN3CCN(c4ccc(NC5CCC(=O)NC5=O)cc4Cl)CC3)CC2)ccc1Nc1ncc2c(n1)N(C(C)C)CC(F)(F)C(=O)N2C] |
| 20 | 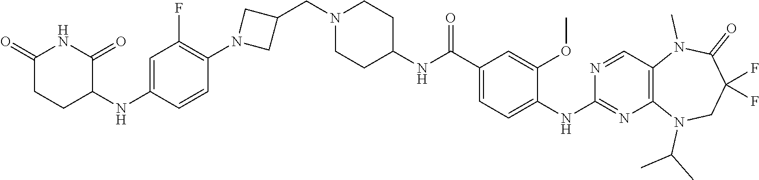 [SMILES: COc1cc(C(=O)NC2CCN(CC3CN(c4ccc(NC5CCC(=O)NC5=O)cc4F)C3)CC2)ccc1Nc1ncc2c(n1)N(C(C)C)CC(F)(F)C(=O)N2C] |
| 21 | 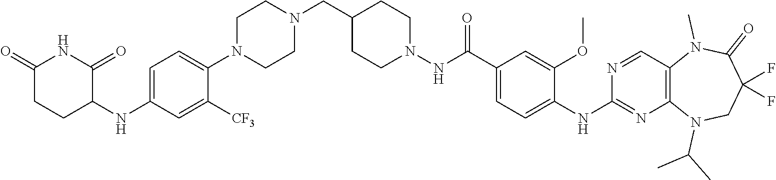 [SMILES: COc1cc(C(=O)NN2CCC(CN3CCN(c4ccc(NC5CCC(=O)NC5=O)cc4C(F)(F)F)CC3)CC2)ccc1Nc1ncc2c(n1)N(C(C)C)CC(F)(F)C(=O)N2C] |
| 22 | 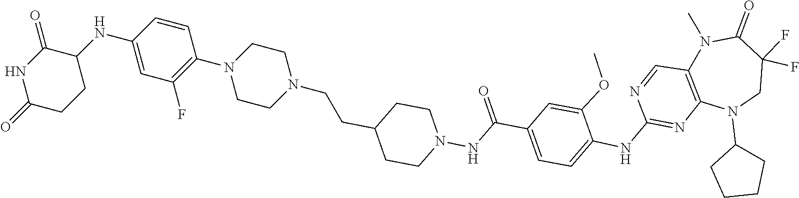 [SMILES: COc1cc(C(=O)NN2CCC(CCN3CCN(c4ccc(NC5CCC(=O)NC5=O)cc4F)CC3)CC2)ccc1Nc1ncc2c(n1)N(C1CCCC1)CC(F)(F)C(=O)N2C] |
| 23 | 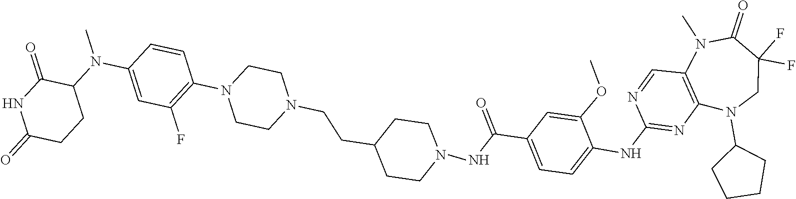 [SMILES: COc1cc(C(=O)NN2CCC(CCN3CCN(c4ccc(N(C)C5CCC(=O)NC5=O)cc4F)CC3)CC2)ccc1Nc1ncc2c(n1)N(C1CCCC1)CC(F)(F)C(=O)N2C] |
| 24 | 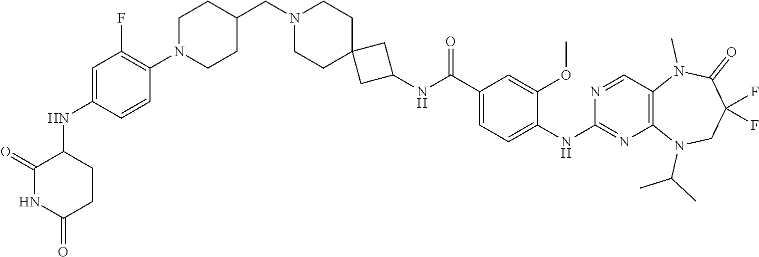 [SMILES: COc1cc(C(=O)NC2CC3(CCN(CC4CCN(c5ccc(NC6CCC(=O)NC6=O)cc5F)CC4)CC3)C2)ccc1Nc1ncc2c(n1)N(C(C)C)CC(F)(F)C(=O)N2C] |
| 25 | 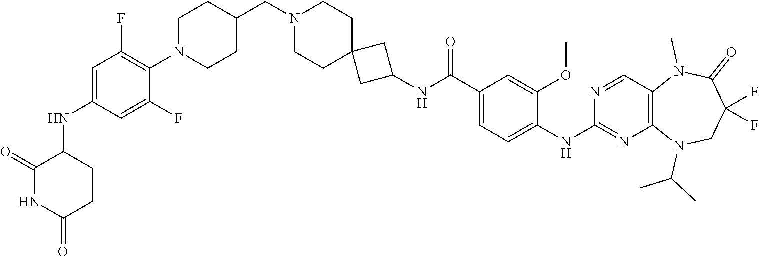 [SMILES: COc1cc(C(=O)NC2CC3(CCN(CC4CCN(c5c(F)cc(NC6CCC(=O)NC6=O)cc5F)CC4)CC3)C2)ccc1Nc1ncc2c(n1)N(C(C)C)CC(F)(F)C(=O)N2C] |
| 26 | 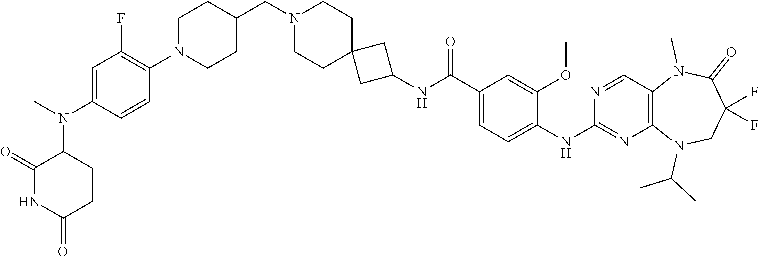 [SMILES: COc1cc(C(=O)NC2CC3(CCN(CC4CCN(c5ccc(N(C)C6CCC(=O)NC6=O)cc5F)CC4)CC3)C2)ccc1Nc1ncc2c(n1)N(C(C)C)CC(F)(F)C(=O)N2C] |
| 27 | 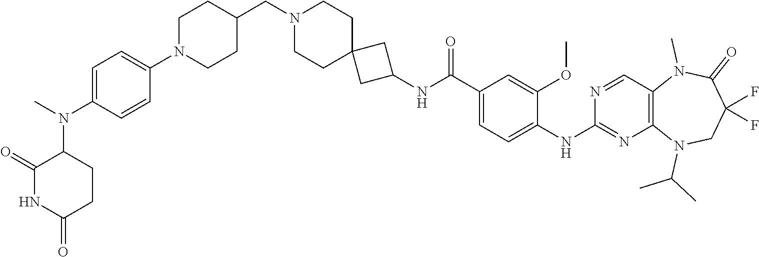 [SMILES: COc1cc(C(=O)NC2CC3(CCN(CC4CCN(c5ccc(N(C)C6CCC(=O)NC6=O)cc5)CC4)CC3)C2)ccc1Nc1ncc2c(n1)N(C(C)C)CC(F)(F)C(=O)N2C] |
| 28 | 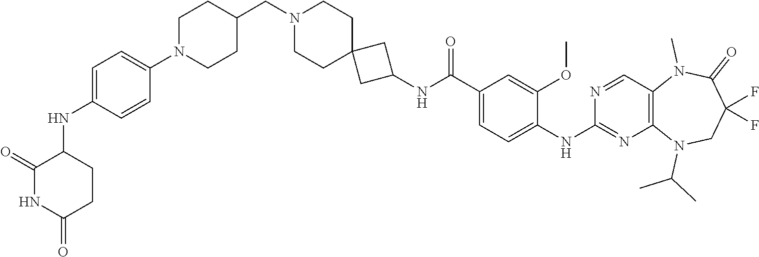 [SMILES: COc1cc(C(=O)NC2CC3(CCN(CC4CCN(c5ccc(NC6CCC(=O)NC6=O)cc5)CC4)CC3)C2)ccc1Nc1ncc2c(n1)N(C(C)C)CC(F)(F)C(=O)N2C] |
| 29 | 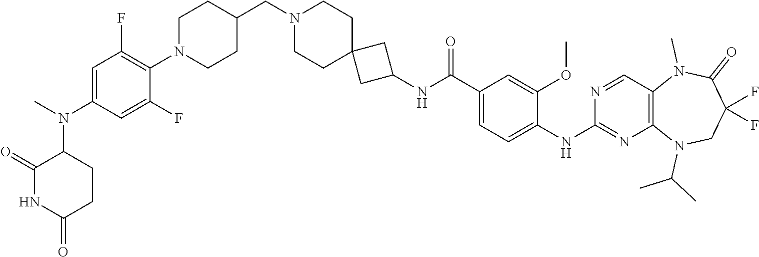 [SMILES: COc1cc(C(=O)NC2CC3(CCN(CC4CCN(c5c(F)cc(N(C)C6CCC(=O)NC6=O)cc5F)CC4)CC3)C2)ccc1Nc1ncc2c(n1)N(C(C)C)CC(F)(F)C(=O)N2C] |
| 30 | 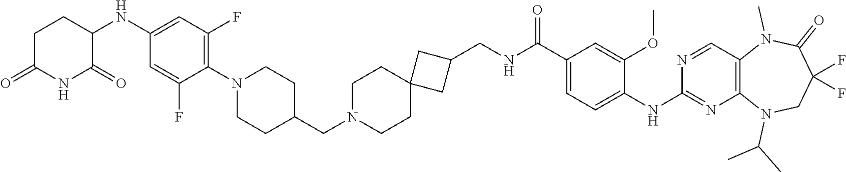 [SMILES: COc1cc(C(=O)NCC2CC3(CCN(CC4CCN(c5c(F)cc(NC6CCC(=O)NC6=O)cc5F)CC4)CC3)C2)ccc1Nc1ncc2c(n1)N(C(C)C)CC(F)(F)C(=O)N2C] |
| 31 | 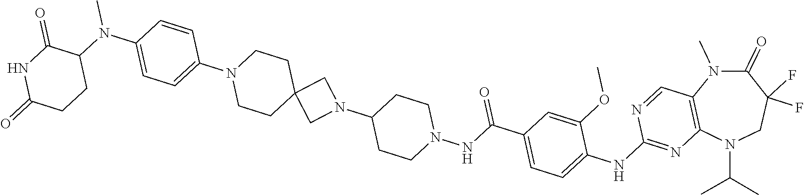 [SMILES: COc1cc(C(=O)NN2CCC(N3CC4(CCN(c5ccc(N(C)C6CCC(=O)NC6=O)cc5)CC4)C3)CC2)ccc1Nc1ncc2c(n1)N(C(C)C)CC(F)(F)C(=O)N2C] |
| 32 | 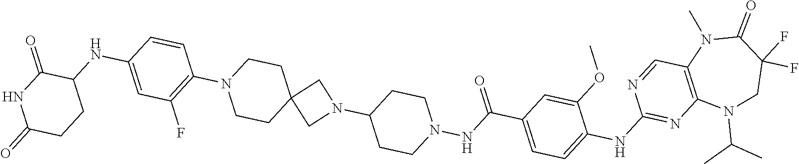 [SMILES: COc1cc(C(=O)NN2CCC(N3CC4(CCN(c5ccc(NC6CCC(=O)NC6=O)cc5F)CC4)C3)CC2)ccc1Nc1ncc2c(n1)N(C(C)C)CC(F)(F)C(=O)N2C] |
| 33 | 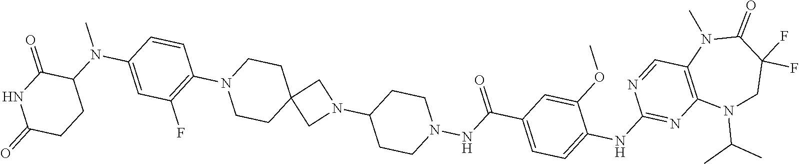 [SMILES: COc1cc(C(=O)NN2CCC(N3CC4(CCN(c5ccc(N(C)C6CCC(=O)NC6=O)cc5F)CC4)C3)CC2)ccc1Nc1ncc2c(n1)N(C(C)C)CC(F)(F)C(=O)N2C] |
| 34 | 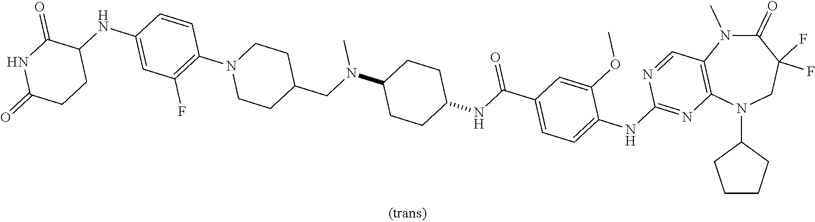 |
| 35 | 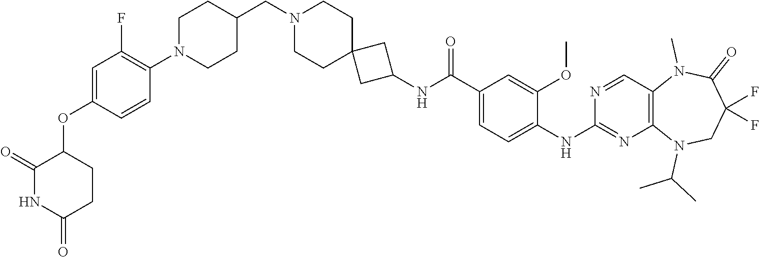 [SMILES: COc1cc(C(=O)NC2CC3(CCN(CC4CCN(c5ccc(OC6CCC(=O)NC6=O)cc5F)CC4)CC3)C2)ccc1Nc1ncc2c(n1)N(C(C)C)CC(F)(F)C(=O)N2C] |
| 36 | 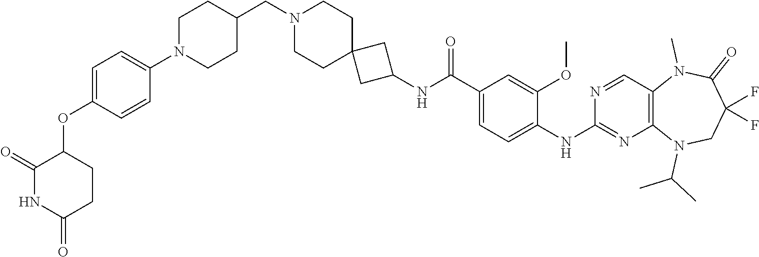 [SMILES: COc1cc(C(=O)NC2CC3(CCN(CC4CCN(c5ccc(OC6CCC(=O)NC6=O)cc5)CC4)CC3)C2)ccc1Nc1ncc2c(n1)N(C(C)C)CC(F)(F)C(=O)N2C] |
| 37 | 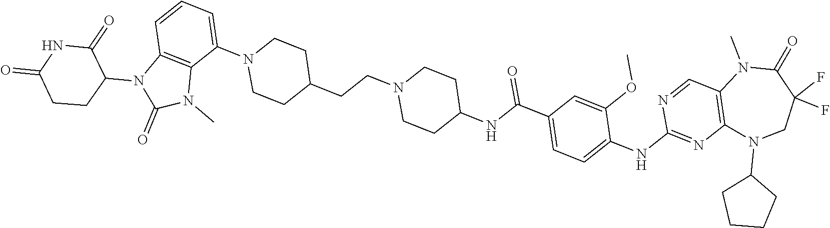 [SMILES: COc1cc(C(=O)NC2CCN(CCC3CCN(c4cccc5c4n(C)c(=O)n5C4CCC(=O)NC4=O)CC3)CC2)ccc1Nc1ncc2c(n1)N(C1CCCC1)CC(F)(F)C(=O)N2C] |
| 38 | 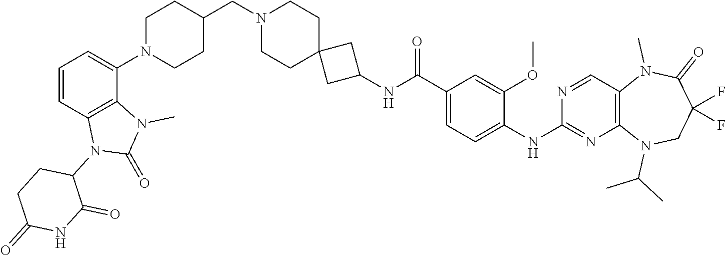 [SMILES: COc1cc(C(=O)NC2CC3(CCN(CC4CCN(c5cccc6c5n(C)c(=O)n6C5CCC(=O)NC5=O)CC4)CC3)C2)ccc1Nc1ncc2c(n1)N(C(C)C)CC(F)(F)C(=O)N2C] |
| 39 | 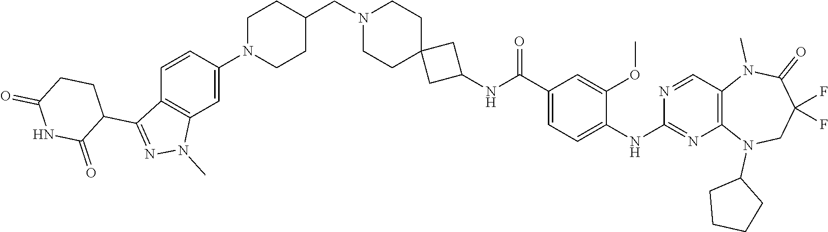 [SMILES: COc1cc(C(=O)NC2CC3(CCN(CC4CCN(c5ccc6c(C7CCC(=O)NC7=O)nn(C)c6c5)CC4)CC3)C2)ccc1Nc1ncc2c(n1)N(C1CCCC1)CC(F)(F)C(=O)N2C] |
| 40 | 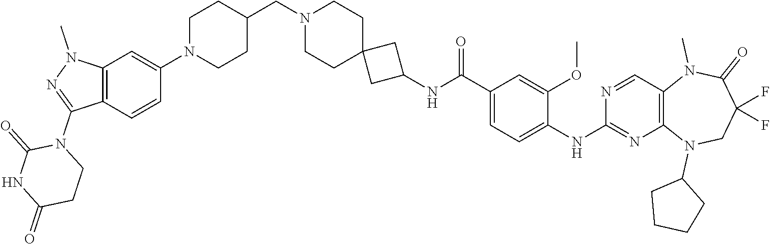 [SMILES: COc1cc(C(=O)NC2CC3(CCN(CC4CCN(c5ccc6c(N7CCC(=O)NC7=O)nn(C)c6c5)CC4)CC3)C2)ccc1Nc1ncc2c(n1)N(C1CCCC1)CC(F)(F)C(=O)N2C] |
| 41 | 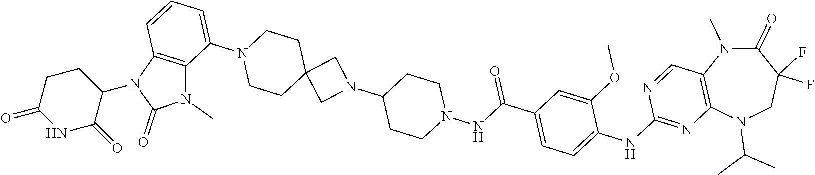 [SMILES: COc1cc(C(=O)NN2CCC(N3CC4(CCN(c5cccc6c5n(C)c(=O)n6C5CCC(=O)NC5=O)CC4)C3)CC2)ccc1Nc1ncc2c(n1)N(C(C)C)CC(F)(F)C(=O)N2C] |
| 42 | 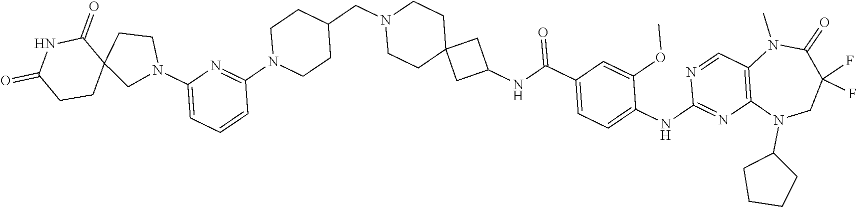 [SMILES: COc1cc(C(=O)NC2CC3(CCN(CC4CCN(c5cccc(N6CCC7(CCC(=O)NC7=O)C6)n5)CC4)CC3)C2)ccc1Nc1ncc2c(n1)N(C1CCCC1)CC(F)(F)C(=O)N2C] |
| 43 | 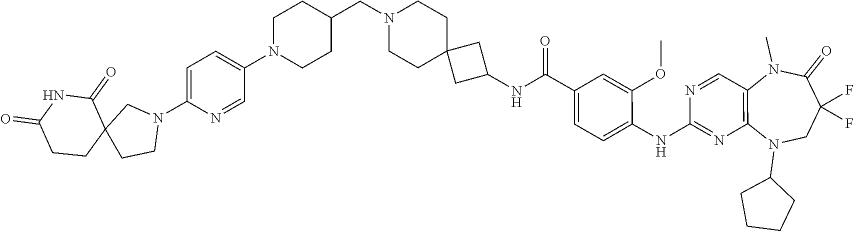 [SMILES: COc1cc(C(=O)NC2CC3(CCN(CC4CCN(c5ccc(N6CCC7(CCC(=O)NC7=O)C6)nc5)CC4)CC3)C2)ccc1Nc1ncc2c(n1)N(C1CCCC1)CC(F)(F)C(=O)N2C] |
| 44 | 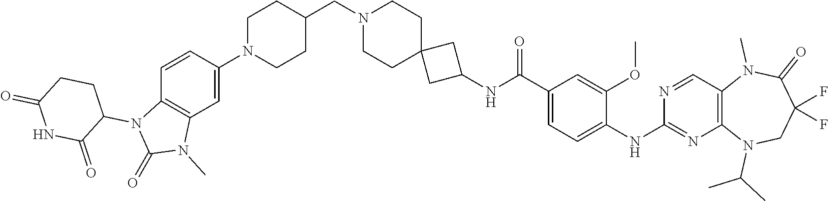 [SMILES: COc1cc(C(=O)NC2CC3(CCN(CC4CCN(c5ccc6c(c5)n(C)c(=O)n6C5CCC(=O)NC5=O)CC4)CC3)C2)ccc1Nc1ncc2c(n1)N(C(C)C)CC(F)(F)C(=O)N2C] |

9. A pharmaceutical composition comprising the compound, the stereoisomer thereof or the pharmaceutically acceptable salt thereof according to claim 1.

10. A pharmaceutical composition for preventing or treating PLK1-related disease comprising the compound, the stereoisomer thereof or the pharmaceutically acceptable salt thereof according to claim 1.

11. The pharmaceutical composition according to claim 10, wherein the PLK1-related disease is selected from a cancer, a benign tumor, or a neurological disorder.

12. The pharmaceutical composition according to claim 11, wherein the cancer or benign tumor is one or more selected from squamous cell carcinoma, small cell lung cancer, non-small cell lung cancer, lung adenocarcinoma, lung squamous cell carcinoma, peritoneal cancer, skin cancer, skin or intraocular melanoma, rectal cancer, anal muscle cancer, esophageal cancer, small intestine cancer, endocrine cancer, parathyroid cancer, adrenal cancer, soft tissue sarcoma, urethral cancer, chronic or acute leukemia, lymphocytic lymphoma, hepatocellular carcinoma, gastrointestinal cancer, gastric cancer, pancreatic cancer, glioblastoma, cervical cancer, ovarian cancer, liver cancer, bladder cancer, liver tumor, breast cancer, colon cancer, colorectal cancer, endometrial or uterine cancer, salivary gland cancer, kidney cancer, prostate cancer, vulvar cancer, thyroid cancer, head and neck cancer, brain cancer, osteosarcoma, Barrett's esophagus, colon adenoma and polyp, breast fibroadenoma and cyst, monoclonal gammopathy of undetermined significance (MGUS), monoclonal lymphocytosis, solid tumor, blood cancer, bone cancer, large cell lymphoma, adrenocorticoid tumor, t cell lymphoma/leukemia, neuroendocrine cancer, neuroendocrine tumor, cholangiocarcinoma, neuroblastoma, glioblastoma, and glioma.

13. The pharmaceutical composition according to claim 11, wherein the neurological disorder is selected from a central nervous system disease, a neurodegenerative disease, Alzheimer's disease, Parkinson's disease, multiple sclerosis, Huntington's disease, senile dementia, epilepsy, Lou Gehrig, stroke, or nerve damage and axonal degeneration-related disorders following brain or spinal cord injury.

14. A method for treating or preventing PLK1-related disease, comprising administering to the subject in need thereof a therapeutically effective amount of the compound, the stereoisomer thereof or the pharmaceutically acceptable salt thereof according to claim 1.

15. The method for treating or preventing PLK1-related disease according to claim 14, wherein the compound, the stereoisomer thereof or the pharmaceutically acceptable salt thereof induces degradation for PLK1 protein.

16-17. (canceled)
